(19) United States
(12) Patent Application Publication
(10) Pub. No.: US 2026/0035677 A1
(43) Pub. Date: Feb. 5, 2026

(54) TECHNOLOGIES FOR GENETIC MODIFICATION

(71) Applicant: Peter Biotherapeutics, Inc., Boston, MA (US)

(72) Inventors: Dirk Herman Antonius Hondmann, East Sandwich, MA (US); Chenzhong Kuang, Lexington, MA (US); Yan Xiao, Lexington, MA (US)

(21) Appl. No.: 18/840,358

(22) PCT Filed: Feb. 21, 2023
(86) PCT No.: PCT/US23/13517

§ 371 Date: Aug. 21, 2024

Related U.S. Application Data
(60) Provisional application No. 63/312,512, filed on Feb. 22, 2022.

(51) Int. Cl. C12N 9/14

(52) U.S. Cl. CPC C12N 9/14; C07K 2319/81; C12Y 306/04012

(57) ABSTRACT

The present disclosure provides technologies for genetic modification that use a helicase beta-wing element (HbW element). Provided technologies enable genetic modification without a need for introduction of one or more breaks into any genetic material being modified.

### CROSS-REFERENCE TO RELATED APPLICATIONS

The present application is a United States national stage application, filed under 35 U.S.C. § 371, of International Application No. PCT/US2023/013517, filed on Feb. 21, 2023, which claims the benefit and priority to U.S. Provisional patent application No. 63/312,512, filed on Feb. 22, 2022, the entire disclosures of both of which are incorporated herein by reference.

### SEQUENCE LISTING

The present application contains a Sequence Listing which has been submitted electronically in XML file format and is hereby incorporated by reference in its entirety. Said XML copy, created on Apr. 23, 2025, is named 2013051-0013.xml and is 226,082 bytes in size.

### BACKGROUND

Gene editing and genome engineering hold great promise for the study of gene function and for the creation of new therapies, e.g., to treat human disease. There is an ongoing need for compositions and methods to that can perform a wide variety of gene editing and editing in diverse cell types.

### SUMMARY

The present disclosure provides technologies (e.g., systems, compositions, methods, etc.) for modification of a polynucleotide (e.g., a DNA and/or RNA polynucleotide). The present disclosure provides novel technologies useful for sequence-specific modification of a polynucleotide. In some embodiments, the present disclosure provides novel technologies to achieve genetic modification without a need to introduce one or more breaks into a target site where a modification will occur. In particular, the present disclosure provides novel compositions for polynucleotide modification that comprise a polynucleotide modification agent (also referred to as a fusion molecule) that includes a helicase beta-wing element (HbW element) and a sequence-specific binding element.

The present disclosure demonstrates that provided technologies (e.g., polynucleotide modification agent and systems) are highly versatile. For example, in some embodiments, the present disclosure demonstrates that provided technologies are capable of genetically modifying diverse target polynucleotide sequences (e.g., different genes in a genome). In some embodiments, provided technologies are capable of introducing diverse types of polynucleotide modifications (e.g., insertions, deletions, substitutions, etc.) into a target site. In some embodiments, provided technologies are capable of genetically modifying a variety of different cell types. In some embodiments, provided technologies are capable of genetically modifying a cellular genome in vitro (e.g., in isolated and/or cultured cells) and/or in vivo (e.g., in an organism).

In some embodiments, provided technologies have one or more beneficial characteristics. For example, the present disclosure recognizes a limitation of previous technologies in that prior methods rely on polynucleotide replication to introduce a modification of a polynucleotide. In some embodiments, provided technologies are useful for genetic modification of non-replicating cells. In some embodiments, provided technologies are useful for genetic modification of primary, non-replicating human cells.

In some embodiments, the present disclosure recognizes that provided technologies are capable of editing a polynucleotide sequence with a high degree of accuracy (e.g., with low off target insertions and/or deletions).

The present disclosure further demonstrates that provided technologies, e.g., polynucleotide modification agent and systems are effective when administered via any of a variety of modalities. In some embodiments, provided technologies are capable of genetically modifying a cellular genome when delivered as a plasmid, mRNA and/or protein.

The present disclosure also encompasses an insight that gene modification technologies that include non-human components may be burdened by immunogenic responses in a subject. In some embodiments, the present disclosure provides polynucleotide modification agents (e.g., HbW fusion molecules) for genetic modification that are comprised of human sequences. In some embodiments, the present disclosure provides polynucleotide modification agents for genetic modification that include only human sequences.

In some embodiments, the present disclosure provides nucleic acids encoding a polynucleotide modification agent of the present disclosure. In some embodiments, the present disclosure provides isolated nucleic acids encoding a polynucleotide modification agent of the present disclosure. In some embodiments, the present disclosure provides one or more nucleic acid sequences encoding a polynucleotide modification agent of the present disclosure.

In some embodiments, the present disclosure provides vectors that include such nucleic acids. In some embodiments, the present disclosure provides vectors that include one or more nucleic acid sequences encoding a polynucleotide modification agent of the present disclosure.

In some embodiments, the present disclosure provides compositions that include a polynucleotide modification agent described herein. In some embodiments, the present disclosure provides compositions that include nucleic acids encoding polynucleotide modification agent described herein. In some embodiments, the present disclosure provides compositions that include vectors (e.g., viral vectors) comprising nucleic acids encoding polynucleotide modification agent described herein.

In some embodiments, provided compositions are pharmaceutical compositions that include (i) a polynucleotide modification agent or nucleic acid or vector encoding the same, and (ii) a pharmaceutically or physiologically acceptable carrier.

In some embodiments, the present disclosure provides combinations comprising (i) a polynucleotide modification agent or nucleic acid or vector encoding the same, and (ii) a sequence modification polynucleotide.

In some embodiments, the present disclosure provides kits that include a polynucleotide modification agent as described herein. In some embodiments, provided kits include a composition comprising a polynucleotide modification agent. In some embodiments, the present disclosure provides compositions that include nucleic acids encoding polynucleotide modification agent described herein. In some embodiments, the present disclosure provides compositions that include vectors comprising nucleic acids encoding polynucleotide modification agent described herein. In some embodiments, composition is a pharmaceutical composition comprising (i) a polynucleotide modification agent, nucleic acid encoding a polynucleotide modification agent, and/or a vector comprising such a nucleic acid, and (ii) a pharmaceutically or physiologically acceptable carrier. In some embodiments, provided kits further comprise a sequence modification polynucleotide. In some embodiments, provided kits include a first composition comprising a polynucleotide modification agent and a second composition comprising a sequence modification polynucleotide.

In some embodiments, the present disclosure provides genetic modification systems comprising (i) a polynucleotide modification agent or nucleic acid or vector encoding the same, and (ii) a sequence modification polynucleotide.

In some embodiments, the present disclosure provides methods of making and/or using a polynucleotide modification agent as described herein.

In some embodiments, provided are methods that include contacting a cell or population of cells with (i) a polynucleotide modification agent as described herein; and (ii) a sequence modification polynucleotide as described herein.

In some embodiments, provided are methods that include contacting DNA with (i) a polynucleotide modification agent as described herein; and (ii) a sequence modification polynucleotide as described herein.

In some embodiments, the present disclosure provides methods of characterizing a polynucleotide modification agent described herein, comprising measuring one or more of binding efficiency, binding affinity, sequence modification efficiency, and stability of at least one element of the polynucleotide modification agent.

In accordance with various embodiments, provided polymeric modification agents and compositions are useful in modifying one or more sequence elements in a polynucleotide. In some embodiments, the polynucleotide is or comprises DNA. In some embodiments, the polynucleotide is or comprises RNA (e.g., mRNA). In embodiments where the encoding nucleic acid is RNA (e.g., mRNA), the RNA may be 5′ capped and/or 3′ polyadenylated. In some embodiments, the modification is achieved via a system comprising one or more polymeric modification agents. In some embodiments, a system for genetic modification comprises a polynucleotide modification agent comprising one or more nucleotide binding elements and, optionally, a sequence modification polynucleotide comprising a nucleotide sequence used, in some way, to modify (e.g., via substitution, addition, deletion, etc.) one or more nucleotides at a target site.

In some embodiments, the present disclosure provides polynucleotide modification agents comprising a helicase beta-wing element (“HbW element”) and a sequence-specific binding element, wherein the HbW element is or comprises a helicase beta-wing.

In some embodiments, a HbW element comprises a helicase beta-wing domain that comprises an anti-parallel beta-sheet. In some embodiments, a HbW element can comprise a helicase beta-wing derived from a helicase from any source. For example, in some embodiments, a HbW element is or comprises a polypeptide derived from a prokaryotic helicase. In some embodiments, a HbW element is or comprises a polypeptide derived from a eukaryotic helicase. In some embodiments, a HbW element is or comprises a polypeptide derived from a human helicase.

In some embodiments, a HbW element of a polynucleotide modification agent described herein comprises a helicase beta-wing polypeptide with a mammalian sequence derived from a mammalian helicase polypeptide (e.g., a human helicase polypeptide). In some embodiments, a HbW element is or comprises a polypeptide derived from mammalian BLM helicase, mammalian WRN helicase, and/or mammalian RECQ1 helicase.

In some embodiments, a HbW element is or comprises a polypeptide with a human sequence. In some embodiments, a HbW element is or comprises a helicase beta-wing polypeptide with a human sequence derived from a human helicase polypeptide. In some embodiments, a HbW element is or comprises a helicase beta-wing polypeptide derived from human BLM helicase, human WRN helicase and/or human RECQ1.

In some embodiments, a HbW element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 1-3. In some embodiments, a HbW element is or comprises polypeptide sequence that is at least 90%, at least 95%, at least 96%, at least 97%, at least 98% at least 99%, or 100% identical to a sequence set forth in any one of SEQ ID NOs: 1-3. In some embodiments, a HbW element comprises a polypeptide sequence as set forth in any one of SEQ ID NOs: 1-3. In some embodiments, a HbW element consists of a polypeptide sequence as set forth in any one of SEQ ID NOs: 1-3.

In some embodiments, a sequence-specific binding element of a polynucleotide modification agent described herein comprises one or more Zinc Finger polypeptides; TALE-polypeptides; helix-loop-helix polypeptides; helix-turn-helix polypeptides; CAS-polypeptides; leucine zipper polypeptides; beta-scaffold polypeptides; homeo-domain polypeptides; high-mobility group box polypeptides, or a characteristic portion of any thereof and/or combination thereof.

In some embodiments, a sequence-specific binding element comprises a polypeptide with a human sequence.

In some embodiments, a sequence-specific binding element is or comprises a zinc finger array comprising polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NOs: 5 or 6. In some embodiments, a sequence-specific binding element is or comprises a zinc finger array comprising polypeptide sequence that is at least 90%, at least 95%, at least 96%, at least 97%, at least 98% at least 99%, or 100% identical to a sequence set forth in SEQ ID NOs: 5 or 6. In some embodiments, a sequence-specific binding element comprises a zinc finger array comprising a polypeptide sequence as set forth in SEQ ID NOs: 5 or 6. In some embodiments, a sequence-specific binding element comprises a zinc finger array that consists of a polypeptide sequence as set forth in SEQ ID NOs: 5 or 6.

In some embodiments, a sequence-specific binding element is or comprises a zinc finger array comprising polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 16-17 and 42-52. In some embodiments, a sequence-specific binding element is or comprises a zinc finger array comprising polypeptide sequence that is at least 90%, at least 95%, at least 96%, at least 97%, at least 98% at least 99%, or 100% identical to a sequence set forth in any one of SEQ ID NOs: 16-17 and 42-52. In some embodiments, a sequence-specific binding element comprises a zinc finger array comprising a polypeptide sequence as set forth in any one of SEQ ID NOs: 16-17 and 42-52. In some embodiments, a sequence-specific binding element comprises a zinc finger array that consists of a polypeptide sequence as set forth in any one of SEQ ID NOs: 16-17 and 42-52.

In some embodiments, a sequence-specific binding element comprises a zinc finger polypeptide comprising a zinc finger array. In some embodiments, a sequence-specific binding element comprises a zinc finger polypeptide comprising at least five zinc finger arrays. In some embodiments, a sequence-specific binding element is or comprises a zinc finger polypeptide comprising at least six, seven, eight, nine, ten, or eleven, or more, zinc finger arrays. In some embodiments, the zinc finger arrays comprise at least one alpha helix engineered to comprise a modified amino acid sequence that differs from that of its corresponding wild type sequence.

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NOs: 5 or 6. In some embodiments, a sequence-specific binding element further comprises a zinc finger linker sequence between zinc finger arrays comprising a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 14 or 15.

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 16-17 and 42-52. In some embodiments, a sequence-specific binding element further comprises a zinc finger linker sequence between zinc finger arrays comprising a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NO: 14-15 and 28-41.

In some embodiments, a sequence-specific binding element targets human ApoE, wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NO: 4, 23 and 25. In some embodiments, a sequence-specific binding element targets human ApoE, wherein the sequence specific binding element is or comprises polypeptide sequence that is at least 90%, at least 95%, at least 96%, at least 97%, at least 98% at least 99%, or 100% identical to a sequence set forth in any one of SEQ ID NOs: 4, 23 and 25. In some embodiments, a sequence-specific binding element targets human ApoE, wherein the sequence specific binding element comprises a polypeptide sequence as set forth in any one of SEQ ID NOs: 4, 23 and 25. In some embodiments, a sequence-specific binding element targets human ApoE, wherein the sequence specific binding element consists of a polypeptide sequence as set forth in any one of SEQ ID NOs: 4, 23 and 25.

In some embodiments, a sequence-specific binding element targets EGFPDP2, wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NO: 19 and 21. In some embodiments, a sequence-specific binding element targets EGFPDP2, wherein the sequence specific binding element is or comprises polypeptide sequence that is at least 90%, at least 95%, at least 96%, at least 97%, at least 98% at least 99%, or 100% identical to a sequence set forth in any one of SEQ ID NOs: 19 and 21. In some embodiments, a sequence-specific binding element targets EGFPDP2, wherein the sequence specific binding element comprises a polypeptide sequence as set forth in any one of SEQ ID NOs: 19 and 21. In some embodiments, a sequence-specific binding element targets EGFPDP2, wherein the sequence specific binding element consists of a polypeptide sequence as set forth in any one of SEQ ID NOs: 19 and 21.

In some embodiments, a sequence-specific binding element targets BAF chromatin remodeling complex subunit BCL11A (BCL11A), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 70. In some embodiments, a sequence-specific binding element targets BCL11A, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 70. In some embodiments, a sequence-specific binding element targets BCL11A, wherein the sequence specific binding element comprises a polypeptide sequence as set forth in any one of SEQ ID NO: 70. In some embodiments, a sequence-specific binding element targets BCL11A, wherein the sequence specific binding element consists of a polypeptide sequence as set forth in any one of SEQ ID NO: 70.

In some embodiments, a sequence-specific binding element targets DNA polymerase gamma (PolG), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 78. In some embodiments, a sequence-specific binding element targets PolG, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 78. In some embodiments, a sequence-specific binding element targets PolG, wherein the sequence specific binding element comprises a polypeptide sequence as set forth in any one of SEQ ID NO: 78. In some embodiments, a sequence-specific binding element targets PolG, wherein the sequence specific binding element consists of a polypeptide sequence as set forth in any one of SEQ ID NO: 78.

In some embodiments, a sequence-specific binding element targets metabolism of cobalamin associated C (MMACHC), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 86. In some embodiments, a sequence-specific binding element targets MMACHC, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 86. In some embodiments, a sequence-specific binding element targets MMACHC, wherein the sequence specific binding element comprises a polypeptide sequence as set forth in any one of SEQ ID NO: 86. In some embodiments, a sequence-specific binding element targets MMACHC, wherein the sequence specific binding element consists of a polypeptide sequence as set forth in any one of SEQ ID NO: 86.

In some embodiments, a sequence-specific binding element targets methylmalonyl-CoA mutase (MMUT), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 94. In some embodiments, a sequence-specific binding element targets MMUT, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 94. In some embodiments, a sequence-specific binding element targets MMUT, wherein the sequence specific binding element comprises a polypeptide sequence as set forth in any one of SEQ ID NO: 94. In some embodiments, a sequence-specific binding element targets MMUT, wherein the sequence specific binding element consists of a polypeptide sequence as set forth in any one of SEQ ID NO: 94.

In some embodiments, a sequence-specific binding element targets phenylalanine hydroxylase (PAH), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 102. In some embodiments, a sequence-specific binding element targets PAH, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 102. In some embodiments, a sequence-specific binding element targets PAH, wherein the sequence specific binding element comprises a polypeptide sequence as set forth in any one of SEQ ID NO: 102. In some embodiments, a sequence-specific binding element targets PAH, wherein the sequence specific binding element consists of a polypeptide sequence as set forth in any one of SEQ ID NO: 102.

In some embodiments, a sequence-specific binding element targets CF transmembrane conductance regulator (CFTR), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 110. In some embodiments, a sequence-specific binding element targets CFTR, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 110. In some embodiments, a sequence-specific binding element targets CFTR, wherein the sequence specific binding element comprises a polypeptide sequence as set forth in any one of SEQ ID NO: 110. In some embodiments, a sequence-specific binding element targets CFTR, wherein the sequence specific binding element consists of a polypeptide sequence as set forth in any one of SEQ ID NO: 110.

In some embodiments, a sequence-specific binding element targets dystrophin (DMD), wherein the sequence specific binding element is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth SEQ ID NO: 122. In some embodiments, a sequence-specific binding element targets DMD, wherein the sequence specific binding element is or comprises polypeptide sequence that is at least 90%, at least 95%, at least 96%, at least 97%, at least 98% at least 99%, or 100% identical to a sequence set forth in any one of SEQ ID NO: 122. In some embodiments, a sequence-specific binding element targets DMD, wherein the sequence specific binding element comprises a polypeptide sequence as set forth in any one of SEQ ID NO: 122. In some embodiments, a sequence-specific binding element targets DMD, wherein the sequence specific binding element consists of a polypeptide sequence as set forth in any one of SEQ ID NO: 122.

In some embodiments, a polynucleotide modification agent further comprises a linker. In some embodiments, a linker is or comprises a polypeptide. In some embodiments, a linker is or comprises a polypeptide with a human sequence.

In some embodiments, a linker is or comprises a polypeptide between 2 and 100 amino acids in length. In some embodiments, a linker comprises a polypeptide that is at least 2, at least 3, at least 4, at least 5, at least 6, at least 7, at least 8, at least 9, at least 10, at least 15, at least 20, at least 25, at least 30, at least 35, at least 40, at least 45, or at least 50 amino acids in length. In some embodiments, a linker comprises a polypeptide that is 4 to 80 amino acids in length. In some embodiments, a linker comprises a polypeptide that is 5 to 70 amino acids in length. In some embodiments, a linker comprises a polypeptide that is 5 to 60 amino acids in length. In some embodiments, a linker comprises a polypeptide that is 5 to 50 amino acids in length. In some embodiments, a linker comprises a polypeptide that is 10 to 100 amino acids in length. In some embodiments, a linker comprises a polypeptide that is 10 to 50 amino acids in length.

In some embodiments, a linker is or comprises a polypeptide between 0.2 kD and 10 kD in size. In some embodiments, a linker comprises a polypeptide that is at least 0.2 kD, at least 0.3 kD, at least 0.4 kD, at least 0.5 kD, at least 0.6 kD, at least 0.7 kD, at least 0.8 kD, at least 0.9 kD, at least 1 kD, at least 1.5 kD, at least 2 kD, at least 2.5 kD, at least 3 kD, at least 3.5 kD, at least 4 kD, at least 4.5 kD, or at least 5 kD in size. In some embodiments, a linker comprises a polypeptide that is 0.4 kD to 8 kD in size. In some embodiments, a linker comprises a polypeptide that is 0.5 kD to 7 kD in size. In some embodiments, a linker comprises a polypeptide that is 0.5 kD to 6 kD in size. In some embodiments, a linker comprises a polypeptide that is 0.5 kD to 5 kD in size. In some embodiments, a linker comprises a polypeptide that is 1 kD to 10 kD in size. In some embodiments, a linker comprises a polypeptide that is 1 kD to 5 kD in size.

In some embodiments, a linker is or comprises a polypeptide sequence that is derived from a helicase polypeptide. In some embodiments, a linker is or comprises a polypeptide sequence that is derived from a human helicase polypeptide. In some embodiments, a linker is or comprises a polypeptide sequence derived from a human helicase polypeptide selected from a WRN helicase, a BLM helicase, and a REQ1 helicase.

In some embodiments, a linker is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 7-9. In some embodiments, a linker is or comprises polypeptide sequence that is at least 90%, at least 95%, at least 96%, at least 97%, at least 98% at least 99%, or 100% identical to a sequence set forth in any one of SEQ ID NOs: 7-9. In some embodiments, a linker comprises a polypeptide sequence as set forth in any one of SEQ ID NOs: 7-9. In some embodiments, a linker consists of a polypeptide sequence as set forth in any one of SEQ ID NOs: 7-9.

In some embodiments, a linker is or comprises an amino acid sequence of SEQ ID NO. 26. In some embodiments, a linker is or comprises a glycine-serine linker. In some embodiments, a linker is or comprises a sequence of SEQ ID NO: 27. In some embodiments, a linker is or comprises a glycine-serine linker comprising 2, 3, 4, 5, 6, 7, 8, 9 or more repeats (e.g., of SEQ ID NO: 27).

In some certain embodiments, a polynucleotide modification agent is or comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 55, 58, 61, 67, 75, 83, 91, 99, 107, and 118. In some embodiments, a polynucleotide modification agent is or comprises polypeptide sequence that is at least 90%, at least 95%, at least 96%, at least 97%, at least 98% at least 99%, or 100% identical to a sequence set forth in any one of SEQ ID NOs: 55, 58, and 61. In some embodiments, a polynucleotide modification agent comprises a polypeptide sequence as set forth in any one of SEQ ID NOs: 55, 58, 61, 67, 75, 83, 91, 99, 107, and 118. In some embodiments, a polynucleotide modification agent consists of a polypeptide sequence as set forth in any one of SEQ ID NOs: 55, 58, 61, 67, 75, 83, 91, 99, 107, and 118.

In some certain embodiments, a polynucleotide modification agent is encoded by a cDNA sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 54, 57, 60, 66, 74, 82, 90, 98, 106, and 117. In some embodiments, a polynucleotide modification agent is encoded by a cDNA sequence that is at least 90%, at least 95%, at least 96%, at least 97%, at least 98% at least 99%, or 100% identical to a sequence set forth in any one of SEQ ID NOs: 54, 57, 60, 66, 74, 82, 90, 98, 106, and 117. In some embodiments, a polynucleotide modification agent is encoded by a cDNA sequence as set forth in any one of SEQ ID NOs: 54, 57, 60, 66, 74, 82, 90, 98, 106, and 117.

In some embodiments, provided polynucleotide modification agents do not comprise a linker.

In some embodiments, provided polynucleotide modification agents lack nuclease function.

In some embodiments, a HbW element of provided polynucleotide modification agents interacts with a target site. In some embodiments, a sequence-specific binding element of provided polynucleotide modification agents binds to a landing site. In some embodiments, a landing site is adjacent to a target site.

In some embodiments, a sequence-specific binding element of a polynucleotide modification agent binds to the landing site with a binding affinity characterized by a dissociation constant of 10 E−6 M or lower. In some embodiments, a sequence-specific binding element of a polynucleotide modification agent binds to the landing site with a binding affinity characterized by a dissociation constant of 10 E−7 M, 10 E−8 M, 10 E−9 M, 10 E−10 M, or lower.

In some embodiments, a sequence-specific binding element of a polynucleotide modification agent binds to a single strand of polynucleotide.

In some embodiments, a HbW element of a polynucleotide modification agent breaks one or more hydrogen bonds within a target site of a polynucleotide. In some embodiments, a HbW element inserts between strands of a polynucleotide. In some embodiments, a HbW element or any other portion of a polynucleotide modification agent does not catalyze single and/or double-stranded DNA breaks.

In some embodiments, a polynucleotide modification agent provided herein includes two or more helicase beta-wing elements (“HbW elements”). In some embodiments, a polynucleotide modification agent includes two or more HbW elements and at least one linker. In some embodiments, a polynucleotide modification agent includes two or more HbW elements and at least two linkers. In some embodiments, a polynucleotide modification agent includes a sequence specific binding element, a first linker, a first HbW element, a second linker, and a second HbW element.

In some embodiments, a polynucleotide modification agent includes, in order from N terminus to C-terminus: a sequence specific binding element, a first linker, a first HbW element, a second linker, and a second HbW element. In some embodiments, a polynucleotide modification agent includes, in order from N terminus to C-terminus: a first HbW element, a first linker, a second HbW element, a second linker, and a sequence specific binding element. In some embodiments, a polynucleotide modification agent includes, in order from N terminus to C-terminus: a first HbW element, a first linker, a sequence specific binding element, a second linker, and a second HbW element.

In some embodiments, a polynucleotide modification agent of the present disclosure does not cause modification of a non-target site.

In accordance with various embodiments, provided herein are sequence modification polynucleotides. In some embodiments, a sequence modification polynucleotide: (i) binds specifically to a target sequence in a population of cells of the subject; and (ii) has a sequence difference relative to the target sequence.

In some embodiments, a sequence modification polynucleotide (i) binds specifically to one strand of the DNA at a target site; and (ii) has a mismatch or other DNA sequence difference relative to the target site, so that usage of the sequence modification polynucleotide incorporates the sequence modification into a complement of the one strand. In some embodiments, the incorporation of the sequence modification into the complement of the one strand occurs simultaneously or after the HbW element interacts with the DNA.

In some embodiments, a sequence modification polynucleotide comprises a sequence that is capable of being incorporated into a copy of a human gene selected from: human ApoE, human BCL11A, and human DMD. In some embodiments, the incorporating occurs during DNA replication or DNA synthesis.

For example, in some embodiments, a sequence modification polynucleotide is capable of modifying human ApoE and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 10 or 11. In some embodiments, a sequence modification polynucleotide is capable of modifying human ApoE and comprises sequence that is at least 90%, at least 95%, at least 96%, at least 97%, at least 98% at least 99%, or 100% identical to a sequence set forth in SEQ ID NO: 10 or 11. In some embodiments, a sequence modification polynucleotide is capable of modifying human ApoE and comprises a sequence as set forth in SEQ ID NO: 10 or 11. In some embodiments, a sequence modification polynucleotide is capable of modifying human ApoE and consists of a sequence as set forth in SEQ ID NO: 10 or 11.

In some embodiments, a sequence modification polynucleotide is capable of modifying human BCL11A and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 12. In some embodiments, a sequence modification polynucleotide is capable of modifying human BCL11A and comprises a sequence that is at least 90%, at least 95%, at least 96%, at least 97%, at least 98% at least 99%, or 100% identical to a sequence set forth in SEQ ID NO: 12. In some embodiments, a sequence modification polynucleotide is capable of modifying human BCL11A and comprises a sequence as set forth in SEQ ID NO: 12. In some embodiments, a sequence modification polynucleotide is capable of modifying human BCL11A and consists of a sequence as set forth in SEQ ID NO: 12.

In some embodiments, a sequence modification polynucleotide is capable of modifying human DMD and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 13. In some embodiments, a sequence modification polynucleotide is capable of modifying human DMD and comprises a sequence that is at least 90%, at least 95%, at least 96%, at least 97%, at least 98% at least 99%, or 100% identical to a sequence set forth in SEQ ID NO: 13. In some embodiments, a sequence modification polynucleotide is capable of modifying human DMD and comprises a sequence as set forth in SEQ ID NO: 13. In some embodiments, a sequence modification polynucleotide is capable of modifying human DMD and consists of a sequence as set forth in SEQ ID NO: 13.

In some embodiments, a sequence modification polynucleotide is capable of modifying human PolG and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 72. In some embodiments, a sequence modification polynucleotide is capable of modifying human PolG and comprises a sequence that is at least 90%, at least 95%, at least 96%, at least 97%, at least 98% at least 99%, or 100% identical to a sequence set forth in SEQ ID NO: 72. In some embodiments, a sequence modification polynucleotide is capable of modifying human PolG and comprises a sequence as set forth in SEQ ID NO: 72. In some embodiments, a sequence modification polynucleotide is capable of modifying human PolG and consists of a sequence as set forth in SEQ ID NO: 72.

In some embodiments, a sequence modification polynucleotide is capable of modifying human MMACHC and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 80. In some embodiments, a sequence modification polynucleotide is capable of modifying human MMACHC and comprises a sequence that is at least 90%, at least 95%, at least 96%, at least 97%, at least 98% at least 99%, or 100% identical to a sequence set forth in SEQ ID NO: 80. In some embodiments, a sequence modification polynucleotide is capable of modifying human MMACHC and comprises a sequence as set forth in SEQ ID NO: 80. In some embodiments, a sequence modification polynucleotide is capable of modifying human MMACHC and consists of a sequence as set forth in SEQ ID NO: 80.

In some embodiments, a sequence modification polynucleotide is capable of modifying human MMUT and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 88. In some embodiments, a sequence modification polynucleotide is capable of modifying human MMUT and comprises a sequence that is at least 90%, at least 95%, at least 96%, at least 97%, at least 98% at least 99%, or 100% identical to a sequence set forth in SEQ ID NO: 88. In some embodiments, a sequence modification polynucleotide is capable of modifying human MMUT and comprises a sequence as set forth in SEQ ID NO: 88. In some embodiments, a sequence modification polynucleotide is capable of modifying human MMUT and consists of a sequence as set forth in SEQ ID NO: 88.

In some embodiments, a sequence modification polynucleotide is capable of modifying human PAH and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 96. In some embodiments, a sequence modification polynucleotide is capable of modifying human PAH and comprises a sequence that is at least 90%, at least 95%, at least 96%, at least 97%, at least 98% at least 99%, or 100% identical to a sequence set forth in SEQ ID NO: 96. In some embodiments, a sequence modification polynucleotide is capable of modifying human PAH and comprises a sequence as set forth in SEQ ID NO: 96. In some embodiments, a sequence modification polynucleotide is capable of modifying human PAH and consists of a sequence as set forth in SEQ ID NO: 96.

In some embodiments, a sequence modification polynucleotide is capable of modifying human CFTR and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 104. In some embodiments, a sequence modification polynucleotide is capable of modifying human CFTR and comprises a sequence that is at least 90%, at least 95%, at least 96%, at least 97%, at least 98% at least 99%, or 100% identical to a sequence set forth in SEQ ID NO: 104. In some embodiments, a sequence modification polynucleotide is capable of modifying human CFTR and comprises a sequence as set forth in SEQ ID NO: 104. In some embodiments, a sequence modification polynucleotide is capable of modifying human CFTR and consists of a sequence as set forth in SEQ ID NO: 104.

In accordance with various embodiments, provided composition, combinations, genetic modification systems, and/or kits further comprise at least one additional agent. In accordance with various embodiments, provided methods further comprise contacting a cell, population of cells and/or polynucleotide with at least one additional agent.

In some embodiments, at least one additional agent is or comprises an agent that (i) induces DNA replication and/or (ii) induces DNA strand repair. In some embodiments, at least one additional agent is one that (i) induces DNA replication and/or (ii) induces DNA repair. In some embodiments, provided methods further comprise contacting a cell, population of cells and/or polynucleotide with a DNA modification system that includes one or more of a DNA polymerase, helicase, ligase, recombinase, repair scaffold protein, single strand DNA binding protein, and/or mismatch repair protein.

In some embodiments, at least one additional agent is or comprises enhancing agent and/or an inhibiting agent. In some embodiments, an enhancing and/or inhibiting agent alters DNA recombination events. In some embodiments, an enhancing agent and/or inhibiting agent itself does not contact the DNA. In some embodiments, an enhancing agent and/or inhibiting agent is or comprises RNAi activity. In some embodiments, incorporation of a sequence modification occurs at a greater frequency with enhancing agent and/or inhibiting agent relative to an otherwise identical method that does not include the enhancing agent or inhibiting agent. In some embodiments, incorporation of a sequence modification occurs at least 2 fold, 3 fold, 4 fold, 5 fold, 6 fold, 7 fold, 8 fold, 9 fold, or 10 fold greater frequency with enhancing agent and/or inhibiting agent relative to an otherwise identical method that does not include the enhancing agent or inhibiting agent.

In some embodiments, a contacted cell, population of cells and/or polynucleotide comprise a DNA polynucleotide comprising at least one target site. In some embodiments, a contacted cell, population of cells and/or polynucleotide comprise a DNA polynucleotide comprising a landing site.

In some embodiments, a HbW element of provided polynucleotide modification agents interacts with a target site. In some embodiments, a sequence-specific binding element of provided polynucleotide modification agents binds to a landing site. In some embodiments, a landing site is adjacent to a target site. In some embodiments, a sequence-specific binding element of a polynucleotide modification agent binds to the landing site with a binding affinity characterized by a dissociation constant of 10 E−6 or lower. In some embodiments, a sequence-specific binding element of a polynucleotide modification agent binds to the landing site with a binding affinity characterized by a dissociation constant of 10 E−7 or lower. In some embodiments, a sequence-specific binding element of a polynucleotide modification agent binds to a single strand of polynucleotide. In some embodiments, a HbW element of a polynucleotide modification agent breaks one or more hydrogen bonds within a target site of a polynucleotide. In some embodiments, a HbW element inserts between strands of a polynucleotide. In some embodiments, a polynucleotide modification agent for use in provided methods does not itself catalyze single and/or double-stranded DNA breaks.

In some embodiments, a contacted cell or population of cells are non-replicating and/or post-mitotic.

In some embodiments, a contacted cell or population of cells comprise DNA that is actively replicating.

In some embodiments, contacting is performed ex vivo. In some embodiments, contacting is performed in vitro. In some embodiments, ex vivo or in vitro contacting is of a population of cells and produces a population of cells with comprising at least one modified DNA sequence relative to the population of cells prior to the contacting. In some embodiments, at least a portion of the population of cells is formulated to be administered to a subject in need thereof. In some embodiments, at least a portion of the population of cells is formulated as a composition for administration to a subject. In some embodiments, at least a portion of the population of cells is administered to a subject in need thereof.

In some embodiments, contacting is performed in vivo.

In some embodiments, provided are methods that include administering to a subject (i) a polynucleotide modification agent described herein, and (ii) a sequence modification polynucleotide.

In some embodiments, provided methods that include administering a polynucleotide modification agent to a subject are capable of inducing a change in the target sequence of a population of cells of the subject, wherein the change in the target sequence corresponds to the sequence of the sequence modification polynucleotide. In some embodiments, a population of cells is or comprises: (i) a tissue, (ii) an organ, (iii) a tumor, and/or (iv) a cell-specific cell lineage. In some embodiments, a population of cells is or comprises a cell-specific cell lineage that is or comprises (i) neural cells and/or (ii) neuronal cells.

In some embodiments, a subject is mammal. In some embodiments, a subject is a non-human primate or a human. In some embodiments, a subject is a fetal, infant, child, adolescent, or adult human.

In some embodiments, provided methods modify at least one target sequence. In some embodiments, provided methods modify two or more target sequences. In some embodiments, two or more target sequences modified by methods described herein are associated with different genes. In some embodiments, the different genes are located on the same chromosome. In some embodiments, the different genes are located on different chromosomes.

In some embodiments, two or more target sequences modified by methods described herein are associated with the same gene.

Throughout the description, where systems or compositions are described as having, including, or comprising specific components, or where methods are described as having, including, or comprising specific steps, it is contemplated that, additionally, there are systems or compositions of the present invention that consist essentially of, or consist of, the recited components, and that there are methods according to the present invention that consist essentially of, or consist of, the recited steps.

It should be understood that the order of steps or order for performing certain action is immaterial so long as the invention remains operable. Moreover, two or more steps or actions may be conducted simultaneously, unless specified otherwise.

These and other features and advantages provided in the present disclosure will be more fully understood from the following detailed description taken together with the accompanying claims.

### BRIEF DESCRIPTION OF THE DRAWING

FIG. 1 is a schematic of representative complex that includes a DNA helicase during a replication event.

FIG. 2 is a schematic of a polynucleotide modification agent that includes a sequence specific binding element, a linker, and a helicase beta wing (HbW) element.

FIG. 3 is an exemplary schematic of a polynucleotide modification agent, with a sequence specific binding element comprising zinc finger domains (e.g., with at least four zinc finger domains), a linker, and a HbW element.

FIG. 4 illustrates certain steps as they may occur via genetic conversion mediated by an exemplary polynucleotide modification agent described herein. Panel A shows a polynucleotide modification agent before binding at a specific target site in a genome. Panel B shows a polynucleotide modification agent binding at a specific target site in a genome and DNA strand separating. Panel C shows a donor template that has a desired DNA modification annealing to its complementary DNA strand. Panel D shows creation of a mismatch mutation, which can integrate into a genome. Panel E shows an integrated DNA modification introduced by steps including those shown in Panels A-D.

FIG. 5 shows multiple amino acid sequence alignments of exemplary HbW elements based on fragments of human helicases (e.g., WRN based, BLM, and RECQ1).

FIG. 6. illustrates targeting and editing at codon 112 of human endogenous ApoE, as well as ddPCR detection of T→C conversion in B-cells

FIG. 7 demonstrates T→C genetic conversion at codon 112 of human ApoE by ddPCR analysis of dots representing droplets, containing indicated C (upper panel) or T alleles (lower panel).

FIG. 8 shows successful T→C conversion in B-cell pools by Sanger sequencing.

FIG. 9 shows Single Nucleotide Polymorphisms (SNP) analysis by next generation sequencing of edited B-cells using pb106 (WRN-based construct). Panel A shows overviews of SNPs at each position of the targeting region of codon 112 site of human ApoE. Panel B shows an enlarged, trimmed view in the region adjacent to codon 112 site of human ApoE.

FIG. 10 shows Single Nucleotide Polymorphisms (SNP) analysis by next generation sequencing of edited B-cells using pb110 (BLM-based construct). Panel A shows overviews of SNPs at each position of the targeting region of codon 112 site of human ApoE. Panel B shows an enlarged, trimmed view in the region adjacent to codon 112 site of human ApoE.

FIG. 11 shows Single Nucleotide Polymorphisms (SNP) analysis by next generation sequencing of edited B-cells using pb111 (RECQ1-based construct). Panel A shows overviews of SNPs at each position of the targeting region of codon 112 site of human ApoE. Panel B shows an enlarged, trimmed view in the region adjacent to codon 112 site of human ApoE.

FIG. 12 shows insertion and deletion (Indels) analysis by next generation sequencing of edited B-cells using pb106 (WRN-based construct). Panel A shows overviews of InDels at each position of the targeting region of codon 112 site of human ApoE. Panel B shows an enlarged, trimmed view in the region adjacent to codon 112 site of human ApoE.

FIG. 13 shows insertion and deletion (Indels) analysis by next generation sequencing of edited B-cells using pb110 (BLM-based construct). Panel A shows overviews of InDels at each position of the targeting region of codon 112 site of human ApoE. Panel B shows an enlarged, trimmed view in the region adjacent to codon 112 site of human ApoE.

FIG. 14 shows insertion and deletion (Indels) analysis by next generation sequencing of edited B-cells using pb111 (RECQ1-based construct). Panel A shows overviews of InDels at each position of the targeting region of codon 112 site of human ApoE. Panel B shows an enlarged, trimmed view in the region adjacent to codon 112 site of human ApoE.

FIG. 15 shows insertion and deletion (Indels) analysis by next generation sequencing of edited B-cells illustrated as histograms. An x-axis indicates the number of deleted nucleotides (expressed as negative numbers), no insertions or deletions (indicated by 0) respectively insertions (expressed by positive numbers). A y-axis indicates the number of sequence reads obtained for each InDel. Panel A shows overviews of InDels at each position of the targeting region of codon 112 site of human ApoE using an exemplary polynucleotide modification agent, pb 106. Panel B shows overviews of InDels at each position of the targeting region of codon 112 site of human ApoE using an exemplary polynucleotide modification agent, pb 110. Panel C shows overviews of InDels at each position of the targeting region of codon 112 site of human ApoE using an exemplary polynucleotide modification agent, pb 111.

FIG. 16 shows editing efficiency at codon 112 site of ApoE in B cells using exemplary polynucleotide modification agent constructs pb 106, pb110, and pb 111.

FIG. 17 illustrates targeting and editing at codon 112 of human endogenous ApoE, as well as ddPCR detection of T→C conversion in human hepatocytes.

FIG. 18 illustrates a view of human hepatocytes in culture as observed using a phase contrast microscope at 40-fold visual amplification.

FIG. 19 demonstrates T→C genetic conversion at codon 112 of human ApoE by ddPCR analysis of dots representing droplets, containing indicated C (upper panel) or T alleles (lower panel), showing untreated human hepatocytes (HHC), human B-cells edited using pb 6, human hepatocytes edited using pb 6 (HHC/pb 6) respectively human hepatocytes edited using an exemplary polynucleotide modification agent, pb 111 (HHC/pb 111).

FIG. 20 is a chromatogram from Sanger sequencing of “wildtype” apoE with a nucleotide “T” as indicated.

FIG. 21 is a Sanger sequencing chromatogram of zinc finger helicase beta-wing mediated gene-edited codon 112 of ApoE with a “T-to-C” conversion indicated.

FIG. 22 shows Single Nucleotide Polymorphisms (SNP) analysis by next generation sequencing of unedited human hepatocytes. Panel A shows overviews of SNPs at each position of the targeting region of codon 112 site of human ApoE. Panel B shows an enlarged, trimmed view in the region adjacent to codon 112 site of human ApoE.

FIG. 23 shows Single Nucleotide Polymorphisms (SNP) analysis by next generation sequencing of edited human hepatocytes using an exemplary polynucleotide modification agent, pb111 (RECQ1-based construct). Panel A shows overviews of SNPs at each position of the targeting region of codon 112 site of human ApoE. Panel B shows an enlarged, trimmed view in the region adjacent to codon 112 site of human ApoE.

FIG. 24 shows insertion and deletion (Indels) analysis by next generation sequencing of unedited human hepatocytes. Panel A shows overviews of Indels at each position of the targeting region of codon 112 site of human ApoE. Panel B shows an enlarged, trimmed view in the region adjacent to codon 112 site of human ApoE.

FIG. 25 shows insertion and deletion (Indels) analysis by next generation sequencing of edited human hepatocytes using an exemplary polynucleotide modification agent, pb111 (RECQ1-based construct). Panel A shows overviews of Indels at each position of the targeting region of codon 112 site of human ApoE. Panel B shows an enlarged, trimmed view in the region adjacent to codon 112 site of human ApoE.

FIG. 26 shows insertion and deletion (Indels) analysis by next generation sequencing of unedited and edited human hepatocytes illustrated as histograms. An x-axis indicates the number of deleted nucleotides (expressed as negative numbers), no insertions or deletions (indicated by 0) respectively insertions (expressed by positive numbers). A y-axis indicates the number of sequence reads obtained for each Indel. Panel A shows overviews of Indels at each position of the targeting region of codon 112 site of unedited human hepatocytes. Panel B shows overviews of Indels at each position of the targeting region of codon 112 site of human ApoE using an exemplary polynucleotide modification agent, pb 111.

FIG. 27 shows editing efficiency at codon 112 site of ApoE in human hepatocytes using an exemplary polynucleotide modification agent, pb 111.

FIG. 28 provides an exemplary Bcl11A gene editing strategy. Panel A illustrates an exemplary targeting site of Bcl11A. Panel B provides an exemplary donor template. Panel C provides an exemplary genetic conversion in a Bcl11A gene sequence, from a GATAA-motif (here shown as its complementary sequence TTATC) to a “GAATTC” sequence.

FIG. 29 provides a schematic illustrating targeting and editing at a GATAA-motif in human Bcl11A, as well as a schematic of digital droplet PCR-based (ddPCR) detection of TTATC to GAATTC conversion.

FIG. 30 shows an example of successful genetic TTATC to GAATTC conversion in human Bcl11A by ddPCR. ddPCR analysis of dots representing droplets, containing indicated GAATTC sequence (upper panels) or TTATC sequence (lower panels).

FIG. 31 shows an example of Bcl11A TTATC to GAATTC gene editing frequency.

FIG. 32 depicts chromatograms from Sanger sequencing of untargeted control and edited with an exemplary polynucleotide modification agent, panels A and B, respectively. Positions of the gene edits are indicated in the dotted boxes.

FIG. 33 shows an exemplary PolG gene editing strategy. Panel A illustrates an exemplary targeting site of human PolG. Panel B provides an exemplary donor template; this is a (mostly) homologous donor template is used containing a “CTTAACTAAC” sequence. Panel C provides an exemplary genetic conversion of a sequence TCTGGCCAAT to CTTAACTAAC.

FIG. 34 provides a schematic illustrating targeting and editing in human PolG, as well as a schematic of digital droplet PCR-based (ddPCR) detection of TCTGGCCAAT to CTTAACTAAC conversion of PolG sequence.

FIG. 35 shows an example of successful genetic TCTGGCCAAT to CTTAACTAAC conversion in human PolG by ddPCR in B cells, HEK293 cells, and HepG2 cells. ddPCR analysis of dots representing droplets, containing indicated CTTAACTAAC sequence (upper panels) or wild-type sequence (lower panels).

FIG. 36 depicts a Sanger sequencing chromatogram of sequence that has edited PolG with an exemplary polynucleotide modification agent (SS-HbW agent). Positions of the gene edits are indicated by triangles underneath the chromatogram.

FIG. 37 shows an exemplary MMACHC gene editing strategy. Panel A illustrates an exemplary targeting site of human MMACHC. Panel B provides an exemplary donor template; this is a (mostly) homologous donor template is used containing a “GAGAGTGA” sequence. Panel C provides an exemplary genetic conversion of a sequence CCGTGTTAG to GAGAGTGA.

FIG. 38 provides a schematic illustrating targeting and editing in human MMACHC, as well as a schematic of digital droplet PCR-based (ddPCR) detection of CCGTGTTAG to GAGAGTGA conversion of MMACHC sequence.

FIG. 39 shows an example of successful genetic CCGTGTTAG to GAGAGTGA conversion in human MMACHC by ddPCR. ddPCR analysis of dots representing droplets, containing indicated GAGAGTGA sequence (upper panels) or wild-type sequence (lower panels).

FIG. 40 depicts a Sanger sequencing chromatogram of sequence that has edited MMACHC with an exemplary polynucleotide modification agent (SS-HbW agent), with the editing nucleotide sequence indicated by a bracket underneath the chromatogram.

FIG. 41 shows an exemplary MMUT gene editing strategy. Panel A illustrates an exemplary targeting site of human MMUT. Panel B provides an exemplary donor template; this is a (mostly) homologous donor template is used containing a “TAAATGC” sequence. Panel C provides an exemplary genetic conversion of a sequence CATGTGT to TAAATGC.

FIG. 42 provides a schematic illustrating targeting and editing in human MMUT, as well as a schematic of digital droplet PCR-based (ddPCR) detection of CATGTGT to TAAATGC conversion of MMUT sequence.

FIG. 43 shows an example of successful genetic CATGTGT to TAAATGC conversion in human MMUT by ddPCR. ddPCR analysis of dots representing droplets, containing indicated TAAATGC sequence (upper panels) or wild-type sequence (lower panels).

FIG. 44 shows an exemplary PAH gene editing strategy. Panel A illustrates an exemplary targeting site of human PAH. Panel B provides an exemplary donor template; this is a (mostly) homologous donor template, containing two indicated single nucleotide changes. Panel C provides the two single nucleotide changes of the genetic conversion.

FIG. 45 provides a schematic illustrating detection of targeted conversion of a PAH gene sequence.

FIG. 46 depicts a Sanger sequencing chromatogram of sequence that has edited with an exemplary polynucleotide modification agent (SS-HbW agent), with the editing nucleotide sequence indicated by a bracket underneath the chromatogram.

FIG. 47 shows an exemplary single nucleotide polymorphism (SNP) analysis by next generation sequencing of untargeted and Zinc Finger Helicase edited cells. Panel B shows an enlarged view of the indicated region for the untargeted results; Panel C shows an enlarged view for the pb 116 edited cells.

FIG. 48 shows an exemplary insertion and deletion (“indel”) analysis by next generation sequencing of untargeted and pb 116 edited cells.

FIG. 49 shows overall indel frequencies in untargeted and pb116 edited cells.

FIG. 50 shows an exemplary CFTR gene editing strategy. Panel A illustrates an exemplary targeting site of human CFTR. Panel B provides an exemplary donor template; this is a (mostly) homologous donor template is used containing a “ATG” sequence. Panel C provides an exemplary genetic conversion of a sequence CTT to ATG.

FIG. 51 provides a schematic illustrating detection of targeted conversion of a human CFTR sequence.

FIG. 52 shows an exemplary single nucleotide polymorphism (SNP) analysis by next generation sequencing of untargeted and Zinc Finger Helicase edited cells. Panel B shows an enlarged view of the indicated region for the untargeted results; Panel C shows an enlarged view for the pb 120 edited cells.

FIG. 53 shows overall indel frequencies at each nucleotide position at a target site in human CFTR in untargeted and pb 120 edited cells.

FIG. 54 provides an exemplary schematic showing steps involved in production of mRNA encoding an exemplary Zinc Finger Helicase. A plasmid encoding such a Zinc Finger Helicase can be linearized and used to produce mRNA by means of in vitro transcription and translation using mixtures and processes known to those skilled in the art.

FIG. 55 shows an example of successful genetic T to C conversion in human ApoE by ddPCR, as obtained following electroporation of an exemplary ApoE codon 112 targeting, mRNA encoded polynucleotide modification agent (e.g., a Zinc Finger Helicase agent).

FIG. 56 shows an example of apoE T to C gene editing frequency, using Zinc Finger Helicase mRNA produced from a Pst1 linearized pb 121 plasmid respectively Xho1 linearized pb121 plasmid.

FIG. 57 shows an example of successful genetic TTATC to GAATTC conversion in human Bcl11A by ddPCR, as obtained following electroporation of an exemplary Bcl11A targeting, mRNA encoded polynucleotide modification agent (e.g., a Zinc Finger Helicase agent).

FIG. 58 shows an example of successful genetic TCTGGCCAAT to CTTAACTAAC conversion in human PolG by ddPCR, as obtained following electroporation of an exemplary PolG targeting, mRNA encoded polynucleotide modification agent (e.g., a Zinc Finger Helicase agent).

FIG. 59 illustrates successful genetic T to C conversion when an exemplary Zinc Finger Helicase (pT7) is added extracellularly following electroporation of an exemplary oligonucleotide template (POP 33), as shown in column 2.

FIG. 60 shows a negative control (column 1) as and positive control (column 2). FIG. 8.2 also illustrates that direct extracellular addition of oligonucleotide template POP 33 (column 3) or extracellular addition of a purified Zinc Finger Helicase protein (pT7) and a oligonucleotide template POP 33 (column 4) did not result in genetic conversion.

FIG. 61 provides a representative schematic showing an exemplary Zinc Finger Helicase protein and an exemplary oligonucleotide being incubated under appropriate conditions, such that a protein-oligonucleotide complex (“protein-DNA complex”) can be formed. Such a protein-DNA complex can be added extracellular to cells, either as a single event, or, as illustrated here, as multiple events. After an incubation period, chromosomal DNA can be extracted and analyzed for genetic conversion.

FIG. 62 shows an example of successful genetic T to C conversion in human apoE by ddPCR, as obtained following extraocular addition of an exemplary apoE codon 112 targeting Zinc Finger Helicase protein (pT2) and an exemplary oligonucleotide (POP 33) that had been incubated under appropriate conditions before addition to cells.

FIG. 63 shows gene editing frequency for an exemplary genetic conversion of T to C in ApoE that was obtained after 4 subsequential extracellular additions of exemplary ApoE codon 112 targeting Zinc Finger Helicase protein (pT2, pT8) and an exemplary oligonucleotide (POP 33) that had been incubated under appropriate conditions before addition to cells.

FIG. 64 shows an exemplary single nucleotide polymorphism (SNP) analysis by next generation sequencing of an exemplary Zinc Finger Helicase protein (pT2)-oligonucleotide (POP 342) complex edited cells. Panel B shows an enlarged view of the pT2-POP342 edited cells.

FIG. 65 shows an exemplary insertion and deletion (indel) analysis by next generation sequencing of an exemplary Zinc Finger Helicase protein (pT2)-oligonucleotide (POP 342) complex edited cells. Panel B shows an enlarged view of the pT2-POP342 edited cells.

FIG. 66 shows an example of successful genetic T to C conversion in human apoE by next generation sequencing, as obtained following extraocular addition of an exemplary apoE codon 112 targeting Zinc Finger Helicase protein (pT2) and an exemplary oligonucleotide (POP 342) that had been incubated under appropriate conditions before addition to cells. In this example the cell medium contained serum.

FIG. 67 shows an exemplary insertion and deletion (indel) analysis by next generation sequencing of an exemplary Zinc Finger Helicase protein (pT2)-oligonucleotide (POP 33) complex edited cells. Panel B shows an enlarged view of the pT2-POP33 edited cells. In this example the cell medium contained serum.

FIG. 68 shows an exemplary Dystrophin (DMD) gene editing strategy. Panel A illustrates an exemplary targeting site of human DMD. Panel B provides an exemplary donor template; this is a (mostly) homologous donor template is used containing an insertion of 2 nucleotides “GA”, which is inserted within a “TTA (GA) CTCT” sequence. Panel C provides an exemplary genetic conversion of a sequence, insertion of “GA”.

FIG. 69 illustrates targeting and editing of human Dystrophin/DMD, as well as a schematic of digital droplet PCR-based (ddPCR) detection of TTACTCT to TTAGACTCT conversion.

FIG. 70 depicts a Sanger sequencing chromatogram demonstrating successful gene editing of DMD using an exemplary Zinc Finger Helicase protein (pT15) oligonucleotide complex. Panel A shows a sequence of a segment of “wild-type” human Dystrophin/DMD. Panel B is a chromatogram from Sanger sequencing, with positions of gene edits indicated by a dashed box.

FIG. 71 shows an exemplary single nucleotide polymorphism (SNP) analysis by next generation sequencing of an exemplary Zinc Finger Helicase protein (pT15)-oligonucleotide complex edited cells. Panel B shows an enlarged view of the pT12-oligonucleotide edited cells.

FIG. 72 provides a schematic showing an exemplary liquid nanoparticle (LNP) containing a chemically modified mRNA (cmRNA) encoding a Zinc Finger Helicase, as well as a single stranded oligonucleotide (ssODN). Such exemplary LNPs were used for in vivo testing in cell culture, as well as in an in vivo mouse model study.

FIG. 73 shows examples of successful genetic T to C conversion in human apoE by ddPCR, as obtained following extracellular addition of an exemplary LNP containing mRNA encoding an apoE codon 112 targeting Zinc Finger Helicase (pb121) and an exemplary oligonucleotide (POP 33) or combination of oligonucleotides (POP 358+POP 362). In this example LNPs were added to HepG2 cells as a single event (1×) or on three subsequential days (3×).

FIG. 74 shows examples of successful genetic T to C conversion in human apoE by Sanger sequencing, as obtained following extraocular addition of an exemplary LNP containing mRNA encoding an apoE codon 112 targeting Zinc Finger Helicase (pb121) and an exemplary oligonucleotide (POP 33). In this example LNPs were added to HepG2 cells on three subsequential days before chromosomal DNA was extracted for genetic analysis.

FIG. 75 illustrates an exemplary injection plan for in vivo testing of various exemplary LNP formulations in a mouse model.

FIG. 76 shows tissues obtained as part of the in vivo experiments.

FIG. 77 shows examples of successful genetic T to C conversion in human hepatocyte apoE by ddPCR, as obtained following intravenous injection of exemplary LNPs containing mRNA encoding an apoE codon 112 targeting Zinc Finger Helicase (pb121) and an exemplary oligonucleotide (POP 33) or combination of oligonucleotides (POP 358+POP 362).

FIG. 78 shows an example of apoE T to C gene editing frequency, as obtained following intravenous injection of exemplary LNPs containing mRNA encoding an apoE codon 112 targeting Zinc Finger Helicase (pb121) and an exemplary oligonucleotide (POP 33) or combination of oligonucleotides (POP 358+POP 362).

FIG. 79 shows examples of successful genetic T to C conversion in human hepatocyte apoE by next generation, as obtained following intravenous injection of exemplary LNPs in 4 different mice.

FIG. 80 shows examples of successful genetic conversion in human hepatocyte GBA by next generation, as obtained following intravenous injection of exemplary LNPs in 2 different mice.

### CERTAIN DEFINITIONS

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. In some instances, explicit definitions of certain terms are provided herein; meanings of these and other terms in particular instances throughout this specification will be clear to those skilled in the art from context.

As used herein, the term “adjacent” in refers to elements that are situated near, close to, or adjoining. In the context of a polynucleotide, two elements (e.g., components, molecules, sequences, etc.) are adjacent when they are situated near, close to, or adjoining in a linear polynucleotide and/or within a 3D architecture of a folded polynucleotide (e.g., 3D folded chromosomal genomic region). In some embodiments, adjacent polynucleotide sequences (e.g., genomic sequences, mRNA sequences, etc.) are two sequences that are situated near, close to, or adjoining in a linear polynucleotide (e.g., DNA) sequence and/or within a 3D architecture of a folded polynucleotide (e.g., 3D folded chromosomal genomic region). In some embodiments, an agent, molecule, or element described herein is adjacent when it comes into sufficiently close molecular proximity to a second agent, molecule, or element. For example, in some embodiments, two agents, molecules, or elements are considered adjacent when they bind to polynucleotide sequences that situated near, close to, or adjoining.

As used herein, the term, “affinity” is a measure of the tightness with a particular agent (e.g., polynucleotide modification agent and/or a SSB element thereof) binds to its target or binding partner (e.g., polynucleotide landing site and/or target sequence). Affinities can be measured in different ways. In some embodiments, affinity is measured by a quantitative assay. In some such embodiments, concentration of an agent and/or its target or binding partner may be varied in a quantitative assay. In some such embodiments, affinity may be compared to a reference under comparable conditions (e.g., concentrations). In some embodiments affinities can be determined using gene conversion methods. In some such embodiments, affinity may be compared to references or controls.

As used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

As used herein, the term “binding site” refers to a nucleic acid sequence within a nucleic acid molecule that is intended to be bound by an element (e.g., a sequence specific binding element) in a sequence-specific manner. In some embodiments, a SSB element (or portion thereof) binds to a binding site. In some embodiments, a binding site is a site at which an element of an agent, e.g., polynucleotide modifying agent, e.g., a SSB-HbW molecule, binds. In some embodiments, a binding site is intended to be sequence-specific but does not have to have 100% complementarity with an agent that binds to a binding site. For example, overall binding at a binding site is sequence-specific, which means that there is substantial sequence specificity of a given element for a binding site. For instance, for a given element to bind at a binding site, in some embodiments, there may be at least 15 nucleotides that are sequence-specific although the 15 nucleotides do not necessarily need to be contiguous with one another to confer specificity.

As used herein, the term “gene conversion” refers to a change in a sequence of a polynucleotide. In some embodiments, a change may be one or more of a substitution, deletion or addition of a nucleotide. In some such embodiments, a gene conversion is used to change one or more point mutations that exist in a particular gene via, e.g., a sequence modification polynucleotide. In some embodiments, a gene conversion results in a genomic genotype change that corresponds to a phenotypic change. For example, in some embodiments, a gene conversion changes a genotype from a pathogenic genotype to a functional (i.e., less pathogenic or non-pathogenic) phenotype. In some embodiments, no conversion occurs (either because no conversion has been attempted or because in a situation where one or more conversions are occurring, a particular polynucleotide is not modified). In some such embodiments, a polynucleotide and/or a cell comprising it may be referred to as “unconverted.”

As used herein, the term “gene editing” refers to a change in a sequence of a polynucleotide that corresponds to a gene or segment of a gene. In some embodiments, a change may be one or more of a substitution, deletion or addition of a nucleotide. In some such embodiments, a gene editing is used to change one or more point mutations that exist in a particular gene via, e.g., a sequence modification polynucleotide. In some embodiments, gene editing results in a genomic genotype change that corresponds to a phenotypic change. For example, in some embodiments, gene editing changes a genotype from a pathogenic genotype to a functional (i.e., less pathogenic or non-pathogenic) phenotype. In some instances, no editing occurs (either because no editing has been attempted or because in a situation where one or more edits are occurring, a particular polynucleotide is not modified). In some such embodiments, a polynucleotide and/or a cell comprising it may be referred to as “unedited.”

As used herein, the term “genetic modification” refers to a process of gene conversion in which genetic material (e.g., a polynucleotide such as, e.g., DNA, RNA, etc.) has a difference in its sequence (e.g., genomic sequence, transcript sequence, etc.) as compared to an initial sequence (e.g., before a modification, or in a daughter cell as compared to a parent cell, etc.) at a targeted locus and/or loci. In some embodiments, a genetic modification occurs in a cell (e.g., a daughter cell). In some embodiments, a genetic modification is made using one or more technologies (e.g., systems) as described herein. In some embodiments, a genetic modification may be at least one of a substitution, deletion, addition or change to molecular structure of a given nucleotide at a given target site or sites. In some embodiments, a genetic modification results in a change in a polynucleotide but no change in a corresponding polypeptide. In some embodiments, a genetic modification results in a change in a polynucleotide and a change in a corresponding polypeptide (i.e., a change in an amino acid corresponding to a triplet nucleotide). In some instances where no genetic modification occurs, genetic material and/or a cell comprising such genetic material may be referred to as “unconverted.”

As used herein, the term “helicase” is an enzyme capable of separating annealed strands in a polynucleotide, such as DNA or RNA, typically by breaking hydrogen bonds between the bases of two polynucleotide strands. An enzyme is said to have helicase activity if it is capable of separating annealed strands in a polynucleotide, such as DNA or RNA. In some embodiments, a helicase is a protein, e.g., an enzyme that can associate with a polynucleotide and break hydrogen bonds connecting nucleobase residues on opposing strands within a polynucleotide. In some embodiments, a helicase is part of a complex involving other enzymes, proteins or other biomolecules. In some embodiments, a helicase breaks hydrogen bonds within a specific polynucleotide of a particular sequence, which is also referred to herein as a “target site.” In some embodiments, a helicase causes a strand separation in a polynucleotide. In some such embodiments, such strand separations can involve a single basepair or a multitude of basepairs. Helicases can be naturally existing macromolecules or parts thereof; they can be modified versions thereof or can be designed or engineered. In some embodiments, helicases have a 3-dimensional fold in which certain amino acids form a catalytic core that can perform hydrogen bond cleavage and/or strand separation. In some embodiments, helicase or helicase-like domains can be incorporated into larger macromolecules. In some embodiments, a protein or enzyme can have helicase activity and/or function as a helicase, even if it is not named as a “helicase.” In some embodiments, proteins or enzymes can have helicase activity and/or function, and also may have additional functions or functionalities. For example, as non-limiting example, enzymes involved in DNA repair may display helicase functionality. As another non-limiting example human Rad4 may separate DNA strands as part of its working mechanism, as part e.g. of a nucleotide excision repair complex.

As used herein, the term “helicase beta-wing (HbW)” refers to a portion of a polypeptide with helicase activity that is involved in strand separation of a polynucleotide. In some embodiments, a helicase beta-wing comprises a structure of two or more amino acid beta-sheets. In some embodiments, a helicase beta-wing is a segment of a human helicase. In some embodiments, a helicase beta-wing is a segment of a non-human helicase. In some embodiments, a helicase beta-wing is a designed or non-natural molecule that can act as helicase beta-wing.

As used herein, the term “humanized” refers to a process to increase and/or maximize similarity of a protein or fusion protein to sequences present in a human genome and/or proteome. In some embodiments humanized proteins will be partially homologous to naturally existing human proteins and partially distinct from naturally existing human proteins. Such a partially homologous sequence, as used herein, is considered “humanized”. As non-limiting example, in some embodiments, a zinc finger linker between two zinc finger segments may comprise a naturally occurring human amino acid sequence TGEKP (SEQ ID NO: 14), whereas in some other embodiments a partially homologous linker sequence TGSQKP (SEQ ID NO: 15) may be used. Such a partially homologous sequence, as used herein, is considered “humanized”. As another non-limiting example, in some embodiments a naturally occurring zinc finger amino acid sequence of FQCRICMRNFSRSDHLTTHIRTH (SEQ ID NO: 16) may be replaced by a partially homologous amino acid sequence FQCRICMRNFSRSSALTRHIRTH (SEQ ID NO: 17). As used herein, proteins (e.g., fusion proteins) that are composed of one or more humanized polypeptide sequences, domains, and/or elements are also considered “humanized”.

As used herein, the term “landing site” refers to a nucleic acid sequence to which a sequence-specific binding element is targeted (e.g., binds). In some embodiments, a landing site may overlap with a target site (e.g., have nucleotides that are part of both a landing site and a target site). In some embodiments, a landing site may comprise a target site or a portion thereof. In some embodiments, a landing site may be in relatively close proximity (e.g., adjacent) to a target site. In some embodiments, a landing site may be a distance away from a target site. In some such embodiments, where a landing site is a distance away from a target site, it is still considered a landing site as long as cellular modification processes enable modification of a target site.

As used herein, the term “nuclease” is an enzyme capable of cleaving one or more bonds in a polynucleotide, typically by hydrolyzing one or more phosphodiester bonds between individual nucleotides. In some embodiments, a nuclease is a protein, e.g., an enzyme that can bind a polynucleotide and cleave a phosphodiester bond connecting nucleotide residues within the polynucleotide. In some embodiments, a nuclease is site-specific. In some such embodiments, such a nuclease binds and/or cleaves a specific phosphodiester bond within a specific polynucleotide of a particular sequence, which is also referred to herein as a “target site.” In some embodiments, a nuclease causes a break in a polynucleotide. In some such embodiments, such breaks can be single-stranded or double-stranded in that a single-stranded break is a break that occurs in a single-polynucleotide strand (in a single or double-stranded molecule) and a double-stranded break is one that occurs between at least two nucleotides on one strand and the complementary nucleotides on an opposite strand of a double-stranded molecule. Nucleases can be naturally existing macromolecules or parts thereof; they can be modified versions thereof or can be designed or engineered. In some embodiments, nucleases have a 3-dimensional fold in which certain amino acids form a catalytic core that can perform catalytic hydrolysis. In some embodiments, nuclease or nuclease-like domains can be incorporated into larger macromolecules.

As used herein, the term “nucleic acid” refers to any element that is or may be incorporated into a polynucleotide chain. In some embodiments, a nucleic acid may be incorporated into a polynucleotide chain via phosphodiester linkage. In some embodiments, nucleic acids are polymers of deoxyribonucleotides or ribonucleotides. In some such embodiments, deoxyribonucleotides or ribonucleotides may be synthetic oligonucleotides. As will be clear from context, in some embodiments, “nucleic acid” refers to an individual nucleic acid residue (e.g., a nucleotide and/or nucleoside); in some embodiments, “nucleic acid” refers to a polynucleotide comprising individual nucleic acid residues. In some embodiments, a polymer or deoxyribonucleotides and/or ribonucleotides can be single-stranded or double-stranded and in in linear or circular form. Polynucleotides comprised of nucleic acids can also contain synthetic or chemically modified analogues of ribonucleotides, in which a sugar, phosphate and/or base units are modified. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs. In some embodiments, a nucleic acid comprises one or more modified sugars as compared with those in natural nucleic acids. In some embodiments, a polynucleotide is comprised of at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, a polynucleotide is partly or wholly single stranded molecule. In some embodiments, polynucleotide is partly or wholly double stranded.

As used herein, the term “polynucleotide” refers to any polymeric chain of nucleic acids. In some embodiments, a polynucleotide is or comprises RNA. In some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded. In some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

As used herein, the term “replicating cells”, refers to cells and cell types that have an inherent ability to multiply. For example, as non-limiting example various stem and progenitors cell types have an inherent ability to divide and yield a multitude of cells. Replicating cells may result in the generation of more than one cells of the same type as the original cell, or cells may have a different character. For example, hematopoietic stem cell replication may results in propagation of such stems cells and/or other derived cell types, such as B-cells, NK cells, red blood cells etc. may be the result of cell replication.

As used herein the term “non-replicating cells”, refers to cell types that under typical physiological conditions are considered to be post-mitotic and have lost the ability to replicate. As non-limiting example, neurons are generally regarded as post-mitotic cells that do not replicate.

As used herein, the term “sample” refers to a portion or aliquot of a material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, an organism is a pathogen (e.g., an infectious pathogen, e.g., a bacterial pathogen, a viral pathogen, a parasitic pathogen, etc.). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., broncheoalveolar, ductal, nasal, ocular, oral, uterine, vaginal, or other washing or lavage). In some embodiments, a biological sample is or comprises cells obtained from an individual. In some embodiments, a sample is a primary sample in that it is obtained directly from a source of interest by any appropriate means. In some embodiments, as will be clear from context, a sample refers to a preparation that is obtained by processing (e.g., by removing one or more components of and/or by adding one or more agents to) a primary sample. For example, processing a sample for testing to extract genetic material for genetic analyses such as by, e.g., applying one or more solutions, separating components using a semi-permeable membrane, etc. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample is used to design one or more polynucleotide modification agents and/or sequence modification polynucleotides as provided herein.

As used herein, the term “sequence modification polynucleotide” refers to a polynucleotide that has substantial homology with a target sequence (e.g., a genomic sequence, a transcript, etc.), but is not identical to that target sequence. In some embodiments a sequence modification polynucleotide may have properties equivalent to a wild-type polynucleotide, but may be chemically modified and/or use synthetic or chemically modified building blocks. In some embodiments, a sequence modification polynucleotide is used in conjunction with a polynucleotide modifying agent (e.g., a SSB-HbW molecule) in order to achieve sequence modification at a target site. For example, in some embodiments, a sequence modification polynucleotide is a donor template in that such a polynucleotide provides one or more nucleic acids for incorporation into a given sequence (e.g., a genomic sequence, a transcript, etc.). In some embodiments, a sequence modification polynucleotide is a correction template in that it is used in a cellular process (e.g., a replication process) as a “guide” of sorts by cellular machinery in order to make a change (e.g., a substitution, deletion, addition) to a given polynucleotide (e.g., DNA, mRNA, etc.), In some embodiments, a sequence modification polynucleotide may contain a “wild-type” nucleic acid sequence that is almost entirely identical or homologous to a variant sequence except for one or two nucleotides (i.e., point mutations, substitutions, etc.) that is/are regarded as changed relative to the wild type sequence (i.e., a variant sequence). In some embodiments, a sequence modification polypeptide such as a donor template may differ by only a single nucleotide relative to a wild-type sequence. In some embodiments, a sequence modification polypeptide may have two or more nucleotide differences relative to a wild-type sequences. In some such embodiments, such a polypeptide may have multiple nucleotides differences in a target sequence as compared to a wild-type sequence. A sequence modification polynucleotide may be at least about 10 nucleotides to at least about 20 kb in length. In some embodiments, a sequence modification polynucleotide is or comprises a template which itself is not necessarily incorporated into, e.g., a replicating nucleic acid strand, but the sequence of the sequence modification polynucleotide is reflected in a replicated nucleic acid strand (e.g., a nucleic acid strand is edited after contact with a sequence modification polynucleotide even if the physical sequence modification polynucleotide itself is not incorporated into the strand). In some embodiments, a sequence modification polynucleotide has or comprises a sequence that is at least 50%, 60%, 70%, 75%, 80%, 85%, 90%, 95%, 96%, 97%, 98%, 99%, 99.1%, 99.2%, 99.3%, 99.4%, 99.5%, 99.6%, 99.7%, 99.85, or 99.9% or greater identical to a target sequence and/or target site. In some embodiments, a sequence modification polynucleotide has or comprises a sequence that is at most approximately 99.9%, 99.8%, 99.7%, 99.6%, 99.5%, 99.4%, 99.3%, 99.2%, 99.1%, 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 1%, or 0% identical to a target site or sequence as provided herein. In some embodiments, identity is over a particular size or length of target size or sequence. In some embodiments, identity does not refer to a contiguous sequence. In some embodiments, identity does refer to a contiguous sequence.

As used herein, the term “sequence-specific binding” refers to an event that occurs when a macromolecule (e.g., a protein, peptide, polypeptide, nucleotide comprising protein) interacts with a polynucleotide (e.g., DNA, RNA, etc.), and at least a sub-set (e.g., 2, 3, 4, 5, 6, 7, 8, 9, 10 or more) of contacts between a macromolecule and a polypeptide is sequence-specific in that expected portions of each molecule interact with one another (e.g., Arginine interacting with Guanidine; other exemplary interactions will be known to those of skill in the art and can be found, for instance, in various descriptions throughout the literature describing DNA recognition codes for zinc fingers). As is understood by those of skill in the art, not every interaction between every portion of each molecule needs to be sequence specific; however the overall interaction between two molecules interacts, generally, in a manner that is sequence-specific. In some embodiments an overall dissociation constant for interaction will be 10 E−6 or less. As will be appreciated by those of skill in the art, a smaller dissociation constant indicates stronger binding. In some embodiments, sequence-specific binding will entail interaction in which at least three base pairs or nucleotides are bound with sufficient affinity and selectivity, such that other sequences will be bound at levels less than 50% of a desired or targeted DNA sequence.

As used herein, the term “subject” refers to an organism. In some embodiments, a subject is an individual organism. A subject may be of any chromosomal gender and at any stage of development, including prenatal development. In some embodiments, a subject is comprised of, either wholly or partially, eukaryotic cells (e.g., an insect, a fly, a nematode). In some embodiments, a subject is a vertebrate. In some embodiments, a subject is a mammal. In some embodiments, a mammal is a human, including prenatal human forms. In some embodiments, a subject is an individual to whom a genetic modification system or component thereof is to be administered. In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been and/or will be administered.

As used herein, the term “target cell” or “targeted cell” refers to a cell that has been contacted with at least one polynucleotide modification agent (e.g., a SSB-HbW molecule), and optionally at least one sequence modification polynucleotide. In some embodiments, a target cell comprises at least one nucleic acid change at a target site as compared to the same cell prior to the application of the at least one polynucleotide modification agent and at least one sequence modification polynucleotide, or, in some embodiments, as compared to another targeted cell or an untargeted cell. In some embodiments, a target cell does not comprise a nucleic acid change at a target site as compared to an untargeted cell. In some embodiments, a targeted cell may have one or more nucleic acid differences as compared to an untargeted cell, but is still not an edited cell as the one or more differences may not be at or within a target site. A targeted cell may or may not be an edited cell. In some embodiments, a targeted cell is an edited cell in that its nucleic acid sequence has been successfully edited in a specific and intended way, e.g., reflecting a designed genetic change based upon a supplied sequence modification polynucleotide. In some embodiments, an edited cell has a specific nucleotide sequence in which technologies of the present disclosure are used to make one or more nucleotide modifications (e.g., substitutions, additions, deletions, etc.) relative to, for example, a control cell or a targeted cell that is not an edited cell. For example, in some embodiments, an untargeted cell or a targeted but unedited cell, does not reflect a specific sequence (i.e., is not edited) provided using a sequence modification polynucleotide. In some embodiments, a targeted, edited cell may have one or more additional changes in addition to changes introduced via a sequence modification polynucleotide (e.g., SNP). In some embodiments, a targeted but unedited cell and/or an untargeted cell may have one or more genetic changes as compared to an earlier version of a cell or a control, but does not have or comprise a particular sequence provided by a sequence modification polynucleotide. For example, in some embodiments, one or more SNPs may be detected but such SNPs may not be in a vicinity of a target site.

As used herein, the term “target sequence” refers to a particular sequence comprising one or more nucleic acids to be modified using technologies of the present disclosure. In some embodiments, a target sequence is or comprises one or more nucleotides. In some embodiments, a target sequence is wholly naturally-occurring. In some embodiments, a target sequence is or comprises one or more synthetic nucleotides or components. In some embodiments, a target sequence is or comprises both naturally occurring or synthetic components (e.g., nucleic acid residues, etc.).

As used herein, the term “target site” refers to a location (e.g., a particular genome, chromosome, chromosomal position, etc.) of a given nucleic acid sequence within a nucleic acid molecule that comprises a target sequence, which target sequence is intended to be modified by a genetic modification system described herein (e.g., a system comprising a SSB-HbW agent). For example, in some embodiments, a target site is or comprises a nucleotide that is targeted for a change (e.g., replacement via substitution, removal, addition, etc.). In some such embodiments, a target site is a sequence-specific target site. In some embodiments, a target site is a structure specific target site. In some embodiments, a target site is both sequence and target specific. In some embodiments, a target site is non-sequence and/or non-structure specific. In some embodiments, a target site compromises a sequence associated with a disease, disorder or condition. In some embodiments, a target site is or comprises a polynucleotide sequence, e.g., a DNA sequence, that comprises a point mutation associated with a disease, disorder or condition. In some such embodiments, a target site may be or comprise an error site (e.g., a site where presence of one or more nucleotides is associated with existence, development or risk of a disease, disorder, or condition).

As used herein, the terms “treat” or “treatment” refer to any technology as provided herein that is used to partially or completely alleviate, ameliorate, relieve, inhibit, prevent, delay onset of, reduce severity of, and/or reduce incidence of one or more symptoms or features of a disease, disorder, and/or condition. In some embodiments of the present disclosure a treatment may be or comprise changing a genotype in a subject. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment refers to administration of a therapy (e.g., composition, pharmaceutical composition, e.g., SSB-HbW molecule and/or sequence modification polynucleotide and/or enhancing and/or inhibiting agent, etc.) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

Standard techniques may be used for recombinant DNA, oligonucleotide synthesis, and tissue culture and transformation (e.g., electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION

The present disclosure provides technologies (e.g., systems, agents, methods, etc.) related to gene and/or genome editing. Gene editing and genomic engineering hold great promise. As will be appreciated by those of skill in the art, such technologies have a wide array of applications. For instance, many types of editing or engineering could be useful in treating one or more diseases, disorders or conditions. The present disclosure recognizes that an ideal approach to gene editing would encompass features such as being (1) safe and with few to no off-target effects; (2) versatile ability to convert all types of variants (e.g., differences relative to wild-type) to a desired genotype (e.g., a wild-type genotype, a codon-optimized genotype, etc.); (3) versatile ability to be used in multiple cell types, including replicating and non-replicating cell types; (4) be sufficiently effective to be of practical use; and (5) use non-immunogenic components. None of the currently existing methods for gene editing and genomic engineering fulfills all five criteria.

The present disclosure appreciates that a challenge with many currently available gene editing approaches is that they use components that are of bacterial, viral or other non-human origin. For example, prior methods have included viral or bacterial components, which can introduce foreign (e.g., viral) molecules into a eukaryotic or human host. Other recently developed methods such as, e.g., “CRISPR/CAS”, “base editors” or “prime editors” make use of fusion molecules that include bacterial proteins. The use of such components may result in an immunogenic response, such as antibody reactions or production of cytotoxic T-cells. Immunogenic responses in an individual can result in negative consequences, e.g. rendering a potential therapy becoming less effective; or gene edited cells, that still harbor a foreign component, may become target of cytotoxic T-cell and/or other immune responses. If gene editing methods are needed that avoid the introduction of non-immunogenic components to target cells, none of these methods provide an appropriate choice. For example, when multiple rounds of genome modification would be beneficial (e.g. when a large organ is being targeted, and e.g. when it would be beneficial or desirable to provide a treatment in multiple doses, or, e.g. when it would be desirable to provide a therapy at different ages within the same individual), immune responses may limit the effectiveness of such a therapy.

The present disclosure provides innovative technologies (e.g., systems, compositions, methods) for genetic modification that overcome limitations of current technologies. The present disclosure provides technologies that include polynucleotide modification agents that are composed of sequences from a human genome and/or proteome. In some embodiments, provided polynucleotide modification agents are composed of sequences that exist within a human genome and/or sequences that share significant homology with human sequences such that potentially harmful immune responses can be avoided. In some embodiments, methods of the present disclosure use polynucleotide modification agents that are derived from human sequences. In some embodiments, a polynucleotide modification agent may comprise one or more elements that are derived from a human sequence. In some embodiments, all elements of a polynucleotide modification agent may be derived from a human sequence. In some embodiments, the present disclosure provides humanized polynucleotide modification agents. In some embodiments, a polynucleotide modification agent is a fusion protein that is composed of two or more human protein sequences. In some embodiments, a polynucleotide modification agent is a fusion protein that is composed of two or more mammalian protein sequences.

The present disclosure recognizes that, among other things, it would be advantageous to be able to achieve gene and/or genome editing or engineering without needing to introduce single stranded or double stranded breaks to a genome in a target cell. In some embodiments, provided polynucleotide modification agents are capable of gene or genome editing without having to introduce one or more breaks in, e.g., a polynucleotide chain. In some embodiments, a polynucleotide modification agent is a sequence-specific binding molecule that, in combination with a sequence modification polynucleotide, can be introduced into a cell to achieve genetic modification (e.g., DNA modification, RNA modification) without the administered agent creating single- or double-stranded breaks in endogenous polynucleotides (e.g., DNA, etc.).

The present disclosure provides the insight that if, for example, double stranded DNA is able to be separated into single stranded DNA at or close to a target site, there would be an opportunity for a genetic modification agent to bind to a (partially) complementary sequence (e.g., substitution, deletion, addition), such that a DNA sequence modification could be introduced into a molecule comprising target site. Without being bound by any particular theory, the present disclosure contemplates that one way to achieve a genetic modification without inducing a break is, for example, to make a modification at a target site by providing a polynucleotide modification agent that associates (e.g., binds) at or near a landing or target site that can result in DNA strand separation and also provides another molecule which acts as a template or donor to achieve a nucleotide change.

The present disclosure provides versatile technologies (e.g., systems, compositions, methods) for genetic modification of multiple cell types, including replicating and non-replicating cell types. In some embodiments, provided polynucleotide modification agents can bind to polynucleotide and promote strand separation at or close to the binding site. Without being bound to any theory, the present disclosure recognizes that binding of a sequence modification polynucleotide to a (partially) complementary sequence to a separated strand can result in a sequence modification sequence becoming incorporated in a genome. In some embodiments, provided technologies are capable of genetically modifying non-dividing or post-mitotic cells.

### Polynucleotide Modification Agents

The present disclosure provides polynucleotide modification agents for gene or genome editing. In some embodiments, a polynucleotides modification agent is a sequence-specific binding molecule that, in combination with a sequence modification polynucleotide, can be introduced into a cell to achieve genetic modification (e.g., DNA modification and/or RNA modification) without the administered agent introducing bacterial or viral molecules into a cell (e.g., by avoiding bacterial components such as a CAS-protein etc.). In some embodiments, a polynucleotides modification agent is a fusion protein that comprises two or more mammalian protein sequences. In some embodiments, a polynucleotides modification agent is a fusion protein that comprises two or more human protein sequences.

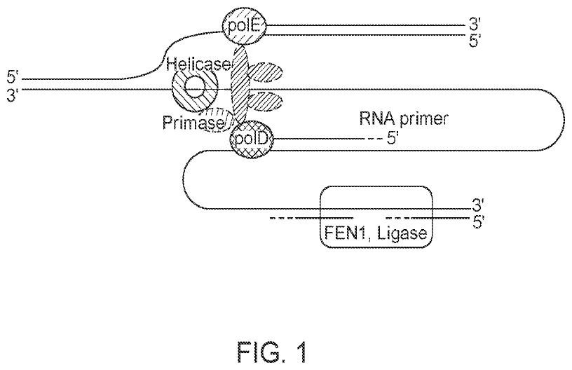

Without being bound by any particular theory, the present disclosure contemplates that a polynucleotide modification agent (e.g., a helicase beta-wing containing fusion molecule) will facilitate a sequence modification (e.g., via a sequence modification polynucleotide). For example, as will be appreciated by one of skill in the art, FIG. 1 illustrates a schematic of a DNA replication. Generally, during DNA replication, a replication complex “unwinds” a double-helical conformation of a given DNA molecule and as this unwinding occurs, both a “leading” and “lagging” single strands are present, and each being replicated via replication machinery. It is generally understood that a function of helicase is to separate strands in double stranded DNA. The present disclosure appreciates that helicase is involved in generating single strands and, in particular, that a certain portion of helicase may be involved in facilitating separating of strands.

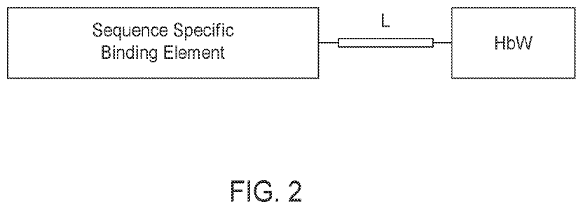

In some embodiments, the present disclosure provides polynucleotide modification agents comprising a sequence specific binding element and an element with helicase activity. In some embodiments, the present disclosure provides polynucleotide modification agents comprising a sequence-specific binding element (“SSB element” or “SSBE”) and a helicase beta-wing element (“HbW element”). FIG. 2 provides a schematic of an exemplary polynucleotide modification agent comprising a HbW element.

The present disclosure provides the insight that separation of a double stranded polynucleotide (e.g., DNA) at or close to a target site, provides an opportunity for sequence modification (e.g., with a sequence modification template (e.g., donor template)). Accordingly, the present disclosure, provides technologies (e.g., systems, compositions, methods) that use helicase functionality (e.g., DNA double strand separation) to generate a single strand segment at or close to a target site. In some embodiments, exposure of a single strand, makes it available for binding to a sequence modification polynucleotide.

As is provided herein, in some embodiments, the present disclosure describes the development and use of a DNA strand separating agent that can bind specifically and strongly enough to a polynucleotide molecule, e.g., a DNA molecule, such that single stranded DNA can be locally exposed. In some such embodiments, a single-stranded polynucleotide (e.g., a strand of DNA) may be exposed.

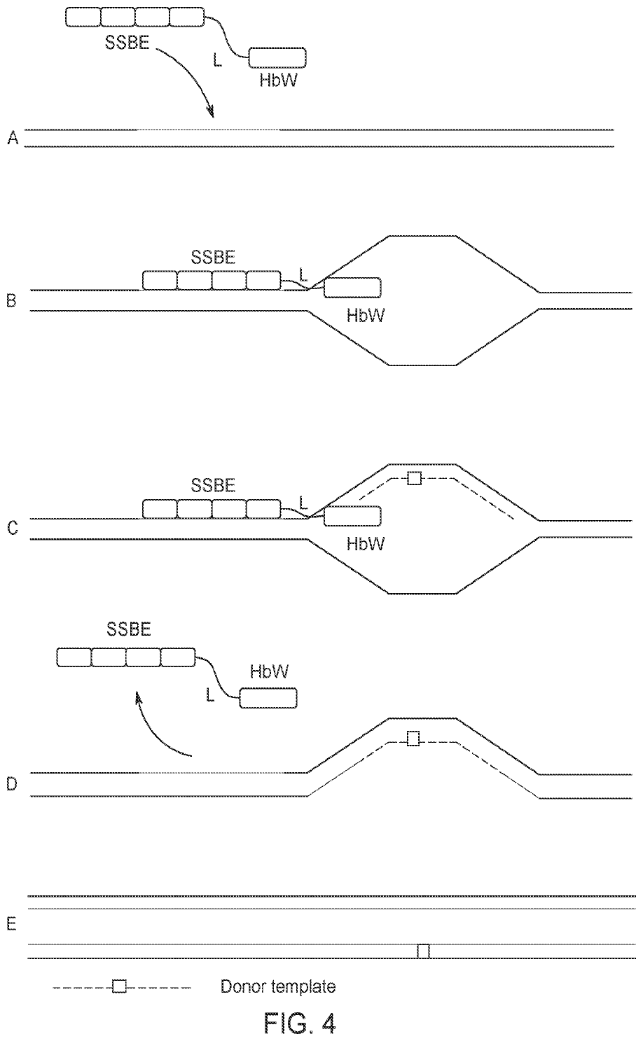

Thus, by way of non-limiting example, in some embodiments, the present disclosure provides a sequence specific binding element of a polynucleotide modification agent that can bind specifically and strongly enough to a DNA molecule such that a polynucleotide modification agent can separate DNA strands. In some such embodiments, a single stranded DNA segment is exposed and another polynucleotide can bind, such as a DNA modification polynucleotide (see, e.g., FIG. 4).

In some embodiments, a polynucleotide modification agent is a DNA strand separating agent. In some such embodiments, a DNA strand separating agent is engineered to, for example, reversibly bind to a nucleotide sequence (e.g., a landing site, a binding site, etc.), in a sequence-specific manner. In some embodiments, a DNA strand separating agent is an agent that is or comprises one or more components that bind(s) to a landing site, binding site, and/or target site. In some embodiments, a DNA strand separating agent comprises a component that, e.g., results in presence of single strand DNA. In some embodiments, a DNA strand separating agent is or comprises a polynucleotide modification agent comprising a HbW element, as provided herein.

In some embodiments, one or more elements of a polynucleotide modification agent are derived from a human sequence and include modifications to that sequence that enable functionality. In some embodiments, a polynucleotide modification agent comprises a SSB element comprising one or more zinc finger sequences and a strand separating element derived from a helicase comprising a “beta-wing” structure in combination with a number of amino acid substitutions, insertions and/or deletions that enable functionality of a polynucleotides modification agent.

In some embodiments, the present disclosure provides a polynucleotide modification agent comprising a fusion protein comprising a HbW element and a sequence specific binding element. In some such embodiments, a polynucleotide modification agent binds to a binding site (e.g., a landing site). In some such embodiments, a landing site may be the same the target site. In some embodiments, a landing site overlaps (i.e., shares one or more nucleic acid residues) with a target site. In some embodiments, a landing site and a target site do not overlap at all.

### SSB-HbW Agents

In some embodiments, a polynucleotide modification agent is or comprises a SSB-HbW agent (see, e.g., FIG. 2). In some embodiments, a SSB-HbW agent comprises at least two elements, a sequence specific binding “SSB” element and a helicase beta wing “HbW” element, with an optional linker “L” element. In some embodiments, a SSB-HbW agent has or comprises a structure set forth as SSB-L-HbW. The present disclosure also provides, among other things, methods of making and using disclosed agents. In some such embodiments, a SSB-HbW agent reversibly binds to double-stranded DNA, in a sequence specific manner. In some embodiments, a SSB-HbW agent may be ordered with SSB, L, and HbW elements placed consecutively. Thus, as described herein, in some embodiments, a SSB-HbW agent can be schematically represented as SSB-L-HbW or HbW-L-SSB.

In some embodiments, polynucleotide modification agents provided herein comprise a first domain comprising a sequence-specific DNA binding element that binds to a DNA strand; “L” is an optional linker element between segments “SSBE” and “HbW”; and “HbW” is a second domain that comprises a helicase beta-wing sequence, structure or functionality. In some embodiments, an HbW element is or comprises a polynucleotide that interacts with a different polynucleotide than a SSB element. In some such embodiments, an HbW element interacts with a polynucleotide on the same molecule as a SSB element. In some embodiments, an HbW element interacts with a polynucleotide on a different molecule as a SSB element of a single SSB-HbW agent. In certain aspects, the three elements are able to be reversibly bound (SSBE and HbW) or associated (L) to a polynucleotide (e.g., DNA) molecule.

In some embodiments, a SSB-HbW agent may be or comprise a polypeptide. In some such embodiments, where a SSB-HbW is a polypeptide, a SSB element can be located at either an N-terminal or C-terminal portion of a polypeptide, with an HbW-element located at an opposite location (e.g., C-terminal or N-terminal location). In some embodiments, where a SSB-HbW agent (e.g., polypeptide) comprises one or more L elements, such L elements are located in between SSB elements and HbW elements.

In some embodiments, a given SSB-HbW agent may have more than one each of a given SSB, L, or HbW element. For example, in some embodiments, a SSB element may be fused or otherwise connected to one or more L elements, which may each be fused or otherwise connected to one or more HbW elements.

In some embodiments, one or more of the SSB, L, and/or HbW elements are in an order different from SSB-L-HbW. In some embodiments, where more than one unit of any particular element is present, one of skill in the art will understand that a numeral may be used to indicate a number of a particular element, e.g., SSB-L₂-HbW₂ or SSB-L₂-HbW₂ or SSB(LHbW)₂, indicates a SSB element with two L elements bound to the SSB and two HbW elements, wherein the HbW elements may each be bound to the same or different L element. In some embodiments, an arrangement may also be shown as HbW-L-SSB-L-HbW, which would indicate that a single SSB element has two separate L elements bound to it, each of which has an HbW element bound to the L element. In some embodiments, a single SSB element may have more than one L element and more than one HbW element bound at a given time. In some embodiments, a single L element may have two HbW elements bound at the same time. In some embodiments, an HbW element may have at either end, a sequence that functions as a linker. For example, in some embodiments, a given HbW element may have a sequence at an N or C-terminus a sequence that functions as a linker such that a polymeric agent (e.g., SSB-HbW molecule) is represented as SSB-HbWn, where n may be, e.g., an L element.

In some embodiments, a SSB element is comprised of multiple components or DNA binding elements. For example, in some embodiments, a SSB element is “hybrid” comprising zinc-finger components and additional sequences.

In some embodiments, a SSB-HbW molecule has an overall dissociation constant in the same order as the lowest dissociation constant of any given component of the molecule (e.g., of a SSB element and/or a HbW element). For example, in some embodiments, a SSB element and an HbW element of a given SSB-HbW molecule may have dissociation constants of 10 E−6 or less and 10 E−3 or less, respectively and, in such embodiments, a dissociation constant of a SSB-HbW molecule would be consistent with the lowest dissociation constant of a component of such a molecule.

As described herein, technologies provided by the present disclosure (e.g., systems, methods, compositions, etc.) achieve one or more genetic modifications at one or more target sites. Accordingly, for example, in some embodiments, a SSB-HbW agent binds at a target site in a target genome wherein a SSB element binds to one strand of a DNA double helix in a sequence-specific manner and an HbW element results in strand separation in a DNA molecule (see, e.g., FIG. 4). In some such embodiments, when a sequence modification polynucleotide is present (such as illustrated in, e.g., FIG. 4 where a single stranded oligonucleotide has a desired DNA modification), the sequence modification polynucleotide can anneal to its complementary strand and create a sequence mismatch. In some embodiments, one or more intrinsic DNA repair processes in a given cell can result in a genetic modification by incorporating the desired alteration (e.g., the sequence of the sequence modification polynucleotide). Thus, gene editing can be accomplished without having to induce or cause, e.g., a DNA strand break with nuclease activity of a SSB-HbW agent itself.

In some embodiments, a SSB-HbW agent comprises a first domain, an optional linker, and a second domain. In some embodiments, a first domain is capable of binding to a DNA sequence (e.g., a SSB element, e.g., a zinc finger protein or a Leucine zipper protein), and a second domain (e.g., an HbW element) is able to interact with a polynucleotide (e.g., a DNA double helix), for example, in resulting in DNA strand separation. In some such embodiments, a first domain binds in a sequence-specific manner and a second domain interacts in a non-sequence specific manner. In some embodiments, binding of a SSB-HbW agent can result in DNA strand separation at or close to the binding or target site. For example, in some embodiments, in the context of DNA as a target site, binding of such a SSB-HbW agent can result in DNA strand separation and thus enabling a polynucleotide to bind to exposed single stranded DNA sequences. For example, in some embodiments, when a polynucleotide contains one or more nucleotides that are different from that of an original host cell, this may result in DNA conversion. The present disclosure contemplates that, in some embodiments, SSB-HbW agents as described herein may be useful for targeted editing of a polynucleotide (e.g., DNA, RNA, etc.) without directly or indirectly causing single or double stranded breaks at or near a target site.

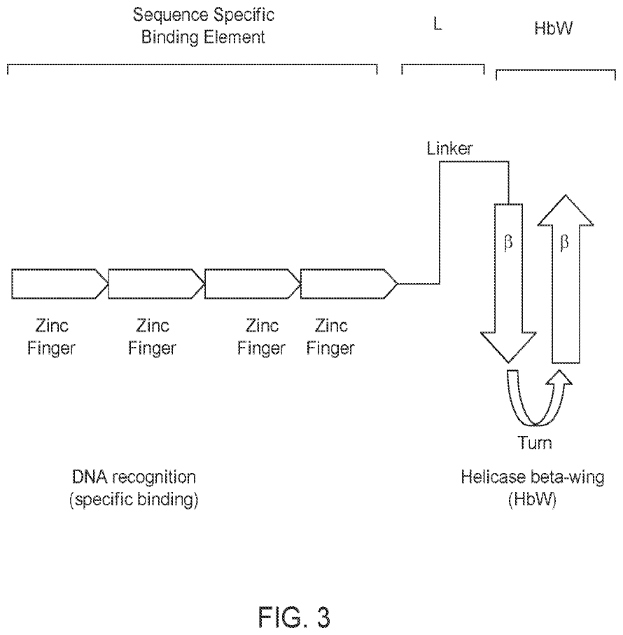

In some embodiments, a SSB-HbW agent can be or comprise a polypeptide (e.g., a protein). For example, a SSB-HbW agent, may, in some embodiments, comprise a SSB element comprising an array of at least 4 zinc fingers that can recognize a target site (e.g., a DNA target site) and an HbW element may be or comprise a helicase beta-wing (see, e.g., FIG. 3). In some embodiments, an HbW element of such a SSB-HbW agent is based on a structure from a helicase.

In some embodiments, a SSB-HbW agent directly interacts with a DNA molecule. In some embodiments, interaction of a SSB-HbW agent with a DNA molecule opens an opportunity that a modification oligonucleotide can anneal to a (partially) complementary single stranded DNA sequence that is (temporarily) exposed. SSB-HbW binding can induce the appearance of single stranded DNA in the vicinity of a SSB-HbW binding site and thus expose single stranded DNA at a conversion site.

The present disclosure contemplates that cells containing both a SSB-HbW agent and a sequence modification polynucleotide or donor polynucleotide can thus generate a DNA conversion.

In some embodiments, polynucleotide modification agents of the present disclosure and uses thereof, e.g., SSB-HbW agents, are designed to lack nuclease activity. In some embodiments, lack of nuclease activity avoids creating DNA breaks that typically result in Non-Homologous End-Joining (NHEJ). In some embodiments, when both a SSB-HbW agent and a sequence modification polynucleotide are present in a cell, gene conversion can be achieved with only (very) low levels of background damage generated via NHEJ mediated DNA conversion processes.

### Modularity of Design of SSB-HbW

Among other things, the present disclosure provides technologies (e.g., systems, methods, compositions, etc.) such that various elements of a SSB-HbW agent can be modular in design. For example, in some embodiments, as provided herein, a SSB element may be or comprise a zinc finger array, a leucine zipper, etc. As will be apparent by those of skill in the art, such modularity provides for a versatile and effective gene editing system, wherein, among other things and in contrast to a majority of available gene editing systems, SSB-HbW-based technologies as described herein do not depend on creation of double- or single strand DNA breaks to induce gene conversion and use components that are designed to avoid or minimize immune reactions in a human host.

For example, in some embodiments, a SSB-HbW agent is designed with a zinc finger array as a SSB element. For example, in some embodiments, different types of SSB elements can be used. In some embodiments, other types of SSB elements in a given SSB-HbW containing system can be functional, assuming that they provide sequence specific nucleotide (e.g., DNA) binding.

### Sequence Specific Binding Elements

In some embodiments, the present disclosure provides a SSB-HbW agent, which includes a sequence specific binding (SSB) element (SSBE). As used herein, a “SSB element” refers to a sequence-specific polynucleotide (e.g., DNA) binding element. In some embodiments, a SSB element is a domain capable of binding to a sequence (e.g., a nucleotide sequence, e.g., a landing site, e.g., a binding site) specifically on a single strand of a polynucleotide (e.g., such as a single strand of a DNA molecule, or on an RNA transcript, etc.).

In some embodiments, a SSB element can be or comprise a naturally occurring sequence (e.g., represented by a polynucleotide) or a characteristic portion thereof, or a complement of a naturally occurring sequence or a characteristic portion thereof. In some embodiments, a SSB element can be or comprise one or more engineered (i.e., synthetic) nucleotides or characteristic portion(s) thereof. In some such embodiments, an engineered sequence (e.g., a sequence substantially composed of synthetic or engineered nucleotides) is analogous or corresponds to a naturally occurring sequence; however, any given engineered sequence is “produced by the hand of man.”

In some embodiments SSB elements can include one or more of Zinc Finger proteins or domains, TALE-proteins or domains, Helix-loop-helix proteins or domains, Helix-turn-helix proteins or domains, Cas-proteins or domains (e.g., Cas9, dCas9, etc.), Leucine Zipper proteins or domains, beta-scaffold proteins or domains, Homeo-domain proteins or domains, High-mobility group box proteins or domains or characteristic portions thereof or combinations and/or parts thereof.

In some embodiments, a SSB element is or comprises, for example, zinc-finger proteins, leucine zipper proteins or domains, or other nucleotide (e.g., DNA) binding proteins. By way of non-limiting example, a SSB element may be or comprise one or more Zinc Finger proteins or domains; TALE-proteins or domains; Helix-loop-helix proteins or domains; Helix-turn-helix proteins or domains; CAS-proteins or domains; Leucine Zipper proteins or domains; beta-scaffold proteins or domains; Homeo-domain proteins or domains; High-mobility group box proteins or domains or characteristic portions thereof or combinations and/or parts thereof. In some embodiments, a SSB element is or comprises a non-bacterial or non-viral polynucleotide binding domain. In some embodiments, a SSB element is or comprises a mammalian polynucleotide binding domain.

The present disclosure encompasses a recognition that SSB elements can be designed to target any desired polynucleotide sequence.

In some embodiments, a SSB element may be or comprise more than seven zinc finger modules. As will be understood by those of skill in the art, working with and using zinc finger arrays can present several technological and methodological challenges. By way of non-limiting example, the present disclosure provides a SSB-HbW agent, wherein the SSB element comprises nine zinc finger modules (see, e.g., Example 2). In some embodiments, such a SSB-HbW agent is used to successfully modify genetic material in a cell (e.g., a base change in a target sequence of a cell).

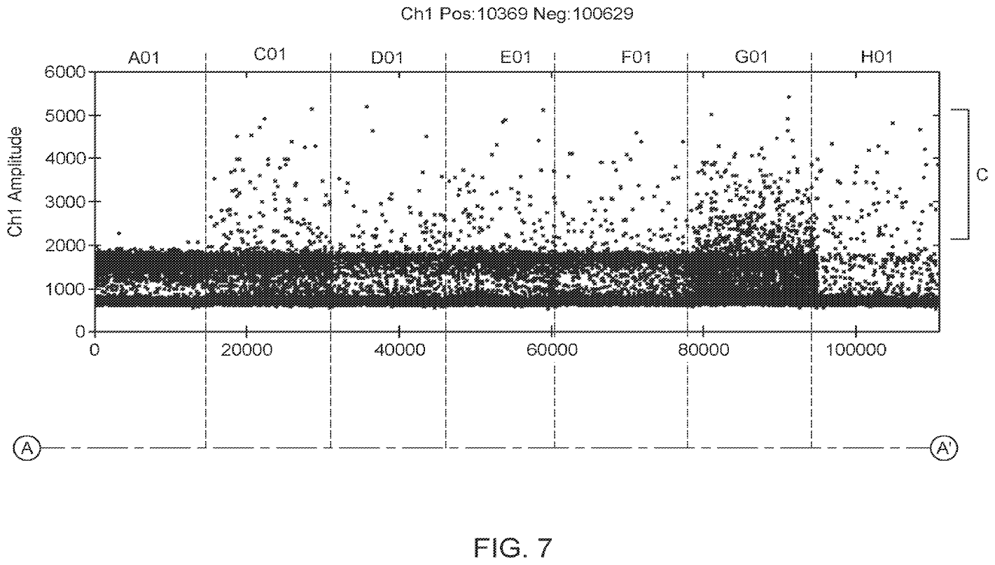

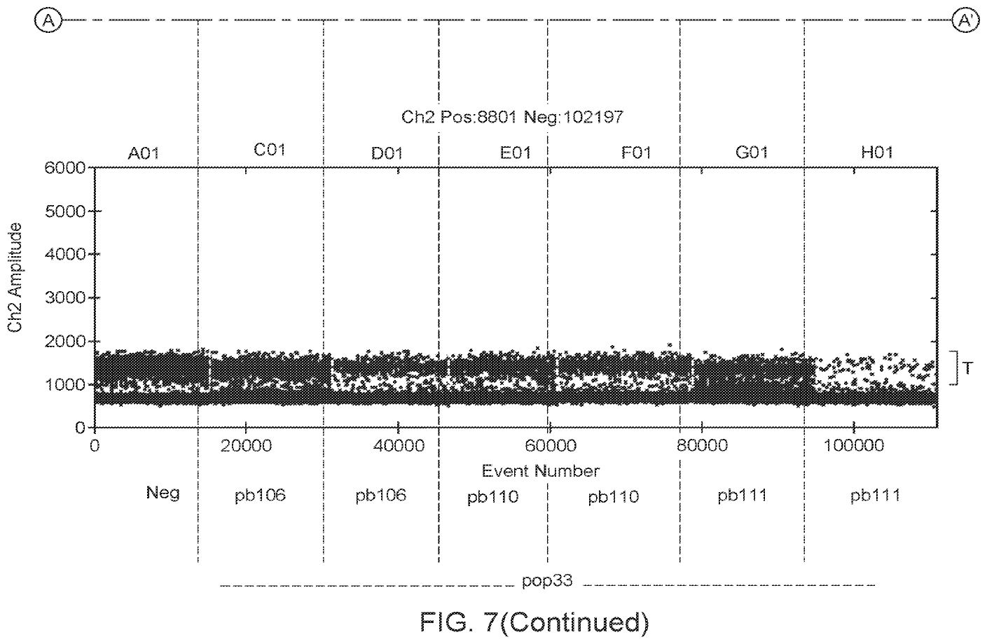

In some embodiments, a SSB element is or comprises a sequence specific recognition element. In some such embodiments, a SSB element can be designed to not only recognize a specific sequence, but also to bind to that specific sequence within a context of a certain genome. For example, in some embodiments, a SSB element is or comprises an array of 4 zinc-finger modules, each of which is designed to recognize a 3-nucleotide sequence (see, e.g., FIG. 7). For example, in some such embodiments a target site is a 12-nucleotide sequence.

In some embodiments, a designed binding sequence (e.g., a sequence that binds to, e.g., a binding site and/or a landing site) can range from 9 nucleotides (e.g., when using 3 zinc finger domains) to larger than 33 nucleotides in length (e.g., using 11 or more zinc-finger modules). In some embodiments, a SSB element can be or comprise a designed zinc finger array, containing a number of zinc fingers (e.g., 1, 2, 3, 4, 5, 6, 7, 8, 9, 10, 11, 12, 13, 14, 15, etc.), wherein each zinc finger is designed to recognize and bind three consecutive nucleotides. For example, if a target site (e.g., on a target molecule, e.g., a target DNA strand, on RNA molecule e.g., an RNA molecule with loop structure and base pairing, etc.) is 9 bp in length, a SSB element can be designed to be or comprise three zinc finger arrays. If, for example, a target site is 33 bp in length, then a SSB element can be designed to be or comprise eleven zinc fingers.

In some embodiments, a SSB element is or comprises a sequence specific DNA recognition element that is engineered not only to recognize a specific sequence, but also to bind to that specific DNA sequence (e.g., target site) with sufficient affinity.

Without being bound by any particular theory, the present disclosure considers that, in some embodiments, a dissociation constant of 10 E−6 (M) or lower may confer sufficient binding strength for a given SSB element to bind and/or stay bound to a particular sequence. In some embodiments, a SSB element binds to a target sequence with affinity characterized by a dissociation constant of at least 10 E−6 M, 10 E−7 M, 10 E−8 M, 10 E−9 M, 10 E−10 M, 10 E−11 M, 10 E−12 M, 10 E−13 M, 10 E−14 M or 10 E−15 M.

In some embodiments, a SSB element can also be or comprise naturally occurring or designed factors with ability to provide both sequence specific recognition and binding. For example, In some embodiments, a SSB element can be or comprise a helix-loop-helix proteins or domains; helix-turn-helix proteins or domains; leucine zipper proteins or domains; beta-scaffold proteins or domains; homeo-domain proteins or domains; high-mobility group box proteins or domains or characteristic portions thereof or combinations and/or parts thereof, etc.

In some embodiments, a SSB-HbW agent may be encoded in, e.g., DNA, RNA, chemically modified, and/or or synthetic nucleotides. In some embodiments, a given SSB-HbW agent can be or comprise a SSB element at the 5′ end or at the 3′ end of a given molecule.

In some embodiments, SSB elements are binding elements that are typically folded macromolecules that adapt a 3D structure that recognizes a double or single-stranded polynucleotide (e.g., a DNA molecule). In some embodiments, a sequence recognized by a SSB element is at least 9 nucleotides in length.

In some embodiments, SSB elements can be engineered or designed such that a polynucleotide (e.g., DNA) recognition sequence is different from that of an original or a naturally occurring polynucleotide (e.g., DNA) binding element. In some embodiments, a SSB element can be designed such that it binds with higher affinity and/or selectivity to a sequence that is, in at least one nucleotide, changed compared to an original polynucleotide binding sequence. In some embodiments, a SSB element can be engineered, designed or selected to recognize a specific sequence (e.g., a DNA sequence, an RNA sequence, e.g., an mRNA sequence, etc.). In some embodiments, a SSB element can be designed, engineered and/or selected to have high or low binding affinity for a specific sequence (e.g., a target sequence, e.g., a DNA sequence, an RNA sequence, etc.). In some embodiments, a SSB element can be designed, engineered and/or selected to have high or low affinity for non-sequence specific DNA binding. In some embodiments, binding affinity can be measured in vitro, mimicking conditions that are similar to in vivo conditions in a cell. In some embodiments, binding affinity and/or selectivity can be measured in vitro using assays known to those of skill in the art such as e.g., DNA-protein interaction assays. In some embodiments, sequence selectivity can be measured in vitro, mimicking conditions that are similar to in vivo conditions in a cell. In some embodiments, affinity and selectivity can be measured in vivo using reporter-assays typical for DNA-protein interactions.

In some embodiments, a sequence recognized by a SSB element is or comprises a sequence between about 5 to about 40 nucleotides. In some embodiments, a sequence recognized by a SSB element is or comprises a sequence between about 5-10, 10-15, 15-20, 20-25, 25-30, 30-35, 35-40 or more nucleotides. In some embodiments, number of nucleotides involved in specificity may occur in groups of three (e.g., in zinc finger contexts, e.g., 9, 12, 15, 18, 21, 24, 27, 30, 33 or more nucleotides of specificity with each three nucleotides corresponding to one zinc finger). In some embodiments, sequence-specificity of a SSB element has approximately at least 15-20 nucleotides of specificity. In some embodiments, a SSB element has at least about 9, 10, 11, 12, 13, 14, 15, 16, 17, 18, 19, 20, 21, 22, 23, 24, 25, 26, 27, 28, 29, 30, 31, 32, 33 nucleotides of specificity (i.e., nucleotides of complementarity with a binding site target). In some such embodiments, nucleotides that are involved in sequence specificity do not need to be contiguous with one another; that is, in some embodiments, even if a SSB element has, e.g., 18 nucleotides of specificity with which it recognizes where to bind, those 18 nucleotides are not necessarily contiguous with one another. As will be understood to those of skill in the art and dependent upon context, in some embodiments, it may be desirable to design longer recognition sequences (e.g., longer than 15-20 nucleotides).

### Zinc Finger-Based SSB Elements

Zinc finger proteins have been studied extensively. A large number of naturally occurring proteins containing zinc fingers exist in nature. In many of these proteins zinc fingers are involved in some type of interaction with nucleic acids and/or other proteins. Protein chemistry and crystal structure experiments have elucidated many aspects of zinc finger structures and mechanisms by which they can bind to other molecules. An archetypical zinc finger structure that is often involved in DNA binding and DNA sequence recognition, comprises an alpha-helix structure with two anti-parallel beta-sheets that are oriented into a three-dimensional confirmation by a coordinating zinc atom. In these structures said zinc-atom interacts with cysteine and/or histidine amino acid side chains. Specific amino acid side chains protrude from an alpha helix structure and these amino acids side chains are involved in (preferential) sequence specific binding (Choo and Klug, 1994, Proc Natl Acad Sci USA 91 11163-11167, Elrod-Erickson, et al., 1996, Structure 4 1171-1180, each of which is herein incorporated by reference in its entirety).

In some embodiments, zinc finger proteins have an ability to be used as modular units of approximately 30 amino acids, with each unit potentially able to bind to a DNA-triplet sequence. In some embodiments, zinc finger proteins can been combined into arrays of two or more zinc fingers, thus allowing for larger DNA sequences (i.e., additional DNA triplets) to be recognized and bound by Zn fingers/Zn-containing proteins (Choo and Klug, 1994, Proc Natl Acad Sci USA 91 11168-11172, which is herein incorporated by reference in its entirety).

Many sequence specific interactions between zinc fingers and DNA are known in the art. A number of studies have described how specific amino acid side chains in specific positions of alpha helices of zinc fingers allow for either more- or less-specific interactions and binding to specific nucleotides in a DNA molecule (Klug, 2010, Annu Rev Biochem 79 213-231, which is herein incorporated by reference in its entirety). Accordingly, such features may be incorporated when designing zinc finger units or zinc finger containing domains. Thus, in some embodiments, the present disclosure provides agents that incorporate zinc fingers and/or one or more features of zinc fingers that can be used to design or develop agents or approaches that preferentially recognize specific DNA sequences (Choo and Klu, 1997, Curr Opin Struct Biol 7 117-125; Klug, 2005, Proc. Japan Acad. 81 87-102; Sera and Uranga, 2002, Biochemistry 41 7074-7081, Zhu, et al. 2013. Nucleic Acids Res 41 2455-2465, each of which is herein incorporated by reference in its entirety).

In some embodiments, zinc fingers can influence behavior of adjacent zinc fingers. Accordingly, a series of preselected and pretested zinc finger dimers have been described (Isalan, et al. 1997. Proc Natl Acad Sci USA 94 5617-5621; Moore, et al, 2001, Proc Natl Acad Sci USA 98 1437-1441, each of which is herein incorporated by reference in its entirety) and a number of methods for the evaluation of interactions can be found in literature (Isalan, et al, 1998, Biochemistry 37 12026-12033, which is incorporated by reference in its entirety). Thus, in some embodiments, when designing or selecting zinc finger arrays for use in one or more technologies of the present disclosure, such interactions, dimers, and/or methods can be taken into consideration. The present disclosure also recognizes that zinc finger array design principles as are known in the art may not always be sufficient to accurately predict how well a given zinc finger array will work for a given purposes (e.g., as a SSB element of a SSB-HbW agent used as a DNA strand separation molecule for sequence modification). Accordingly, among other things, the present disclosure provides agents and assays that may be used to design, evaluate and optimize zinc finger arrays for use in accordance with the present disclosure.

In some embodiments, cysteine and/or histidine amino acid side-chains interact with the zinc atom. Zinc finger structure can function, amongst others, in protein-DNA interaction. As illustration, in some embodiments specific zinc finger amino acid side-chains may interact with DNA or other polynucleotides. In some embodiments, zinc finger-DNA interactions can be dependent on a DNA nucleotide sequence, in other embodiments interactions may be non-sequence specific, e.g. as illustration by interacting with a DNA backbone. In some embodiments, zinc finger motifs comprise an alpha helix. In some such embodiments, specific amino acids comprised in an alpha helix may interact preferentially with specific DNA nucleotides. As illustration, in some embodiments, amino acid positions in a zinc finger alpha helix may be numbered. In some embodiments, specific amino acids at specific alpha helix positions may have a preferential binding to a specific nucleotide (A, C, G or T) in a DNA molecule. As illustration, an arginine amino acid at position +6 in a zinc finger alpha helix may preferentially bind to a G-nucleotide in a DNA target sequence.

In some embodiments, a polynucleotide modification agent described herein includes a SSB element including at least five, six, seven, eight, nine, ten, or eleven zinc finger arrays. In some embodiments, a SSB element comprises one, two, three, or four zinc finger arrays. In some embodiments, a SSB element comprises 11, 12, 13, 14, 15, 16, 17, 18, 19, 20, 21, 22, 23, 24, 25, 26, 27, 28, 29, 30, 31, 32, 33, 34, 35, 36, 37, 38, or 39 zinc finger arrays.

In some embodiments, a SSB element includes at least one zinc finger array that includes at least one alpha helix engineered to include a modified amino acid sequence that differs from that of its corresponding wild type sequence. For example, in some embodiments, a zinc finger array comprises (i) one amino acid substitution mutation at a position selected from −1, +1, +2, +3, +4, +5, or +6 in the alpha helix; (ii) two amino acid substitution mutations at positions selected from −1, +1, +2, +3, +4, +5, or +6 in the alpha helix; (iii) three amino acid substitution mutations at positions selected from −1, +1, +2, +3, +4, +5, or +6 in the alpha helix; (iv) four amino acid substitution mutations at positions selected from −1, +1, +2, +3, +4, +5, or +6 in the alpha helix (v) five amino acid substitution mutation at positions selected from −1, +1, +2, +3, +4, +5, or +6 in the alpha helix; (vi) six amino acid substitution mutations at positions selected from −1, +1, +2, +3, +4, +5, or +6 in the alpha helix; or (vii) an amino acid substitution mutation at each position in the alpha helix, wherein the one or more amino acid substitutions in the alpha helix differ from that of its corresponding wild type sequence.

In some embodiments, a zinc finger array as described herein comprises zinc finger amino acid sequences: FQCRICMRNFS(X7)HIRTH (SEQ ID NO: 5) or FACDICGRKFA(X7)HTKIH (SEQ ID NO: 6). In some such embodiments, X7 represents a sequence of seven amino acids, wherein X can be any amino acids, which can be modified to enable (preferential) sequence specific binding to a specific DNA target sequence.

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 5 or 6.

In some embodiments, a SSB element includes at least four zinc finger arrays, and further includes comprises a sequence between the zinc finger arrays. In some embodiments, a zinc finger linker between two zinc finger segments comprises a “canonical” five amino acid sequence TGEKP (SEQ ID NO: 14). In some embodiments, a zinc finger linker contains the same amino acid sequence as present in a human zinc finger protein. In some embodiments, a zinc finger linker may be designed to be different from a naturally present linker. In some embodiments, a zinc finger linker between two zinc finger segments comprises a partially homologous linker sequence TGSQKP (SEQ ID NO: 15). In some embodiments, a zinc finger linker may be a variation of a five amino acid canonical linker, e.g. TGERP (SEQ ID NO: 28), or TGDKP (SEQ ID NO: 29) or TGQKP (SEQ ID NO: 30). In some embodiments, a zinc finger linker may comprise six amino acids or more. In some embodiments, a zinc finger linker may be a non-canonical linker. In some embodiments, a zinc finger linker comprises an amino acid sequence of FVGQQLK (SEQ ID NO: 31), ACQKPFE (SEQ ID NO: 32), AEERPYK (SEQ ID NO: 33), TKEKPYQ (SEQ ID NO: 34), NAKKSYQ (SEQ ID NO: 35), TGQKPFQ (SEQ ID NO. 36), TGEKPYK (SEQ ID NO: 37), TGKRAYE (SEQ ID NO. 38), TGEKPYE (SEQ ID NO: 39), SGERTYR (SEQ ID NO: 40), and/or TGQKPYG (SEQ ID NO: 41).

In some embodiments, a SSB element includes a naturally occurring zinc finger amino acid sequence: FQCRICMRNFSRSDHLTTHIRTH (SEQ ID NO: 16). In some embodiments, a SSB element includes an amino acid sequence: FQCRICMRNFSRSSALTRHIRTH (SEQ ID NO: 17). In some embodiments, a SSB element includes an exemplary human zinc finger array sequence as provided in Table 1, wherein underlined amino acids indicated Cys and His residues that coordinate zinc finger residues, bolded amino acids indicate the alpha helix, and the space indicates one or more intervening amino acids.

TABLE 1
Exemplary human zinc finger arrays

| SEQ ID NO: | Sequence |
|---|---|
| 42 | CNACRKLFS SKSRLQSHLRRH |
| 43 | CHSCGRAFGE KWKLDKHQKTH |
| 44 | CENCGNAYK QKSNLFQHQKMH |
| 45 | CKTCGKAFS WKSSCINHEKIH |
| 46 | CNECEKSER QNSTLIQHKKVH |
| 47 | CTDCGKAFI YKSDLVKHQRIH |
| 48 | CSICEKAFS QKSNVIDHEKIH |
| 49 | CDLCGNTFI QKKNLIQHKKIH |
| 50 | CNRCGKAFF QKSNLHSHQKTH |
| 51 | CSECGKTFI RKLNLSLHKKTH |
| 52 | CSECGKAFA DRSYLVRHQKRIHSR |

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 16, 17 and 42-52.

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NOs: 5 or 6. In some embodiments, a sequence-specific binding element further comprises a zinc finger linker sequence between zinc finger arrays comprising a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NO: 14-15 and 28-41.

In some embodiments, a sequence-specific binding element comprises at least four, five, six, seven, eight, nine, ten, or eleven, or more, zinc finger arrays, and where each zinc finger array comprises a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NOs: 16-17 and 42-52. In some embodiments, a sequence-specific binding element further comprises a zinc finger linker sequence between zinc finger arrays comprising a polypeptide sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in any one of SEQ ID NO: 14-15 and 28-41.

In some embodiments, a sequence-specific binding element comprises a zinc finger array that targets a sequence in a gene that is involved or being studied for its role in a disease or condition. In some embodiments, a sequence-specific binding element comprises a zinc finger array that targets a sequence in a human gene that is involved or being studied for its role in a disease or condition in humans. In some embodiments, a sequence-specific binding element comprises a zinc finger array that targets a sequence in a gene that is involved or being studied for its role in cancer, an inborn error of metabolism, a metabolic disorder, an autoimmune disease, an immunodeficiency, cystic fibrosis, hemophilia, sickle cell anemia, Huntington's disease, muscular dystrophy, a neurodegenerative disease, blindness or other ocular disease, congenital lung disease, among others.

In some embodiments, a SSB element comprises a zinc finger array that targets a sequence of a gene selected from B-cell lymphoma/leukemia 11 A (BCL11 A) gene, a dystrophin gene (DMD), metabolism of cobalamin associated C (MMACHC), a DNA polymerase γ gene (PolG), a methylmalonyl CoA mutase gene (MMUT), a phenylalanine hydroxylase gene (PAH), a cystic fibrosis transmembrane conductance regulator (CFTR) gene, a Kruppel-like factor 1 gene, a mammalian beta globin gene, a mammalian gamma globin gene, a C—C chemokine receptor type (CCR)5 gene, a chemokine (C—X—C motif) receptor 4 (CXCR4) gene, a protein phosphatase 1 regulatory subunit 12C (PPP1R12C) gene, an hypoxanthine phosphoribosyltransferase (HPRT) gene, an albumin gene, a Factor VIII gene, a Factor IX gene, a leucine-rich repeat kinase 2 (LRRK2) gene, a huntingtin (Htt) gene, a rhodopsin (RHO) gene, a surfactant protein B gene (SFTPB), a T-cell receptor alpha (TRAC) gene, a T-cell receptor beta (TRBC) gene, a programmed cell death 1 (PD1) gene, a cytotoxic T-lymphocyte antigen 4 (CTLA-4) gene, an human leukocyte antigen (HLA) A gene, an HLA B gene, an HLA C gene, an HLA-DPA gene, an HLA-DQ gene, an HLA-DRA gene, a Low-molecular mass protein-7 (LMP7) gene, a Transporter associated with antigen processing (TAP)1 gene, a TAP2 gene, a tapasin gene (TAPBP), a class II major histocompatibility complex transactivator (CUTA) gene, a glucocorticoid receptor gene (GR), an interleukin 2 receptor gamma (IL2RG) gene. and a regulatory factor X 5 (RFX5) gene.

In some certain embodiments, a SSB element comprising a zinc finger array targets a sequence of a gene selected from: EGFPDP2, ApoE, Bcl11A, DMD, PolG, MMACHC, MMUT, PAH, CFTR, MMA, and PKU. In some embodiments, a SSB element comprises a zinc finger protein comprising a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in Table 2 below.

TABLE 2
Exemplary human zinc finger protein sequences

| SEQ | | |
|---|---|---|
| ID | | |
| NO: | Target | Amino Acid Sequence |
| 19 | EGFPDP2 | FQCRICMRNFSRSSALTRHIRTHTGEKPFACDICGRKFARSDTLTRHTKIHTGSQKPFQCR |
| | | ICMRNFSDRSNLTRHIRTHTGEKPFACDICGRKFARSDNLTRHTKIHTGSQKPFQCRICMR |
| | | NFSRSDHLTRHIRTHTG |
| 21 | EGFPDP2 | FQCRICMRNFSDRSNLTRHIRTHTGEKPFACDICGRKFARSDHLTRHTKIHTGSQKPFQCR |
| | | ICMRNFSDRSNLTRHIRTHTGEKPFACDICGRKFARSDSLSEHTKIHTGSQKPFQCRICMR |
| | | NFSRSSNLTRHIRTHTGEKPFACDICGRKFARSDSLTRHTKIH |
| 4 | ApoE | MAAMAERPFQCRICMRNFSRSSDLTRHIRTHTGEKPFACDICGRKFARSDTLTRHTKIHTG |
| | | SQKPFQCRICMRNFSQSGDLSEHIRTHTGEKPFACDICGRKFATSGHLTTHTKIHTGSQKP |
| | | FQCRICMRNFSDSSHLTTHIRTHTGEKPFACDICGRKFARSSHLTTHTKIHTGSQKPFQCR |
| | | ICMRNFSDRSDLTRHIRTHTGEKPFACDICGRKFADRSDLTRHTKIHTGSQKPFQCRICMR |
| | | NFSRSDTLTRHIRTHTG |
| 23 | ApoE | FQCRICMRNFSRSSDLTRHIRTHTGEKPFACDICGRKFARSDTLTRHTKIHTGSQKPFQCR |
| | | ICMRNFSQSGDLSEHIRTHTGEKPFACDICGRKFATSGHLTTHTKIHTGSQKPFQCRICMR |
| | | NFSDSSHLTTHIRTHTGEKPFACDICGRKFARSSHLTTHTKIHTGSQKPFQCRICMRNFSD |
| | | RSDLTRHIRTHTGEKPFACDICGRKFADRSDLTRHTKIHTGSQKPFQCRICMRNFSRSDTL |
| | | TRHIRTHTG |
| 25 | ApoE | MAAMAERPFQCRICMRNFSDRSHLTRHIRTHTGEKPFACDICGRKFARSDNLTRHTKIHTG |
| | | SQKPFQCRICMRNFSDSSHLSEHIRTHTGEKPFACDICGRKFADRSDLTRHTKIHTGSQKP |
| | | FQCRICMRNFSRSDHLTRHIRTHTGEKPFACDICGRKFADRSDLTRHTKIHTGSQKPFQCR |
| | | ICMRNFSRSDNLSEHIRTHTGEKPFACDICGRKFAESSNLTTHTKIHTGSQKPFQCRICMR |
| | | NFSRSSSLTRHIRTHTGEKPFACDICGRKFAQSSDLTRHTKIHTGSQKPFQCRICMRNFSR |
| | | SDSLSEHIRTHTG |
| 70 | Bcl11A | MAAMAERPFQCRICMRNFSRSSNLTRHIRTHTGEKPFACDICGRKFARSDALSEHTKIHTG |
| | | SQKPFQCRICMRNFSDSSALTTHIRTHTGEKPFACDICGRKFADSSDLSEHTKIHTGSQKP |
| | | FQCRICMRNFSQSGNLSQHIRTHTGEKPFACDICGRKFADRSDLTRHTKIHTGSQKPFQCR |
| | | ICMRNFSRSDNLTRHIRTHTG |
| 78 | PolG | MAAMAERPFQCRICMRNFSTSGNLTRHIRTHTGEKPFACDICGRKFARSDALSQHTKIHTG |
| | | SQKPFQCRICMRNFSRSSALTTHIRTHTGEKPFACDICGRKFARSSDLTTHTKIHTGSQKP |
| | | FQCRICMRNFSRSSNLSQHIRTHTGEKPFACDICGRKFARSSNLSQHTKIHTGSQKPFQCR |
| | | ICMRNFSRSSALSQHIRTHTGEKPFACDICGRKFARSSNLTRHTKIHTGSQKPFQCRICMR |
| | | NFSRSDHLSEHIRTHTG |
| 86 | MMACHC | MAAMAERPFACDICGRKFADDRSHLTRHTKIHTGSQKPFQCRICMRNFSRSDALSEHIRTH |
| | | TGEKPFACDICGRKFATSGNLSEHTKIHTGSQKPFQCRICMRNFSDSSNLTTHIRTHTGEK |
| | | PFACDICGRKFADRSDLTRHTKIHTGSQKPFQCRICMRNFSRSDALTRHIRTHTGEKPFAC |
| | | DICGRKFATSGHLTTHTKIHTGSQKPFQCRICMRNFSRSSNLSEHIRTHTGEKPFACDICG |
| | | RKFADRSNLTRHTKIHTGSQKPFQCRICMRNFSRSDALTRHIRTHTG |
| 94 | MMUT | MAAMAERPFACDICGRKFADRSSHLTRHTKIHTGSQKPFQCRICMRNFSRSDNLTRHIRTH |
| | | TGEKPFACDICGRKFARSDALTRHTKIHTGSQKPFQCRICMRNFSTSGHLTTHIRTHTGEK |
| | | PFACDICGRKFAQSGDLTTHTKIHTGSQKPFQCRICMRNFSRSSALTTHIRTHTGEKPFAC |
| | | DICGRKFADSSALTTHTKIHTGSQKPFQCRICMRNFSQSGSLSQHIRTHTGEKPFACDICG |
| | | RKFARSSNLSEHTKIHTGSQKPFQCRICMRNFSDSSHLTRHIRTHTGEKPFACDICGRKFA |
| | | DRSNLTRHTKIHTGSQKPFQCRICMRNFSRSDALTTHIRTHTG |
| 102 | PAH | MAAMAERPFQCRICMRNFSTSGDLSQHIRTHTGEKPFACDICGRKFAQSGHLTRHTKIHTG |
| | | SQKPFQCRICMRNFSQSGSLTTHIRTHTGEKPFACDICGRKFADRSALTRHTKIHTGSQKP |
| | | FQCRICMRNFSRSDSLTTHIRTHTGEKPFACDICGRKFATSGALTRHTKIHTGSQKPFQCR |
| | | ICMRNFSRSDSLTRHIRTHTG |
| 110 | CFTR | MAAMAERPFACDICGRKFADQSGNLTRHTKIHTGSQKPFQCRICMRNFSRSDNLSEHIRTH |
| | | TGEKPFACDICGRKFADSSALSQHTKIHTGSQKPFQCRICMRNFSQSGSLSQHIRTHTGEK |
| | | PFACDICGRKFADRSHLTRHTKIHTGSQKPFQCRICMRNFSQSGDLSEHIRTHTGEKPFAC |
| | | DICGRKFARSSALTRHTKIHTGSQKPFQCRICMRNFSRSDSLSQHIRTHTG |
| 122 | DMD | MAAMAERPFACDICGRKFADQSGNLTRHTKIHTGSQKPFQCRICMRNFSRSDNLSQHIRTH |
| | | TGEKPFACDICGRKFATSGDLSQHTKIHTGSQKPFQCRICMRNFSTSGSLTRHIRTHTGEK |
| | | PFACDICGRKFARSDALTRHTKIHTGSQKPFQCRICMRNFSTSGDLSEHIRTHTGEKPFAC |
| | | DICGRKFAQSGNLSEHTKIHTGSQKPFQCRICMRNFSQSGDLSQHIRTHTGEKPFACDICG |
| | | RKFARSSALTRHTKIHTGSQKPFQCRICMRNFSRSDALSEHIRTHTG |

In some embodiments, a SSB element comprising a zinc finger array targets a sequence of EGFPDP2. For example, in some embodiments, a target sequence 5′-GGGGAGGACGCGGTG-3′ (SEQ ID NO: 18) of EGFPDP2 is targeted by a zinc finger array that comprises a zinc finger protein sequence of SEQ ID NO: 19. In some embodiments, a SSB element targets EGFPDP2 and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 19.

In some embodiments, a target sequence 5′-GTGGAGCTGGACGGGGAC-3′ (SEQ ID NO: 20) of EGFPDP2 is targeted by a zinc finger array that comprises a zinc finger protein sequence of SEQ ID NO: 21. In some embodiments, a SSB element targets EGFPDP2 and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 21.

In some embodiments, a SSB element comprising a zinc finger array targets a sequence of human ApoE (e.g., at codon 112). For example, in some embodiments, a target sequence 5′-GCGGCCGCCTGGTGCAGTACCGCGGCG-3′ (SEQ ID NO: 22) of ApoE is targeted by a zinc finger array that comprises a zinc finger protein sequence of SEQ ID NO: 23. In some embodiments, a SSB element targets ApoE and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 23.

In some embodiments, a SSB element targets ApoE and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 4.

In some embodiments, a SSB element comprising a zinc finger array targets a sequence of human ApoE (e.g., at codon 158). For example, in some embodiments, a target sequence 5′-CTGGCAGTGTACCAGGCCGGGGCCCGCGAGGGC-3′ (SEQ ID NO: 24) of ApoE is targeted by a zinc finger array that comprises a zinc finger protein sequence of SEQ ID NO: 25. In some embodiments, a SSB element targets ApoE and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 25.

In some embodiments, a SSB element comprising a zinc finger array targets a sequence of Bcl11A. For example, in some embodiments, a target sequence 5′-GAGGCCAAACCCTTCCTGGAG-3′ (SEQ ID NO: 64) of Bcl11A is targeted by a zinc finger array that comprises a zinc finger protein sequence of SEQ ID NO: 70. In some embodiments, a SSB element targets Bcl11A and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 70.

In some embodiments, a SSB element comprising a zinc finger array targets a sequence of PolG. For example, in some embodiments, a target sequence 5′-CGGGAGATGAAGAAGTCGTTGATGGAT-3′ (SEQ ID NO: 71) of PolG is targeted by a zinc finger array that comprises a zinc finger protein sequence of SEQ ID NO: 78. In some embodiments, a SSB element targets PolG and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 78.

In some embodiments, a SSB element comprising a zinc finger array targets a sequence of MMACHC. For example, in some embodiments, a target sequence 5′-GTGGACCAGTGTGTGGCCTACCATCTGGGC-3′ (SEQ ID NO: 79) of MMACHC is targeted by a zinc finger array that comprises a zinc finger protein sequence of SEQ ID NO: 86. In some embodiments, a SSB element targets MMACHC and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 86.

In some embodiments, a SSB element comprising a zinc finger array targets a sequence of MMUT. For example, in some embodiments, a target sequence 5′-TTGGACGGCCAGATATTCTTGTCATGTGTGGAGGGG-3′ (SEQ ID NO: 87) of MMUT is targeted by a zinc finger array that comprises a zinc finger protein sequence of SEQ ID NO: 94. In some embodiments, a SSB element targets MMUT and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 94.

In some embodiments, a SSB element comprising a zinc finger array targets a sequence of PAH. For example, in some embodiments, a target sequence 5′-GTGGTTTTGGTTTAGGAACT-3′ (SEQ ID NO: 95) of PAH is targeted by a zinc finger array that comprises a zinc finger protein sequence of SEQ ID NO: 102. In some embodiments, a SSB element targets PAH and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 102.

In some embodiments, a SSB element comprising a zinc finger array targets a sequence of CFTR. For example, in some embodiments, a target sequence 5′-ATGGTGCCAGGCATAATCCAGGAA-3′ (SEQ ID NO: 103) of CFTR is targeted by a zinc finger array that comprises a zinc finger protein sequence of SEQ ID NO: 110. In some embodiments, a SSB element targets CFTR and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 110.

In some embodiments, a SSB element comprising a zinc finger array targets a sequence of DMD. For example, in some embodiments, a target sequence 5′-CTGGTGACACAACCTGTGGTTACTAAGGAA-3′ (SEQ ID NO: 119) of DMD is targeted by a zinc finger array that comprises a zinc finger protein sequence of SEQ ID NO: 122. In some embodiments, a SSB element targets DMD and comprises a zinc finger protein sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence of SEQ ID NO: 122.

### Other Sequence Specific Binding Elements

The present disclosure contemplates that in some embodiments, in addition to zinc fingers, a number of other proteins, protein domains and designed proteins exist or can be developed for use as part of or as sequence specific binding elements (e.g., DNA sequence specific binding domains). These include but are not limited to helix-loop-helix proteins or domains, helix-turn-helix proteins or domains, homeo-domain proteins or domains, beta-scaffold proteins or domains, high-mobility group box proteins or domains, leucine zipper proteins or domains and other types of naturally occurring and/or designed proteins and any combinations thereof.

In some embodiments, a polynucleotide (e.g., DNA) binding element needs to be of sufficient size and structure to recognize and bind to a desired sequence. For example, in some embodiments, within a context of genome editing a binding element sequence is specific within the genome of a target organism. In some embodiments, a binding element sequence is semi-specific for the genome of a target organism; for example, to be semi-specific, in some embodiments, a mammalian cell requires a sequence of at least 15 nucleotides of homology, but preferentially a larger number. That is, specificity of a given SSB-HbW agent may be combinatorial and can come from one or more sequence-specific components of the molecule (e.g., a SSB element, a SSB element and an HbW element, etc.).

### Helicases

Helicase enzymes have been studied extensively. A large number of naturally occurring helicases exist in nature. Helicases are involved in DNA replication, having a special role in strand separation of double-stranded DNA molecules and other processes involving strand separation of double stranded polynucleotides. Protein chemistry and crystal structure experiments have elucidated many aspects of helicase structures and mechanisms by which they can break hydrogen bonding of nucleobases. As examples, human BLM helicase, human WRN helicase and human RECQ1 helicase comprise a structure that is involved in DNA strand separation, comprising a “beta-wing” structure, referred to herein as a helicase beta wing (“HbW”) element or domain. In some embodiments, a HbW element or domain comprises an anti-parallel beta-sheet. In crystal structures of these helicase bound to DNA, said “beta-wing” structures are observed at strand separated DNA loci.

In some embodiments, technologies described herein include a portion of a helicase that is involved in strand separation of a polynucleotide, referred to herein as a “helicase beta-wing element” or “HbW element”. In some embodiments an HbW element comprises a “beta-wing” sequence or structure. In some embodiments an HbW-element interacts with a DNA molecule to which a SSB element is bound. In some embodiments an HbW element results in breaking of hydrogen bonds within a specific polynucleotide close to a particular sequence, which is also referred to herein as a “target site.” In some embodiments, a HbW domain results in strand separation in a polynucleotide. In some such embodiments, such strand separations can involve a single basepair or a multitude of basepairs. In some embodiments, an HbW element may interact with DNA, RNA, mRNA, etc. In some embodiments, an HbW element is present within the same molecule as a given SSB element. In some embodiments, an HbW element can be or comprise a naturally occurring sequence or characteristic portion thereof. In some embodiments, an HbW element can be, or comprise an engineered sequence or characteristic portion thereof. In some such embodiments, an engineered sequence is analogous or corresponds to a naturally occurring sequence; however, any given engineered sequence is “produced by the hand of man.”

In some embodiments, HbW elements can be derived from a human helicase or from a helicase from other organisms. Helicase beta-wing structures can be identified, for example by studying crystal structures, amino acid sequence alignments, biochemical experiments and other methods known to those skilled in the art. For example, a crystal structure of WRN helicase identified a beta-wing structure essentially contained in an amino acid sequence: VSRYNKFMKICALTKKG (SEQ ID NO: 1). As another non-limiting example, a crystal structure of BLM Helicase identified a beta-wing structure essentially contained in an amino acid sequence DLYINANDQAIAYVMLG (SEQ ID NO: 2). As another non-limiting example, a crystal structure of RECQ1 Helicase identified a beta-wing structure essentially contained in an amino acid sequence DYSFTAYATISYLKIG (SEQ ID NO: 3). As illustration of amino acid sequence alignment as approach to identify potential beta-wing structures Pike et al (2015) shows amino acid sequence alignments of human helicases BLM, WRN, RECQ1 and bacterial RecQ (Pike et al., Proceedings of the National Academy of Sciences April 2015, 112 (14) 4286-4291, which is hereby incorporated by reference in its entirety).

In some embodiments, a HbW element comprises an amino acid sequence that is at least 80% identical to a sequence of any one of SEQ ID NOs: 1, 2, and 3, and where the HbW element has helicase activity. In some embodiments, a HbW element comprises a sequence that is at least 85% identical to a sequence of any one of SEQ ID NOs: 1, 2, and 3, and where the HbW element has helicase activity. In some embodiments, a HbW element comprises a sequence that is at least 90% identical to a sequence of any one of SEQ ID NOs: 1, 2, and 3, and where the HbW element has helicase activity.

In some embodiments, a HbW element comprises an amino acid sequence with up 4 amino acid substitutions in any one of SEQ ID NOs: 1, 2, and 3, wherein the HbW element has helicase activity. In some embodiments, a HbW element comprises an amino acid sequence with up 3 amino acid substitutions in any one of SEQ ID NOs: 1, 2, and 3, wherein the HbW element has helicase activity. In some embodiments, a HbW element comprises an amino acid sequence with up 2 amino acid substitutions in any one of SEQ ID NOs: 1, 2, and 3, wherein the HbW element has helicase activity. In some embodiments, a HbW element comprises an amino acid sequence with a single amino acid substitution in any one of SEQ ID NOs: 1, 2, and 3, wherein the HbW element has helicase activity.

In some embodiments, a HbW element comprises an amino acid sequence of any one of SEQ ID NOs: 1, 2, and 3. In some embodiments, a HbW element consists of an amino acid sequence of any one of SEQ ID NOs: 1, 2, and 3.

In some embodiments, HbW elements are derived from naturally occurring helicases. In some embodiments, a HbW element can comprise a helicase beta-wing derived from a helicase from any source. For example, in some embodiments, a HbW element is or comprises a polypeptide derived from a prokaryotic helicase. In some embodiments, a HbW element is or comprises a polypeptide derived from a eukaryotic helicase.

In some embodiments, HbW elements are derived from naturally occurring mammalian helicases. In some embodiments, HbW elements are derived from naturally occurring human helicases.

In some embodiments, a helicase beta-wing element is or comprises an amino acid sequence of SEQ ID NO: 1, or a truncated version thereof. In some embodiments, the truncated version has helicase activity.

In some embodiments, a helicase beta-wing element is or comprises an amino acid sequence of SEQ ID NO: 2, or a truncated version thereof. In some embodiments, the truncated version has helicase activity.

In some embodiments, a helicase beta-wing element is or comprises an amino acid sequence of SEQ ID NO: 3, or a truncated version thereof. In some embodiments, the truncated version has helicase activity.

In some embodiments, HbW elements are designed or experimentally derived (e.g., through mutagenesis of a naturally occurring beta wing structure or sequence) helicase beta-wing structures or sequences that have immunological properties that avoid an immune reaction by a human host.

In some embodiments, HbW elements are designed or experimentally derived (e.g. through mutagenesis of a naturally occurring beta wing structure or sequence) helicase beta-wing structures or sequences.

In some embodiments, polynucleotide modification agents of the present disclosure comprise an HbW element that interacts with a nucleic acid molecule to which a SSB element is bound. In some such embodiments, a HbW element interacts with a polynucleotide molecule in a non-sequence-specific manner. In some embodiments, a HbW element binds to a polynucleotide in a sequence-specific manner (e.g., sequence specificity of such a polynucleotide modification agent is contributed by both a SSB element and a HbW element).

The present disclosure provides the insight that gene editing may be accomplished without reliance on nuclease activity to introduce breaks into one or more polynucleotide strands to be edited. The present disclosure contemplates that, in some embodiments, other designs of HbW elements are also possible, providing that such designs provide for sufficient DNA strand separation ability and that they have little to no inherent nuclease activity.

### Linkers

The present disclosure also provides linkers or “L elements” useful in the context of provided technologies.

In some embodiments, provided polynucleotide modification agents include a linker or “L element”. In some embodiments, an L element may be optionally used to connect (link) at least one “SSB element” and at least one “HbW element.” In some embodiments, an L element comprises amino acid residues. In some embodiments, provided by the present disclosure, an L element can function as a linker domain between a SSB domain and an HbW domain.

Though the present disclosure generally provides L elements to connect SSB and HbW elements, in some embodiments, L elements may also provide additional properties, such as, e.g., orientation of an entire SSB-HbW molecule. In some embodiments, linkers may be composed of, or comprise sequences present in a human proteome and/or genome. In some embodiments, for instance, an L element may comprise one or more components that confer additional sequence or structure specificity (e.g., addition of an arginine to facilitate binding to G, addition of hydrophobic amino acids, addition of certain polar amino acids, e.g., lysine, which may, in some embodiments, have a greater affinity for a negatively charged molecule (e.g., DNA), etc.)

In some embodiments, an L element is an optional component in a polynucleotide medication agent comprising a SSB element and/or an HbW element.

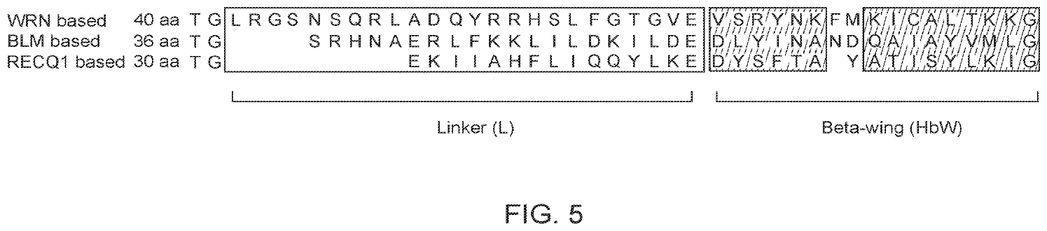

An L element can be an existing, naturally occurring, engineered, designed and/or selected molecule. In some embodiments, a linker sequence is a linker found in nature or analogous to a linker found in nature. In some embodiments, a linker is a sequence comprised in a helicase molecule. In some embodiments, a linker comprises a sequence that is derived from the same molecule as a beta-wing sequence. In some embodiments, a linker is a sequence comprised in a naturally occurring human molecule (see e.g. FIG. 5).

In some embodiments, a linker is, comprises or is a truncated version of a WRN based linker. In some embodiments, a WRN based linker is, comprises or is a truncated version of an amino acid sequence of LRGSNSQRLADQYRRHSLFGTGVE (SEQ ID NO: 7).

In some embodiments, a linker is, comprises or is a truncated version of a BLM based linker. In some embodiments, a BLM based linker is, comprises or is a truncated version of an amino acid sequence of SRHNERLFKKLILDKILDE (SEQ ID NO: 8).

In some embodiments, a linker is, comprises or is a truncated version of a RECQ1 based linker. In some embodiments, a RECQ1 based linker is, comprises or is a truncated version of an amino acid sequence of EKIIAHFLIQQYLKE (SEQ ID NO: 9).

In some embodiments, a linker comprises a sequence that is at least 80% identical to a sequence of any one of SEQ ID NOs: 7, 8, and 9. In some embodiments, a linker comprises a sequence that is at least 85% identical to a sequence of any one of SEQ ID NOs: 7, 8, and 9. In some embodiments, a linker element comprises a sequence that is at least 90% identical to a sequence of any one of SEQ ID NOs: 7, 8, and 9.

In some embodiments, a linker comprises an amino acid sequence with up 4 amino acid substitutions in any one of SEQ ID NOs: 7, 8, and 9. In some embodiments, a linker comprises an amino acid sequence with up 3 amino acid substitutions in any one of SEQ ID NOs: 7, 8, and 9. In some embodiments, linker comprises an amino acid sequence with up 2 amino acid substitutions in any one of SEQ ID NOs: 7, 8, and 9. In some embodiments, a linker comprises an amino acid sequence with a single amino acid substitution in any one of SEQ ID NOs: 7, 8, and 9.

In some embodiments, a linker comprises an amino acid sequence of any one of SEQ ID NOs: 7, 8, and 9. In some embodiments, a linker consists of an amino acid sequence of any one of SEQ ID NOs 7, 8, and 9.

In certain embodiments, when using an amino acid linker this element can be a 4 amino-acid linker (e.g., LRGS as in SEQ ID NO: 26). However, longer or shorter linkers may be used as required on a case-by-case manner.

In some embodiments, an L element is short (e.g., 10 or fewer amino acids) linker. In some such embodiments, a short linker has approximately 7, 6, 5, 4, 3 or 2 amino acids. For example, in some embodiments, a short linker is or comprises an amino acid sequence of LRGS (SEQ ID NO. 26). In some embodiments, a linker may be or comprise a sequence of GGGGSn (SEQ ID NO: 27), wherein n is 1 or 2.

In some embodiments, linkers comprise nucleic acid residues. In some embodiments, a linker is short (e.g., 21, 18, 15, 12, 9, 6 nucleic acids or less). In some such embodiments, a short linker has approximately 21, 18, 15, 12, 9 or fewer nucleic acids. In some embodiments, nucleic acids are modified nucleic acids, e.g., locked nucleic acids, oligonucleotides, etc.

In some embodiments, a linker is a synthetic linker. In some embodiments, a linker comprises a sequence that cannot be found in nature and has no homology to any linker found in nature. In some embodiments, a linker may be or comprise a combination of natural linkers, but arranged in patterns not found in nature, e.g., connecting one or more natural linkers that are not found in such an arrangement in nature, e.g., generating a linker comprising repeats of a natural linker, wherein the linker comprising repeats is not itself found in nature.

The present disclosure illustrates that linkers of different length can be used, and is not intended to limit the length or size of useful linkers. When using amino acid-based linkers, a linker may be of any length and an appropriate length will be known to those of skill in the art and dependent upon context.

In some embodiments, a linker may be flexible, semi-flexible, semi-rigid, or rigid. For example, in some embodiments, a flexible linker may be or comprise an amino acid sequence comprising repeats of a sequence GGGGS (SEQ ID NO: 27), represented by GGGGSn. For example, in some embodiments, an L element may comprise a sequence of GGGGSn, where n may be 1, 2, 3, 4, 5, 6, 7, 8 or more. In certain some embodiments, an L element may comprise a sequence of GGGGSn, where n is 6.

In some embodiments, an L element has no function other than to link one or more SSB elements to one or more HbW elements. In some embodiments, an L element does have a function beyond simply linking (e.g., positioning one or both of a SSB element and/or an HbW element to support a particular application or modification, serving as a site for action of an enhancing agent). In some embodiments, a primary function of an L element is to link a SSB element with an HbW element. In some embodiments, in addition to serving a linker function, an L element may have additional features or functions. For example, in some embodiments, an L element may facilitate or participate in orientation of a given SSB-HbW molecule relative to one or more molecules (e.g., DNA, RNA, etc.) to which it is bound. In some embodiments, such additional features or functions may serve to enhance overall impact or functionality of a given SSB-HbW molecule. In some embodiments, an L element may impact binding strength of a SSB-HbW molecule. For example, in some embodiments, an L element may increase binding strength of a given SSB-HbW molecule. For instance, by way of non-limiting example, if an L element is or comprises one or more basic amino acid residues it may serve to interact more strongly with a negatively charged molecule (e.g., a DNA backbone). In some embodiments, an L element may contribute to sequence specificity or sequence specific interactions of a given SSB-HbW molecule with a given target. In accordance with various embodiments, an L element may be of any application-appropriate length and composition. For example, in some embodiments, an L element will be long enough to allow that both elements “SSB” and “HbW” are simultaneously bound and/or interacting with a DNA molecule. In some embodiments, an L element is between 1 and 100 amino acids (e.g., 1-50, 2-20, 2-10, 2-5, 2-4 amino acids or longer). In some embodiments, an L element is flexible. In some embodiments, an L element is semi-flexible. In some embodiments, an L element is rigid.

In some embodiments, a linker (e.g., a flexible linker, a semi-flexible linker, etc.) can be designed to have a more specific structure which will be well-within the ability of one of skill in the art.

In some embodiments, linkers can be selected and/or designed based on domains occurring in proteins found in nature. In some embodiments, linkers can be selected or designed to have a certain geometry that provides a specific orientation or spacing between a SSB-domain and an HbW-domain.

In some such embodiments, when a SSB element is located at a 5′ end of encoding nucleotides, and the SSB-HbW molecule comprises an L element, its L element is located at or adjacent to a 3′ end of such a SSB-element encoding sequence. In some embodiments, when a SSB element is located at a 3′ end of encoding nucleotides and the SSB-HbW molecule comprises an L element, its L element is located or adjacent to a 5′ end of a SSB element.

### Exemplary Embodiments of SSB-HbW Agents

In some embodiments, a SSB-HbW agent described herein includes one or more elements as described above. Exemplary combinations of elements are described below. Elements are depicted in brackets [ ], reading from left to right as N-terminus to C-terminus of polypeptide, or 5′ to 3′ of an encoding nucleic acid.
[WRN helicase beta-wing]-[WRN based linker]-[Zinc finger based SSB]
[BLM helicase beta-wing]-[BLM based linker]-[Zinc finger based SSB]
[RECQ1 helicase beta-wing]-[RECQ1 based linker]-[Zinc finger based SSB]
[WRN helicase beta-wing]-[non-helicase based amino acid linker]-[Zinc finger based SSB]
[BLM helicase beta-wing]-[non-helicase based amino acid linker]-[Zinc finger based SSB]
[RECQ1 helicase beta-wing]-[non-helicase based amino acid linker]-[Zinc finger based SSB]
[WRN helicase beta-wing]-[Gly-Ser linker]-[Zinc finger based SSB]
[BLM helicase beta-wing]-[Gly-Ser based linker]-[Zinc finger based SSB] [RECQ1 helicase beta-wing]-[Gly-Ser based linker]-[Zinc finger based SSB]
[BLM helicase beta-wing]-[WRN based linker]-[Zinc finger based SSB]
[RECQ1 helicase beta-wing]-[WRN based linker]-[Zinc finger based SSB]
[WRN helicase beta-wing]-[BLM based linker]-[Zinc finger based SSB] [RECQ1 helicase beta-wing]-[BLM based linker]-[Zinc finger based SSB]
[WRN helicase beta-wing]-[RECQ1 based linker]-[Zinc finger based SSB]
[BLM helicase beta-wing]-[RECQ1 based linker]-[Zinc finger based SSB]
[WRN helicase beta-wing]-[Zinc finger based SSB]
[BLM helicase beta-wing]-[Zinc finger based SSB]
[RECQ1 helicase beta-wing]-[Zinc finger based SSB]
[Zinc finger based SSB]-[WRN based linker]-[WRN helicase beta-wing]
[Zinc finger based SSB]-[BLM based linker]-[BLM helicase beta-wing]
[Zinc finger based SSB]-[RECQ1 based linker]-[RECQ1 helicase beta-wing]
[Zinc finger based SSB]-[non-helicase based amino acid linker]-[WRN helicase beta-wing]
[Zinc finger based SSB]-[non-helicase based amino acid linker]-[BLM helicase beta-wing]
[Zinc finger based SSB]-[non-helicase based amino acid linker]-[RECQ1 helicase beta-wing]
[Zinc finger based SSB]-[Gly-Ser based linker]-[WRN helicase beta-wing]
[Zinc finger based SSB]-[Gly-Ser based linker]-[BLM helicase beta-wing] [Zinc finger based SSB]-[Gly-Ser based linker]-[RECQ1 helicase beta-wing]
[Zinc finger based SSB]-[WRN based linker]-[BLM helicase beta-wing]
[Zinc finger based SSB]-[WRN based linker]-[RECQ1 helicase beta-wing]
[Zinc finger based SSB]-[BLM based linker]-[WRN helicase beta-wing] [Zinc finger based SSB]-[BLM based linker]-[RECQ1 helicase beta-wing]
[Zinc finger based SSB]-[RECQ1 based linker]-[WRN helicase beta-wing]
[Zinc finger based SSB]-[RECQ1 based linker]-[BLM helicase beta-wing]
[Zinc finger based SSB]-[WRN helicase beta-wing]
[Zinc finger based SSB]-[BLM helicase beta-wing]
[Zinc finger based SSB]-[RECQ1 helicase beta-wing]

### Other Modification Agents

### Sequence Modification Polynucleotides

Technologies of the present disclosure make use of sequence modification polynucleotides (e.g., donor templates, e.g., correction templates) that contain a desired genetic modification relative to a sequence of a target site. In some embodiments, a sequence modification polynucleotide is a donor template. In some embodiments, a sequence modification polynucleotide is a correction template. In some embodiments, a sequence modification polynucleotide can be in the form of a single stranded DNA polynucleotide. In some such embodiments, lengths of single stranded DNA oligonucleotide can range from short (e.g., at least about 12 nucleotides) to long (e.g., up to multiple kilobases). In some embodiments, a sequence modification polynucleotide can be a double stranded DNA molecule. In some such embodiments, lengths of double stranded DNA molecules can range from short (e.g., at least about 12 nucleotides) to long (e.g., multiple kilobases). In some embodiments, a double-stranded DNA molecule may be in the form of (an) artificial chromosome(s) or portion thereof. In some embodiments, a sequence modification polynucleotide can be a plasmid. In some embodiments, a sequence modification polynucleotide can comprise chemically modified nucleobases.

In some embodiments, various approaches may be used to create a molecule that can act as a sequence modification polynucleotide (e.g., donor template, e.g., correction template), for example, such as by creation of a temporary single-stranded DNA structure by reverse transcription or, for example, in situations that could trigger sister-chromatid exchange. In some such embodiments, technologies provided by the present disclosure could be used for DNA modification.

In some embodiments, a sequence modification polynucleotide is a donor template. In general, a donor template is any polynucleotide sequence having sufficient complementarity with a target site to hybridize with such a target site and result in gene conversion at such a target site. In some embodiments, the present disclosure further provides for inclusion of a sequence modification polynucleotide comprising or encoding a genetic modification or modifications, that, when constitutively integrated at target site in a genome, has a therapeutic effect. For example, in some embodiments, administration of a sequence modification polynucleotide into a host cell, in combination with a SSB-HbW molecule, results in a genetic modification.

In some such embodiments, a sequence modification polynucleotide may range from 20-nucleotide to 250-nucleotide in length, or more in a single-stranded formation (e.g., a single stranded DNA formation). In some embodiments, degree of complementarity between a sequence modification polynucleotide and its corresponding target site, when optimally aligned using a suitable alignment algorithm, is about or more than about 50%, 60%, 75%, 80%, 85%, 90%, 95%, 97.5%, 99%, or more. For example, in some embodiments, a sequence modification polynucleotide may differ by only one or two bases relative to a target site. However, in some embodiments as will be understood based on context, a sequence modification polynucleotide may differ by many bases relative to a target site, for instance, in cases of genome engineering that may introduce new sites and/or structures (e.g., visualizable or trackable tags, cre-lox recombination sites, creation of indels, etc.). In some such embodiments, therefore, a portion of a sequence modification polynucleotide will have a high degree of complementarity with a given target site at one or more particular portions of the sequence modification polynucleotide (e.g., homology arms), but will differ more substantially in other areas (e.g., sites being inserted, etc.)

In some embodiments, optimal alignment may be determined by using of any suitable algorithm for aligning sequences, a non-limiting example of which includes Vector NTI (Life Technologies, Waltham, MA).

In some embodiments, a sequence modification polynucleotide comprises a sequence that is capable of being incorporated into a polynucleotide sequence (e.g., a gene). The present disclosure encompasses a recognition that sequence modification polynucleotides can be engineered for modifying any desired locus.

In some embodiments, a sequence modification polynucleotide is for modification of a gene that is expressed in a target cell (e.g., is being expressed substantially at the time of administration). Without wishing to be bound by theory, expressed genes may be present in a cell in more “open” or “accessible” chromatin structures or conformations.

In some embodiments, a sequence modification polynucleotide is for modification of a gene that is associated with a disease (e.g., a disease in mammals, e.g., a disease in humans). In some embodiments, a sequence modification polynucleotide is for modification of a human gene that is involved or being studied for its role in a disease or condition in humans. In some embodiments, a sequence modification polynucleotide is for modification of a gene that is that is involved or being studied for its role in cancer, an inborn error of metabolism, a metabolic disorder, an autoimmune disease, an immunodeficiency, cystic fibrosis, hemophilia, sickle cell anemia, Huntington's disease, muscular dystrophy, a neurodegenerative disease, blindness or other ocular disease, congenital lung disease, among others.

In some embodiments, a sequence modification polynucleotide is for modification of a disease associated gene selected from the group consisting of a B-cell lymphoma/leukemia 11 A (BCL11 A) gene, a dystrophin gene (DMD), MMACHC, a DNA polymerase γ gene (PolG), a methylmalonyl CoA mutase gene (MMUT), a phenylalanine hydroxylase gene (PAH), a cystic fibrosis transmembrane conductance regulator (CFTR) gene, a Kruppel-like factor 1 gene, a mammalian beta globin gene, gamma globin gene, a C—C chemokine receptor type (CCR)5 gene, a chemokine (C—X—C motif) receptor 4 (CXCR4) gene, a protein phosphatase 1, regulatory subunit 12C (PPP1R12C) gene, an hypoxanthine phosphoribosyltransferase (HPRT) gene, an albumin gene, a Factor VIII gene, a Factor IX gene, a leucine-rich repeat kinase 2 (LRRK2) gene, a huntingtin (Htt) gene, a rhodopsin (RHO) gene, a surfactant protein B gene (SFTPB), a T-cell receptor alpha (TRAC) gene, a T-cell receptor beta (TRBC) gene, a programmed cell death 1 (PD1) gene, a cytotoxic T-lymphocyte antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with antigen processing (TAP)1 gene, a TAP2 gene, a tapasin gene (TAPBP), a class II major histocompatibility complex transactivator (CUTA) gene, a glucocorticoid receptor gene (GR), an interleukin 2 receptor gamma (IL2RG) gene and an regulatory factor X 5 (RFX5) gene.

In some certain embodiments, a sequence modification polynucleotide comprises a sequence that is capable of being incorporated into a gene selected from: ApoE, Bcl11A, DMD, PolG, MMACHC, MMUT, PAH, CFTR, MMA, and PKU. In some embodiments, a sequence modification polynucleotide comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in Table 3 below.

TABLE 3
Exemplary Sequence Modification Polynucleotides

| SEQ | | |
|---|---|---|
| ID | | |
| NO: | Target | DNA Sequence |
| 10 | Human | CCCCGGTGGCGGAGGAGACGCGGGCACGGCTGTCCAAGGAGCTGCAGGCGGCGCA |
| | ApoE | GGCCCGGCTGGGCGCGGACATGGAGGACGTGCGCGGCCGCCTGGTGCAGTACCGC |
| | | GGCGAGGTGCAGGCCATGCTCGGCCAGAGCACCGAGGAGC |
| 11 | Human | CCCCGGTGGCGGAGGAGACGCGGGCACGGCTGTCCAAGGAGCTGCAGGCGGCGCA |
| | ApoE | GGCCCGGCTGGGCGCGGACATGGAGGACGTGCGCGGCCGCCTGGTGCAGTACCGC |
| | | GGCGAGGTGCAGGCCATGC |
| 12 | Human | CTCTTAGACATAACACACCAGGGTCAATACAACTTTGAAGCTAGTCTAGTGCAAG |
| | Bcl11A | CTAACAGTTGCTTGAATTCACAGGCTCCAGGAAGGGTTTGGCCTCTGATTAGGGT |
| | | GGGGGCGTGGGTGGGGTAGAAGAGGACTGGC |
| 13 | Human | TAATTTTTCTTTTTCTTCTTTTTTCCTTTTTGCAAAAACCCAAAATATTTTAGCT |
| | DMD | CCTACTCAGACTGTTAGACTCTGGTGACACAACCTGTGGTTACTAAGGAAACTGC |
| | | CATCTCCAAACTAGAAATGCCATCTTCC |
| 72 | Human | TGGCAGAGGCACAGGGCACTTATGAGGAGCTCCAGCGGGAGATGAAGAAGTCGTT |
| | PolG | GATGGACTTAACTAACGATGCCTGCCAGCTGCTCTCAGGAGAGAGGTAGCCAGGC |
| | | CTTGGGTGGGCAGGATCTAG |
| 80 | Human | GCTGCCACCTCCGAATGCTGACTGACCCAGTGGACCAGTGTGTGGCCTACCATCT |
| | MMACHC | GGGGAGAGTGAAGAGAGGTGAGGAAGGCTCAGTTTTCCCCCAGCTCCCAAACCTA |
| | | CAGCTGCCTCCAGTTCCTCCA |
| 88 | Human | AAAACCCTAGTTCCTGAACTCATCAAAGAACTTAACTCCCTTGGACGGCCAGATA |
| | MMUT | TTCTTGTTAAATGCGGAGGGGTGATACCACCTCAGGTATTTTTTATCTCTATTTT |
| | | TCTAGTACTGTGATGGGAAT |
| 96 | Human | CCCTTCACTCAAGCCTGTGGTTTTGGTCTTAGGAACTTTGCTGCCACAATACCTT |
| | PAH | GGCCCTTCTCAGTTCGCTGCGACCCATACACCCAAAGGATTGAGGTCTTGGACAA |
| | | TACCCAGCAGCTTAAGATTT |
| 104 | Human | GAATTTCATTCTGTTCTCAGTTTTCCTGGATTATGCCTGGCACCATTAAAGAAAA |
| | CFTR | TATCATATGTGGTGTTTCCTATGATGAATATAGATACAGAAGCGTCATCAAAGCA |
| | | TGCCAACTAGAAGAGGTAAG |

For example, in some embodiments, a sequence modification polynucleotide is capable of modifying human apolipoprotein E (ApoE) and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 10 or 11. In some embodiments, a sequence modification polynucleotide is capable of modifying human ApoE and comprises sequence that is at least 90%, at least 95%, at least 96%, at least 97%, at least 98% at least 99%, or 100% identical to a sequence set forth in SEQ ID NO: 10 or 11. In some embodiments, a sequence modification polynucleotide is capable of modifying human ApoE and comprises or consists of a sequence as set forth in SEQ ID NO: 10 or 11.

In some embodiments, a sequence modification polynucleotide is capable of modifying human BCL11A and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 12. In some embodiments, a sequence modification polynucleotide is capable of modifying human BCL11A and comprises a sequence that is at least 90%, at least 95%, at least 96%, at least 97%, at least 98% at least 99%, or 100% identical to a sequence set forth in SEQ ID NO: 12. In some embodiments, a sequence modification polynucleotide is capable of modifying human BCL11A and comprises or consists of a sequence as set forth in SEQ ID NO: 12.

In some embodiments, a sequence modification polynucleotide is capable of modifying human DMD and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 13. In some embodiments, a sequence modification polynucleotide is capable of modifying human DMD and comprises a sequence that is at least 90%, at least 95%, at least 96%, at least 97%, at least 98% at least 99%, or 100% identical to a sequence set forth in SEQ ID NO: 13. In some embodiments, a sequence modification polynucleotide is capable of modifying human DMD and comprises or consists of a sequence as set forth in SEQ ID NO: 13.

In some embodiments, a sequence modification polynucleotide is capable of modifying human PolG and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 72. In some embodiments, a sequence modification polynucleotide is capable of modifying human PolG and comprises a sequence that is at least 90%, at least 95%, at least 96%, at least 97%, at least 98% at least 99%, or 100% identical to a sequence set forth in SEQ ID NO: 72. In some embodiments, a sequence modification polynucleotide is capable of modifying human PolG and comprises or consists of a sequence as set forth in SEQ ID NO: 72.

In some embodiments, a sequence modification polynucleotide is capable of modifying human MMACHC and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 80. In some embodiments, a sequence modification polynucleotide is capable of modifying human MMACHC and comprises a sequence that is at least 90%, at least 95%, at least 96%, at least 97%, at least 98% at least 99%, or 100% identical to a sequence set forth in SEQ ID NO: 80. In some embodiments, a sequence modification polynucleotide is capable of modifying human MMACHC and comprises or consists of a sequence as set forth in SEQ ID NO: 80.

In some embodiments, a sequence modification polynucleotide is capable of modifying human MMUT and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 88. In some embodiments, a sequence modification polynucleotide is capable of modifying human MMUT and comprises a sequence that is at least 90%, at least 95%, at least 96%, at least 97%, at least 98% at least 99%, or 100% identical to a sequence set forth in SEQ ID NO: 88. In some embodiments, a sequence modification polynucleotide is capable of modifying human MMUT and comprises or consists of a sequence as set forth in SEQ ID NO: 88.

In some embodiments, a sequence modification polynucleotide is capable of modifying human PAH and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 96. In some embodiments, a sequence modification polynucleotide is capable of modifying human PAH and comprises a sequence that is at least 90%, at least 95%, at least 96%, at least 97%, at least 98% at least 99%, or 100% identical to a sequence set forth in SEQ ID NO: 96. In some embodiments, a sequence modification polynucleotide is capable of modifying human PAH and comprises or consists of a sequence as set forth in SEQ ID NO: 96.

In some embodiments, a sequence modification polynucleotide is capable of modifying human CFTR and comprises a sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence set forth in SEQ ID NO: 104. In some embodiments, a sequence modification polynucleotide is capable of modifying human CFTR and comprises a sequence that is at least 90%, at least 95%, at least 96%, at least 97%, at least 98% at least 99%, or 100% identical to a sequence set forth in SEQ ID NO: 104. In some embodiments, a sequence modification polynucleotide is capable of modifying human CFTR and comprises or consists of a sequence as set forth in SEQ ID NO: 104.

### Other or Additional Agents

In some embodiments, one or more additional agents may be used in conjunction with any technology described herein. For example, in some embodiments, an agent induces polynucleotide production or replication. For instance, in some embodiments, an agent induces DNA replication.

In some embodiments, an additional agent is an agent that (i) induces DNA replication and/or (ii) induces DNA repair and/or (iii) influences DNA repair pathways. In some such embodiments, as non-limiting example, RNAi or other technologies may be used to reduce or increase cellular levels of Mismatch Repair (MMR) factors, such as MSH2, MSH3, MSH6, and MLH1. In some embodiments, provided methods further comprise contacting a cell or population of cells with a DNA modification system that includes one or more of a DNA polymerase, helicase, ligase, recombinase, repair scaffold protein, single strand DNA binding protein, and/or mismatch repair protein.

### Polynucleotide Modification Systems

In some embodiments, the present disclosure provides polynucleotide modification systems. As used herein, a polynucleotide modification system refers to an editing system that modifies (e.g., changes via deletion, addition, substitution, etc.) a given polynucleotide (e.g., DNA, RNA, mRNA, etc.). In some embodiments, a polynucleotide modification system modifies (e.g., changes via deletion, addition, substitution, etc.) a given polynucleotide (e.g., DNA, RNA, mRNA, etc.) in a cell without causing a single and/or double-stranded break in a given polynucleotide (e.g., DNA, RNA, etc.) being modified. In some embodiments, a polynucleotide modification system is a zinc finger helicase beta-wing mediated DNA modification system comprising (i) a modifying agent (e.g., a SSB-HbW molecule) and (ii) a sequence modification polynucleotide. In some such embodiments, the modifying agent binds to, e.g., double-stranded DNA. In some embodiments, binding of, e.g., a modifying agent, e.g., a SSB-HbW molecule, results in strand separation at or close to a SSB-HbW binding site. In some embodiments, binding of, e.g., a modifying agent, e.g., a SSB-HbW molecule, results in strand separation at or close to a SSB-HbW binding site and binding of a sequence modification polynucleotide to a (partially) complementary sequence. In some embodiments, binding of, e.g., a polynucleotide modifying agent, e.g., a SSB-HbW molecule, results in strand separation at or close to the SSB-HbW binding site and binding of a sequence modification polynucleotide to a (partially) complementary sequence and, without being bound to any theory, such binding results in (part of) a sequence modification sequence becoming incorporated in a genome.

In some embodiments, a gene editing system (e.g., comprising a SSB-HbW fusion) provides methods of a targeted genetic (e.g., DNA) modification. As described herein, targeted genetic (e.g., DNA) modifications are, but are not limited to, changes that include insertions, deletions and/or substitutions (e.g., point mutations). In some embodiments, these methods may include transfection of a cell with one or more components of a gene editing system described herein. In some such embodiments, a gene editing system comprises both a SSB-HbW agent and a sequence modification polynucleotide, also referred to herein as a zinc finger helicase beta-wing gene editing system.

In some embodiments, the present disclosure provides zinc finger helicase beta-wing gene editing based methods comprising a SSB-HbW agent and a sequence modification polynucleotide. In some such embodiments, a zinc finger helicase beta-wing gene editing system is capable of efficiently generating an intended nucleic acid modification at a target site, while limiting formation of off-target mutations. Certain characteristics of zinc finger helicase beta-wing gene editing provide for low risk in gene editing (i.e., low risk of off-target events) and, accordingly, provide increased safety for development of therapies applicable for use in human subjects.

In some embodiments, a polynucleotide modification system comprises a nucleic acid encoding a polynucleotide modification agent (e.g., SSB-HbW agent) and a sequence modification polynucleotide. In some embodiments, a polynucleotide modification system comprises DNA encoding a polynucleotide modification agent (e.g., SSB-HbW agent). In some embodiments, a polynucleotide modification system comprises RNA (e.g., mRNA) encoding a polynucleotide modification agent (e.g., SSB-HbW agent).

In some embodiments, a polynucleotide modification system comprises a polypeptide polynucleotide modification agent (e.g., SSB-HbW agent) and a sequence modification polynucleotide. In some embodiments, the polypeptide polynucleotide modification agent and the sequence modification polynucleotide are provided in the same composition.

In some embodiments, the present disclosure recognizes that a zinc finger helicase beta-wing gene editing system, as provided herein is capable of modifying a nucleic acid sequence with a low incidence of indels. An “indel”, as used herein, refers to an insertion or deletion of (a) nucleotide base(s) within a nucleic acid. Such insertions or deletions can lead to frame shift mutations within a coding region of gene.

In some embodiments, it is desirable to combine a SSB-HbW agent (e.g., a SSB-HbW molecule) with a sequence modification polynucleotide (e.g., a donor template) to efficiently make desired genetic modifications with low incidences of undesired indels in such a nucleic acid. In some embodiments, a zinc finger helicase beta-wing mediated gene editing system is capable of generating a desired gene conversion while achieving very low percentages of indels at a target site. In some embodiments undesirable indels frequencies are obtainable at frequencies lower than 2%, ranging from 0.05% to 2%, similar to frequencies observed in an untargeted background. Frequencies and numbers of desired genetic (e.g., DNA) modifications and undesired mutations and indels may be determined using any suitable method, for example by methods used in examples below.

### Compositions

Among other things, the present disclosure provides compositions. In some embodiments, a composition comprises an agent as described herein. In some embodiments, an agent is a polynucleotide modification agent (e.g., a SSB-HbW molecule). In some embodiments, an agent is a sequence modification polynucleotide, an enhancing agent, an inhibiting agent, etc. In some embodiments, a composition comprises one or more polynucleotide modification agents and/or sequence modification polynucleotides as described herein. In some embodiments, a composition comprises a plurality of polynucleotide modification agent and/or sequence modification polynucleotides.

In some embodiments, a composition comprises a polynucleotide encoding a polynucleotide modification agent or a portion thereof. In some embodiments, a composition comprises a polynucleotide modification agent comprising a sequence encoding a SSB-HbW molecule or a portion thereof.

In some embodiments, a composition comprises an agent encoding a sequence modification polynucleotide (e.g., a correction template, a donor template). In some embodiments, a composition comprises an agent comprising a sequence encoding an enhancing and/or inhibiting agent, e.g., an siRNA, or portion thereof. In some such embodiments, an enhancing agent and/or inhibiting agent is used to, e.g., modify cellular machinery such as, for example DNA replication machinery.

In some embodiments, a composition comprises at least two agents, e.g., a polynucleotide modification agent and a sequence modification polynucleotide, or at least three agents, e.g., a polynucleotide modification agent, a sequence modification polynucleotide, and an enhancing agent/inhibiting agent, etc.

In some embodiments, a composition comprises a cell.

In some embodiments, a composition is or comprises a construct or a vector. In some such embodiments, a construct or vector can encode one or more agents or portions thereof, as described herein. In some embodiments, a vector is or comprises a viral vector (e.g., an adenoviral, adenoviral-associated, or lentiviral vector).

In some embodiments, polynucleotide modification agent and/or sequence modification polynucleotides described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, a composition comprises a polypeptide polynucleotide modification agent (e.g., polypeptide HbW-SSB). In some embodiments, a polypeptide polynucleotide modification agent (e.g., polypeptide HbW-SSB) is contacted with a sequence modification polynucleotide. In some embodiments, a composition comprises a polypeptide polynucleotide modification agent (e.g., polypeptide HbW-SSB) and a sequence modification polynucleotide.

In some embodiments, a composition is or comprises a pharmaceutical composition.

### Pharmaceutical Compositions

Pharmaceutical compositions of the present disclosure include at least one polynucleotide modification agent described herein. For example, in some embodiments, pharmaceutical compositions may comprise a SSB-HbW molecule. In some embodiments, a pharmaceutical composition may comprise a sequence modification polynucleotide. For example, a pharmaceutical composition of the present disclosure comprising one or more agents (e.g., a modification agent, e.g., a SSB-HbW molecule and/or a sequence modification polynucleotide) as described herein, may be provided in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; and preservatives. In some embodiments, compositions of the present disclosure are formulated for intravenous administration. Any compositions described herein can be, e.g., a pharmaceutical composition.

In some embodiments, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations may be administered in a variety of dosage forms such as injectable solutions, injectable gels, drug-release capsules, and the like.

Compositions provided herein can be, e.g., formulated to be compatible with their intended route of administration. In some embodiments, compositions provided herein may be formulated to be compatible with any suitable route of administration, for example, those used or contemplated in the context of gene and cell therapy, including, e.g., intravenous, intermuscular, intrathecal, intraperitoneal, intra-tumor, ocular delivery, inner ear injection etc. A non-limiting example of an intended route of administration is intravenous administration. In some embodiments, administration may occur ex vivo and cells may be provided post-administration, to a subject in need thereof.

In some embodiments, a composition provided herein further comprises lipid nanoparticles, polyplexes (PLX), lipidated polyplexes (LPLX), or liposomes. In some embodiments, a composition comprises a SSB-HbW molecule that is fully or partially encapsulated within the lipid nanoparticles, polyplexes (PLX), lipidated polyplexes (LPLX), or liposomes. In some embodiments, a composition comprises a sequence modification polynucleotide that is fully or partially encapsulated within the lipid nanoparticles, polyplexes (PLX), lipidated polyplexes (LPLX), or liposomes. In some embodiments, a composition comprises a polynucleotide modification system (e.g., a SSB-HbW molecule and a sequence modification polynucleotide) that is fully or partially encapsulated within the lipid nanoparticles, polyplexes (PLX), lipidated polyplexes (LPLX), or liposomes.

In some embodiments, a composition provided herein further comprises lipid nanoparticles. In some embodiments, a composition comprises a SSB-HbW molecule that is fully or partially encapsulated within the lipid nanoparticles. In some embodiments, a composition comprises a sequence modification polynucleotide that is fully or partially encapsulated within the lipid nanoparticles. In some embodiments, a composition comprises a polynucleotide modification system (e.g., a SSB-HbW molecule and a sequence modification polynucleotide) that is fully or partially encapsulated within the lipid nanoparticles.

### Kits

Also provided are kits including any compositions described herein. In some embodiments, a kit can include a solid composition (e.g., a lyophilized composition including at least one agent as described herein) and/or a liquid for solubilizing a lyophilized composition. In some embodiments, a kit comprises instructions for use.

In some embodiments, provided are kits comprising a polynucleotide modification agent as described herein (e.g., as a composition, e.g., as a pharmaceutical composition). In some embodiments, a kit further comprises a sequence modification polynucleotide (e.g., as a composition, e.g., as a pharmaceutical composition). In some embodiments, a kit further comprises instructions for use.

In some embodiments, a kit comprises a polynucleotide modification agent as described herein (e.g., as a composition, e.g., as a pharmaceutical composition) and instructions for designing a sequence modification polynucleotide.

In some embodiments, a kit can include a pre-loaded syringe including any compositions described herein.

In some embodiments, a kit includes a vial comprising any of the compositions described herein (e.g., formulated as an aqueous composition, e.g., an aqueous pharmaceutical composition).

In some embodiments, a kit can include instructions for performing any methods described herein.

### Methods of Making

In some embodiments, compositions, agents or systems of the present disclosure are prepared by any methods known to one of skill in the art. In some such embodiments, such preparations are formulated for delivery into a subject.

In some embodiments, compositions are prepared using any standard synthesis and/or purification system that will be known to one of skill in the art. For example, in some embodiments as described herein, one or more methods may include techniques such as de novo gene synthesis, DNA fragment assembly, PCR, mutagenesis, Gibson assembly, molecular cloning, standard single-stranded DNA synthesis, PCR, molecular cloning, digestion by restriction enzymes, small RNA molecule synthesis, cloning into plasmids with U6 promoter for RNA transcription, etc.

### Methods of Characterization

In some such embodiments, a gene modification system provided herein including one or more polynucleotide modifying agents (e.g., a SSB-HbW molecule) and/or sequence modification polynucleotides of the present disclosure may be tested and/or characterized by one or more assays.

In some embodiments, the ability of provided gene modification systems to carry out gene conversions can be demonstrated using reporter constructs such as by using a green fluorescent protein reporter construct that allows for detection of gene conversion by fluorescence detection. By way of non-limiting example, the present disclosures contemplates that in some embodiments other types of reporter constructs can be used, such as, but not limited to reporters based on fluorescent detection, bioluminescence detection, the usage of antibiotics markers, markers that make use of antibody detection and/or use of a phenotypical feature.

In some embodiments, methods in accordance with the present disclosure can be utilized in cell types in which a distinguishable sequence modification polynucleotide (e.g., donor template) can be efficiently analyzed if it has integrated into a targeted genome. Accordingly, in some embodiments, the present disclosure provides methods for evaluation of gene editing effects, e.g., on-target correction and off-targets mutations. In some embodiments, the present disclosure provides methods applicable for evaluating editing effects as compared to other gene editing technologies including, but not limited to, engineered nucleases and nickases.

In some embodiments, analysis and/or identification of cells containing a desired genetic modification (e.g., gene conversion) may be performed in a single cell, or in a population of cells (e.g., a batch of cells, e.g., several batches or pooled populations of cells, etc.).

In some embodiments, analysis and/or identification of cells containing a desired genetic modification may be performed in (a) specific clone(s).

In some embodiments, analysis and/or identification of cells containing a desired genetic modification may be performed using a digital PCR method.

In some embodiments, analysis and/or identification of cells containing a desired genetic modification may be performed using a PCR method. In some embodiments, analysis and/or identification of cells containing a desired genetic modification may be performed using a Sanger Sequencing method. In some embodiments, analysis and/or identification of cells containing a desired genetic modification (e.g., gene conversion) may be performed using a Next Generation Sequencing method. In some embodiments, analysis and/or identification of cells containing a desired genetic modification may be performed using any appropriate method to determine if one or more changes in one or more nucleotides has occurred. In some such embodiments, the present disclosure provides various methods of characterization, as described herein.

In some embodiments, analysis and/or identification of cells containing a desired genetic modification may be performed using an assay based on functionality.

In some embodiments, analysis and/or identification of cells containing a desired genetic modification may be performed using an assay based on phenotype.

In some embodiments, analysis and/or identification of cells containing a desired genetic modification (e.g., gene conversion) may be performed using features of sequence modification polynucleotides (e.g., correction polynucleotides) or other components that allow identification and potentially selection for corrected cells. This may be done for example by making use of sequence modification polynucleotides (e.g., correction polynucleotides) that contain a dye or chromophore or a chemical modification (e.g., biotin) that allows for detection.

### Methods of Use

Among other things, the present disclosure provides methods and compositions for carrying out targeted genetic conversions (e.g., gene editing, gene conversion and/or gene targeting). The present disclosure provides technologies that, in contrast to previously disclosed methods for gene targeting, are efficient and do not depend on introducing bacterial, viral or non-human/humanized protein or protein fragments. In addition, the present disclosure provides technologies that, in contrast to previously disclosed methods for gene targeting, are efficient and do not depend on introducing polynucleotide (e.g., DNA) breaks into molecules comprising target sites. The present disclosure provides the insight that such technologies reduce risks of immunological responses in a human host that may limit the effectiveness of gene targeting therapies. In addition, the present disclosure provides the insight that such technologies reduce risks of creation of unwanted indels on a target site or mutations at off-target sites. In some embodiments any segment of nucleic acid in a genome of a cell or organism can be targeted in accordance with technologies (e.g., methods) of the present disclosure.

In some embodiments, provided technologies for genetic modification include contacting a cell with a sequence-specific DNA-binding polynucleotide modification agent and a sequence modification template (e.g., donor template). In some embodiments, a sequence modification polynucleotide (e.g., template, e.g., a donor template, e.g., a correction template) carries a genetic modification (e.g., a polynucleotide modification) relative to a sequence of a target site. In some such embodiments, a sequence modification polynucleotide is capable of annealing to one strand of nucleic acid (e.g., when a helicase beta wing structure may have resulted in single stranded DNA) at a target site, e.g., in a genome. In some embodiments, a polynucleotide modification agent (e.g., a fusion protein comprising a sequence specific binding element and a HbW element) and a sequence modification polynucleotide (e.g., donor template, e.g., correction template) will be administered to and/or administered to a cell. In some embodiments, a polynucleotide modification agent and a sequence modification polynucleotide are simultaneously present in a given cell. In some embodiments, in addition to a polynucleotide modification agent and a sequence modification polynucleotide, an enhancing or inhibiting agent (e.g., an siRNA, etc.) may also be administered. In some embodiments, more than one modifying agent, sequence modification polynucleotide and/or enhancing or inhibiting agent, (e.g., siRNA) may be administered to and/or presented to a cell.

Gene conversion and genome engineering can be useful for a wide variety of purposes. As a consequence, many different targets can be selected for gene conversion and/or for genome engineering. For example, in some embodiments a target chosen may be for the purpose of gene conversion or genome engineering to treat diseases (e.g., human diseases). For instance, in some embodiments, monogenic diseases can be targeted by conversion of underlying mutations to corresponding sequences found in a non-affected population. Non-limiting examples of such embodiments include correction of mutations in the HPRT gene in the case of certain forms of Lesch-Nyhan syndrome, correction of certain mutations (e.g., in one or more exons known to have a mutation resulting in a DMD phenotype, e.g., exons 44, 45, 46, 47, 51, 53, etc., e.g., exon 51) in the dystrophin gene in the case of certain forms of muscular dystrophy or, e.g., correction of certain mutations in the case of the CFTR gene in the case of certain forms of Cystic Fibrosis.

In addition to monogenic diseases, gene mutations that are associated with increased risk for certain diseases can be modified to sequences that normalize or reduce that risk. For example, the ApoE gene has several variant alleles and certain variants (i.e., E4) are associated with increased risk for developing Alzheimer's disease, whereas other variants normalize (i.e., E3 allele) or even reduce (i.e. E2 allele) the risk for Alzheimer's diseases. In some embodiments, multigenic diseases could be targeted when multiple gene targets are being addressed either simultaneously or sequentially and either with one or multiple gene editing systems provided herein (e.g., zinc finger helicase beta-wing mediated gene editing systems).

In some embodiments, a gene may silence expression and/or function of another gene and/or protein. For instance, BCL11A is a potent regulator of fetal-to-adult hemoglobin switch after birth. Generally, a higher level of BCL11A is associated with adult hemoglobin, and in patients with sickle cell anemia or β-thalassemia, adult hemoglobin is damaged. Thus, without being bound by any particular theory and by way of non-limiting example, in some embodiments, BCL11A may “silence” fetal hemoglobin (HbF) and in some embodiments, reduction or removal of such “silencing” may increase production of HbF such that symptoms of disorders involving adult beta-hemoglobin, such as β-thalassemia and sickle cell disease may be ameliorated. Accordingly, the present disclosure contemplates that, in some embodiments, decreasing levels of BCL11A using technologies provided by the present disclosure may increase HbF levels.

In some embodiments, provided technologies and systems are for use in a method of treating a disease. In some embodiments, provided technologies and systems are for use in modifying gene that is associated with a disease (e.g., a disease in mammals, e.g., a disease in humans). In some embodiments, provided technologies and systems are for use in modifying gene that is associated with cancer, an inborn error of metabolism, a metabolic disorder, an autoimmune disease, an immunodeficiency, cystic fibrosis, hemophilia, sickle cell anemia, Huntington's disease, muscular dystrophy, a neurodegenerative disease, blindness or other ocular disease, congenital lung disease, among others.

In some embodiments, a disease-associated gene is selected from the group consisting of a B-cell lymphoma/leukemia 11 A (BCL11 A) gene, a dystrophin gene (DMD), MMACHC, a DNA polymerase γ gene (PolG), a methylmalonyl CoA mutase gene (MMUT), a phenylalanine hydroxylase gene (PAH), a cystic fibrosis transmembrane conductance regulator (CFTR) gene, a Kruppel-like factor 1 gene, a mammalian beta globin gene, gamma globin gene, a C—C chemokine receptor type (CCR)5 gene, a chemokine (C—X—C motif) receptor 4 (CXCR4) gene, a protein phosphatase 1, regulatory subunit 12C (PPP1R12C) gene, an hypoxanthine phosphoribosyltransferase (HPRT) gene, an albumin gene, a Factor VIII gene, a Factor IX gene, a leucine-rich repeat kinase 2 (LRRK2) gene, a huntingtin (Htt) gene, a rhodopsin (RHO) gene, a surfactant protein B gene (SFTPB), a T-cell receptor alpha (TRAC) gene, a T-cell receptor beta (TRBC) gene, a programmed cell death 1 (PD1) gene, a cytotoxic T-lymphocyte antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with antigen processing (TAP)1 gene, a TAP2 gene, a tapasin gene (TAPBP), a class II major histocompatibility complex transactivator (CUTA) gene, a glucocorticoid receptor gene (GR), an interleukin 2 receptor gamma (IL2RG) gene and an regulatory factor X 5 (RFX5) gene.

In some embodiments, a target may be selected for the purpose of creating models useful for the study of gene conversion or genome engineering to correct and/or ameliorate human diseases. These models can be cell-based models and/or animal models.

In some embodiments, a target may be selected for the purpose of creating models useful for the study of gene conversion or genome engineering. These models may be cell-based models and/or animal models.

In some embodiments, a target may be selected for the purpose of creating models useful for the study of biological processes. These models may be cell-based and/or animal models.

In some embodiments, a target may be selected for the purpose of creating models useful for the study of disease-causing processes. These models may be cell-based and/or animal models.

In some embodiments, a target may be selected for the purpose of gene conversion or genome engineering in mammalian cell lines involved in production of useful substances or features.

In some embodiments, a target may be selected for the purpose of gene conversion or genome engineering in plant cell lines involved in production of useful substances or features.

In some embodiments, a target may be selected for the purpose of gene conversion or genome engineering in eukaryotic cell lines involved in production of useful substances or features.

In some embodiments, a target may be selected for the purpose of gene conversion or genome engineering in one or more infectious agents (e.g., bacteria, parasite, virus, etc.).

In some embodiments, a target may be selected for the purpose of gene conversion or genome engineering in bacterial cell lines involved in production of useful substances or features.

In some embodiments, a target may be selected for the purpose of gene conversion or genome engineering in prokaryotic cell lines involved in production of useful substances or features.

In some embodiments, a target may be selected for the purpose of gene conversion or genome engineering in virus sequences.

### Genotyping and Design of Polynucleotide Modification Agents and or Sequence Modification Polynucleotides

In some embodiments, the present disclosure provides methods of making a change in genetic material (e.g., of a subject) based on analysis of a sample. For instance, in some embodiments, a sample is obtained. In some such embodiments, a sample may be tested to determine a genotype at one or more target sites and/or to determine a sequence of one or more target sequences using any number of methods known to those of skill in the art. In some embodiments, sequence analysis information is used to design and/or aid in selection of an appropriate SSB-HbW molecule and/or sequence modification polynucleotide that can be used to introduce a sequence modification into genetic material of a sample or of a subject from where a sample was derived. After analysis, a SSB-HbW molecule and/or sequence modification polynucleotide may be introduced or administered such that it is has access to or contact with genetic material to which a modification may be made.

In some embodiments, a sample is obtained or derived from a subject. In some embodiments, a subject is a control subject. In some embodiments, a subject has one or more diseases, disorders or conditions. In some embodiments, such a disease, disorder, or condition has one or more genetic changes associated therewith. In some embodiments, a subject is determined to have one or more genetic changes (e.g., genotype) associated with a particular disease, disorder or condition.

In some embodiments, a subject does not have one or more genetic changes associated with a disease, disorder, or condition, but may have an acquired phenotype that would benefit from a modification in one or more target sites and/or sequences.

In some embodiments, a polynucleotide modification agent (e.g., SSB-HbW molecule) and/or sequence modification polynucleotide are administered or introduced to a subject or sample derived therefrom, in need thereof. In some embodiments, a sample is acquired. In some embodiments, after acquisition, a sample may be optionally further processed (e.g., to purify, expand, test, etc.) to determine genotype information. In some embodiments, after genotypic information is determined, one or more polynucleotide modification agents (e.g., SSB-HbW molecules) and/or sequence modification polynucleotides may be designed to modify one or more target sites and/or target sequences.

In some embodiments, a polynucleotide modification agent (e.g., SSB-HbW molecule) and/or sequence modification polynucleotide is administered or applied such that it contacts genetic material to be modified. In some embodiments, administration or application is ex vivo or in vitro. In some embodiments, administration or application is in vivo. In some embodiments, after genetic material is contacted by one or more polynucleotide modification agents (e.g., SSB-HbW molecules) and/or sequence modification polynucleotides, a change in genotype detectable. In some embodiments, a change in genotype leads to a change in phenotype. In some embodiments, a change in phenotype is a reduction in one or more symptoms or manifestations of a disease, disorder, or condition, or risk thereof.

In some embodiments, after genetic material is contacted by one or more polynucleotide modification agents (e.g., SSB-HbW molecules) and/or sequence modification polynucleotides, a no change in genotype detectable. In some such embodiments, one or more of the genetic material, polynucleotide modification agent (e.g., SSB-HbW molecule) and/or sequence modification polynucleotide is a control sequence designed to demonstrate no negative impact of administration of any composition comprising one or more polynucleotide modification agents and/or sequence modification polynucleotides.

In some embodiments, a sample does not come from a subject in need of treatment. For example, in some embodiments, as sample may be or comprise an infectious agent. In some such embodiments, a subject may be suffering from or at risk of infection from such an infectious agent. Accordingly, in some embodiments, a polynucleotide modification agent (e.g., SSB-HbW molecule) and/or sequence modification polynucleotide may be designed to inhibit or otherwise incapacitate one or more features of an infectious agent, such that risk of infection is eliminated or ameliorated. In certain embodiments of this disclosure (a) desired genetic modifications may entail a single nucleotide change, for example, in a particular gene. In certain embodiments of this disclosure a desired genetic modification may entail multiple nucleotide changes.

In certain embodiments of this disclosure a desired genetic modification may entail other forms of DNA editing.

In certain embodiments of this disclosure the desired genetic modification may entail other forms of genomic engineering.

In some embodiments, activity of a SSB-HbW molecule results in a genetic conversion of a point mutation via use of a sequence modification polynucleotide. In some embodiments, a genetic converting activity requires a complete genetic modification system (e.g., a zinc finger helicase beta wing mediated DNA modification system) including a polynucleotide modification agent (e.g., SSB-HbW molecule) and sequence modification polynucleotide. For example, if a target site comprises a T→C point mutation and is associated with a risk predisposition for a disease or a disorder, in some embodiments, a target sequence comprises a C→T point mutation, wherein such a genetic conversion from C to T results in a sequence that is not associated with a risk factor with a disease or a disorder. In some embodiments, a target sequence encodes a protein and wherein a point mutation is in a codon and results in a change in an amino acid encoded by a mutant codon as compared to a wild-type codon. In some embodiments, a disease or disorder is Alzheimer's disease.

In some embodiments, genetic modification (e.g., gene conversion) can be demonstrated at a site naturally occurring within a mammalian genome. For example, in some embodiments, codon 112 of human ApoE, which comprises a point mutation that, in some embodiments, can increase predisposition to Alzheimer's disease, can be targeted and converted using a gene modification system comprising a SSB-HbW molecule and a sequence modification polynucleotide (see, e.g., Example 2)

In some embodiments, genetic modification (e.g., gene conversion) can be demonstrated at a number of different sites that are naturally occurring within a mammalian genome. For example, in some embodiments, codon 158 of human ApoE can be targeted and converted using a gene modification system comprising a SSB-HbW molecule and a sequence modification polynucleotide.

In some embodiments, the present disclosure contemplates that any site within a genome can be modified. For example, as described above and herein, in some embodiments, a cell can harbor one or more point mutations in its genome. In some such embodiments, for example, one or more point mutations can exist, e.g., T-to-C or C-to-T. By way of non-limiting example, point mutations at codons 112 and 158 in the human ApoE gene can result in C112R and R158C amino acid mutations, respectively. In some such embodiments, changing one or more of these point mutations using a SSB-HbW molecule and sequence modification polynucleotide can change one or more nucleotides in codon 112 and/or 158, resulting in a change of an ApoE isoform from pathogenic to non-pathogenic, e.g., from more likely to develop Alzheimer's disease to less likely to develop Alzheimer's disease, e.g., based on an ApoE genotype. For example, in accordance with the present disclosure, a genetic modification can be made at ApoE codon 112 to achieve a C to T gene conversion (see, e.g., Example 2). The present disclosure contemplates that in some embodiments, any number of cell lines or primary cell cultures may be used and such cells will be known and/or understood by those of skill in the art dependent upon context.

The present disclosure provides the insight that successful correction of pathogenic gene variants (such as mutations) in genes associated with one or more diseases, disorders and/or conditions provides new strategies for gene correction. In some embodiments, a genetic modification system (e.g., a zinc finger helicase beta wing mediated DNA modification system) can be used to correct other mutations associated with any disease, disorder and/or condition.

In some embodiments, sequence-specific and site-specific genetic modification approaches comprising, e.g., a polynucleotide modification agent (e.g., a SSB-HbW molecule), a sequence modification polynucleotide and/or systems such as a zinc finger helicase beta wing mediated DNA modification system which comprises both a SSB-HbW molecule and a sequence modification polynucleotide can be used to modify genes in such a way that certain gene functions are eliminated or abolished. For example, in some embodiments, a zinc finger helicase beta wing mediated DNA modification system may be used for generation of premature stop codons (TAA, TAG, TGA) to abolish protein functions, for example, in cancers.

In some embodiments, such technologies may be used, for example, in laboratory or research settings to design new cell lines for use in, e.g., development of therapeutics or screening of disease states or, e.g., screening of compound, etc.

In some embodiments, the present disclosure provides new methods and reagents for gene conversion and genome engineering. For instance, as illustrated in Example 2 a SSB-HbW-based gene-editing system can yield important advantages such as off-target effects occurring at very low frequencies.

### Methods of Treatment

In some embodiments, technologies of the present disclosure are used to treat subjects with or at risk of a pathogenic phenotype due to an underlying (e.g., inherited, e.g., acquired) genotype. For example, in some embodiments, a subject has a point mutation in an ApoE gene, which produces an allele that generates an isoform that is associated with a higher risk of developing Alzheimer's disease. In some embodiments, technologies of the present disclosure may be used to treat diseases, disorders or conditions that are caused by one or more mutations in at least one target sequence; for example, in some embodiments, a subject may have a mutation in, for example, a CFTR gene, which mutation causes cystic fibrosis. In some embodiments, a subject may have one or more mutations in the human dystrophin gene resulting in muscular dystrophy, e.g., Duchenne muscular dystrophy. For example, in some embodiments, one or more mutations in the dystrophin gene may result in a frame shift such that dystrophin production is reduced or eliminated. In some embodiments, technologies of the present disclosure may introduce one or more genetic modifications such that a functional reading frame is restored and some amount of dystrophin protein (either in full or truncated form) is produced.

In some embodiments, technologies of the present disclosure may be used to treat cancer. For example, in some embodiments, a cancer may be hereditary (e.g., BRCA1 gene mutation) or inherited (e.g., spontaneous mutation causing, e.g., leukemia). In some such embodiments, technologies of the present disclosure may be used to change genotypes of one or more cells comprising a cancer-associated (e.g., cancer causing) genetic sequence.

In some embodiments, technologies of the present disclosure may be used to achieve genetic modifications that result in removal of a gene regulation function. For example, in some embodiments, BCL11A may silence fetal hemoglobin (HbF). In some such embodiments, reduction or removal of such silencing may increase production of HbF such that symptoms of disorders involving adult beta-hemoglobin, such as β-thalassemia and sickle cell disease may be ameliorated. Without being bound by any particular theory, the present disclosure contemplates that, in some embodiments, decreasing levels of BCL11A using technologies provided by the present disclosure may increase HbF levels. In some embodiments, technologies of the current disclosure may be used in immune-related treatments (e.g., immuno-oncology or other immune diseases, disorders or conditions). For example, in some embodiments, genetic modifications may be made to one or more genes involved in immune function and/or immune regulation. In some such embodiments, technologies of the present disclosure may be used to change a genotype of one or more cells or cell types comprising an immune-associated genetic sequence (e.g., T-cell receptor alpha, T-cell receptor beta, PD-1 (i.e., PDCD-1), PD-L1 CTLA-4, TREM2). For example, in some embodiments, the present disclosure contemplates that editing PDCD-1 by introducing a stop codon may decrease or eliminate PD-1 signaling such that, in some embodiments, cancer activities are reduced or eliminated. In some embodiments, a cancer cells, after editing, may become more responsive or may become sensitive to a treatment (as compared to, e.g., prior to editing where, in some embodiments, a cancer cell may not have been sensitive or responsive to a particular treatment).

By way of non-limiting example, for instance, in some embodiments technologies of the present disclosure may be used to support development of cellular technologies that aim to treat cancer-associated conditions or immune-dysbiosis related conditions.

In some embodiments, technologies of the present disclosure may be used to treat one or more infectious diseases, disorders or conditions. For example, in some embodiments, an infectious disease may be caused by bacteria, parasites, and/or viruses. For example, the present disclosure provides technologies that may be used, e.g., to interfere with replication and/or proliferation of a virus or bacteria.

In some embodiments, the present disclosure provides methods of determining a genotype of a subject or a sample as described herein. In some such embodiments, determining a genotype is used in diagnosing and/or treating a subject as described herein.

It will be understood by those in the art that many different changes (e.g., substitutions, deletions, additions, etc.) in any genetic material can result in or risk causing one or more pathogenic phenotypes.

### Administration

In some embodiments, the present disclosure provides means of administration of provided technologies. In some embodiments, provided technologies include a polynucleotide modification agent (e.g., SSB-HbW molecule) in combination with a sequence modification polynucleotide that can be used to generate or induce sequence (e.g., nucleotide) conversions. In some such embodiments, methods comprise delivering one or more sequence modification polynucleotides, such as one or more vectors (e.g., viral vectors) and/or one or more transcripts thereof, and/or one or more proteins transcribed therefrom in accordance with the present disclosure, to a host cell.

By way of non-limiting example, in some embodiments non-viral vector delivery systems include DNA plasmids, RNA (e.g., a transcript of a vector described herein), naked nucleic acid, and/or nucleic acid complexed with a delivery vehicle, such as liposome.

In some embodiments introduction of a polynucleotide modification agent (e.g., SSB-HbW molecule) and polynucleotide template can be performed by transfection. In some embodiments, introduction of polynucleotide modification agent (e.g., SSB-HbW molecule) and sequence modification polynucleotide can be performed by nucleofection. In some embodiments, introduction of a polynucleotide modification agent (e.g., SSB-HbW molecule) and sequence modification polynucleotide can be performed by any known or appropriate route of introduction into a target cell (e.g., a cell comprising at least one target site).

In some embodiments, a target site comprises a small deletion, insertion and/or single nucleotide polymorphism within a coding sequence of a gene. In some embodiments, a target site comprises more than one mutation, for example, a deletion and a point mutation wherein these two mutations are located adjacent to one another. In some embodiments, a deletion is associated with early termination of translation of a gene product (e.g., a protein) because of, e.g., generation of a premature stop codon and/or reading frame shift.

In some embodiments, activity of an agent (e.g., a given SSB-HbW molecule) in combination with a sequence modification polynucleotide of a genetic modification system (e.g., a zinc finger helicase beta wing mediated DNA modification system) results in genetically correcting a deletion, insertion and/or single nucleotide polymorphism to restore an appropriate reading frame and translate into a normal and functional gene product. In some embodiments, activity of a polynucleotide modification agent (e.g., SSB-HbW molecule) in combination with a sequence modification polynucleotide of a genetic modification system (e.g., zinc finger helicase beta wing mediated DNA modification system) results in correction of two mutations simultaneously. In some embodiments “larger” insertions, deletions, gene rearrangements and/or chromosome rearrangements may be involved. For example, in some embodiments, a “larger” change may be, as described herein, in contexts of genome engineering including but not limited to insertions of visualizable or detectable tags, cre-lox components, indels, etc. In some embodiments, for example, gene conversions of one, two, or several nucleotides would not be considered “larger”. In some embodiments other forms of gene repair and/or genome engineering may be performed by using a genetic modification system described herein (e.g., a zinc finger helicase beta wing mediated DNA modification system).

In some embodiments, provided technologies are suitable for administration to a subject. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

### Combination Therapy

In some embodiments, administration can occur in combination with other molecules. For example, in some embodiments, administration can occur in combination with an enhancing agent. In some embodiments, administration can occur in combination with an inhibiting agent.

In some embodiments, an enhancing or inhibiting agent, when administered in conjunction with (e.g., sequentially or simultaneously) a polynucleotide modification agent and/or a sequence modification polynucleotide, may increase or decrease frequency of recombination events in a polynucleotide (e.g., DNA) contacted with the combination of an enhancing and/or inhibiting agent and polynucleotide modification agent, relative to frequency of recombination in a polynucleotide contacted with the polynucleotide modification agent without the enhancing agent.

In some embodiments, administration of combinations may include more than one combination and may, in some embodiments, occur in stages. For example, a polynucleotide modification agent (e.g., SSB-HbW molecule) may be combined with two additional agents, one of which enhances a particular process and another which inhibits a process. In some embodiments, administration may include one or more polynucleotide modification agents (e.g., SSB-HbW molecules) administered in one or more stages or combinations. For instance, by way of non-limiting example, a first combination is administered comprising a particular SSB-HbW molecule combined with an enhancing agent and a second combination is administered following a first combination, wherein the second combination combines the same or a different SSB-HbW molecule with an inhibiting agent.

In some embodiments, any forms of combination therapy that enhances survival of cells that contain (a) desired genetic change(s) may be used.

In some embodiments, other forms of combination therapy that facilitate or provide detection of cells that contain (a) desired genetic change(s) may be used.

In some embodiments, other forms of combination therapy that facilitate or provide identification of cells that contain (a) desired genetic change(s) may be used.

### Cells

In some embodiments, the present disclosure provides technologies (e.g., systems, compositions, methods) for genetic modification of cells. In some embodiments, provided technologies have an advantage of being capable of modifying multiple different cell types, including replicating cell types and/or non-replicating cell types.

In some embodiments, a cell for genetic modification using provided technologies is a replicating cell. As non-limiting example, such cells can be stem or progenitor cells, such as hematopoietic stem cells or muscle satellite cells, or other types of cells such as B-cells, intestine Paneth cells, bone osteoclasts etc.

In some embodiments, a cell for genetic modification using provided technologies is a non-replicating cell. As non-limiting example, such cell types include neurons, myocytes, terminal natural killer cells etc.

In some embodiments, a cell is provided from a cell line, e.g., a stable cell line (e.g., HEK293, e.g., U937, etc.) In some embodiments, a cell is provided from a primary cell culture. In some embodiments, a cell is extracted from a subject in need of treatment. In some embodiments, cells are engineered to stably express exogenous genetic products. In some embodiments, a cell may be an artificial cell. In some embodiments, a cell may be an engineered cell.

In some embodiments, the present disclosure provides technologies that can be used to contact one or more cells. In some embodiments, a cell is in vitro, ex vivo, or in vivo. In some embodiments, a cell (e.g., a mammalian cell) is autologous, meaning the cell is obtained, e.g., from a subject (e.g., a mammal) and cultured ex vivo.

In some embodiments, a cell is a human cell, a mouse cell, a porcine cell, a rabbit cell, a dog cell, a rat cell, a sheep cell, a cat cell, a horse cell, a non-human primate cell, or an insect cell.

In some embodiments, a cell is a stem cell. In some embodiments, a cell is a progenitor or precursor cell. In some embodiments, a cell is a differentiated cell. In some embodiments, a cell is a specialized cell type (e.g., a neuron, a cardiac cell, a kidney cell, an islet cell, etc.). In some embodiments, a cell is a post-mitotic cell (e.g., neuron).

In some embodiments, a cell is transiently or non-transiently transfected with one or more vectors comprising a sequence encoding a polynucleotide modification agent (e.g., SSB-HbW molecule) and/or a sequence modification polynucleotide. In some embodiments, a cell is transfected in a substantially similar state as it occurs or exists in a subject. In some such embodiments, such a transfection may occur in vitro, ex vivo, or in vivo. In some embodiments, a cell is derived from one or more cells taken from a subject, such as development or a stable cell line and/or a primary cell culture. A wide variety of cell lines for tissue culture are known in the art. Examples of cells lines include, but are not limited to, HEK293 and U937. Cell lines are available from a variety of sources known to those with skill in the art, for example, the American Type Culture Collection (ATCC) (Manassas, VA, USA). In some embodiments, a cell transfected with one or more components of genetic modification system described herein (e.g., a zinc finger helicase beta wing mediated DNA modification system) may be used establish a new cell line comprising one or more genetic modifications (e.g., any conceivable genetic modification including but not limited to loss-of-function, gain-of-function, insertion, deletion including one or more changes to create cellular models of known diseases, e.g., Alzheimer's disease or various genotypically-characterized cancers, using, e.g., known pathological mutations etc.)

In some embodiments, the present disclosure further provides cells produced by such methods and organisms (such as animals, plants, or fungi) comprising or produced from such cells as described herein. In some embodiments, for example, a SSB-HbW molecule in combination with a sequence modification polynucleotide such as a donor template, comprise an exemplary genetic modification system (e.g., a zinc finger helicase beta wing mediated DNA modification system). In some embodiments, such an exemplary genetic modification system (e.g., a zinc finger helicase beta wing mediated DNA modification system) is delivered to a cell. In some such embodiments, delivery is achieved by contacting a cell with one or more components of a zinc finger helicase beta wing mediated DNA modification system, e.g., one or more agents of the present disclosure (e.g., one or more modification agents and/or one or more sequence modification polynucleotides). In some embodiments, such methods can be used to administer nucleic acid encoding components of a genetic modification system (e.g., a zinc finger helicase beta wing mediated DNA modification system) to cells in culture (e.g., in vitro or ex vivo), or in a host organism (e.g., in vivo or ex vivo).

### EXEMPLARY NUMBERED EMBODIMENTS

Embodiment 1. A polynucleotide modification agent comprising a helicase beta-wing element (“HbW element”) and a sequence-specific binding element, wherein the HbW element is or comprises a helicase beta-wing.

Embodiment 2. The polynucleotide modification agent of embodiment 1, wherein the HbW element is or comprises a helicase beta-wing polypeptide having an anti-parallel beta-sheet structure.

Embodiment 3. The polynucleotide modification agent of embodiment 1 or 2, wherein the HbW element is or comprises a polypeptide derived from a prokaryotic or eukaryotic helicase.

Embodiment 4. The polynucleotide modification agent of embodiment 1 or 2, wherein the HbW element is or comprises a helicase beta-wing polypeptide with a mammalian sequence derived from a mammalian helicase polypeptide.

Embodiment 5. The polynucleotide modification agent of any one of embodiments 1 to 4, wherein the HbW element is or comprises a helicase beta-wing polypeptide derived from BLM helicase, WRN helicase or RECQ1.

Embodiment 6. The polynucleotide modification agent of any one of embodiments 1 to 5, wherein the HbW element is or comprises a polypeptide with a human sequence.

Embodiment 7. The polynucleotide modification agent of any one of embodiments 1 to 6, wherein the HbW element is or comprises a helicase beta-wing polypeptide with a human sequence derived from a human helicase polypeptide.

Embodiment 8. The polynucleotide modification agent of any one of embodiments 1 to 7, wherein the HbW element is or comprises a helicase beta-wing polypeptide derived from human BLM helicase, human WRN helicase or human RECQ1.

Embodiment 9. The polynucleotide modification agent of any one of embodiments 1 to 8, wherein
(i) the HbW element is or comprises a polypeptide sequence that is at least 80%, at least 85%, at least 90%, at least 95%, at least 96%, at least 97%, at least 98% at least 99%, or 100% identical to a sequence set forth in any one of SEQ ID NOs: 1 to 3; and/or
(i) the HbW element is or comprises a polypeptide sequence with up to 2 amino acid substitutions to a sequence set forth in any one of SEQ ID NOs: 1 to 3.

Embodiment 10. The polynucleotide modification agent of any one of embodiments 1 to 9, wherein the sequence-specific binding element is or comprises one or more Zinc Finger polypeptides; TALE-polypeptides; helix-loop-helix polypeptides; helix-turn-helix polypeptides; CAS-polypeptides; leucine zipper polypeptides; beta-scaffold polypeptides; homeo-domain polypeptides; high-mobility group box polypeptides, or a characteristic portion of any thereof and/or combination thereof.

Embodiment 11. The polynucleotide modification agent of any one of embodiments 1 to 10, wherein the sequence-specific binding element comprises a polypeptide with a human sequence.

Embodiment 12. The polynucleotide modification agent of any one of embodiments 1 to 11, wherein the sequence-specific binding element is or comprises a zinc finger polypeptide comprising one or more zinc finger arrays.

Embodiment 13. The polynucleotide modification agent of any one of embodiments 1 to 12, wherein the sequence-specific binding element is or comprises a zinc finger polypeptide comprising at least five, six, seven, eight, nine, ten, or eleven, or more, zinc finger arrays.

Embodiment 14. The polynucleotide modification agent of embodiment 12 or 13, wherein the zinc finger arrays comprise at least one alpha helix engineered to comprise a modified amino acid sequence that differs from that of its corresponding wild type sequence.

Embodiment 15. The polynucleotide modification agent of any one of embodiments 12 to 14, wherein the sequence-specific binding element comprises one or more zinc finger arrays comprising a polypeptide sequence that is at least 80%, at least 85%, at least 90%, at least 95%, at least 96%, at least 97%, at least 98% at least 99%, or 100% identical to a sequence set forth in SEQ ID NO: 5 or 6.

Embodiment 16. The polynucleotide modification agent of any one of embodiments 1 to 15, wherein the sequence-specific binding element targets a gene sequence.

Embodiment 17. The polynucleotide modification agent of any one of embodiments 1 to 16, wherein the sequence-specific binding element targets a mammalian gene sequence.

Embodiment 18. The polynucleotide modification agent of any one of embodiments 1 to 17, wherein the sequence-specific binding element targets a human gene sequence.

Embodiment 19. The polynucleotide modification agent of any one of embodiments 1 to 18, wherein the sequence-specific binding element targets a sequence in a gene associated with a disease.

Embodiment 20. The polynucleotide modification agent of any one of embodiments 1 to 19, wherein the sequence-specific binding element targets a sequence in ApoE, Bcl11A, DMD, EGFPDP2, PolG, MMACHC, MMUT, PAH, CFTR.

Embodiment 21. The polynucleotide modification agent of any one of embodiments 1 to 20, further comprising a linker.

Embodiment 22. The polynucleotide modification agent of embodiment 21, wherein the linker is or comprises a polypeptide.

Embodiment 23. The polynucleotide modification agent of embodiment 21 or 22, wherein the linker is or comprises a polypeptide with a human sequence.

Embodiment 24. The polynucleotide modification agent of any one of embodiment 21 to 23, wherein the linker is or comprises a polypeptide between 2 and 100 amino acids in length or 0.2 kD and 10 kD in size.

Embodiment 25. The polynucleotide modification agent of any one of embodiments 21 to 24, wherein the linker is or comprises:
(i) a polypeptide derived from a human helicase polypeptide; and/or
(ii) a polypeptide sequence that is at least 80%, at least 85%, at least 90%, at least 95%, at least 96%, at least 97%, at least 98% at least 99%, or 100% identical to a sequence set forth in any one of SEQ ID NOs: 7-9.

Embodiment 26. The polynucleotide modification agent of any one of embodiments 21 to 25, wherein:
(i) the HbW element is derived from human BLM helicase and the linker is derived from human BLM helicase,
(ii) the HbW element is derived from human WRN helicase and the linker is derived from human WRN helicase, or
(iii) the HbW element is derived from human RECQ1helicase and the linker is derived from human RECQ1helicase helicase.

Embodiment 27. The polynucleotide modification agent of any one of embodiments 21 to 25, comprising:
(i) the HbW element comprises a sequence of SEQ ID NO: 1, or a sequence with 1, 2, or 3 substitutions in SEQ ID NO: 1 and the linker comprises a sequence of SEQ ID NO: 7 or sequence with 1, 2, or 3 substitutions in SEQ ID NO: 7;
(ii) the HbW element comprises a sequence of SEQ ID NO: 2, or a sequence with 1, 2, or 3 substitutions in SEQ ID NO: 2 and the linker comprises a sequence of SEQ ID NO: 8 or sequence with 1, 2, or 3 substitutions in SEQ ID NO: 8,
(iii) the HbW element comprises a sequence of SEQ ID NO: 3, or a sequence with 1, 2, or 3 substitutions in SEQ ID NO: 3 and the linker comprises a sequence of SEQ ID NO: 9 or sequence with 1, 2, or 3 substitutions in SEQ ID NO: 9.

Embodiment 28. The polynucleotide modification agent of any one of embodiments 1 to 27, wherein the polynucleotide modification agent does not comprise a linker.

Embodiment 29. The polynucleotide modification agent of any one of embodiments 1 to 28, wherein the polynucleotide modification agent lacks nuclease function.

Embodiment 30. The polynucleotide modification agent of any one of embodiments 1 to 29, wherein the HbW element interacts with a target site and wherein the sequence-specific binding element binds to a landing site.

Embodiment 31. The polynucleotide modification agent of embodiment 30, wherein the landing site is adjacent to the target site.

Embodiment 32. The polynucleotide modification agent of any one of embodiments 30 or 31, wherein the sequence-specific binding element binds to the landing site with a binding affinity characterized by a dissociation constant of 10 E−6 or lower.

Embodiment 33. The polynucleotide modification agent of any one of embodiments 1 to 31, wherein sequence-specific binding element binds to a single strand of polynucleotide.

Embodiment 34. The polynucleotide modification agent of any one of embodiments 1 to 32, wherein
a) the HbW element breaks one or more hydrogen bonds within a target site of a polynucleotide; and/or
b) the HbW element inserts between strands of a polynucleotide.

Embodiment 35. The polynucleotide modification agent of any one of embodiments 1 to 34, wherein the agent does not cause modification of a non-target site.

Embodiment 36. A nucleic acid encoding the polynucleotide modification agent of any one of embodiments 1 to 35.

Embodiment 37. A vector comprising the nucleic acid of embodiment 36.

Embodiment 38. A composition comprising the polynucleotide modification agent of any one of embodiments 1 to 35, the nucleic acid of embodiment 36, or the vector of embodiment 37.

Embodiment 39. A pharmaceutical composition comprising (i) the polynucleotide modification agent of any one of embodiments 1 to 35, the nucleic acid of embodiment 36, or the vector of embodiment 37, and (ii) a pharmaceutically or physiologically acceptable carrier.

Embodiment 40. A combination comprising (i) the polynucleotide modification agent of any one of embodiments 1 to 35, the nucleic acid of embodiment 36, or the vector of embodiment 37, and (ii) a sequence modification polynucleotide.

Embodiment 41. The combination of embodiment 40, wherein the polynucleotide modification agent is a polypeptide.

Embodiment 42. The combination of embodiment 40, wherein the polynucleotide modification agent is encoded on DNA.

Embodiment 43. The combination of embodiment 40, wherein the polynucleotide modification agent is encoded on RNA (e.g., mRNA).

Embodiment 44. The combination of any one of embodiments 40 to 43, wherein the sequence modification polynucleotide comprises a sequence that is capable of being incorporated into a copy of a gene in a genome.

Embodiment 45. The combination of any one of embodiments 40 to 44, wherein the sequence modification polynucleotide comprises a sequence that is capable of being incorporated into a copy of a disease-associated gene.

Embodiment 46. The combination of any one of embodiments 40 to 45, wherein the sequence modification polynucleotide comprises a sequence that is capable of being incorporated into a copy of a gene selected from: ApoE, BCL11A, DMD, PAH, PolG, MMACHC, MMUT, and CFTR.

Embodiment 47. The combination of any one of embodiments 44 to 46, wherein the gene is a mammalian gene.

Embodiment 48. The combination of any one of embodiments 44 to 47, wherein the gene is a human gene.

Embodiment 49. The combination of any one of embodiments 44 to 48, wherein the incorporating occurs during DNA replication or DNA synthesis.

Embodiment 50. The combination of any one of embodiments 44 to 49, wherein the sequence modification polynucleotide comprises a sequence that is at least 80%, at least 85%, at least 90%, at least 95%, at least 96%, at least 97%, at least 98% at least 99%, or 100% identical to a sequence set forth in any one of SEQ ID NOs: 10-13, 72, 80, 88, 96, and 104.

Embodiment 51. A kit comprising the composition of embodiment 38 or the pharmaceutical composition of embodiment 39.

Embodiment 52. The kit of embodiment 51, further comprising a sequence modification polynucleotide.

Embodiment 53. The kit of embodiment 52, further comprising at least one additional agent, wherein the at least one additional agent is or comprises an agent that (i) induces DNA replication and/or (ii) induces DNA strand repair.

Embodiment 54. A method comprising contacting a cell or population of cells with (i) the polynucleotide modification agent of any one of embodiments 1 to 35; and (ii) a sequence modification polynucleotide.

Embodiment 55. The method of embodiment 54, wherein the cell or population of cells comprise a DNA polynucleotide comprising at least one target site.

Embodiment 56. The method of embodiment 55, wherein the sequence modification polynucleotide:
(i) binds specifically to one strand of the DNA at a target site; and
(ii) has a mismatch or other DNA sequence difference relative to the target site, so that usage of the sequence modification polynucleotide incorporates the sequence modification into a complement of the one strand.

Embodiment 57. The method of embodiment 56, wherein the incorporation of the sequence modification into the complement of the one strand occurs simultaneously or after the HbW element interacts with the DNA.

Embodiment 58. The method of any one of embodiments 54 to 57, wherein the cell or population of cells are non-replicating and/or post-mitotic.

Embodiment 59. The method of any one of embodiments 54 to 57, wherein the cell or population of cells comprise DNA that is actively replicating.

Embodiment 60. The method of any one of embodiments 54 to 57, wherein the polynucleotide modification agent does not itself catalyze single and/or double-stranded DNA breaks.

Embodiment 61. The method of any one of embodiments 54 to 60, further comprising contacting the cell or population of cells with an enhancing agent and/or an inhibiting agent.

Embodiment 62. The method of embodiment 61, wherein the enhancing and/or inhibiting agent alters DNA recombination events, and wherein the enhancing agent and/or inhibiting agent itself does not contact the DNA.

Embodiment 63. The method of embodiment 61 of 62, wherein the enhancing agent and/or inhibiting agent is or comprises RNAi activity.

Embodiment 64. The method of any one of embodiments 61 to 63, wherein the incorporation of the sequence modification occurs at a 2 to 10 times greater frequency with enhancing agent and/or inhibiting agent relative to an otherwise identical method that does not include the enhancing agent or inhibiting agent.

Embodiment 65. The method of any one of embodiments 61 to 64, further comprising contacting the cell or population of cells with at least one additional agent that (i) induces DNA replication and/or (ii) induces DNA repair.

Embodiment 66. A method comprising:

contacting DNA with (i) the polynucleotide modification agent of any one of embodiments 1 to 35; and (ii) a sequence modification polynucleotide.

Embodiment 67. The method of embodiment 66, wherein the DNA comprises at least one target site.

Embodiment 68. The method of embodiment 67, wherein the sequence modification polynucleotide:
(i) binds specifically to one strand of the DNA at a target site; and
(ii) has a DNA sequence difference relative to the target sequence.

Embodiment 69. The method of embodiment 68, wherein method induces a change in a target sequence that corresponds to the sequence of the sequence modification polynucleotide.

Embodiment 70. A method comprising:
administering to a subject (i) the polynucleotide modification agent of any one of embodiments 1 to 35; and (ii) a sequence modification polynucleotide.

Embodiment 71. The method of embodiment 70, wherein the sequence modification polynucleotide:
(i) binds specifically to a target sequence in a population of cells of the subject; and
(ii) has a sequence difference relative to the target sequence.

Embodiment 72. The method of embodiment 71, wherein method induces a change in the target sequence of the population of cells of the subject, wherein the change in the target sequence corresponds to the sequence of the sequence modification polynucleotide.

Embodiment 73. The method of embodiment 72, wherein the population of cells is or comprises:
(i) a tissue,
(ii) an organ,
(iii) a tumor, or
(iv) a cell-specific cell lineage.

Embodiment 74. The method of embodiment 73, wherein the population of cells is or comprises a cell-specific cell lineage that is or comprises (i) neural cells and/or (ii) neuronal cells.

Embodiment 75. The method of any one of embodiments 70 to 74, wherein the subject is mammal.

Embodiment 76. The method of any one of embodiments 70 to 75, wherein the subject is a non-human primate or a human.

Embodiment 77. The method of embodiment 76, wherein the subject is a fetal, infant, child, adolescent, or adult human.

Embodiment 78. The method of any one of embodiments 54 to 77, wherein the sequence modification polynucleotide comprises a sequence that specifically targets a disease-associated gene.

Embodiment 79. The method of embodiments 78, wherein the disease-associated gene is a mammalian gene.

Embodiment 80. The method of embodiments 79, wherein the disease-associated gene is a human gene.

Embodiment 81. The method of any one of embodiments 54 to 80, wherein the sequence modification polynucleotide comprises a sequence that specifically targets a sequence within a gene selected from: B-cell lymphoma/leukemia 11 A (BCL11 A) gene, a dystrophin gene (DMD), MMACHC, a DNA polymerase γ gene (PolG), a methylmalonyl CoA mutase gene (MMUT), a phenylalanine hydroxylase gene (PAH), a cystic fibrosis transmembrane conductance regulator (CFTR) gene, a Kruppel-like factor 1 gene, a mammalian beta globin gene, gamma globin gene, a C—C chemokine receptor type (CCR)5 gene, a chemokine (C—X—C motif) receptor 4 (CXCR4) gene, a protein phosphatase 1, regulatory subunit 12C (PPP1R12C) gene, an hypoxanthine phosphoribosyltransferase (HPRT) gene, an albumin gene, a Factor VIII gene, a Factor IX gene, a leucine-rich repeat kinase 2 (LRRK2) gene, a huntingtin (Htt) gene, a rhodopsin (RHO) gene, a surfactant protein B gene (SFTPB), a T-cell receptor alpha (TRAC) gene, a T-cell receptor beta (TRBC) gene, a programmed cell death 1 (PD1) gene, a cytotoxic T-lymphocyte antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with antigen processing (TAP)1 gene, a TAP2 gene, a tapasin gene (TAPBP), a class II major histocompatibility complex transactivator (CUTA) gene, a glucocorticoid receptor gene (GR), an interleukin 2 receptor gamma (IL2RG) gene and a regulatory factor X 5 (RFX5) gene.

Embodiment 82. The method of any one of embodiments 54 to 80, wherein the sequence modification polynucleotide comprises a sequence that specifically targets a sequence within a gene selected from: EGFPDP2, ApoE, Bcl11A, DMD, PolG, MMACHC, MMUT, PAH, CFTR, MMA, and PKU.

Embodiment 83. The method of any one of embodiments 54 to 80, wherein the sequence modification polynucleotide comprises a sequence that specifically targets a sequence within a human gene selected from: human ApoE, human BCL11A, and human DMD.

Embodiment 84. The method of any one of embodiments 54 to 83, wherein the sequence modification polynucleotide comprises a sequence that is at least 80%, at least 85%, at least 90%, at least 95%, at least 96%, at least 97%, at least 98% at least 99%, or 100% identical to a sequence set forth in any one of SEQ ID NOs: 10-13, 72, 80, 88, 96, and 104.

Embodiment 85. The method of any one of embodiments 54 to 83, wherein one target sequence is modified.

Embodiment 86. The method of any one of embodiments 54 to 83, wherein two or more target sequences are modified.

Embodiment 87. The method of embodiment 86, wherein the two or more target sequences are associated with different genes.

Embodiment 88. The method of embodiment 86, wherein the two or more target sequences are associated with the same gene.

Embodiment 89. The method of embodiment 87, wherein the different genes are located on the same chromosome.

Embodiment 90. The method of embodiment 87, wherein the different genes are located on different chromosomes.

Embodiment 91. A method of characterizing the polynucleotide modification agent of any one of embodiments 1 to 35, comprising measuring one or more of binding efficiency, binding affinity, sequence modification efficiency, and stability of at least one element of the polynucleotide modification agent.

The invention is further illustrated in the following Examples.

### EXAMPLES

The following examples are given for the purpose of illustrating various embodiments of the disclosure and are not meant to limit the present disclosure in any fashion. Changes therein and other uses which are encompassed within the spirit of the disclosure as defined by the scope of the claims will occur to those skilled in the art.

### Example 1: SSB-HbW Designs: Generation and Production of Exemplary Polynucleotide Modification Agents

The present example describes the design and production of exemplary polynucleotide modification agents. In particular, the present example describes the production of three exemplary SSB-HbW constructs using exemplary human helicase beta-wing structures in combination with human helicase derived linker sequences.

An aspect of this disclosure is that various elements of a polynucleotide modification agent can be modular in design, as depicted in the schematic of FIG. 2. Thus, each of the elements: SSB element, HbW element, and optional linker element can be separately designed, selected and/or optimized.

Polynucleotide modification agents of the present example include a SSB element that includes a zinc finger array that was designed to be a DNA recognition domain, as illustrated in FIG. 3.

In particular, an exemplary SSB element that includes an array of 9 zinc fingers was specifically designed to recognize a 27-nucleotide sequence on the leading strand of human ApoE, corresponding to:

(SEQ ID NO.: 22)
5′-GCGGCCGCCTGGTGCAGTACCGCGGCG-3′.

An amino acid sequence encoding this exemplary ApoE targeting SSB element comprising 9 zinc finger arrays is:

(SEQ ID NO.: 23)
FQCRICMRNFSRSSDLTRHIRTHTGEKPFACDICGRKFARSDTLTRHTKI
HTGSQKPFQCRICMRNFSQSGDLSEHIRTHTGEKPFACDICGRKFATSGH
LTTHTKIHTGSQKPFQCRICMRNFSDSSHLTTHIRTHTGEKPFACDICGR
KFARSSHLTTHTKIHTGSQKPFQCRICMRNFSDRSDLTRHIRTHTGEKPF
ACDICGRKFADRSDLTRHTKIHTGSQKPFQCRICMRNFSRSDTLTRHIRT
HTG.

Similarly, linker and/or HbW elements of a polynucleotide modification agent can also be separately designed, selected and/or optimized. The present example describes exemplary constructs that include exemplary linker and HbW elements that are both derived from human helicases. FIG. 5 illustrates an alignment of exemplary helicase linker and beta wing domains.

As non-limiting example, a SSB-HbW design using a BLM helicase beta wing amino acid sequence: DLYINANDQAIAYVMLG (SEQ ID NO: 2) and a BLM helicase derived linker SRHNERLFKKLILDKILDE (SEQ ID NO: 8) are combined as linker and HbW elements, respectively, of an exemplary polynucleotide modification agent.

An exemplary polynucleotide modification agent targeting ApoE with BLM helicase derived linker and HbW elements is encoded on plasmid pb110, with a full length DNA sequence of SEQ ID NO: 53; cDNA sequence of SEQ ID. NO: 54; and SSB-HbW amino acid sequence of SEQ ID. NO: 55 (sequences provided in Table 4 below).

Without being bound to any theory in particular, this linker and beta-wing combination, derived from a single human helicase, exemplifies usage of a naturally occurring human linker sequence that contains a combination of amino acids with positively charged sidechains (H, R, K), amino acids with negatively charged amino acids (E, D), amino acids with polar side chains (S,N) and amino acids with non-polar side chains (L, I, F).

As non-limiting example, a SSB-HbW design using a WRN helicase beta wing amino acid sequence: VSRYNKFMKICALTKKG (SEQ ID NO: 1) and a WRN helicase derived linker LRGSNSQRLADQYRRHSLFGTGVE (SEQ ID NO: 7) are combined as linker and HbW elements, respectively, of an exemplary polynucleotide modification agent.

An exemplary polynucleotide modification agent targeting ApoE with WRN helicase linker and HbW elements is encoded on plasmid pb 106, with a full length DNA sequence of SEQ ID NO: 56; cDNA sequence of SEQ ID. NO: 57; and SSB-HbW amino acid sequence of SEQ ID. NO: 58 (sequences provided in Table 4 below). Without being bound to any theory in particular, this linker and beta-wing combination, derived from a single human helicase, exemplifies usage of a naturally occurring human linker sequence that comprises a “LRGS” amino acid sequence, which can be used in synthetic biology as a linker that can be encoded with nucleotides that contain a BamH1 restriction enzyme site.

As non-limiting example, a SSB-HbW design using a RECQ1 helicase beta wing amino acid sequence DYSFTAYATISYLKIG (SEQ ID NO: 3) and a RECQ1 helicase derived linker EKIIAHFLIQQYLKE (SEQ ID NO: 9) are combined as linker and HbW elements, respectively, of an exemplary polynucleotide modification agent.

An exemplary polynucleotide modification agent targeting ApoE with RECQ1 helicase linker and HbW elements is encoded on plasmid pb111, with a full length DNA of SEQ ID NO: 59; cDNA sequence of SEQ ID. NO. 60 (sequences provided in Table 4 below); and SSB-HbW amino acid sequence SEQ ID NO: 61. Without being bound to any theory in particular, this linker and beta-wing combination, derived from a single human helicase, exemplifies a creation of a zinc finger array and linker amino acid fusion sequence of “TGEK” (“TG” zinc finger array last 2 amino acids; “EK” linker first two amino acids), which resembles a canonical zinc finger linker of “TGEKP”. Such linkers and variants thereof are abundant in the human genome and/or proteome and may thus provide a fusion peptide sequence with favorable immunological properties.

TABLE 4
Exemplary polynucleotide modification agents targeting ApoE

| SEQ | | AA/ | |
|---|---|---|---|
| ID | | DNA/ | |
| NO: | Feature | cDNA | Sequence |
| 53 | pb110 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCCAGTGCAGGATCTGTATGCGCAACTTTTCTCGGTCCTCCG |
| | | | ACCTGACCCGGCACATCAGAACCCATACAGGCGAAAAGCCTTTCGCCTGCGACATT |
| | | | TGTGGGAGAAAATTTGCTCGGTCCGACACCCTGACCCGGCATACCAAGATCCACAC |
| | | | CGGCTCTCAGAAACCATTCCAGTGCCGCATTTGTATGCGGAATTTTTCCCAGTCCG |
| | | | GCGACCTGTCCGAGCATATCCGCACTCACACCGGAGAGAAGCCCTTTGCTTGCGAC |
| | | | ATTTGTGGCAGGAAATTTGCTACCTCCGGCCACCTGACCACCCACACTAAGATCCA |
| | | | TACTGGGTCACAGAAACCTTTCCAGTGCCGGATTTGTATGAGAAACTTTAGCGACT |
| | | | CCTCCCACCTGACCACCCACATCAGAACCCATACAGGCGAAAAGCCTTTCGCCTGC |
| | | | GACATTTGTGGGAGAAAATTTGCTCGGTCCTCCCACCTGACCACCCATACCAAGAT |
| | | | CCACACCGGCTCTCAGAAACCATTCCAGTGCCGCATTTGTATGCGGAATTTTTCCG |
| | | | ACCGGTCCGACCTGACCCGGCATATCCGCACTCACACCGGAGAGAAGCCCTTTGCT |
| | | | TGCGACATTTGTGGCAGGAAATTTGCTGACCGGTCCGACCTGACCCGGCACACTAA |
| | | | GATCCATACTGGGTCACAGAAACCTTTCCAGTGCCGGATTTGTATGAGAAACTTTA |
| | | | GCCGGTCCGACACCCTGACCCGGCACATCAGAACACATACTGGATCCCGGCACAAC |
| | | | GCCGAGCGGCTGTTCAAGAAGCTGATCCTGGACAAGATCCTGGACGAGGACCTGTA |
| | | | CATCAACGCCAACGACCAGGCCATCGCCTACGTGATGCTGGGCTGAGCGGCCGCTC |
| | | | GAGTCTAGAGGGCCCGTTTAAACCCGCTGATCAGCCTCGACTGTGCCTTCTAGTTG |
| | | | CCAGCCATCTGTTGTTTGCCCCTCCCCCGTGCCTTCCTTGACCCTGGAAGGTGCCA |
| | | | CTCCCACTGTCCTTTCCTAATAAAATGAGGAAATTGCATCGCATTGTCTGAGTAGG |
| | | | TGTCATTCTATTCTGGGGGGTGGGGTGGGGCAGGACAGCAAGGGGGAGGATTGGGA |
| | | | AGACAATAGCAGGCATGCTGGGGATGCGGTGGGCTCTATGGCTTCTACTGGGCGGT |
| | | | TTTATGGACAGCAAGCGAACCGGAATTGCCAGCTGGGGCGCCCTCTGGTAAGGTTG |
| | | | GGAAGCCCTGCAAAGTAAACTGGATGGCTTTCTCGCCGCCAAGGATCTGATGGCGC |
| | | | AGGGGATCAAGCTCTGATCAAGAGACAGGATGAGGATCGTTTCGCATGATTGAACA |
| | | | AGATGGATTGCACGCAGGTTCTCCGGCCGCTTGGGTGGAGAGGCTATTCGGCTATG |
| | | | ACTGGGCACAACAGACAATCGGCTGCTCTGATGCCGCCGTGTTCCGGCTGTCAGCG |
| | | | CAGGGGCGCCCGGTTCTTTTTGTCAAGACCGACCTGTCCGGTGCCCTGAATGAACT |
| | | | GCAAGACGAGGCAGCGCGGCTATCGTGGCTGGCCACGACGGGCGTTCCTTGCGCAG |
| | | | CTGTGCTCGACGTTGTCACTGAAGCGGGAAGGGACTGGCTGCTATTGGGCGAAGTG |
| | | | CCGGGGCAGGATCTCCTGTCATCTCACCTTGCTCCTGCCGAGAAAGTATCCATCAT |
| | | | GGCTGATGCAATGCGGCGGCTGCATACGCTTGATCCGGCTACCTGCCCATTCGACC |
| | | | ACCAAGCGAAACATCGCATCGAGCGAGCACGTACTCGGATGGAAGCCGGTCTTGTC |
| | | | GATCAGGATGATCTGGACGAAGAGCATCAGGGGCTCGCGCCAGCCGAACTGTTCGC |
| | | | CAGGCTCAAGGCGAGCATGCCCGACGGCGAGGATCTCGTCGTGACCCATGGCGATG |
| | | | CCTGCTTGCCGAATATCATGGTGGAAAATGGCCGCTTTTCTGGATTCATCGACTGT |
| | | | GGCCGGCTGGGTGTGGCGGACCGCTATCAGGACATAGCGTTGGCTACCCGTGATAT |
| | | | TGCTGAAGAGCTTGGCGGCGAATGGGCTGACCGCTTCCTCGTGCTTTACGGTATCG |
| | | | CCGCTCCCGATTCGCAGCGCATCGCCTTCTATCGCCTTCTTGACGAGTTCTTCTGA |
| | | | ATTATTAACGCTTACAATTTCCTGATGCGGTATTTTCTCCTTACGCATCTGTGCGG |
| | | | TATTTCACACCGCATACAGGTGGCACTTTTCGGGGAAATGTGCGCGGAACCCCTAT |
| | | | TTGTTTATTTTTCTAAATACATTCAAATATGTATCCGCTCATGAGACAATAACCCT |
| | | | GATAAATGCTTCAATAATAGCACGTGCTAAAACTTCATTTTTAATTTAAAAGGATC |
| | | | TAGGTGAAGATCCTTTTTGATAATCTCATGACCAAAATCCCTTAACGTGAGTTTTC |
| | | | GTTCCACTGAGCGTCAGACCCCGTAGAAAAGATCAAAGGATCTTCTTGAGATCCTT |
| | | | TTTTTCTGCGCGTAATCTGCTGCTTGCAAACAAAAAAACCACCGCTACCAGCGGTG |
| | | | GTTTGTTTGCCGGATCAAGAGCTACCAACTCTTTTTCCGAAGGTAACTGGCTTCAG |
| | | | CAGAGCGCAGATACCAAATACTGTCCTTCTAGTGTAGCCGTAGTTAGGCCACCACT |
| | | | TCAAGAACTCTGTAGCACCGCCTACATACCTCGCTCTGCTAATCCTGTTACCAGTG |
| | | | GCTGCTGCCAGTGGCGATAAGTCGTGTCTTACCGGGTTGGACTCAAGACGATAGTT |
| | | | ACCGGATAAGGCGCAGCGGTCGGGCTGAACGGGGGGTTCGTGCACACAGCCCAGCT |
| | | | TGGAGCGAACGACCTACACCGAACTGAGATACCTACAGCGTGAGCTATGAGAAAGC |
| | | | GCCACGCTTCCCGAAGGGAGAAAGGCGGACAGGTATCCGGTAAGCGGCAGGGTCGG |
| | | | AACAGGAGAGCGCACGAGGGAGCTTCCAGGGGGAAACGCCTGGTATCTTTATAGTC |
| | | | CTGTCGGGTTTCGCCACCTCTGACTTGAGCGTCGATTTTTGTGATGCTCGTCAGGG |
| | | | GGGCGGAGCCTATGGAAAAACGCCAGCAACGCGGCCTTTTTACGGTTCCTGGGCTT |
| | | | TTGCTGGCCTTTTGCTCACATGTTCTT |
| 54 | pb110 | cDNA | ATGGCGGCGATGGCCGAGCGGCCCTTCCAGTGCAGGATCTGTATGCGCAACTTTTC |
| | | | TCGGTCCTCCGACCTGACCCGGCACATCAGAACCCATACAGGCGAAAAGCCTTTCG |
| | | | CCTGCGACATTTGTGGGAGAAAATTTGCTCGGTCCGACACCCTGACCCGGCATACC |
| | | | AAGATCCACACCGGCTCTCAGAAACCATTCCAGTGCCGCATTTGTATGCGGAATTT |
| | | | TTCCCAGTCCGGCGACCTGTCCGAGCATATCCGCACTCACACCGGAGAGAAGCCCT |
| | | | TTGCTTGCGACATTTGTGGCAGGAAATTTGCTACCTCCGGCCACCTGACCACCCAC |
| | | | ACTAAGATCCATACTGGGTCACAGAAACCTTTCCAGTGCCGGATTTGTATGAGAAA |
| | | | CTTTAGCGACTCCTCCCACCTGACCACCCACATCAGAACCCATACAGGCGAAAAGC |
| | | | CTTTCGCCTGCGACATTTGTGGGAGAAAATTTGCTCGGTCCTCCCACCTGACCACC |
| | | | CATACCAAGATCCACACCGGCTCTCAGAAACCATTCCAGTGCCGCATTTGTATGCG |
| | | | GAATTTTTCCGACCGGTCCGACCTGACCCGGCATATCCGCACTCACACCGGAGAGA |
| | | | AGCCCTTTGCTTGCGACATTTGTGGCAGGAAATTTGCTGACCGGTCCGACCTGACC |
| | | | CGGCACACTAAGATCCATACTGGGTCACAGAAACCTTTCCAGTGCCGGATTTGTAT |
| | | | GAGAAACTTTAGCCGGTCCGACACCCTGACCCGGCACATCAGAACACATACTGGAT |
| | | | CCCGGCACAACGCCGAGCGGCTGTTCAAGAAGCTGATCCTGGACAAGATCCTGGAC |
| | | | GAGGACCTGTACATCAACGCCAACGACCAGGCCATCGCCTACGTGATGCTGGGCTG |
| | | | A |
| 55 | pb110 | AA | MAAMAERPFQCRICMRNFSRSSDLTRHIRTHTGEKPFACDICGRKFARSDTLTRHT |
| | | | KIHTGSQKPFQCRICMRNFSQSGDLSEHIRTHTGEKPFACDICGRKFATSGHLTTH |
| | | | TKIHTGSQKPFQCRICMRNFSDSSHLTTHIRTHTGEKPFACDICGRKFARSSHLTT |
| | | | HTKIHTGSQKPFQCRICMRNFSDRSDLTRHIRTHTGEKPFACDICGRKFADRSDLT |
| | | | RHTKIHTGSQKPFQCRICMRNFSRSDTLTRHIRTHTGSRHNAERLFKKLILDKILD |
| | | | EDLYINANDQAIAYVMLG* |
| 56 | pb106 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCCAGTGCAGGATCTGTATGCGCAACTTTTCTCGGTCCTCCG |
| | | | ACCTGACCCGGCACATCAGAACCCATACAGGCGAAAAGCCTTTCGCCTGCGACATT |
| | | | TGTGGGAGAAAATTTGCTCGGTCCGACACCCTGACCCGGCATACCAAGATCCACAC |
| | | | CGGCTCTCAGAAACCATTCCAGTGCCGCATTTGTATGCGGAATTTTTCCCAGTCCG |
| | | | GCGACCTGTCCGAGCATATCCGCACTCACACCGGAGAGAAGCCCTTTGCTTGCGAC |
| | | | ATTTGTGGCAGGAAATTTGCTACCTCCGGCCACCTGACCACCCACACTAAGATCCA |
| | | | TACTGGGTCACAGAAACCTTTCCAGTGCCGGATTTGTATGAGAAACTTTAGCGACT |
| | | | CCTCCCACCTGACCACCCACATCAGAACCCATACAGGCGAAAAGCCTTTCGCCTGC |
| | | | GACATTTGTGGGAGAAAATTTGCTCGGTCCTCCCACCTGACCACCCATACCAAGAT |
| | | | CCACACCGGCTCTCAGAAACCATTCCAGTGCCGCATTTGTATGCGGAATTTTTCCG |
| | | | ACCGGTCCGACCTGACCCGGCATATCCGCACTCACACCGGAGAGAAGCCCTTTGCT |
| | | | TGCGACATTTGTGGCAGGAAATTTGCTGACCGGTCCGACCTGACCCGGCACACTAA |
| | | | GATCCATACTGGGTCACAGAAACCTTTCCAGTGCCGGATTTGTATGAGAAACTTTA |
| | | | GCCGGTCCGACACCCTGACCCGGCACATCAGAACACATACTGGGCTGAGAGGATCC |
| | | | AACTCCCAGCGGCTGGCCGACCAGTACCGGCGGCACTCCCTGTTCGGCACCGGCGT |
| | | | GGAGGTGTCCCGGTACAACAAGTTCATGAAGATCTGCGCCCTGACCAAGAAGGGCT |
| | | | GAGCGGCCGCTCGAGTCTAGAGGGCCCGTTTAAACCCGCTGATCAGCCTCGACTGT |
| | | | GCCTTCTAGTTGCCAGCCATCTGTTGTTTGCCCCTCCCCCGTGCCTTCCTTGACCC |
| | | | TGGAAGGTGCCACTCCCACTGTCCTTTCCTAATAAAATGAGGAAATTGCATCGCAT |
| | | | TGTCTGAGTAGGTGTCATTCTATTCTGGGGGGTGGGGTGGGGCAGGACAGCAAGGG |
| | | | GGAGGATTGGGAAGACAATAGCAGGCATGCTGGGGATGCGGTGGGCTCTATGGCTT |
| | | | CTACTGGGCGGTTTTATGGACAGCAAGCGAACCGGAATTGCCAGCTGGGGCGCCCT |
| | | | CTGGTAAGGTTGGGAAGCCCTGCAAAGTAAACTGGATGGCTTTCTCGCCGCCAAGG |
| | | | ATCTGATGGCGCAGGGGATCAAGCTCTGATCAAGAGACAGGATGAGGATCGTTTCG |
| | | | CATGATTGAACAAGATGGATTGCACGCAGGTTCTCCGGCCGCTTGGGTGGAGAGGC |
| | | | TATTCGGCTATGACTGGGCACAACAGACAATCGGCTGCTCTGATGCCGCCGTGTTC |
| | | | CGGCTGTCAGCGCAGGGGCGCCCGGTTCTTTTTGTCAAGACCGACCTGTCCGGTGC |
| | | | CCTGAATGAACTGCAAGACGAGGCAGCGCGGCTATCGTGGCTGGCCACGACGGGCG |
| | | | TTCCTTGCGCAGCTGTGCTCGACGTTGTCACTGAAGCGGGAAGGGACTGGCTGCTA |
| | | | TTGGGCGAAGTGCCGGGGCAGGATCTCCTGTCATCTCACCTTGCTCCTGCCGAGAA |
| | | | AGTATCCATCATGGCTGATGCAATGCGGCGGCTGCATACGCTTGATCCGGCTACCT |
| | | | GCCCATTCGACCACCAAGCGAAACATCGCATCGAGCGAGCACGTACTCGGATGGAA |
| | | | GCCGGTCTTGTCGATCAGGATGATCTGGACGAAGAGCATCAGGGGCTCGCGCCAGC |
| | | | CGAACTGTTCGCCAGGCTCAAGGCGAGCATGCCCGACGGCGAGGATCTCGTCGTGA |
| | | | CCCATGGCGATGCCTGCTTGCCGAATATCATGGTGGAAAATGGCCGCTTTTCTGGA |
| | | | TTCATCGACTGTGGCCGGCTGGGTGTGGCGGACCGCTATCAGGACATAGCGTTGGC |
| | | | TACCCGTGATATTGCTGAAGAGCTTGGCGGCGAATGGGCTGACCGCTTCCTCGTGC |
| | | | TTTACGGTATCGCCGCTCCCGATTCGCAGCGCATCGCCTTCTATCGCCTTCTTGAC |
| | | | GAGTTCTTCTGAATTATTAACGCTTACAATTTCCTGATGCGGTATTTTCTCCTTAC |
| | | | GCATCTGTGCGGTATTTCACACCGCATACAGGTGGCACTTTTCGGGGAAATGTGCG |
| | | | CGGAACCCCTATTTGTTTATTTTTCTAAATACATTCAAATATGTATCCGCTCATGA |
| | | | GACAATAACCCTGATAAATGCTTCAATAATAGCACGTGCTAAAACTTCATTTTTAA |
| | | | TTTAAAAGGATCTAGGTGAAGATCCTTTTTGATAATCTCATGACCAAAATCCCTTA |
| | | | ACGTGAGTTTTCGTTCCACTGAGCGTCAGACCCCGTAGAAAAGATCAAAGGATCTT |
| | | | CTTGAGATCCTTTTTTTCTGCGCGTAATCTGCTGCTTGCAAACAAAAAAACCACCG |
| | | | CTACCAGCGGTGGTTTGTTTGCCGGATCAAGAGCTACCAACTCTTTTTCCGAAGGT |
| | | | AACTGGCTTCAGCAGAGCGCAGATACCAAATACTGTCCTTCTAGTGTAGCCGTAGT |
| | | | TAGGCCACCACTTCAAGAACTCTGTAGCACCGCCTACATACCTCGCTCTGCTAATC |
| | | | CTGTTACCAGTGGCTGCTGCCAGTGGCGATAAGTCGTGTCTTACCGGGTTGGACTC |
| | | | AAGACGATAGTTACCGGATAAGGCGCAGCGGTCGGGCTGAACGGGGGGTTCGTGCA |
| | | | CACAGCCCAGCTTGGAGCGAACGACCTACACCGAACTGAGATACCTACAGCGTGAG |
| | | | CTATGAGAAAGCGCCACGCTTCCCGAAGGGAGAAAGGCGGACAGGTATCCGGTAAG |
| | | | CGGCAGGGTCGGAACAGGAGAGCGCACGAGGGAGCTTCCAGGGGGAAACGCCTGGT |
| | | | ATCTTTATAGTCCTGTCGGGTTTCGCCACCTCTGACTTGAGCGTCGATTTTTGTGA |
| | | | TGCTCGTCAGGGGGGCGGAGCCTATGGAAAAACGCCAGCAACGCGGCCTTTTTACG |
| | | | GTTCCTGGGCTTTTGCTGGCCTTTTGCTCACATGTTCTT |
| 57 | pb106 | cDNA | ATGGCGGCGATGGCCGAGCGGCCCTTCCAGTGCAGGATCTGTATGCGCAACTTTTC |
| | | | TCGGTCCTCCGACCTGACCCGGCACATCAGAACCCATACAGGCGAAAAGCCTTTCG |
| | | | CCTGCGACATTTGTGGGAGAAAATTTGCTCGGTCCGACACCCTGACCCGGCATACC |
| | | | AAGATCCACACCGGCTCTCAGAAACCATTCCAGTGCCGCATTTGTATGCGGAATTT |
| | | | TTCCCAGTCCGGCGACCTGTCCGAGCATATCCGCACTCACACCGGAGAGAAGCCCT |
| | | | TTGCTTGCGACATTTGTGGCAGGAAATTTGCTACCTCCGGCCACCTGACCACCCAC |
| | | | ACTAAGATCCATACTGGGTCACAGAAACCTTTCCAGTGCCGGATTTGTATGAGAAA |
| | | | CTTTAGCGACTCCTCCCACCTGACCACCCACATCAGAACCCATACAGGCGAAAAGC |
| | | | CTTTCGCCTGCGACATTTGTGGGAGAAAATTTGCTCGGTCCTCCCACCTGACCACC |
| | | | CATACCAAGATCCACACCGGCTCTCAGAAACCATTCCAGTGCCGCATTTGTATGCG |
| | | | GAATTTTTCCGACCGGTCCGACCTGACCCGGCATATCCGCACTCACACCGGAGAGA |
| | | | AGCCCTTTGCTTGCGACATTTGTGGCAGGAAATTTGCTGACCGGTCCGACCTGACC |
| | | | CGGCACACTAAGATCCATACTGGGTCACAGAAACCTTTCCAGTGCCGGATTTGTAT |
| | | | GAGAAACTTTAGCCGGTCCGACACCCTGACCCGGCACATCAGAACACATACTGGGC |
| | | | TGAGAGGATCCAACTCCCAGCGGCTGGCCGACCAGTACCGGCGGCACTCCCTGTTC |
| | | | GGCACCGGCGTGGAGGTGTCCCGGTACAACAAGTTCATGAAGATCTGCGCCCTGAC |
| | | | CAAGAAGGGCTGA |
| 58 | pb106 | AA | MAAMAERPFQCRICMRNFSRSSDLTRHIRTHTGEKPFACDICGRKFARSDTLTRHT |
| | | | KIHTGSQKPFQCRICMRNFSQSGDLSEHIRTHTGEKPFACDICGRKFATSGHLTTH |
| | | | TKIHTGSQKPFQCRICMRNFSDSSHLTTHIRTHTGEKPFACDICGRKFARSSHLTT |
| | | | HTKIHTGSQKPFQCRICMRNFSDRSDLTRHIRTHTGEKPFACDICGRKFADRSDLT |
| | | | RHTKIHTGSQKPFQCRICMRNFSRSDTLTRHIRTHTGLRGSNSQRLADQYRRHSLF |
| | | | GTGVEVSRYNKFMKICALTKKG* |
| 59 | pb111 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCCAGTGCAGGATCTGTATGCGCAACTTTTCTCGGTCCTCCG |
| | | | ACCTGACCCGGCACATCAGAACCCATACAGGCGAAAAGCCTTTCGCCTGCGACATT |
| | | | TGTGGGAGAAAATTTGCTCGGTCCGACACCCTGACCCGGCATACCAAGATCCACAC |
| | | | CGGCTCTCAGAAACCATTCCAGTGCCGCATTTGTATGCGGAATTTTTCCCAGTCCG |
| | | | GCGACCTGTCCGAGCATATCCGCACTCACACCGGAGAGAAGCCCTTTGCTTGCGAC |
| | | | ATTTGTGGCAGGAAATTTGCTACCTCCGGCCACCTGACCACCCACACTAAGATCCA |
| | | | TACTGGGTCACAGAAACCTTTCCAGTGCCGGATTTGTATGAGAAACTTTAGCGACT |
| | | | CCTCCCACCTGACCACCCACATCAGAACCCATACAGGCGAAAAGCCTTTCGCCTGC |
| | | | GACATTTGTGGGAGAAAATTTGCTCGGTCCTCCCACCTGACCACCCATACCAAGAT |
| | | | CCACACCGGCTCTCAGAAACCATTCCAGTGCCGCATTTGTATGCGGAATTTTTCCG |
| | | | ACCGGTCCGACCTGACCCGGCATATCCGCACTCACACCGGAGAGAAGCCCTTTGCT |
| | | | TGCGACATTTGTGGCAGGAAATTTGCTGACCGGTCCGACCTGACCCGGCACACTAA |
| | | | GATCCATACTGGGTCACAGAAACCTTTCCAGTGCCGGATTTGTATGAGAAACTTTA |
| | | | GCCGGTCCGACACCCTGACCCGGCACATCAGAACACATACTGGGGAGAAGATCATC |
| | | | GCCCACTTCCTGATCCAGCAGTACCTGAAGGAGGACTACTCCTTCACCGCCTACGC |
| | | | CACCATCTCCTACCTGAAGATCGGCTGAGCGGCCGCTCGAGTCTAGAGGGCCCGTT |
| | | | TAAACCCGCTGATCAGCCTCGACTGTGCCTTCTAGTTGCCAGCCATCTGTTGTTTG |
| | | | CCCCTCCCCCGTGCCTTCCTTGACCCTGGAAGGTGCCACTCCCACTGTCCTTTCCT |
| | | | AATAAAATGAGGAAATTGCATCGCATTGTCTGAGTAGGTGTCATTCTATTCTGGGG |
| | | | GGTGGGGTGGGGCAGGACAGCAAGGGGGAGGATTGGGAAGACAATAGCAGGCATGC |
| | | | TGGGGATGCGGTGGGCTCTATGGCTTCTACTGGGCGGTTTTATGGACAGCAAGCGA |
| | | | ACCGGAATTGCCAGCTGGGGCGCCCTCTGGTAAGGTTGGGAAGCCCTGCAAAGTAA |
| | | | ACTGGATGGCTTTCTCGCCGCCAAGGATCTGATGGCGCAGGGGATCAAGCTCTGAT |
| | | | CAAGAGACAGGATGAGGATCGTTTCGCATGATTGAACAAGATGGATTGCACGCAGG |
| | | | TTCTCCGGCCGCTTGGGTGGAGAGGCTATTCGGCTATGACTGGGCACAACAGACAA |
| | | | TCGGCTGCTCTGATGCCGCCGTGTTCCGGCTGTCAGCGCAGGGGCGCCCGGTTCTT |
| | | | TTTGTCAAGACCGACCTGTCCGGTGCCCTGAATGAACTGCAAGACGAGGCAGCGCG |
| | | | GCTATCGTGGCTGGCCACGACGGGCGTTCCTTGCGCAGCTGTGCTCGACGTTGTCA |
| | | | CTGAAGCGGGAAGGGACTGGCTGCTATTGGGCGAAGTGCCGGGGCAGGATCTCCTG |
| | | | TCATCTCACCTTGCTCCTGCCGAGAAAGTATCCATCATGGCTGATGCAATGCGGCG |
| | | | GCTGCATACGCTTGATCCGGCTACCTGCCCATTCGACCACCAAGCGAAACATCGCA |
| | | | TCGAGCGAGCACGTACTCGGATGGAAGCCGGTCTTGTCGATCAGGATGATCTGGAC |
| | | | GAAGAGCATCAGGGGCTCGCGCCAGCCGAACTGTTCGCCAGGCTCAAGGCGAGCAT |
| | | | GCCCGACGGCGAGGATCTCGTCGTGACCCATGGCGATGCCTGCTTGCCGAATATCA |
| | | | TGGTGGAAAATGGCCGCTTTTCTGGATTCATCGACTGTGGCCGGCTGGGTGTGGCG |
| | | | GACCGCTATCAGGACATAGCGTTGGCTACCCGTGATATTGCTGAAGAGCTTGGCGG |
| | | | CGAATGGGCTGACCGCTTCCTCGTGCTTTACGGTATCGCCGCTCCCGATTCGCAGC |
| | | | GCATCGCCTTCTATCGCCTTCTTGACGAGTTCTTCTGAATTATTAACGCTTACAAT |
| | | | TTCCTGATGCGGTATTTTCTCCTTACGCATCTGTGCGGTATTTCACACCGCATACA |
| | | | GGTGGCACTTTTCGGGGAAATGTGCGCGGAACCCCTATTTGTTTATTTTTCTAAAT |
| | | | ACATTCAAATATGTATCCGCTCATGAGACAATAACCCTGATAAATGCTTCAATAAT |
| | | | AGCACGTGCTAAAACTTCATTTTTAATTTAAAAGGATCTAGGTGAAGATCCTTTTT |
| | | | GATAATCTCATGACCAAAATCCCTTAACGTGAGTTTTCGTTCCACTGAGCGTCAGA |
| | | | CCCCGTAGAAAAGATCAAAGGATCTTCTTGAGATCCTTTTTTTCTGCGCGTAATCT |
| | | | GCTGCTTGCAAACAAAAAAACCACCGCTACCAGCGGTGGTTTGTTTGCCGGATCAA |
| | | | GAGCTACCAACTCTTTTTCCGAAGGTAACTGGCTTCAGCAGAGCGCAGATACCAAA |
| | | | TACTGTCCTTCTAGTGTAGCCGTAGTTAGGCCACCACTTCAAGAACTCTGTAGCAC |
| | | | CGCCTACATACCTCGCTCTGCTAATCCTGTTACCAGTGGCTGCTGCCAGTGGCGAT |
| | | | AAGTCGTGTCTTACCGGGTTGGACTCAAGACGATAGTTACCGGATAAGGCGCAGCG |
| | | | GTCGGGCTGAACGGGGGGTTCGTGCACACAGCCCAGCTTGGAGCGAACGACCTACA |
| | | | CCGAACTGAGATACCTACAGCGTGAGCTATGAGAAAGCGCCACGCTTCCCGAAGGG |
| | | | AGAAAGGCGGACAGGTATCCGGTAAGCGGCAGGGTCGGAACAGGAGAGCGCACGAG |
| | | | GGAGCTTCCAGGGGGAAACGCCTGGTATCTTTATAGTCCTGTCGGGTTTCGCCACC |
| | | | TCTGACTTGAGCGTCGATTTTTGTGATGCTCGTCAGGGGGGCGGAGCCTATGGAAA |
| | | | AACGCCAGCAACGCGGCCTTTTTACGGTTCCTGGGCTTTTGCTGGCCTTTTGCTCA |
| | | | CATGTTCTT |
| 60 | pb111 | cDNA | ATGGCGGCGATGGCCGAGCGGCCCTTCCAGTGCAGGATCTGTATGCGCAACTTTTC |
| | | | TCGGTCCTCCGACCTGACCCGGCACATCAGAACCCATACAGGCGAAAAGCCTTTCG |
| | | | CCTGCGACATTTGTGGGAGAAAATTTGCTCGGTCCGACACCCTGACCCGGCATACC |
| | | | AAGATCCACACCGGCTCTCAGAAACCATTCCAGTGCCGCATTTGTATGCGGAATTT |
| | | | TTCCCAGTCCGGCGACCTGTCCGAGCATATCCGCACTCACACCGGAGAGAAGCCCT |
| | | | TTGCTTGCGACATTTGTGGCAGGAAATTTGCTACCTCCGGCCACCTGACCACCCAC |
| | | | ACTAAGATCCATACTGGGTCACAGAAACCTTTCCAGTGCCGGATTTGTATGAGAAA |
| | | | CTTTAGCGACTCCTCCCACCTGACCACCCACATCAGAACCCATACAGGCGAAAAGC |
| | | | CTTTCGCCTGCGACATTTGTGGGAGAAAATTTGCTCGGTCCTCCCACCTGACCACC |
| | | | CATACCAAGATCCACACCGGCTCTCAGAAACCATTCCAGTGCCGCATTTGTATGCG |
| | | | GAATTTTTCCGACCGGTCCGACCTGACCCGGCATATCCGCACTCACACCGGAGAGA |
| | | | AGCCCTTTGCTTGCGACATTTGTGGCAGGAAATTTGCTGACCGGTCCGACCTGACC |
| | | | CGGCACACTAAGATCCATACTGGGTCACAGAAACCTTTCCAGTGCCGGATTTGTAT |
| | | | GAGAAACTTTAGCCGGTCCGACACCCTGACCCGGCACATCAGAACACATACTGGGG |
| | | | AGAAGATCATCGCCCACTTCCTGATCCAGCAGTACCTGAAGGAGGACTACTCCTTC |
| | | | ACCGCCTACGCCACCATCTCCTACCTGAAGATCGGCTGA |
| 61 | pb111 | AA | MAAMAERPFQCRICMRNFSRSSDLTRHIRTHTGEKPFACDICGRKFARSDTLTRHT |
| | | | KIHTGSQKPFQCRICMRNFSQSGDLSEHIRTHTGEKPFACDICGRKFATSGHLTTH |
| | | | TKIHTGSQKPFQCRICMRNFSDSSHLTTHIRTHTGEKPFACDICGRKFARSSHLTT |
| | | | HTKIHTGSQKPFQCRICMRNFSDRSDLTRHIRTHTGEKPFACDICGRKFADRSDLT |
| | | | RHTKIHTGSQKPFQCRICMRNFSRSDTLTRHIRTHTGEKIIAHFLIQQYLKEDYSF |
| | | | TAYATISYLKIG* |

Taken together, this example illustrates that design of a SSB-HbW can be extremely diversified. In this example, a zinc finger array comprising nine zinc fingers designed to recognize a sequence in human ApoE was used. Many other DNA specific binding domains can be used, including other zinc finger arrays. In this example three different linkers derived from human helicase sequences were used. Other linkers can be designed and evaluated for performance, using for example assays as described in this disclosure. As HbW domains, beta-wing structures from human BLM Helicase, human WRN helicase respectively human RECQ1 helicase were used. Other human, humanized or non-human helicase beta-wing can be considered, as well as synthetic molecules that provide similar functionality. The embodiments herein provide exemplary functional polynucleotide modification agents and demonstrates modularity of design, with a potential for wider choices in polynucleotide modification agent designs offering maximum flexibility providing technologies for successful gene editing applications across a variety of situations.

### Example 2: Modification of an Endogenous Genomic Target: Codon 112 of ApoE by SSB-HbW-Based Gene Editing in Human B-Cells

The present example describes sequence specific genetic modification of exemplary cells using technologies described herein. In this example, using human B-cells, human ApoE at codon 112 was targeted and edited by a specifically designed polynucleotide modification agent and a single stranded oligonucleotide template (i.e., a sequence modification polynucleotide). The human ApoE genotype is related to a risk of predisposition for developing Alzheimer's disease. Particularly, codon 112 encodes a critical residue relevant to Alzheimer's risk (or protection). This example describes development of SSB-HbW-based gene editing systems designed to convert a “T” to “C” at codon 112 in ApoE. In addition to being of potential clinical relevance, this target also exemplified usage of a naturally occurring target within a human (or mammalian) genome.

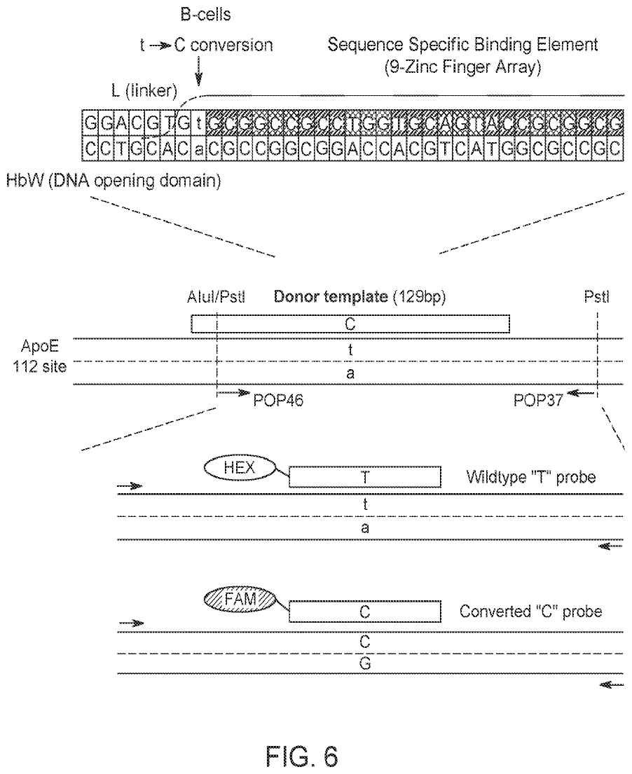

FIG. 6 illustrates an approach taken for this specific embodiment. This specific example aimed at gene editing of an endogenous genomic target around codon 112 of human ApoE in B cells. In this example, three exemplary polynucleotide modification agents, encoded on plasmids pb 106, pb 110, and pb 111, respectively described in Example 1 above, each include a DNA recognition domain which was an array of 9 zinc-fingers, specifically designed to recognize 5′-GCGGCCGCCTGGTGCAGTACCGCGGCG-3′ (SEQ ID NO.: 22), a 27-nucleotide sequence on the leading strand of human ApoE. A targeted nucleotide “T” was displayed as a lowercase letter “t”, 5′ upstream of this binding site. In this embodiment, a donor template was used: a 129-nucleotide single stranded DNA oligonucleotide with a desired T→C substitution roughly located in the middle of this oligonucleotide. This single stranded donor template used herein is provided below as a sequence with an underlined and bold “C” to for T→C conversion.

(SEQ ID NO: 11)
5′-CCCCGGTGGCGGAGGAGACGCGGGCACGGCTGTCCAAGGAGCTGC
AGGCGGCGCAGGCCCGGCTGGGCGCGGACATGGAGGACGTGCGCGGCC
GCCTGGTGCAGTACCGCGGCGAGGTGCAGGCCATGC-3′

Detections of genetic T→C conversion after SSB-HbW-based gene edition were performed by droplet digital PCR (ddPCR). Relative positions of a correction ssODN (i.e., sequence modification polynucleotide) and position of an exemplary primer pair: POP46 and POP37 are also indicated in FIG. 6. One common primer, POP46 was located inside this ssODN template (i.e., sequence modification polynucleotide) sequence, while POP37, located outside. Allele-specific probes conjugated with fluorophores FAM and HEX were designed to distinguish between “C” and “T” respectively. PstI restriction enzyme sites indicated were used in preparations for ddPCR reactions.

POP46-511-Alu-apoE-f forward primer has a sequence of

(SEQ ID NO: 62)
CTGCAGGCGGCGCAGGC

POP37 ApoE reverse primer has a sequence of

(SEQ ID NO: 63)
GGTCATCGGCATCGCGGAGGAG

FIG. 7 demonstrates successful T→C genetic conversion at codon 112 of human ApoE as measured by ddPCR. In this example, after transfection of B cells with plasmids pb 106, pb 110 respectively pb 111 and this 129-nucleotide correction template, cells were allowed to recover and grow on complete culture medium, containing 15% FBS in DMEM, for seven days. After seven days genomic DNA was isolated and used in ddPCR analysis. Raw droplet data are shown as in FIG. 7 where these “C” droplets are displayed in the top panel, while “T” droplets were in the lower one. Untargeted B-cells were used as a negative control, showing neither “C” nor “T” droplets. Untargeted B cells only had “T” droplets, demonstrating homozygous T/T genotype. After B cells were transfected with pb 106, pb 110 respectively pb 111 and ssODN template (i.e., sequence modification polynucleotide), “C” droplets appeared after being targeted and edited by this polynucleotide modification agent in combination with a correcting template, demonstrating successful T→C genetic conversion at codon 112 of human ApoE.

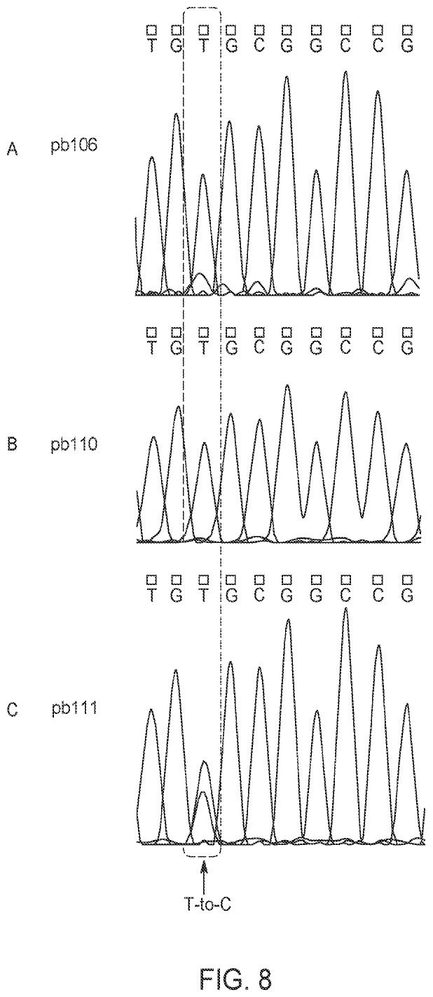

FIG. 8 shows exemplary Sanger sequencing results used to further confirm successful targeting and editing of codon 112 of the human ApoE gene. Genomic DNA was isolated and used as a template on which a 175-bp PCR amplicon surrounding ApoE codon 112 was generated by using a primer set of POP46 and POP37. Amplified PCR products from targeted B cells were analyzed. FIG. 8, panel A shows an exemplary chromatogram of a pb 106 edited B-cell population, showing a “T-to-C” sequence by Sanger sequencing. FIG. 8, panel B shows an exemplary chromatogram of a pb 110 edited B-cell population, showing a “T-to-C” sequence by Sanger sequencing. FIG. 8, panel C shows an exemplary chromatogram of a pb 111 edited B-cell population, showing a “T-to-C” sequence by Sanger sequencing. These results confirm that a polynucleotide modification agent in combination with a sequence modification polynucleotide can successfully target and edit a sequence in an endogenous mammalian gene in mammalian cells to successfully modify a disease-causing genotype.

In the present Example, next generation sequencing was performed to determine, in more detail, gene conversion frequencies and patterns and also potential generation of insertions, deletions, and unintended single nucleotide polymorphisms after SSB-HbW-based gene editing. In order to do so, next generation sequencing of targeted pooled B cells (and untransfected B cells as control) was performed. Genomic DNA was isolated and used as a template on which a 175-bp PCR amplicon surrounding ApoE codon 112 was generated by using a primer set of POP46 and POP37. Amplified PCR products from targeted B cells and control B cells were analyzed for indels and SNPs on an Illumina next generation sequencing platform (GENEWIZ, South Plainfield, NJ).

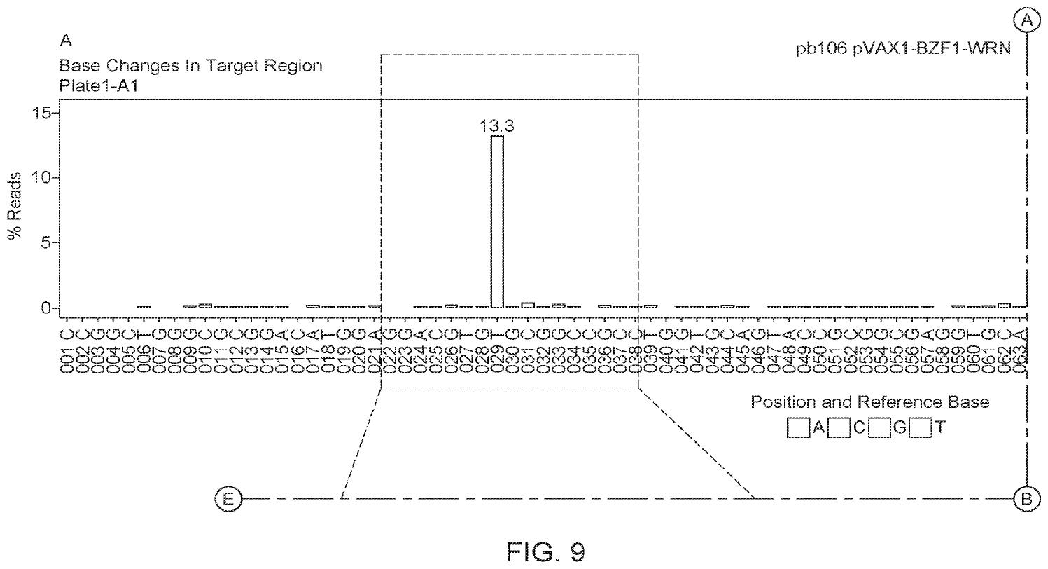

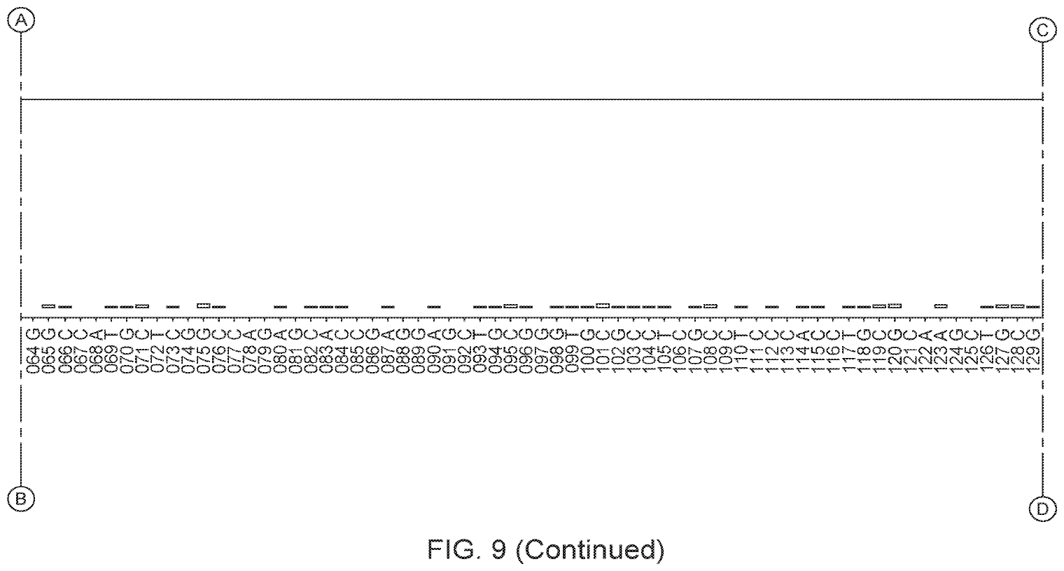

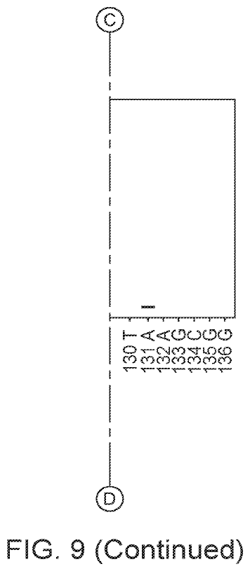

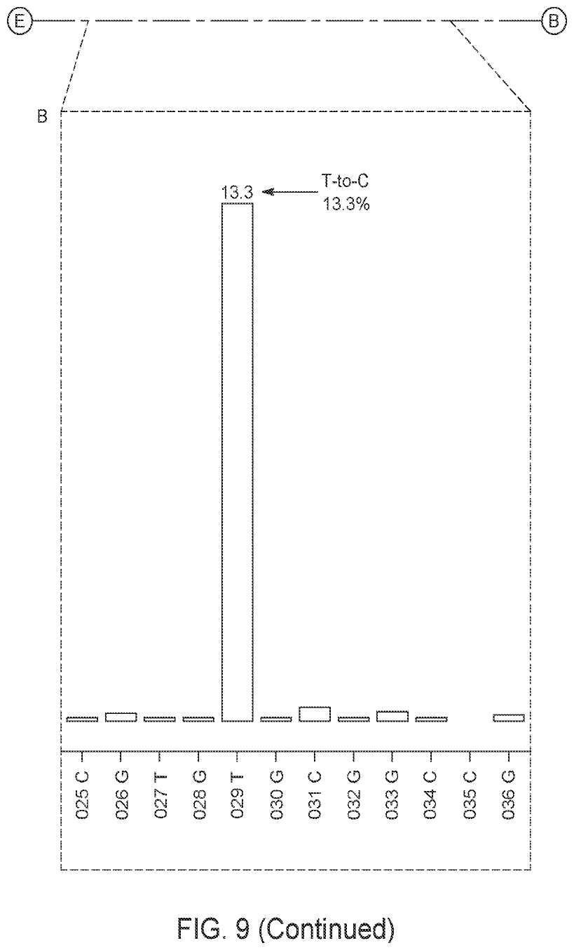

FIG. 9 shows confirmation of detection of single nucleotide T→C conversion at this target site as well as single nucleotide polymorphisms (SNPs) analysis within a target region of surrounding codon 112 of this ApoE locus. Panel A shows overall views of SNPs analysis at these target sites obtained with untargeted B cells, and targeted pooled B cells. Bar graphs plot frequencies of SNPs at each nucleotide position in this 175 bp PCR amplification region. Panel B is a magnified view of the portion close to this gene repair site. In this example cells transfected with pb 106 (WRN derived HbW) and a correction template showed a T-to-C conversion at this expected nucleotide position with a frequency of 13.3%. Compared to non-transfected B cells, no other nucleotide conversions had occurred at a level significantly above background. Comparing to untransfected cells, no obvious unwanted SNPs were detected.

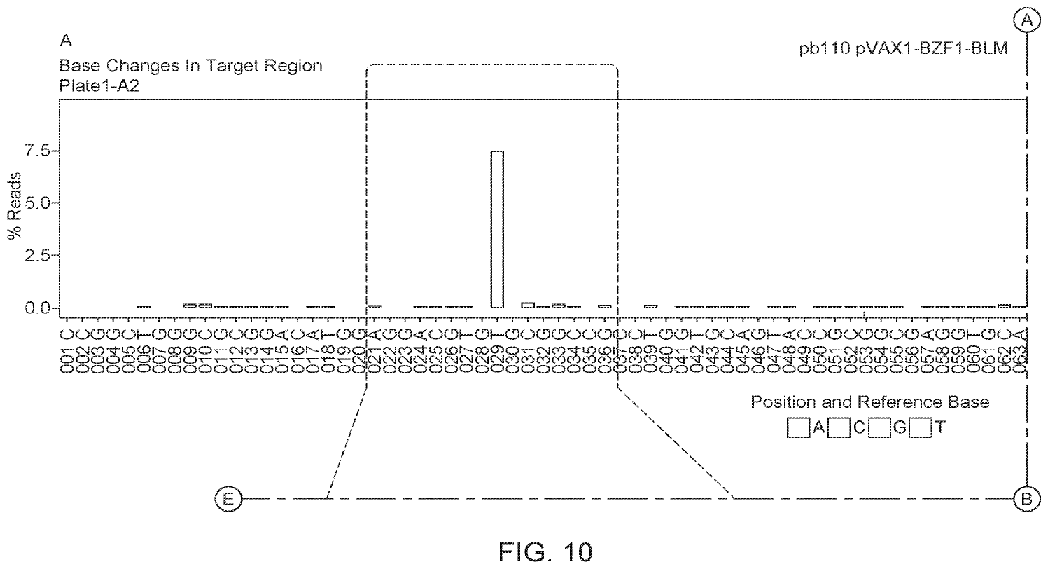

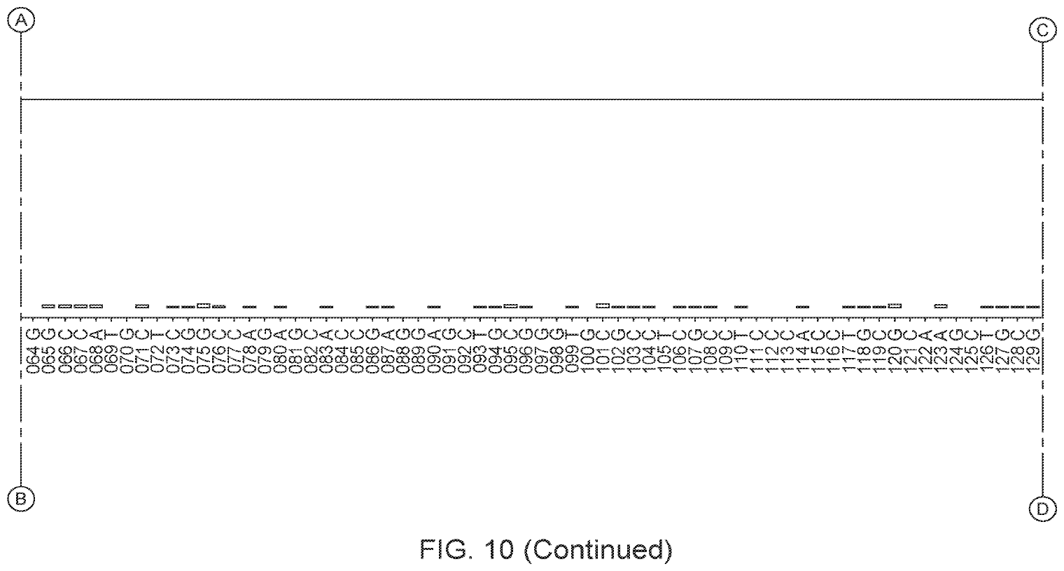

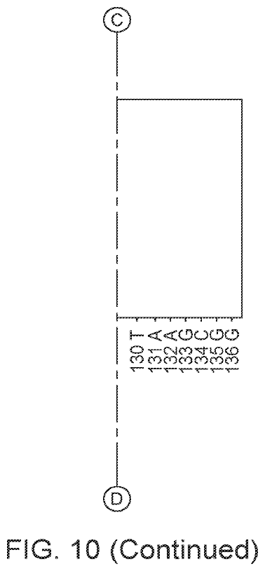

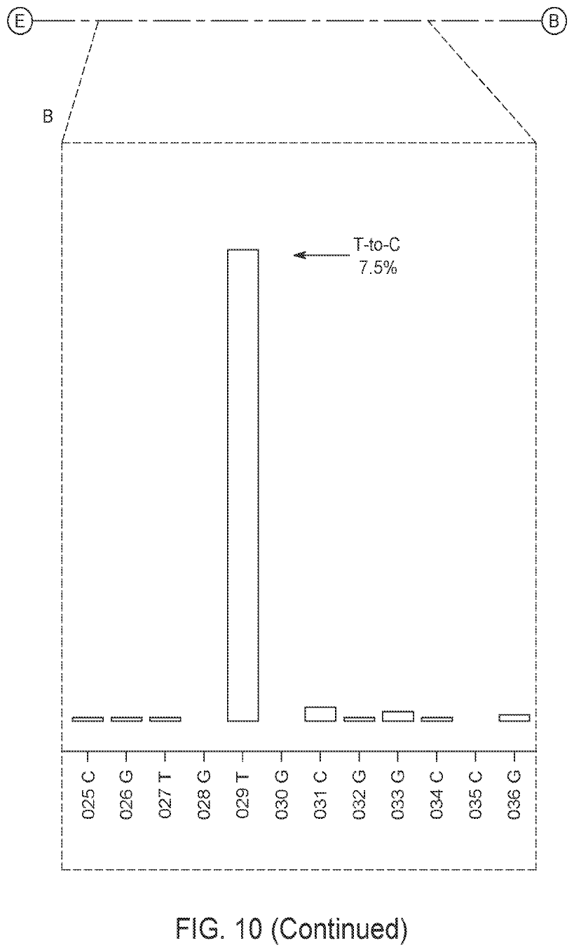

FIG. 10 shows another confirmation of detection of single nucleotide T→C conversion at this target site as well as single nucleotide polymorphisms (SNPs) analysis within a target region of surrounding codon 112 of this ApoE locus. Panel A shows overall views of SNPs analysis at these target sites obtained with untargeted B cells, and targeted pooled B cells. Bar graphs plot frequencies of SNPs at each nucleotide position in this 175 bp PCR amplification region. Panel B is a magnified view of the portion close to this gene repair site. In this example cells transfected with pb 110 (BLM derived HbW) and a correction template showed a T-to-C conversion at this expected nucleotide position with a frequency of 7.5%. Compared to non-transfected B cells, no other nucleotide conversions had occurred at a level significantly above background. Comparing to untransfected cells, no obvious unwanted SNPs were detected.

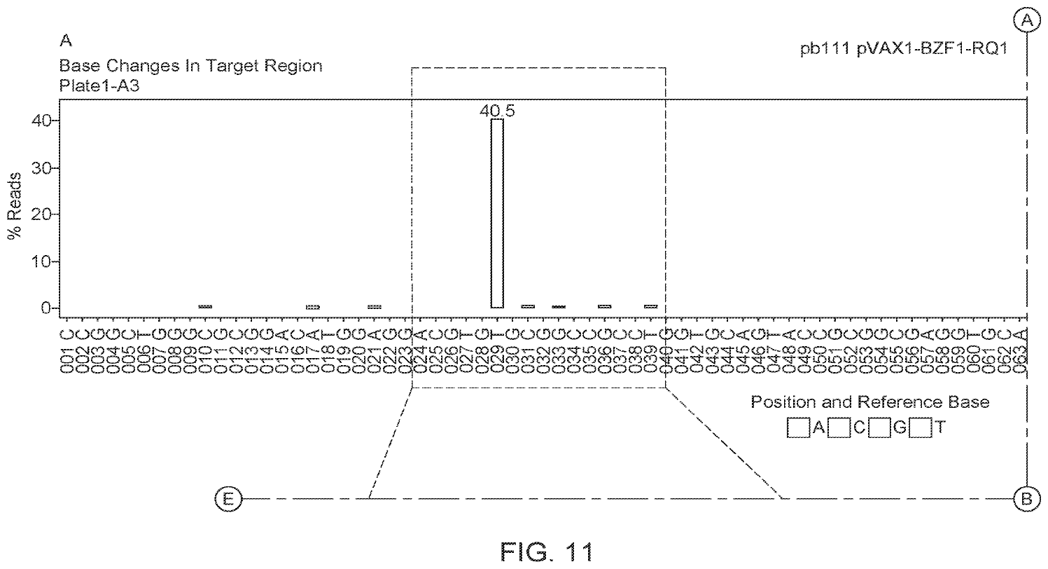

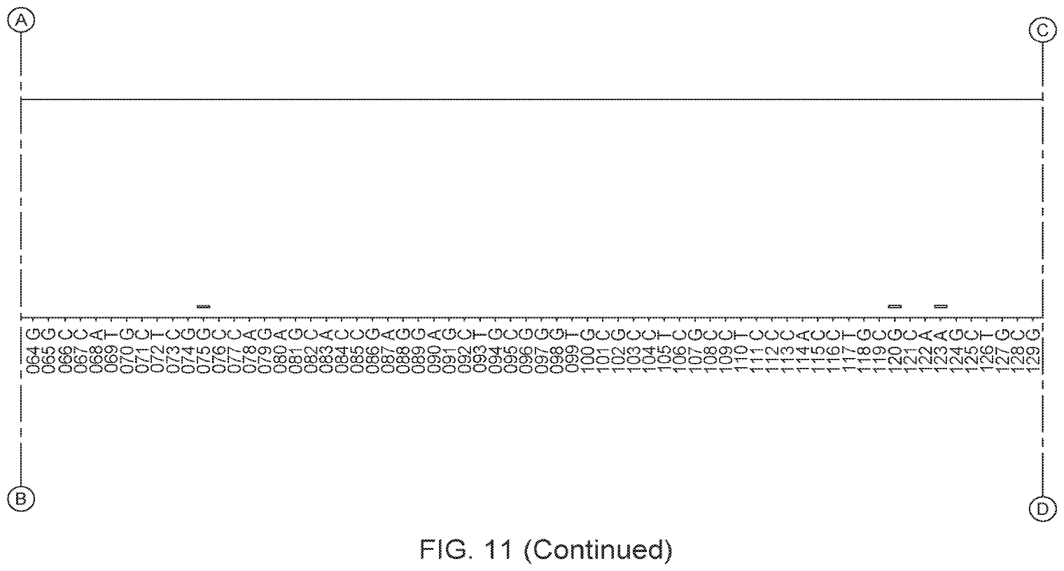

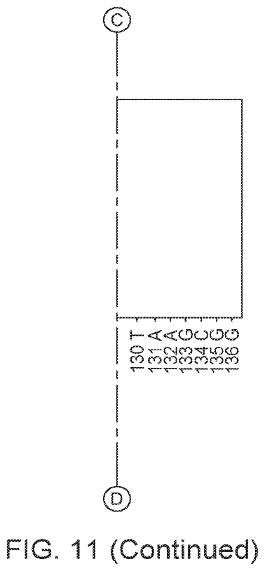

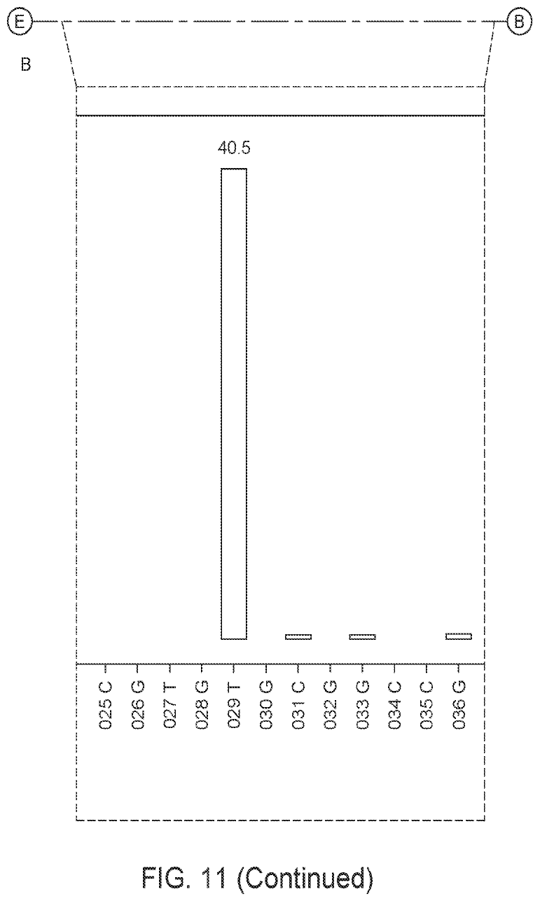

FIG. 11 shows another confirmation of detection of single nucleotide T→C conversion at this target site as well as single nucleotide polymorphisms (SNPs) analysis within a target region of surrounding codon 112 of this ApoE locus. Panel A shows overall views of SNPs analysis at these target sites obtained with untargeted B cells, and targeted pooled B cells. Bar graphs plot frequencies of SNPs at each nucleotide position in this 175 bp PCR amplification region. Panel B is a magnified view of the portion close to this gene repair site. In this example cells transfected with pb 111 (RECQ1 derived HbW) and a correction template showed a T-to-C conversion at this expected nucleotide position with a frequency of 40.5%. Compared to non-transfected B cells, no other nucleotide conversions had occurred at a level significantly above background. Comparing to untransfected cells, no obvious unwanted SNPs were detected.

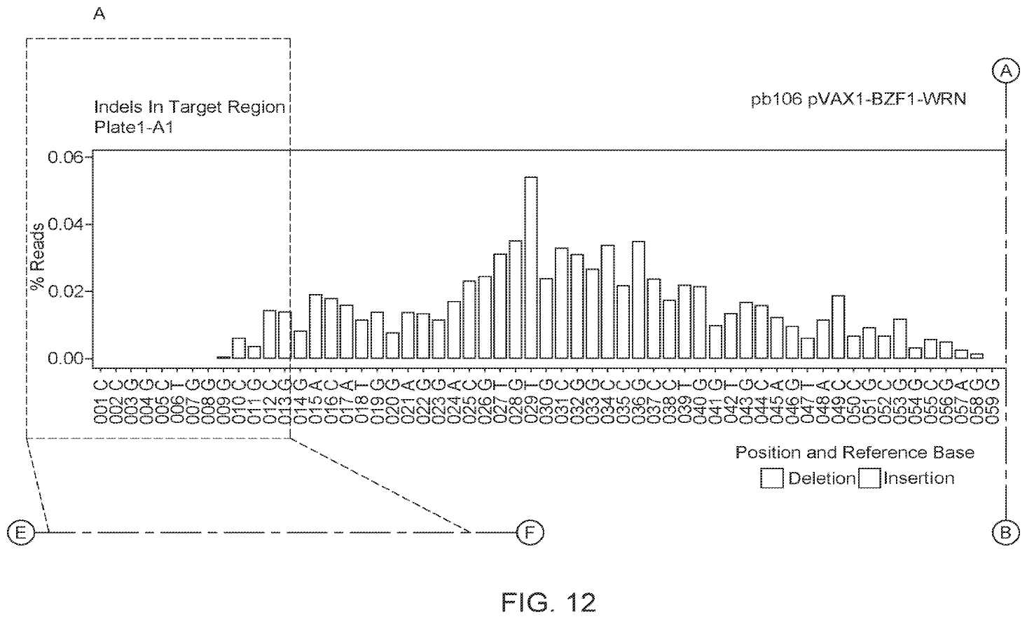

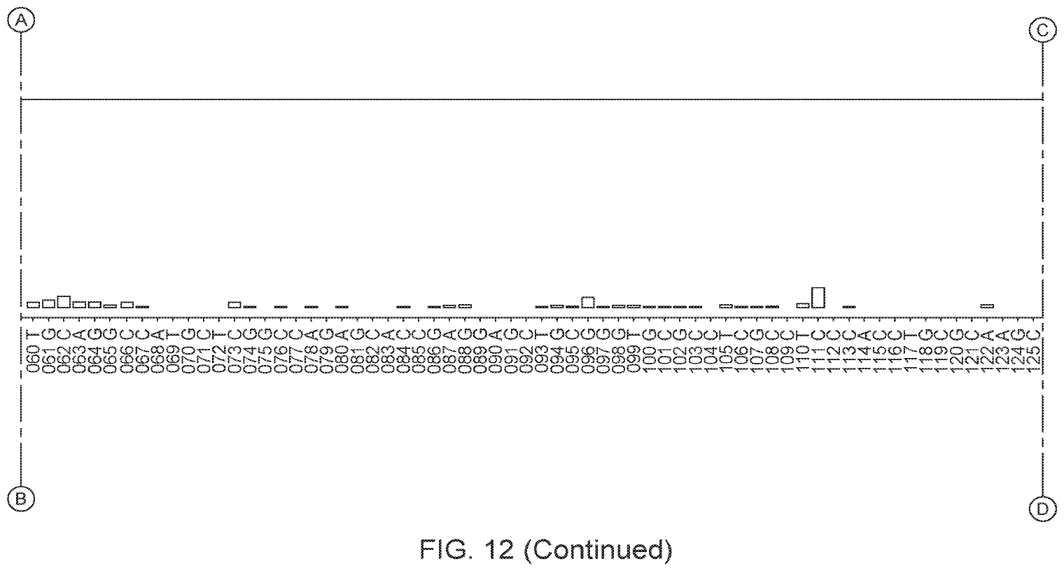

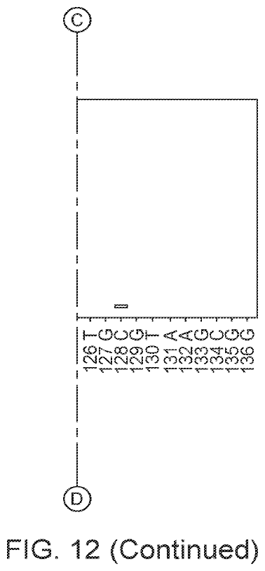

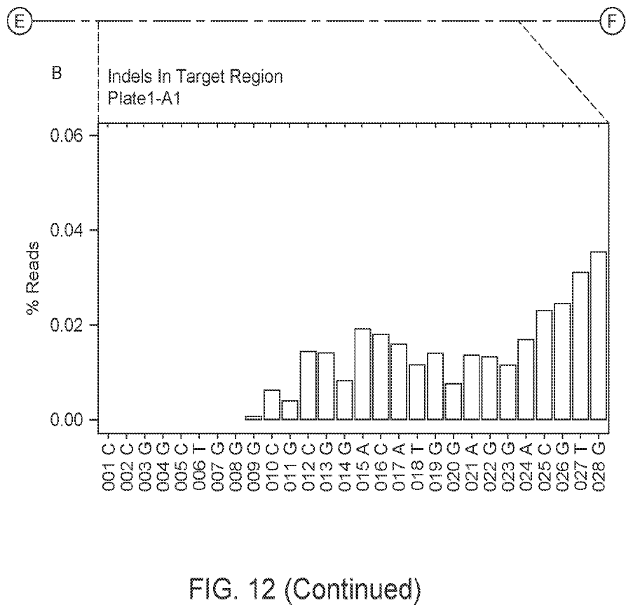

FIG. 12 shows insertion and deletion analysis around codon 112 of ApoE in an example using pb 106 (BLM derived), displayed as a frequency plot of insertions and deletions analysis for targeted pooled B cells. Bar graphs plot frequencies of insertions and deletions at each nucleotide position of this 175 bp PCR amplification region. This indels analysis showed, in general, a very low frequency (<0.06%) of insertions and/or deletions. The highest level of change at any position was a nucleotide insertion of 0.06% at position 29 of this amplicon, which could also be observed with B cell controls and most likely reflected a technical artifact. In addition, patterns and frequencies of indels at each position from both targeted and untransfected B cells were no statistically significantly different and were considered to be within the error range and the detection limitations typical for the PCR and next generation sequencing method used.

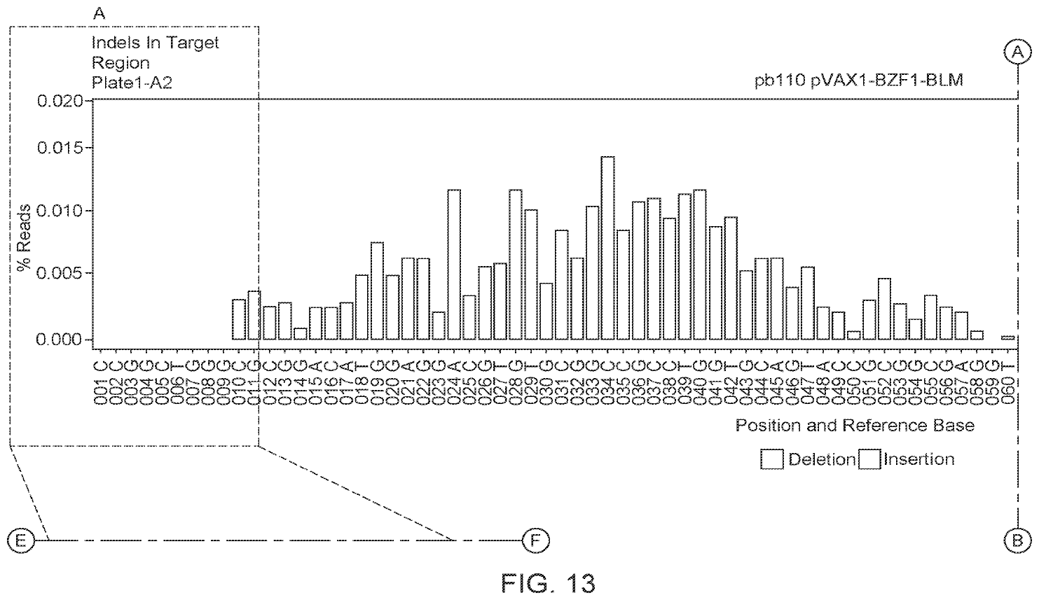

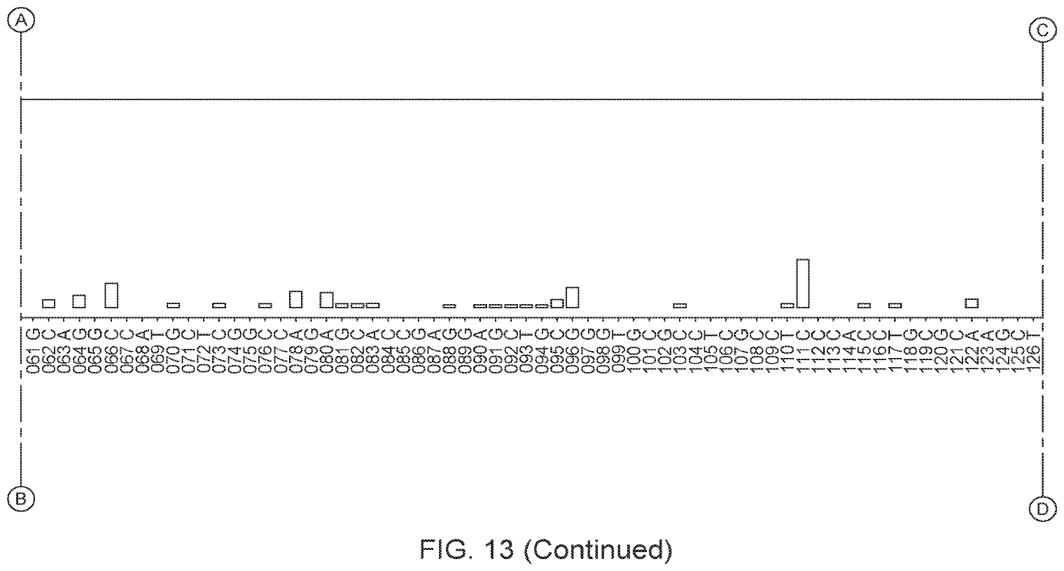

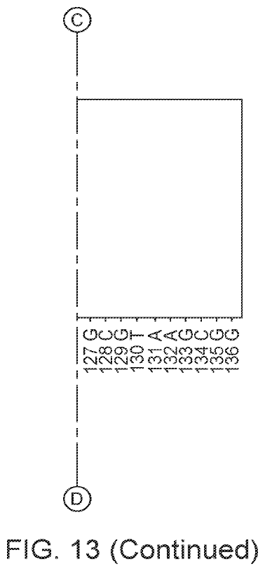

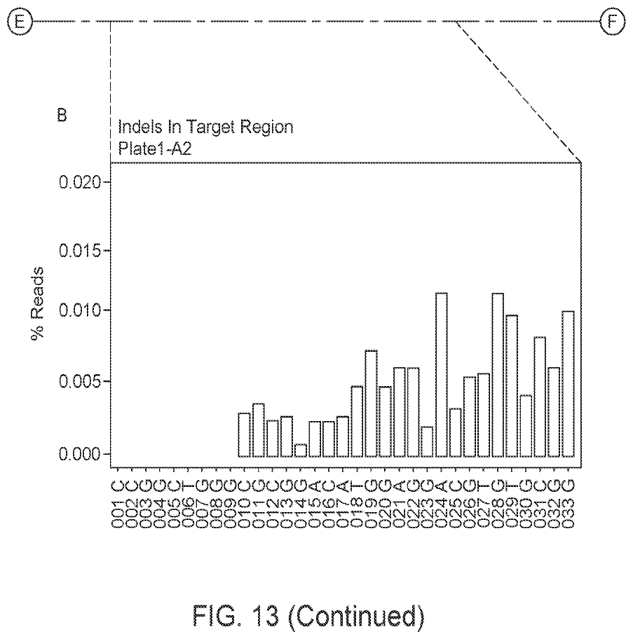

FIG. 13 shows insertion and deletion analysis around codon 112 of ApoE in another example using pb 110 (WRN derived), displayed as a frequency plot of insertions and deletions analysis for targeted pooled B cells. Bar graphs plot frequencies of insertions and deletions at each nucleotide position of this 175 bp PCR amplification region. This indels analysis showed, in general, a very low frequency (<0.02%) of insertions and/or deletions. Patterns and frequencies of indels at each position from both targeted and untransfected B cells were no statistically significantly different and were considered to be within the error range and the detection limitations typical for the PCR and next generation sequencing method used.

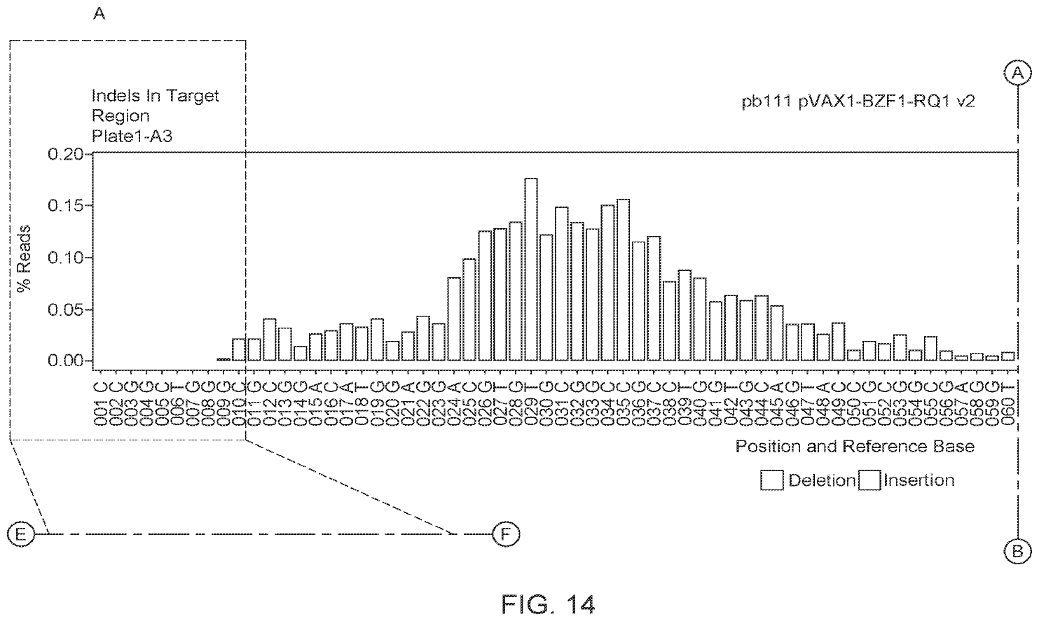

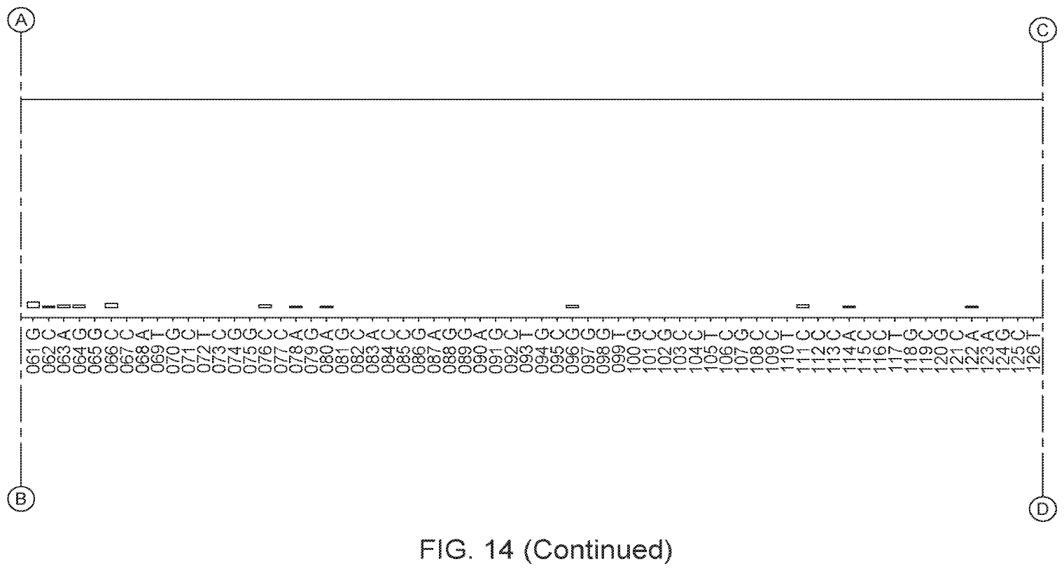

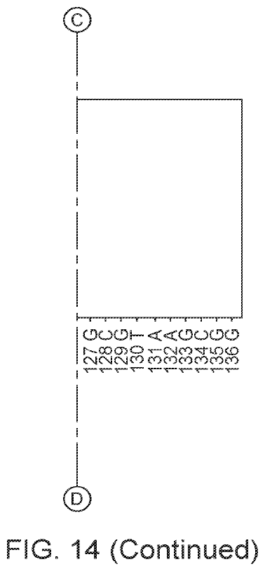

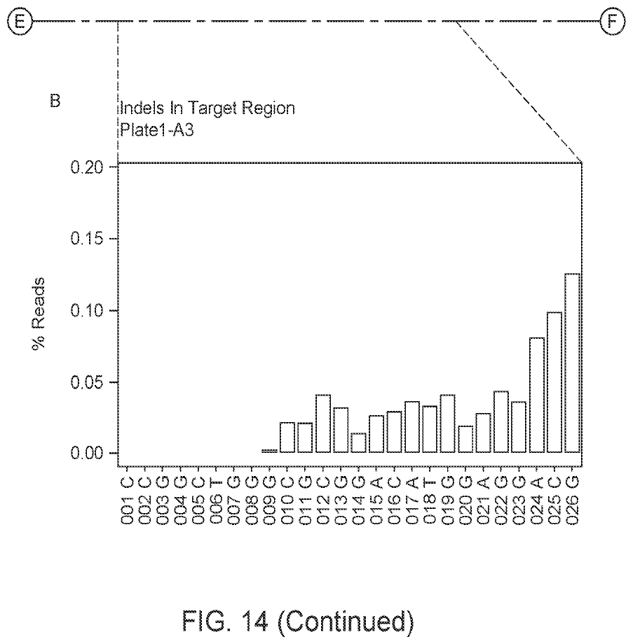

FIG. 14 shows insertion and deletion analysis around codon 112 of ApoE in another example using pb 111 (RECQ1 derived), displayed as a frequency plot of insertions and deletions analysis for targeted pooled B cells. Bar graphs plot frequencies of insertions and deletions at each nucleotide position of this 175 bp PCR amplification region. This indels analysis showed, in general, a very low frequency (<0.2%) of insertions and/or deletions. Patterns and frequencies of indels at each position from both targeted and untransfected B cells were no statistically significantly different and were considered to be within the error range and the detection limitations typical for the PCR and next generation sequencing method used.

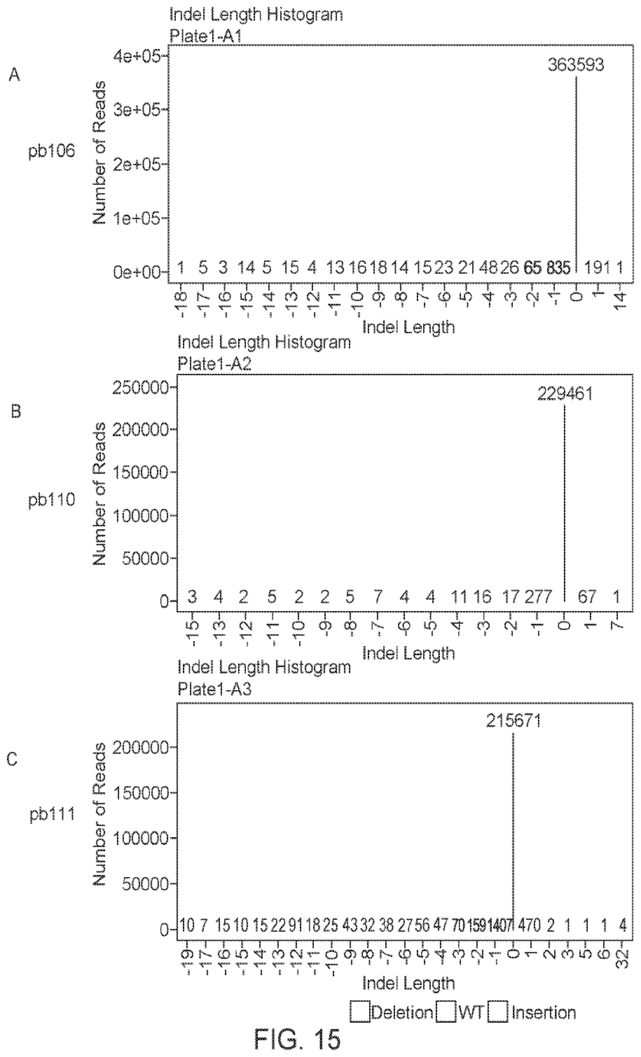

FIG. 15 shows insertion and deletion (Indels) analysis by next generation sequencing of edited B-cells illustrated as histograms. An x-axis indicates the number of deleted nucleotides (expressed as negative numbers), no insertions or deletions (indicated by 0) respectively insertions (expressed by positive numbers). A y-axis indicates the number of sequence reads obtained for each InDel. Panel A shows overviews of InDels at each position of the targeting region of codon 112 site of human ApoE using pb 106. Panel B shows overviews of InDels at each position of the targeting region of codon 112 site of human ApoE using pb 110. Panel C shows overviews of InDels at each position of the targeting region of codon 112 site of human ApoE using pb 111. This example illustrates a low amount of indels generated compared to wild type or single nucleotide changes. In addition, it also illustrates that 1-nucleotide deletions followed by 2-nucleotide deletions are most prevalent type of InDels. A potential source of these observed types of InDels may also arise from non-perfect modification oligonucleotide synthesis. It is well known to those skilled in the art that chemical coupling of nucleotides when synthesizing an oligonucleotide does not achieve 100% coupling efficiency in every molecule at every single synthesis step. One frequently observed imperfection the generation of oligonucleotides that lack 1 or 2 nucleotides. Part of the observed minus 1 and/or minus 2 deletions may have arisen from the incorporation of imperfectly synthesized oligonucleotides, rather than the gene editing mechanism caused these InDels to be formed by cellular processes.

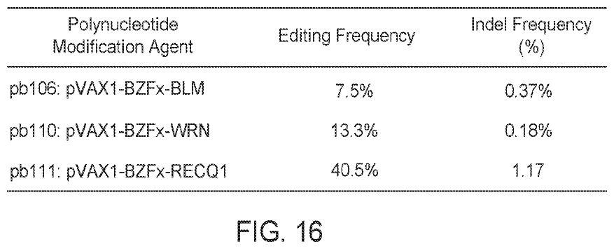

FIG. 16 shows overall indels and editing frequencies using zinc finger helicase beta wing mediated gene editing targeting with pb 106 and a sequence modification polynucleotide, an overall zinc finger helicase beta wing mediated gene editing frequency of 7.5% and an indel frequency of only 0.34% was observed. Using zinc finger helicase beta wing mediated gene editing targeting with pb 110 and a sequence modification polynucleotide, an overall zinc finger helicase beta wing mediated gene editing frequency of 13.3% and an indel frequency of only 0.18% was observed. Using zinc finger helicase beta wing mediated gene editing targeting with pb 111 and a sequence modification polynucleotide, an overall zinc finger helicase beta wing mediated gene editing frequency of 40.5% and an indel frequency of only 1.17% was observed. Taken together, zinc finger helicase beta wing mediated gene editing is able to achieve relatively high gene editing efficiency with very low indel frequencies

Notably, this example demonstrates that provided technologies are capable of genetic modification with very low levels of insertions and deletions. Thus, technologies of the present disclosure are capable of targeted gene conversion without potentially detrimental generation of insertions, deletions and/or undesired single nucleotide polymorphisms.

In addition, this example also illustrates that for specific targets and/or purposes a plurality of zinc finger helicase beta wing constructs can be created and evaluated for their performance, using genetic and genomic assays know to those skilled in the art. In this specific example, three different zinc finger helicase beta wing constructs targeting codon 112 of human ApoE (encoded on pb 106, pb 110 and pb111) were created and evaluated for their performance in gene editing. In this example, a construct using RECQ1 helicase derived sequences yielded higher conversion frequencies compared to a construct using sequences derived from BLM helicase or WRN helicase. In this particular example, it was also observed that a construct using WRN helicase derived sequences had relatively lower indel frequency compared to a construct using sequences derived from BLM helicase or a RECQ1 helicase. This disclosure contemplates that additional variations of linkers and helicase derived sequences can be used to create zinc finger helicase beta wing constructs, which can be evaluated and/or selected for desired characteristics (e.g., editing of particular targets). Without being bound to any particular theory, this disclosure contemplates that such performance test(s) may enable selection of constructs that are particularly suited for a specific purpose (e.g., genome editing purpose).

### Example 3: Modification of an Endogenous Genomic Target: Codon 112 of ApoE by SSB-HbW-Based Gene Editing in Human Hepatocytes

The present example describes sequence specific genetic modification of non-dividing (i.e., non-replicating) cells using an exemplary embodiment of the provided technology. In particular, the present example describes genetic modification of human hepatocyte cells. Human hepatocytes were obtained (Yecuris, Tualatin, OR) and cultured under non-dividing conditions. Specifically, a human ApoE at codon 112 was successfully targeted and edited using an exemplary polynucleotide modification agent and a single stranded oligonucleotide template (i.e., a sequence modification polynucleotide), as described in Example 2.

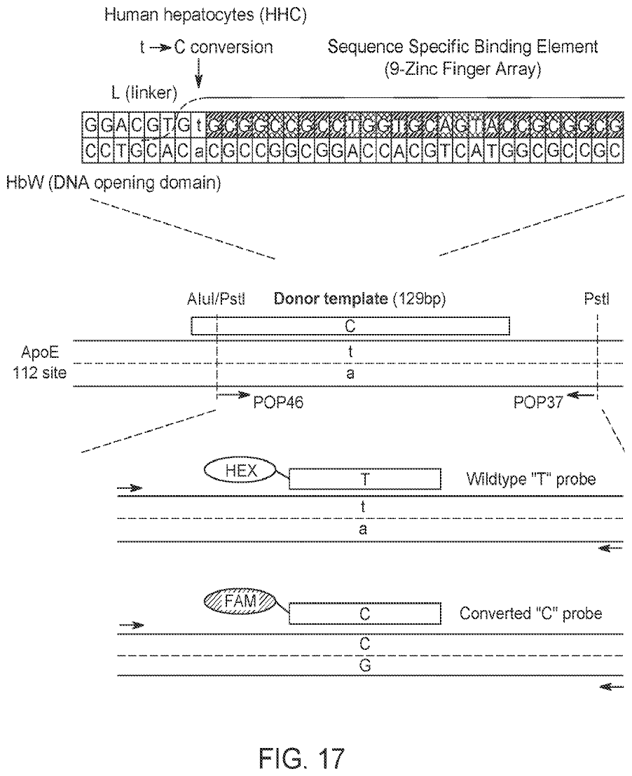

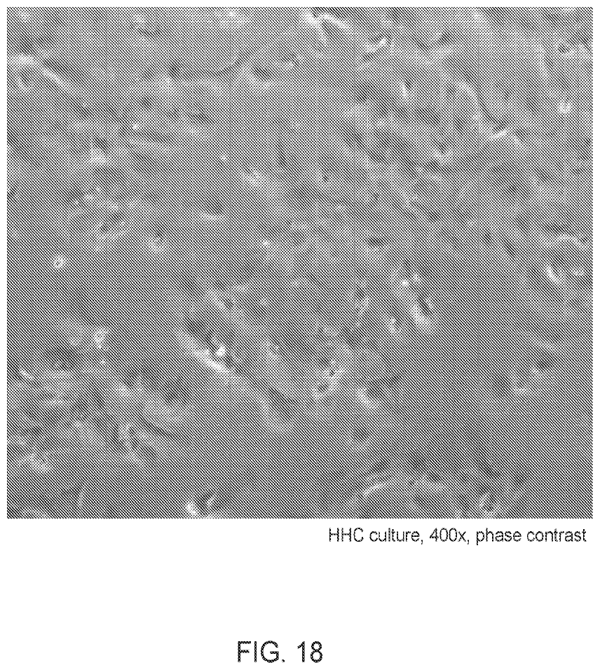

FIG. 17 illustrates an approach taken for this specific embodiment. This specific example aimed at gene editing of an endogenous genomic target around codon 112 of ApoE in human hepatocytes. FIG. 18 illustrates a culture of human hepatocytes.

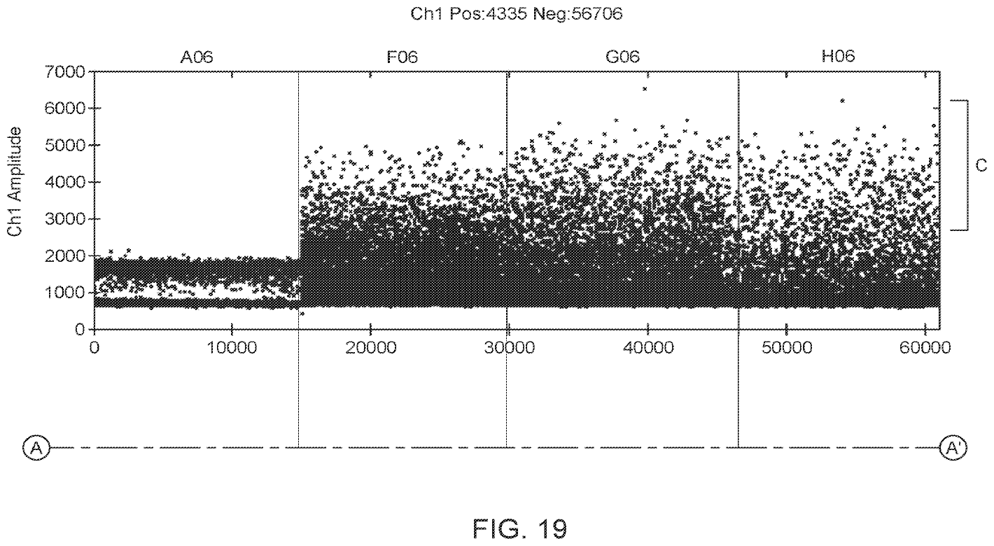

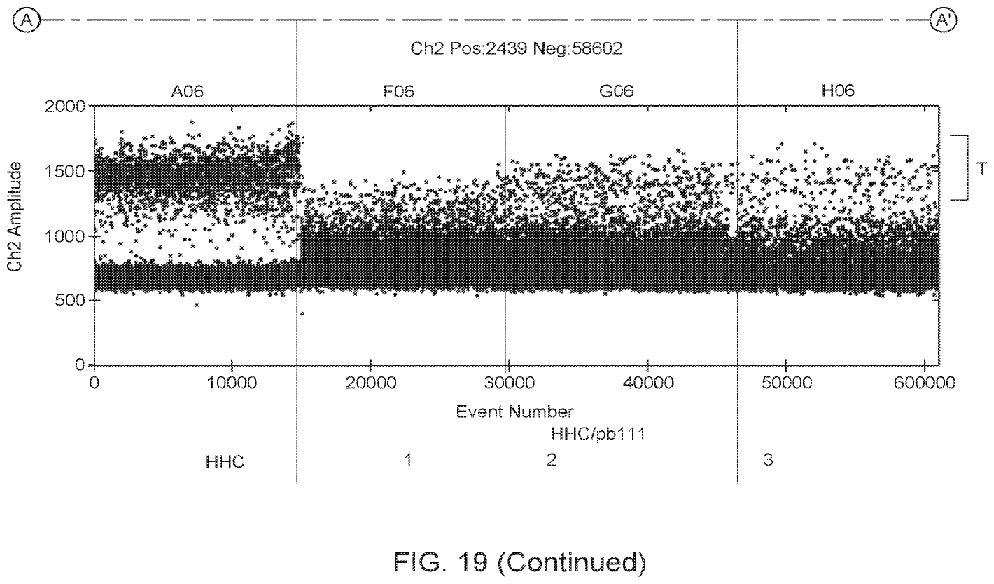

FIG. 19 demonstrates successful T→C genetic conversion at codon 112 of human ApoE as measured by ddPCR. In this example, after transfection of human hepatocytes with plasmid pb 111 and this 129-nucleotide correction template, cells were allowed to recover and grow on complete culture medium, containing 15% FBS in DMEM, for seven days. After seven days genomic DNA was isolated and used in ddPCR analysis. Raw droplet data are shown as in FIG. 19 where these “C” droplets are displayed in the top panel, while “T” droplets were in the lower one. Untargeted hepatocytes (HHC) were used as a negative control, showing neither “C” nor “T” droplets. Untargeted hepatocytes only had “T” droplets, demonstrating homozygous T/T genotype. After hepatocytes were transfected with pb 111 and ssODN template (i.e., sequence modification polynucleotide), “C” droplets appeared after being targeted and edited by this exemplary polynucleotide modification agent in combination with a correcting template, demonstrating successful T→C genetic conversion at codon 112 of human ApoE.

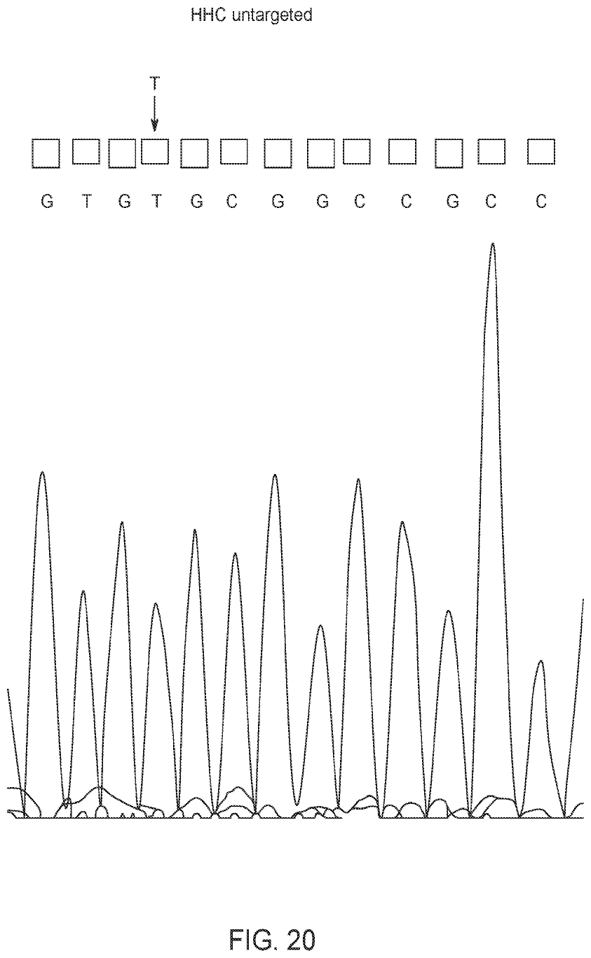

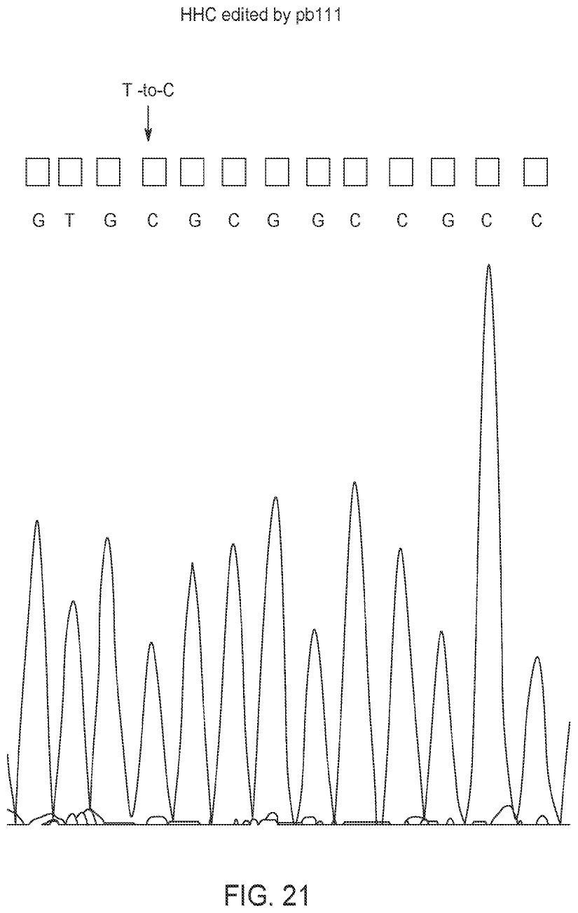

FIG. 20 and FIG. 21 show Sanger sequencing results used to further confirm successful targeting and editing of codon 112 of the human ApoE gene. Genomic DNA was isolated and used as a template on which a 175-bp PCR amplicon surrounding ApoE codon 112 was generated by using a primer set of POP46 and POP37. Amplified PCR products from untargeted and targeted hepatocytes were analyzed. FIG. 20 shows an exemplary chromatogram of an untargeted hepatocyte population, showing no “C” sequence signal by Sanger sequencing. FIG. 21 shows an exemplary chromatogram of a pb 111 edited hepatocytes, showing a “T-to-C” sequence by Sanger sequencing. These results further confirm that a polynucleotide modification agent in combination with a sequence modification polynucleotide can successfully target and edit a sequence in an endogenous mammalian gene in mammalian cells to successfully modify a disease-causing genotype.

In the present Example, next generation sequencing was performed to determine, in more detail, gene conversion frequencies and patterns and potential generation of insertions, deletions, and unintended single nucleotide polymorphisms after SSB-HbW-based gene editing. To do so, next generation sequencing of targeted hepatocytes (and untransfected hepatocytes as control) was performed. Genomic DNA was isolated and used as a template on which a 175-bp PCR amplicon surrounding ApoE codon 112 was generated by using a primer set of POP46 and POP37. Amplified PCR products from targeted hepatocytes and control hepatocytes were analyzed for indels and SNPs on an Illumina next generation sequencing platform (GENEWIZ, South Plainfield, NJ).

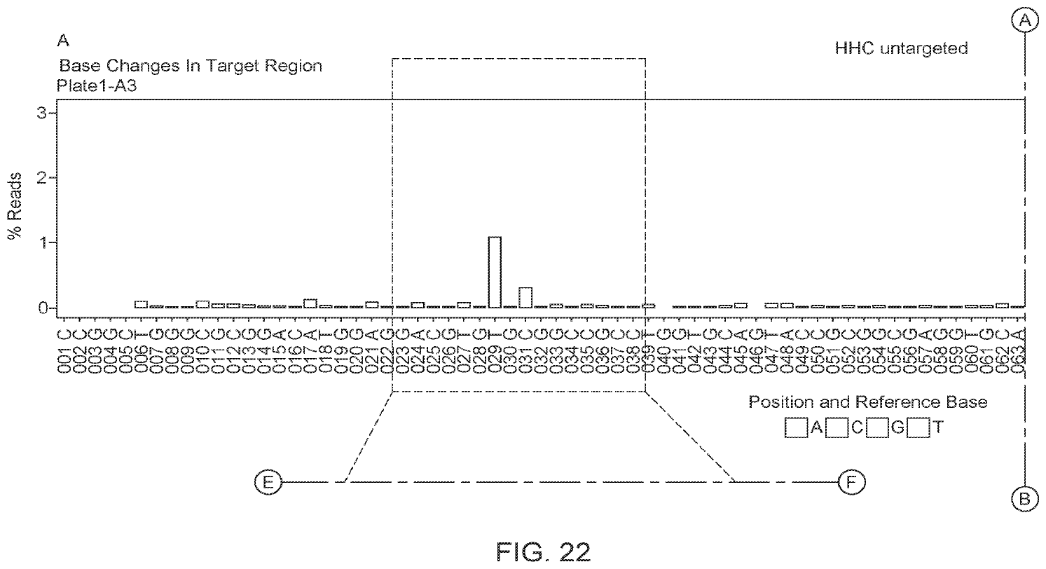

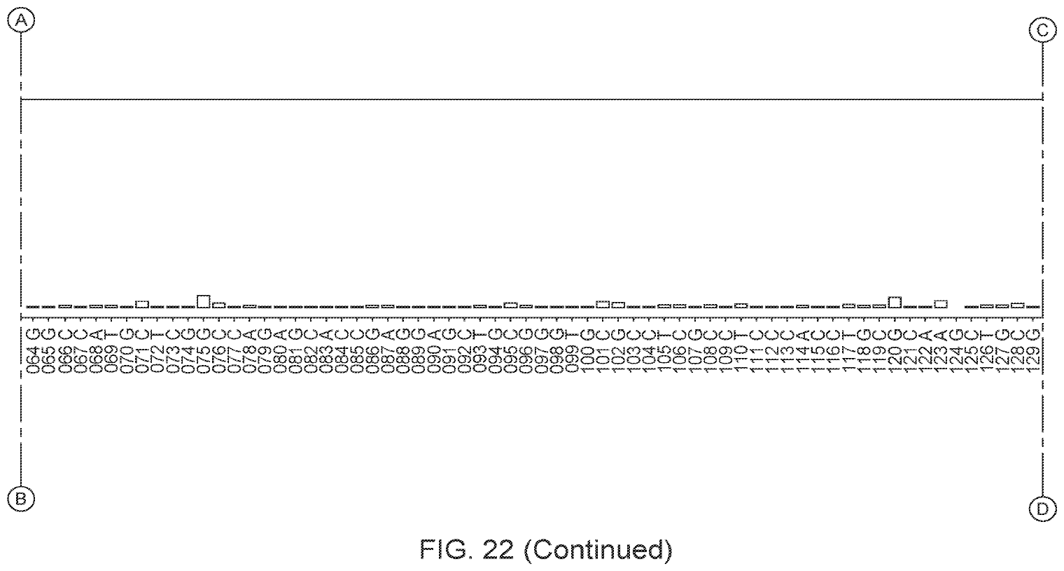

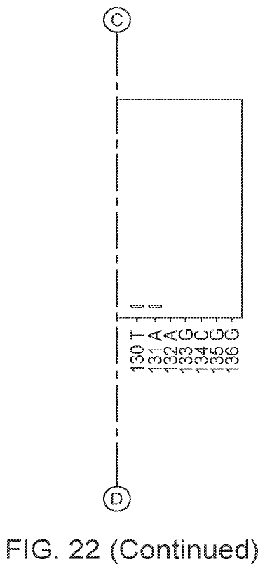

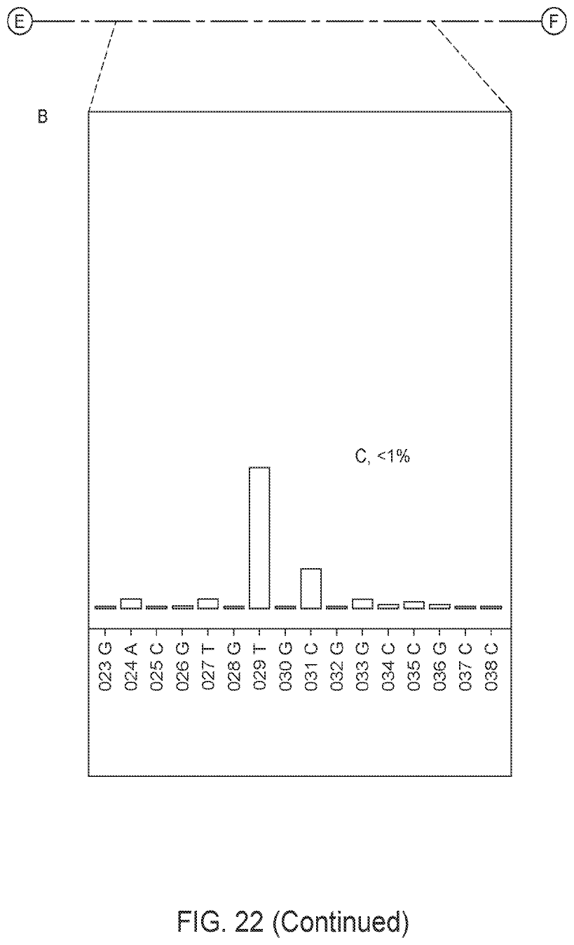

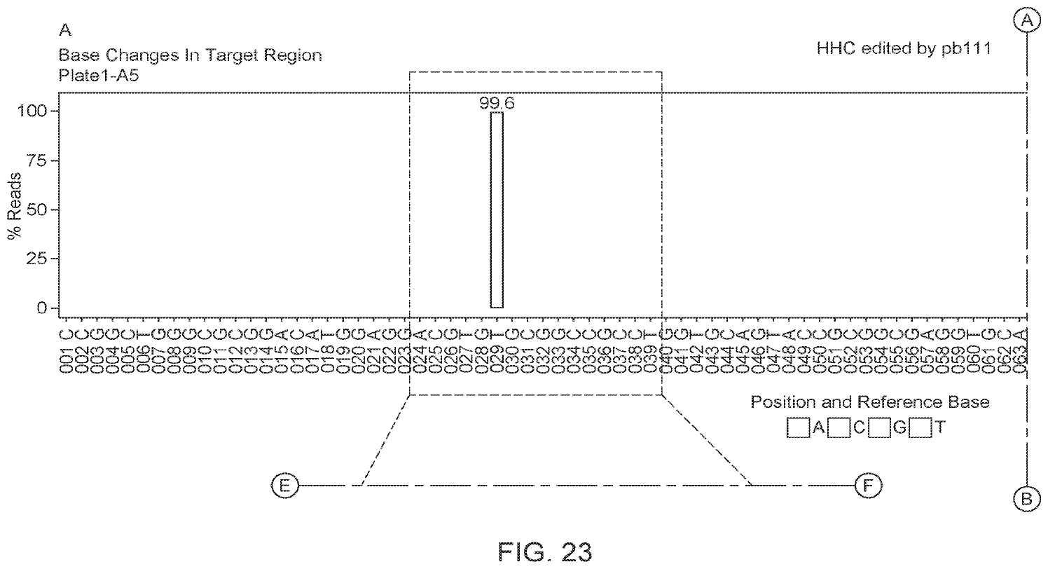

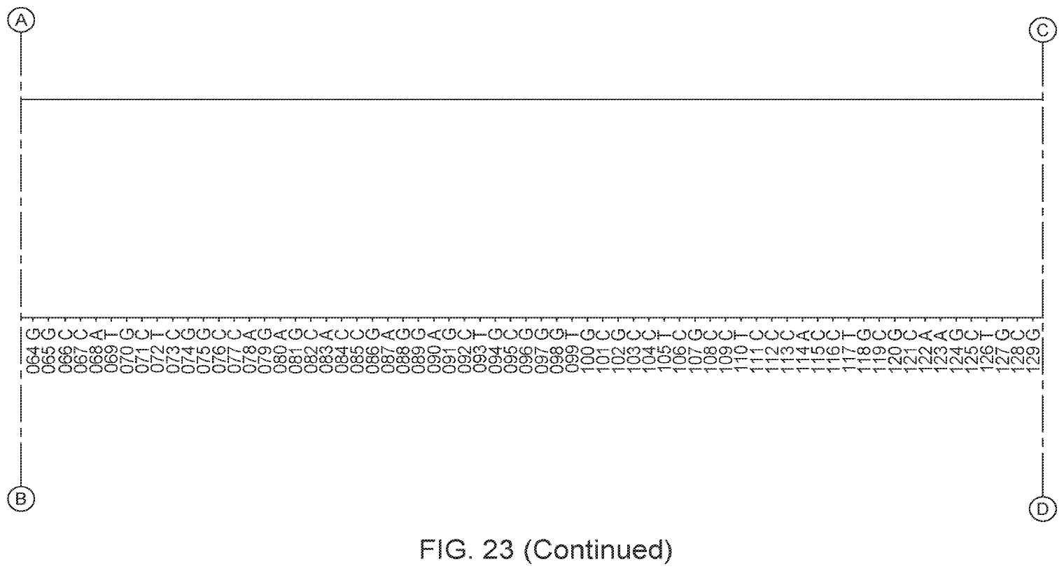

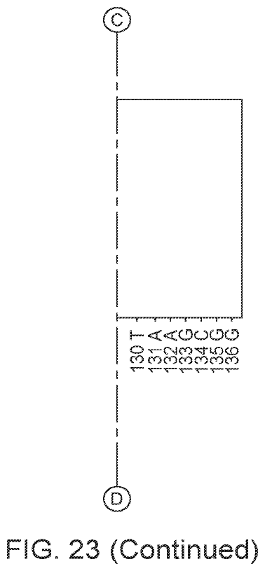

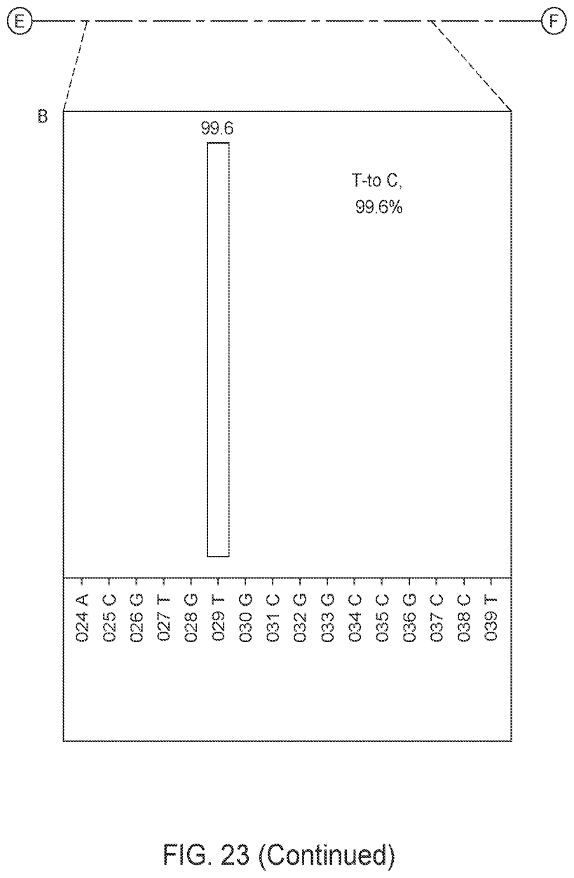

FIG. 22 results obtained with untargeted hepatocytes and serves as a control. FIG. 23 shows confirmation of detection of single nucleotide T→C conversion at this target site as well as single nucleotide polymorphisms (SNPs) analysis within a target region of surrounding codon 112 of this ApoE locus. In FIGS. 22 and 23, panel A shows overall views of SNPs analysis at these target sites obtained with untargeted hepatocytes respectively targeted hepatocytes. Bar graphs plot frequencies of SNPs at each nucleotide position in this 175 bp PCR amplification region. Panel B is a magnified view of the portion close to this gene repair site. In this example cells transfected with pb 111 (RECQ1 derived HbW) and a correction template showed a T-to-C conversion at this expected nucleotide position with a frequency of 99.6%. Compared to non-transfected hepatocytes, no other nucleotide conversions had occurred at a level significantly above background. Comparing to untransfected cells, no obvious unwanted SNPs were detected.

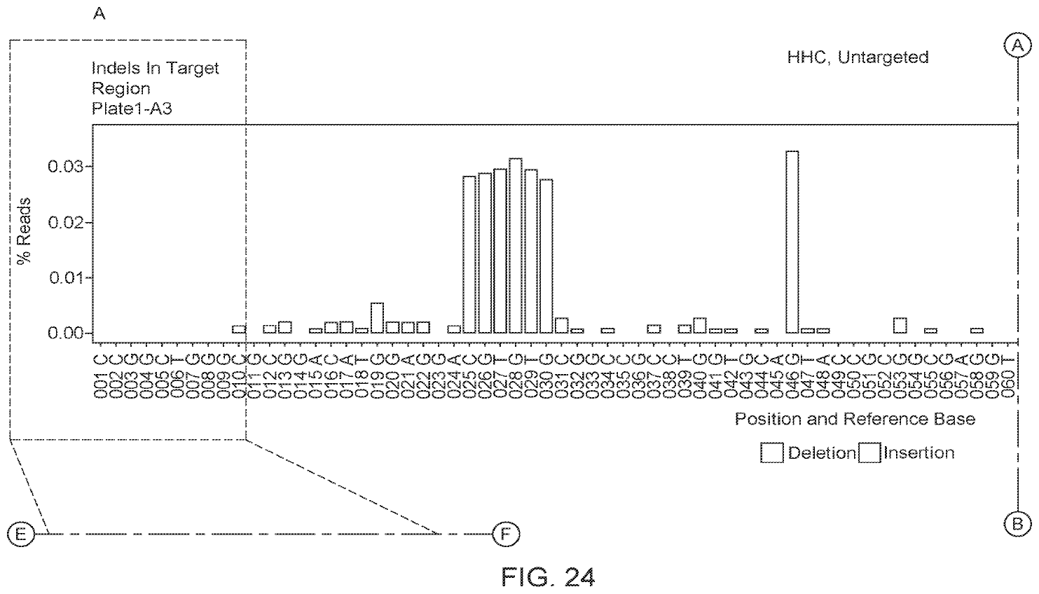

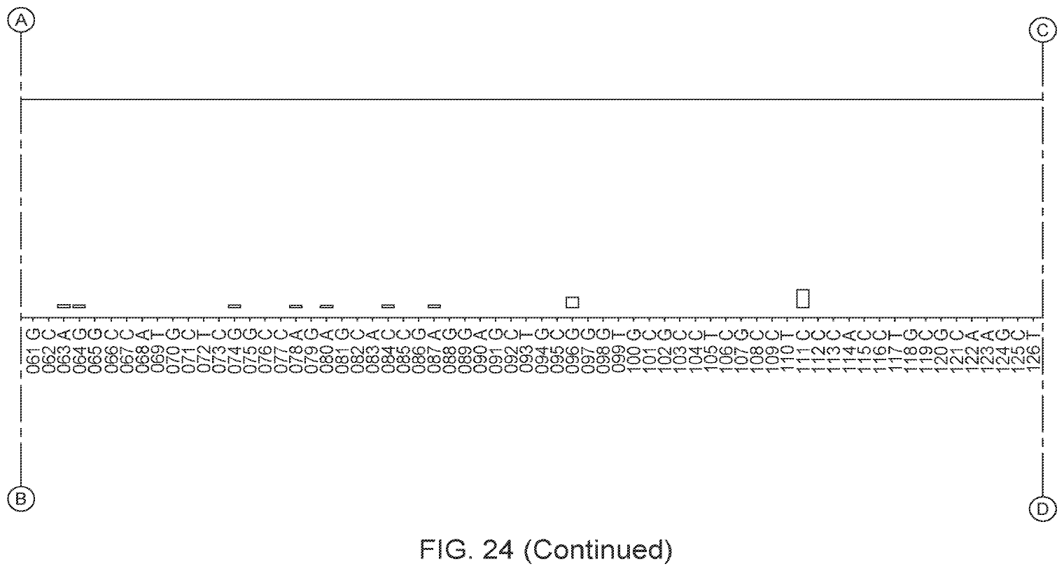

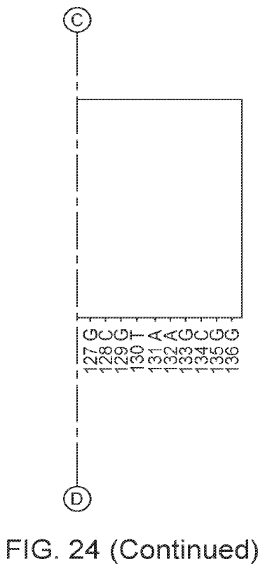

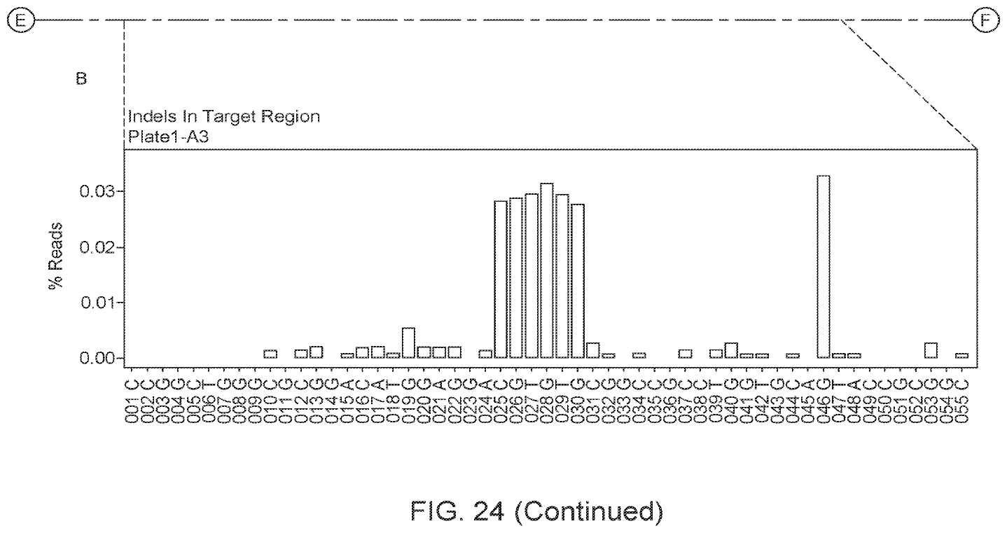

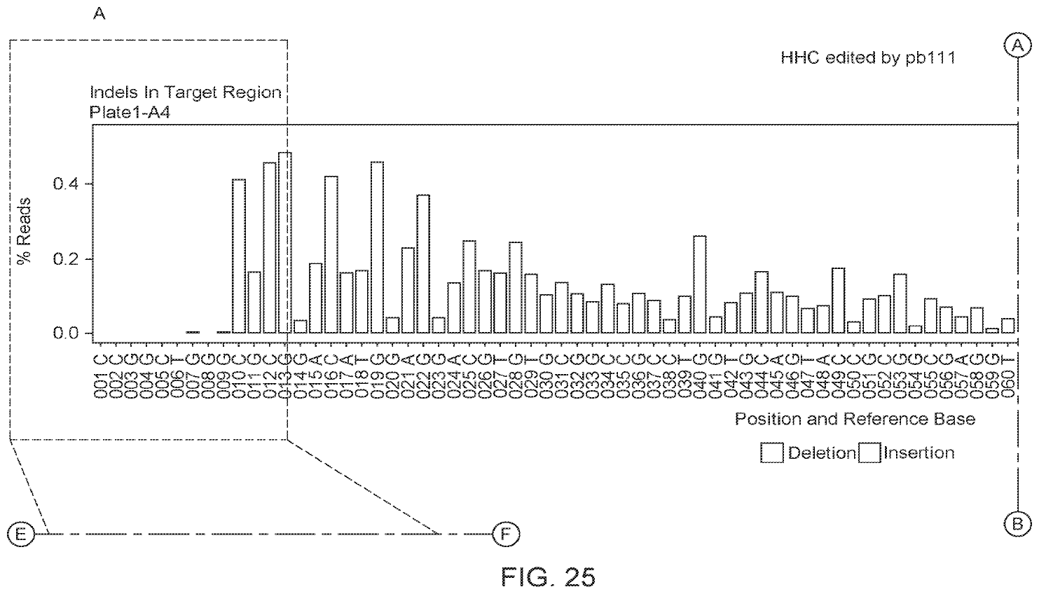

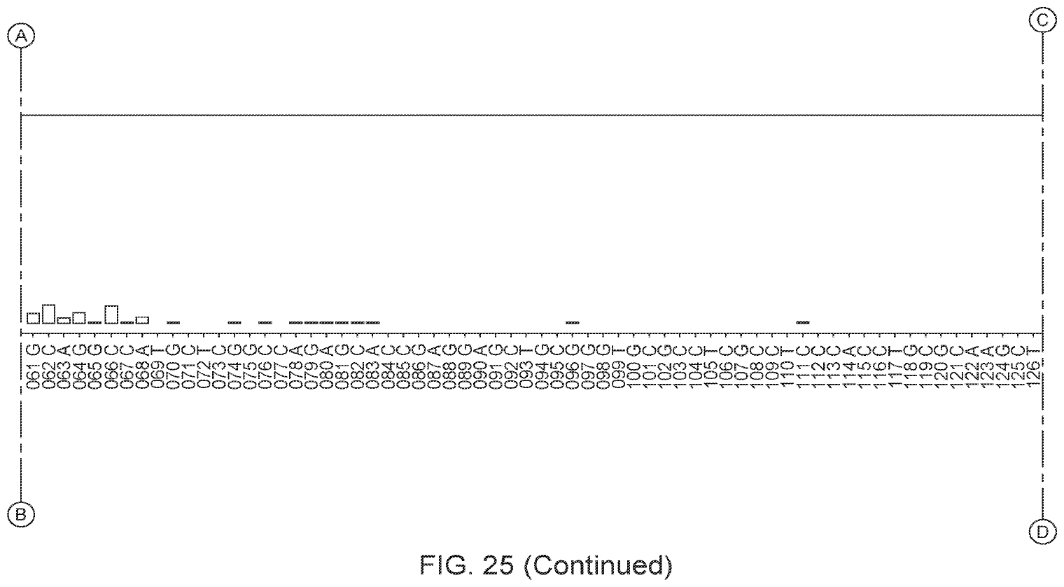

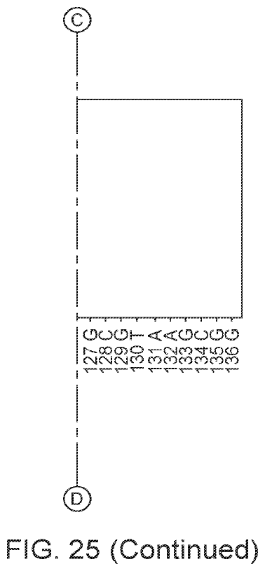

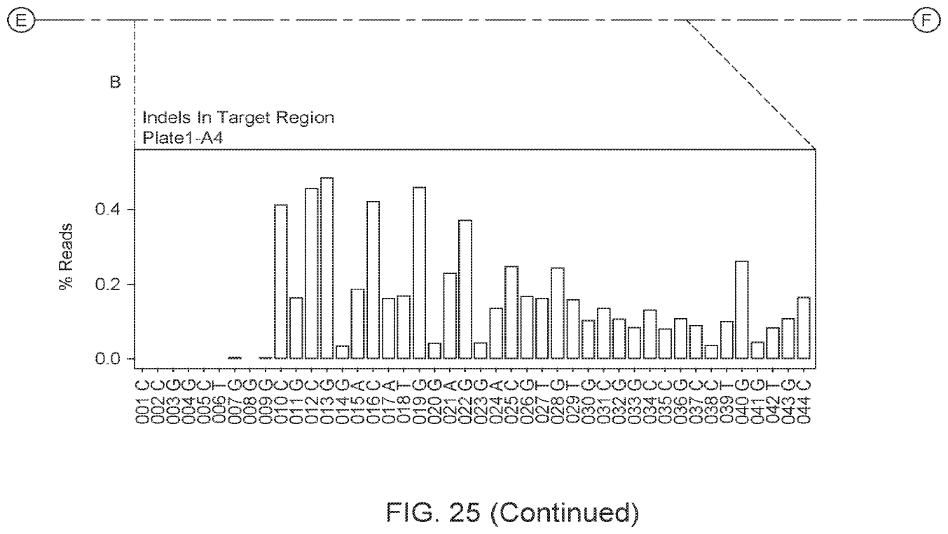

FIG. 24 and FIG. 25 show insertion and deletion analysis around codon 112 of ApoE in a untargeted hepatocyte control (FIG. 24) respectively in an example using pb 111 (RECQ1 derived) (FIG. 25), displayed as a frequency plot of insertions and deletions analysis for pooled hepatocytes. Bar graphs plot frequencies of insertions and deletions at each nucleotide position of this 175 bp PCR amplification region. This indels analysis showed, in general, a very low frequency (<0.5%) of insertions and/or deletions. Patterns and frequencies of indels at each position from both targeted and untransfected hepatocytes were not statistically significantly different and were considered to be within the error range and the detection limitations typical for the PCR and next generation sequencing method used.

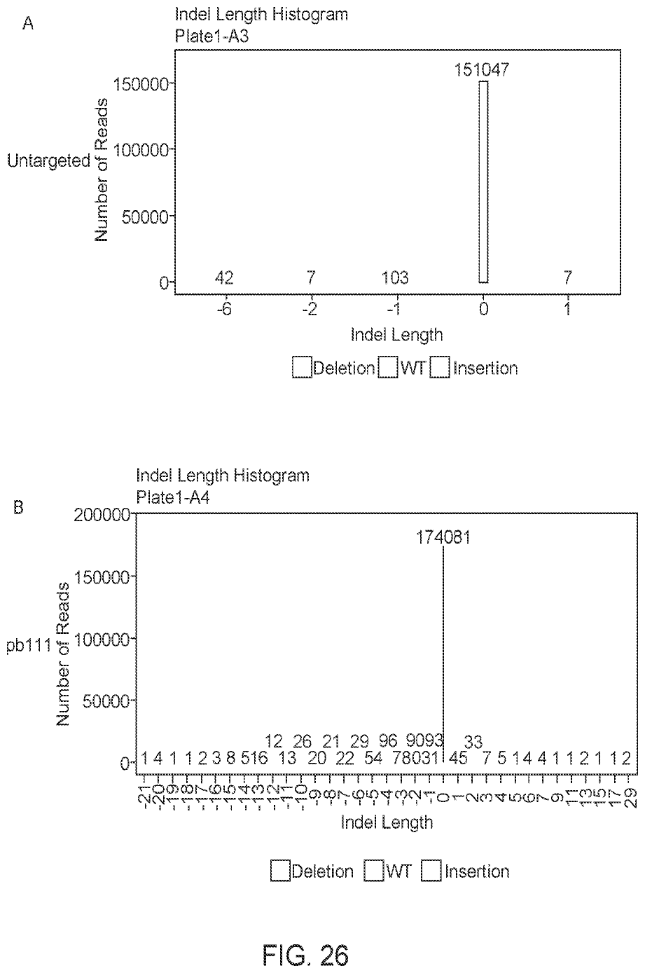

FIG. 26 shows insertion and deletion (Indels) analysis by next generation sequencing of untargeted and edited hepatocytes illustrated as histograms. An x-axis indicates the number of deleted nucleotides (expressed as negative numbers), no insertions or deletions (indicated by 0) respectively insertions (expressed by positive numbers). A y-axis indicates the number of sequence reads obtained for each InDel. Panel A shows overviews of InDels at each position of the targeting region of codon 112 site of untargeted human hepatocytes. Panel B shows overviews of InDels at each position of the targeting region of codon 112 site of human ApoE using pb 111. This example illustrates a low amount of indels generated compared to wild type or single nucleotide changes. In addition, it also illustrates that 1-nucleotide deletions followed by 2-nucleotide deletions are most prevalent type of InDels. As previously discussed, a potential source of these observed types of InDels may also arise from non-perfect modification oligonucleotide synthesis.

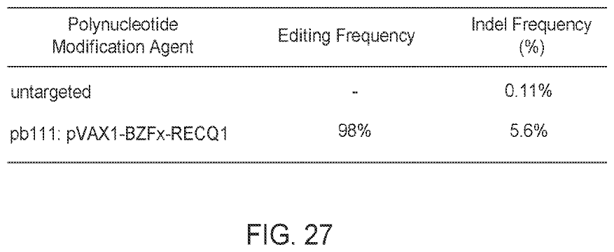

FIG. 27 shows overall indels and editing frequencies in human hepatocytes using zinc finger helicase beta wing mediated gene editing targeting with pb 111 and a sequence modification polynucleotide, an overall zinc finger helicase beta wing mediated gene editing frequency of 98% and an indel frequency of 5.5% was observed. The higher editing frequency in hepatocytes compared to B-cells may have many different causes. Not being bound by one particular theory, since ApoE is highly expressed in hepatocytes, such high expression levels may aid in the accessibility of the editing components. Highly expressed genes often involved more “open” or “accessible” chromatin structures or conformations.

Taken together, zinc finger helicase beta wing mediated gene editing is able to achieve relatively high gene editing efficiency with very low indel frequencies in multiple cell types.

### Example 4: Modification of an Endogenous Genomic Target: Bcl11A by SSB-HbW-Based Gene Editing in Human B-Cells

The present example describes sequence specific genetic modification of a human Bcl11A gene in exemplary cells using technologies described herein. In this example, an enhancer in intron 2 of human BCL11A was targeted and edited by SSB-HbW-based gene editing with a polynucleotide modification agent and a single stranded oligonucleotide template (i.e., a sequence modification polynucleotide). The present disclosure contemplates that, in some embodiments, disruption of this enhancer decreases expression of a transcriptional factor, Bcl11A (Psatha et al., Mol. Ther. Methods Clin. Dev. 2018 Sep. 21; 10:313-326, which is herein incorporated by reference in its entirety), Without being bound by any particular theory, increased production of fetal hemoglobin (HbF) and/or decreased production of adult hemoglobin (e.g., via gene editing of Bcl11A) may ameliorate clinical symptoms of disorders involving adult beta-hemoglobin, such as β-thalassemia and sickle cell disease. Accordingly, in some embodiments, decreasing levels of Bcl11A may increase HbF levels. (Bauer et al., Science, 2013 Oct. 11; 342(6155):253-257, which is herein incorporated by reference in its entirety). Thus, this example describes development of SSB-HbW-based gene editing systems designed to edit a medically relevant gene within a mammalian (e.g., human) genome, specifically a sequence of an enhancer in intron 2 of human Bcl11A

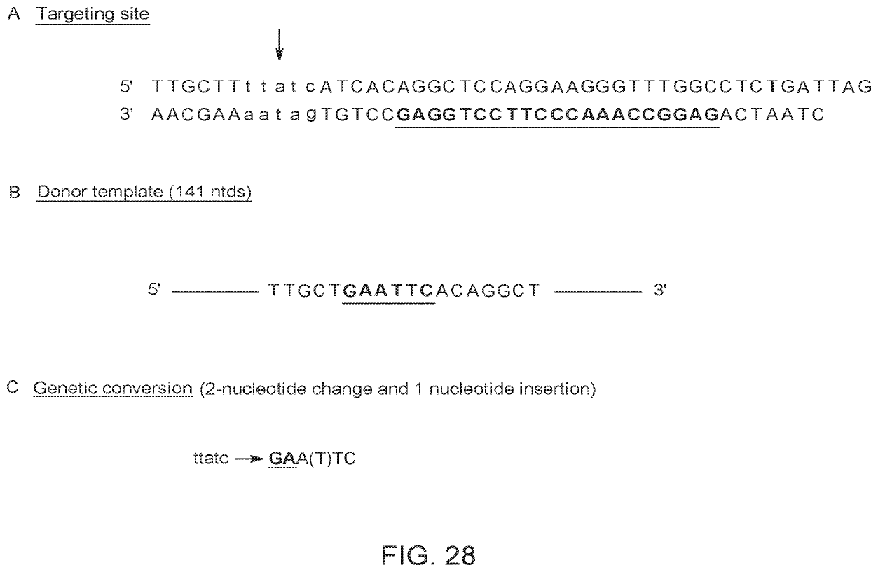

FIG. 28 provides a schematic depicting the approach used in this Example for editing a “GATAA” motif in an enhancer in intron 2 of human Bcl11A in human B cells. FIG. 28, panel A depicts the targeting site of an exemplary polynucleotide modification agents, encoded on plasmid pb112. This construct includes a DNA recognition domain comprising an array of 7 zinc-fingers, specifically designed to recognize 5′-GAGGCCAAACCCTTCCTGGAG-3′ (SEQ ID NO.: 64), a 21-nucleotide sequence on the lagging strand of human Bcl11A. A targeted nucleotide “TTACT” on leading strand of target site was displayed as a lowercase letter “ttact”, 5′ upstream of this binding site. FIG. 28, panel B depicts a sequence modification polynucleotide used was a 140-nucleotide single stranded DNA oligonucleotide containing a TTATC→GAATTC substitution roughly located in the middle of the length of this oligonucleotide. This sequence of this sequence modification polynucleotide used is provided as SEQ ID NO.: 12 (below) with an underlined and bold “GAATTC” to indicate a GAATTC sequence used in this “ttatc-to-GAATC” conversion. FIG. 28, panel C depicts genetic conversion by which “tt” is converted into “GA” and “C” is inserted after “at”, as such a conversion including a two-nucleotide conversion and a nucleotide insertion.

(SEQ ID NO.: 12)
5′-CTCTTAGACATAACACACCAGGGTCAATACAACTTTGAAGCTAGTC
TAGTGCAAGCTAACAGTTGCTTGAATTCACAGGCTCCAGGAAGGGTTTG
GCCTCTGATTAGGGTGGGGGCGTGGGTGGGGTAGAAGAGGACTGGC-3′

Here, a polynucleotide modification agent (encoded on plasmid pb112 (full length DNA (SEQ ID NO.: 65); cDNA (SEQ ID. NO.: 66); amino acid sequence (SEQ ID. NO.: 67)), which has a DNA recognition domain comprised in an array of 7 zinc-fingers, was designed to specifically recognize 5′-GAGGCCAAACCCTTCCTGGAG-3′ (SEQ ID NO.: 64), a 21-nucleotide sequence on the lagging DNA strand (bottom row of nucleotides) of human Bcl11A. Exemplary sequences are provided in Table 5 below.

TABLE 5
Exemplary polynucleotide modification agents targeting Bcl11A

| SEQ | | AA/ | |
|---|---|---|---|
| ID | | DNA/ | |
| NO: | Feature | cDNA | Sequence |
| 65 | pb112 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTGGGGTACCGGC |
| | | | GGCGATGGCGGCGATGGCCGAGCGGCCCTTCCAGTGCAGGATCTGTATGCGCAACT |
| | | | TTTCTCGGTCCTCCAACCTGACCCGGCACATCAGAACCCATACAGGCGAAAAGCCT |
| | | | TTCGCCTGCGACATTTGTGGGAGAAAATTTGCTCGGTCCGACGCCCTGTCCGAGCA |
| | | | TACCAAGATCCACACCGGCTCTCAGAAACCATTCCAGTGCCGCATTTGTATGCGGA |
| | | | ATTTTTCCGACTCCTCCGCCCTGACCACCCATATCCGCACTCACACCGGAGAGAAG |
| | | | CCCTTTGCTTGCGACATTTGTGGCAGGAAATTTGCTGACTCCTCCGACCTGTCCGA |
| | | | GCACACTAAGATCCATACTGGGTCACAGAAACCTTTCCAGTGCCGGATTTGTATGA |
| | | | GAAACTTTAGCCAGTCCGGCAACCTGTCCCAGCACATCAGAACCCATACAGGCGAA |
| | | | AAGCCTTTCGCCTGCGACATTTGTGGGAGAAAATTTGCTGACCGGTCCGACCTGAC |
| | | | CCGGCATACCAAGATCCACACCGGCTCTCAGAAACCATTCCAGTGCCGCATTTGTA |
| | | | TGCGGAATTTTTCCCGGTCCGACAACCTGACCCGGCACATCAGAACACATACTGGG |
| | | | GAGAAGATCATCGCCCACTTCCTGATCCAGCAGTACCTGAAGGAGGACTACTCCTT |
| | | | CACCGCCTACGCCACCATCTCCTACCTGAAGATCGGCTGAGCGGCCGCTCGAGTCT |
| | | | AGAGGGCCCGTTTAAACCCGCTGATCAGCCTCGACTGTGCCTTCTAGTTGCCAGCC |
| | | | ATCTGTTGTTTGCCCCTCCCCCGTGCCTTCCTTGACCCTGGAAGGTGCCACTCCCA |
| | | | CTGTCCTTTCCTAATAAAATGAGGAAATTGCATCGCATTGTCTGAGTAGGTGTCAT |
| | | | TCTATTCTGGGGGGTGGGGTGGGGCAGGACAGCAAGGGGGAGGATTGGGAAGACAA |
| | | | TAGCAGGCATGCTGGGGATGCGGTGGGCTCTATGGCTTCTACTGGGCGGTTTTATG |
| | | | GACAGCAAGCGAACCGGAATTGCCAGCTGGGGCGCCCTCTGGTAAGGTTGGGAAGC |
| | | | CCTGCAAAGTAAACTGGATGGCTTTCTCGCCGCCAAGGATCTGATGGCGCAGGGGA |
| | | | TCAAGCTCTGATCAAGAGACAGGATGAGGATCGTTTCGCATGATTGAACAAGATGG |
| | | | ATTGCACGCAGGTTCTCCGGCCGCTTGGGTGGAGAGGCTATTCGGCTATGACTGGG |
| | | | CACAACAGACAATCGGCTGCTCTGATGCCGCCGTGTTCCGGCTGTCAGCGCAGGGG |
| | | | CGCCCGGTTCTTTTTGTCAAGACCGACCTGTCCGGTGCCCTGAATGAACTGCAAGA |
| | | | CGAGGCAGCGCGGCTATCGTGGCTGGCCACGACGGGCGTTCCTTGCGCAGCTGTGC |
| | | | TCGACGTTGTCACTGAAGCGGGAAGGGACTGGCTGCTATTGGGCGAAGTGCCGGGG |
| | | | CAGGATCTCCTGTCATCTCACCTTGCTCCTGCCGAGAAAGTATCCATCATGGCTGA |
| | | | TGCAATGCGGCGGCTGCATACGCTTGATCCGGCTACCTGCCCATTCGACCACCAAG |
| | | | CGAAACATCGCATCGAGCGAGCACGTACTCGGATGGAAGCCGGTCTTGTCGATCAG |
| | | | GATGATCTGGACGAAGAGCATCAGGGGCTCGCGCCAGCCGAACTGTTCGCCAGGCT |
| | | | CAAGGCGAGCATGCCCGACGGCGAGGATCTCGTCGTGACCCATGGCGATGCCTGCT |
| | | | TGCCGAATATCATGGTGGAAAATGGCCGCTTTTCTGGATTCATCGACTGTGGCCGG |
| | | | CTGGGTGTGGCGGACCGCTATCAGGACATAGCGTTGGCTACCCGTGATATTGCTGA |
| | | | AGAGCTTGGCGGCGAATGGGCTGACCGCTTCCTCGTGCTTTACGGTATCGCCGCTC |
| | | | CCGATTCGCAGCGCATCGCCTTCTATCGCCTTCTTGACGAGTTCTTCTGAATTATT |
| | | | AACGCTTACAATTTCCTGATGCGGTATTTTCTCCTTACGCATCTGTGCGGTATTTC |
| | | | ACACCGCATACAGGTGGCACTTTTCGGGGAAATGTGCGCGGAACCCCTATTTGTTT |
| | | | ATTTTTCTAAATACATTCAAATATGTATCCGCTCATGAGACAATAACCCTGATAAA |
| | | | TGCTTCAATAATAGCACGTGCTAAAACTTCATTTTTAATTTAAAAGGATCTAGGTG |
| | | | AAGATCCTTTTTGATAATCTCATGACCAAAATCCCTTAACGTGAGTTTTCGTTCCA |
| | | | CTGAGCGTCAGACCCCGTAGAAAAGATCAAAGGATCTTCTTGAGATCCTTTTTTTC |
| | | | TGCGCGTAATCTGCTGCTTGCAAACAAAAAAACCACCGCTACCAGCGGTGGTTTGT |
| | | | TTGCCGGATCAAGAGCTACCAACTCTTTTTCCGAAGGTAACTGGCTTCAGCAGAGC |
| | | | GCAGATACCAAATACTGTCCTTCTAGTGTAGCCGTAGTTAGGCCACCACTTCAAGA |
| | | | ACTCTGTAGCACCGCCTACATACCTCGCTCTGCTAATCCTGTTACCAGTGGCTGCT |
| | | | GCCAGTGGCGATAAGTCGTGTCTTACCGGGTTGGACTCAAGACGATAGTTACCGGA |
| | | | TAAGGCGCAGCGGTCGGGCTGAACGGGGGGTTCGTGCACACAGCCCAGCTTGGAGC |
| | | | GAACGACCTACACCGAACTGAGATACCTACAGCGTGAGCTATGAGAAAGCGCCACG |
| | | | CTTCCCGAAGGGAGAAAGGCGGACAGGTATCCGGTAAGCGGCAGGGTCGGAACAGG |
| | | | AGAGCGCACGAGGGAGCTTCCAGGGGGAAACGCCTGGTATCTTTATAGTCCTGTCG |
| | | | GGTTTCGCCACCTCTGACTTGAGCGTCGATTTTTGTGATGCTCGTCAGGGGGGCGG |
| | | | AGCCTATGGAAAAACGCCAGCAACGCGGCCTTTTTACGGTTCCTGGGCTTTTGCTG |
| | | | GCCTTTTGCTCACATGTTCTT |
| 66 | pb112 | cDNA | ATGGCGGCGATGGCCGAGCGGCCCTTCCAGTGCAGGATCTGTATGCGCAACTTTTC |
| | | | TCGGTCCTCCAACCTGACCCGGCACATCAGAACCCATACAGGCGAAAAGCCTTTCG |
| | | | CCTGCGACATTTGTGGGAGAAAATTTGCTCGGTCCGACGCCCTGTCCGAGCATACC |
| | | | AAGATCCACACCGGCTCTCAGAAACCATTCCAGTGCCGCATTTGTATGCGGAATTT |
| | | | TTCCGACTCCTCCGCCCTGACCACCCATATCCGCACTCACACCGGAGAGAAGCCCT |
| | | | TTGCTTGCGACATTTGTGGCAGGAAATTTGCTGACTCCTCCGACCTGTCCGAGCAC |
| | | | ACTAAGATCCATACTGGGTCACAGAAACCTTTCCAGTGCCGGATTTGTATGAGAAA |
| | | | CTTTAGCCAGTCCGGCAACCTGTCCCAGCACATCAGAACCCATACAGGCGAAAAGC |
| | | | CTTTCGCCTGCGACATTTGTGGGAGAAAATTTGCTGACCGGTCCGACCTGACCCGG |
| | | | CATACCAAGATCCACACCGGCTCTCAGAAACCATTCCAGTGCCGCATTTGTATGCG |
| | | | GAATTTTTCCCGGTCCGACAACCTGACCCGGCACATCAGAACACATACTGGGGAGA |
| | | | AGATCATCGCCCACTTCCTGATCCAGCAGTACCTGAAGGAGGACTACTCCTTCACC |
| | | | GCCTACGCCACCATCTCCTACCTGAAGATCGGCTGA |
| 67 | pb112 | AA | MAAMAERPFQCRICMRNFSRSSNLTRHIRTHTGEKPFACDICGRKFARSDALSEHT |
| | | | KIHTGSQKPFQCRICMRNFSDSSALTTHIRTHTGEKPFACDICGRKFADSSDLSEH |
| | | | TKIHTGSQKPFQCRICMRNFSQSGNLSQHIRTHTGEKPFACDICGRKFADRSDLTR |
| | | | HTKIHTGSQKPFQCRICMRNFSRSDNLTRHIRTHTGEKIIAHFLIQQYLKEDYSFT |
| | | | AYATISYLKIG* |

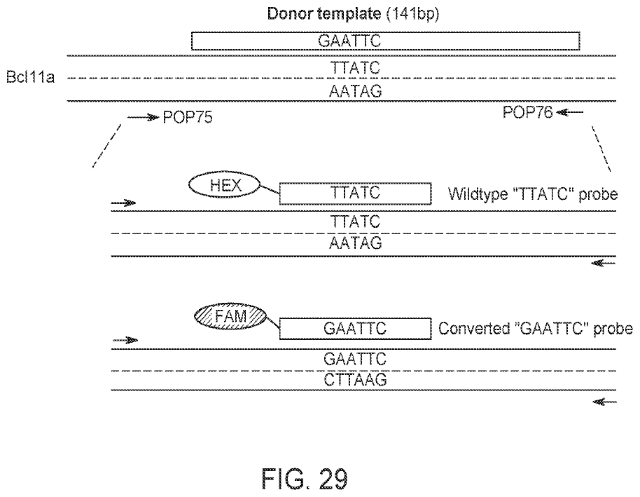

Detections of genetic “ttatc-to-GAATC” conversion after SSB-HbW-based gene edition were performed by droplet digital PCR (ddPCR). Relative positions of a correction ssODN (i.e., sequence modification polynucleotide) and position of an exemplary primer pair: POP75 and POP76 are also indicated in FIG. 29. One common primer, POP75 was located outside this ssODN template (i.e., sequence modification polynucleotide) sequence, while POP76, located inside. Allele-specific probes conjugated with fluorophores FAM and HEX were designed to distinguish between “GAATC” and “ttatc” respectively.

POP75 Bcl11a forward primer has a sequence of
(SEQ ID NO.: 68)
ACTCTTAGACATAACACACC.
POP76 Bcl11a reverse primer has a sequence of
(SEQ ID NO.: 69)
AAGAGAGCCTTCCGAAAGA.

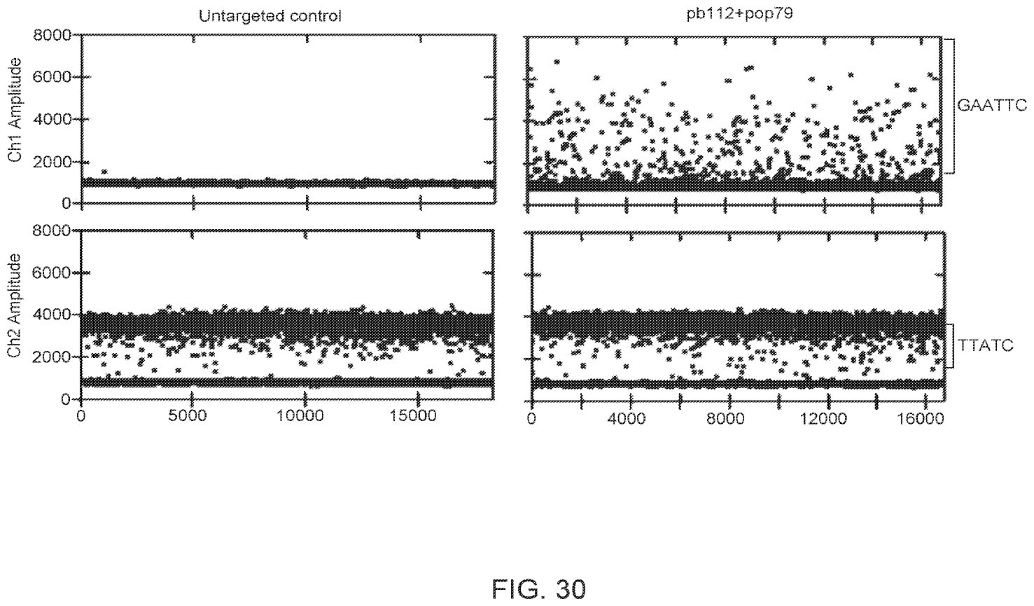

FIG. 30 demonstrates successful “ttatc-to-GAATC” genetic conversion at a GATTA motif of human Bcl11a as measured by ddPCR. In this example, after transfection of B cells with plasmid pb112 and this 141-nucleotide correction template, cells were allowed to recover and grow on complete culture medium, containing 15% FBS in RPMI, for five days. After five days genomic DNA was isolated and used in ddPCR analysis. Raw droplet data are shown as in FIG. 30 where these “GAATTC” droplets are displayed in the top panel in each set, while “ttatc” droplets were in the lower one. Untargeted B-cells were used as a negative control, showing only “ttatc” droplets, but no “GAATTC” droplets. Untargeted B cells only had “ttatc” droplets, demonstrating untargeted wildtype genotype. After B cells were transfected with pb112 and ssODN template (i.e., sequence modification polynucleotide), “GAATTC” droplets appeared after being targeted and edited by this polynucleotide modification agent in combination with a correcting template, demonstrating successful “ttatc-to-GAATC” genetic conversion at this GATAA motif of human Bcl11a.

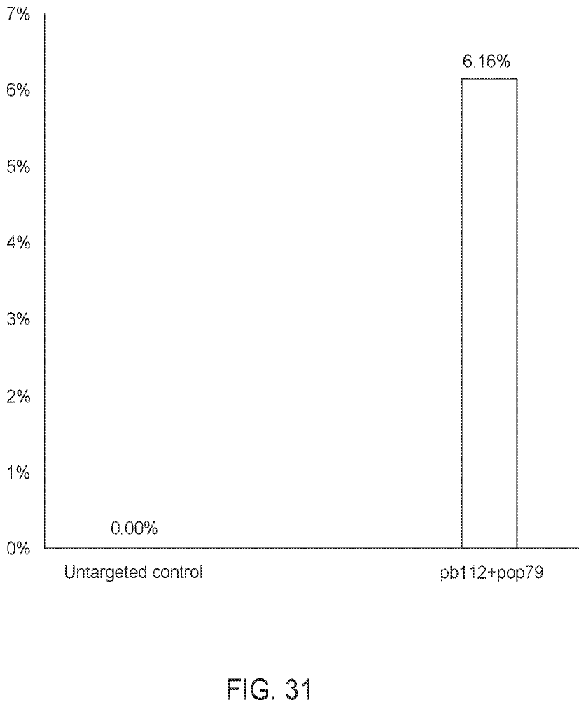

FIG. 31 shows genetic “ttatc-to-GAATTC” conversion frequencies as measured by ddPCR after HbW-based gene editing. It shows editing frequencies corresponding to cellular “ttatc-to-GAATTC” conversion percentages, defined as a percentage of “GAATTC” droplet events divided by the sum of “GAATTC” and “ttatc” droplet events. Here, this HbW-based gene editing achieved a 6.16% genetic conversion frequency compared to a background level of 0.00% of “ttatc-to-GAATTC” conversion.

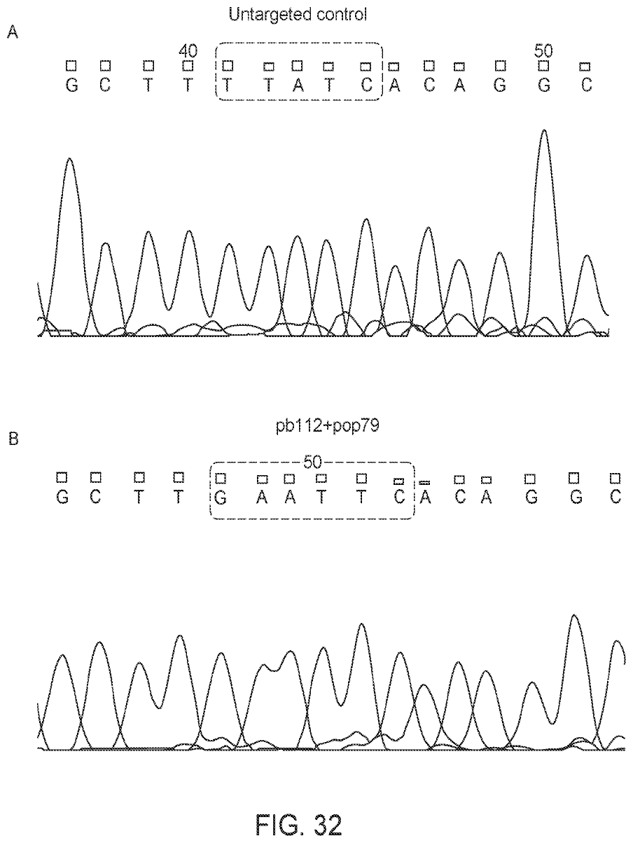

FIG. 32 shows exemplary Sanger sequencing results obtained to further confirm successful targeting and editing of GATAA motif of the human Bcl11A gene. Genomic DNA was isolated and used as a template on which a 197-bp PCR amplicon surrounding Bcl11a GATAA motif was generated by using a primer set of POP75 and POP76. Amplified PCR products from targeted B cells were analyzed. FIG. 32, panel A shows an exemplary chromatogram from untargeted B-cell population, showing a wild type “TTATC” sequence by Sanger sequencing. FIG. 32, panel B shows an exemplary chromatogram of a pb112 edited B-cell population, showing a “ttatc-to-GAATTC” sequence conversion by Sanger sequencing. These results confirm that a polynucleotide modification agent in combination with a sequence modification polynucleotide can successfully target and edit a sequence in an endogenous mammalian gene in mammalian cells to successfully modify a disease-causing genotype.

This Example confirms that SSB-HbW-based gene editing can be used to successfully genetically modify an endogenous disease-associated genotype within a mammalian genome by specifically converting a “GATAA” motif into “GATTCC” in an enhancer in intron 2 of human BCL11A. This example further demonstrates use of SSB-HbW-based gene editing to modify disease-relevant nucleotide targets in mammalian cells by using a SSB-HbW-based gene editing approach and system to genetically modify a human gene.

### Example 5: Modification of an Endogenous Genomic Target: PolG by SSB-HbW-Based Gene Editing in Human B-Cells and Cell Lines

The present example describes sequence specific genetic modification of a human PolG gene in exemplary cells using technologies described herein. In this example, human PolG at or around codon 467 was targeted and edited by SSB-HbW-based gene editing with a polynucleotide modification agent and a single stranded oligonucleotide template (i.e., a sequence modification polynucleotide). DNA polymerase γ (POLY) is the main protein responsible for mitochondrial DNA (mtDNA) replication and mutations in its gene (PolG) are the most frequent cause of mitochondrial disease related to a single nuclear gene. (Silva-Pinheiro et al., Nucleic Acids Research, 2021, Vol. 49, No. 9:5230-5248, which is herein incorporated by reference in its entirety). The amino acid substitution mutation, A467T, is associated with a wide range of mitochondrial disorders, including Alpers syndrome, juvenile spinocerebellar ataxia-epilepsy syndrome, and progressive external ophthalmoplegia, each with vastly different clinical presentations, tissue specificities, and ages of onset. (Chan et al., Journal of Biological Chemistry, 2005, Vol. 280, Issue 36:31341-31346, which is herein incorporated by reference in its entirety). The present disclosure contemplates that, in some embodiments, a polynucleotide modification system as described herein can be used for conversion of nucleotides at or around position of codon A467 of PolG. This example describes development of a specifically designed polynucleotide modification agent (e.g., a SSB-HbW agent) with in combination with a sequence modification polynucleotide (e.g., a specific single stranded oligonucleotide template) to convert a DNA sequence at or around codon A467 of PolG to create amino acid substitution(s).

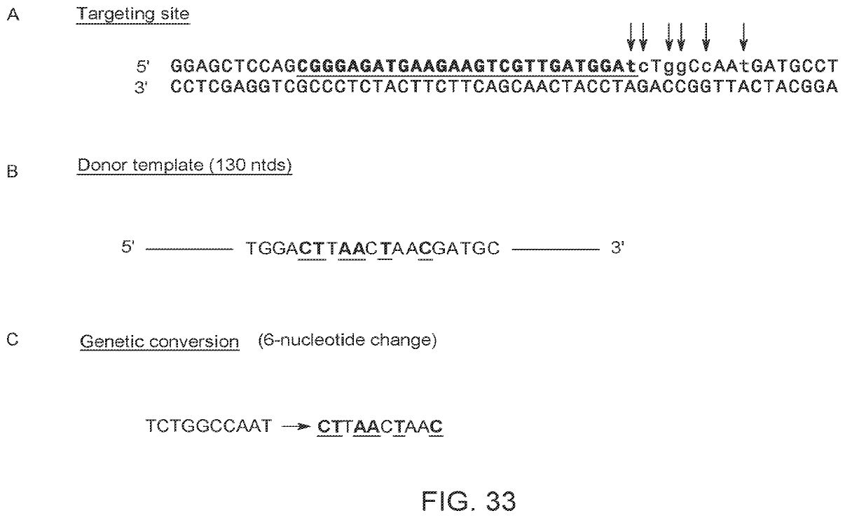

FIG. 33 depicts a schematic gene editing approach taken for gene editing of an endogenous genomic target around codon A467 of human PolG in B cells and cell lines. In this example, an exemplary polynucleotide modification agent, encoded on plasmid pb 125, includes a DNA recognition domain which was an array of 9 zinc-fingers, specifically designed to recognize 5′-CGGGAGATGAAGAAGTCGTTGATGGAT-3′ (SEQ ID NO.: 71), a 27-nucleotide sequence on the leading strand of human PolG, displayed with underlined and bold letters. As depicted in FIG. 33, panel A, targeted nucleotides, displayed as a lowercase letters “tcTggCcAAt”, 3′ upstream of this binding site, converted nucleotide positions are indicated by arrows. In this example, a donor template was used: a 130-nucleotide single stranded DNA oligonucleotide with a desired tcTggCcAAt-to-CTTAACTAAC conversion roughly located in the middle of this oligonucleotide, as depicted in FIG. 33, panel B. The sequence modification polynucleotide used is provided as SEQ ID NO.: 72 (below) with an underlined and bold “CTTAACTAAC” to indicate “tcTggCcAAt-to-CTTAACTAAC” conversion. FIG. 33, panel C depicts 6-nucleotide conversion resulted from successful genetic conversion by SSB-HbW-based gene editing.

(SEQ ID NO.: 72)
5′-TGGCAGAGGCACAGGGCACTTATGAGGAGCTCCAGCGGGAGATGA
AGAAGTCGTTGATGGACTTAACTAACGATGCCTGCCAGCTGCTCTCAG
GAGAGAGGTAGCCAGGCCTTGGGTGGGCAGGATCTAG-3′

Here, a polynucleotide modification agent (encoded on plasmid pb 125 (full length DNA (SEQ ID NO.: 73); cDNA (SEQ ID. NO.: 74); amino acid sequence (SEQ ID. NO.: 75)), which has a DNA recognition domain comprised in an array of 9 zinc-fingers, was designed to specifically recognize 5′-CGGGAGATGAAGAAGTCGTTGATGGAT-3′ (SEQ ID NO.: 71), a 27-nucleotide sequence on the leading DNA strand (top row of nucleotides) of human PolG. Exemplary sequences are provided in Table 6 below.

TABLE 6
Exemplary polynucleotide modification agents targeting PolG

| SEQ | | AA/ | |
|---|---|---|---|
| ID | | DNA/ | |
| NO: | Feature | cDNA | Sequence |
| 73 | pb125 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCCAGTGCAGGATCTGTATGCGCAACTTTTCTACCTCCGGCA |
| | | | ACCTGACCCGGCACATCAGAACCCATACAGGCGAAAAGCCTTTCGCCTGCGACATT |
| | | | TGTGGGAGAAAATTTGCTCGGTCCGACGCCCTGTCCCAGCATACCAAGATCCACAC |
| | | | CGGCTCTCAGAAACCATTCCAGTGCCGCATTTGTATGCGGAATTTTTCCCGGTCCT |
| | | | CCGCCCTGACCACCCATATCCGCACTCACACCGGAGAGAAGCCCTTTGCTTGCGAC |
| | | | ATTTGTGGCAGGAAATTTGCTCGGTCCTCCGACCTGACCACCCACACTAAGATCCA |
| | | | TACTGGGTCACAGAAACCTTTCCAGTGCCGGATTTGTATGAGAAACTTTAGCCGGT |
| | | | CCTCCAACCTGTCCCAGCACATCAGAACCCATACAGGCGAAAAGCCTTTCGCCTGC |
| | | | GACATTTGTGGGAGAAAATTTGCTCGGTCCTCCAACCTGTCCCAGCATACCAAGAT |
| | | | CCACACCGGCTCTCAGAAACCATTCCAGTGCCGCATTTGTATGCGGAATTTTTCCC |
| | | | GGTCCTCCGCCCTGTCCCAGCATATCCGCACTCACACCGGAGAGAAGCCCTTTGCT |
| | | | TGCGACATTTGTGGCAGGAAATTTGCTCGGTCCTCCAACCTGACCCGGCACACTAA |
| | | | GATCCATACTGGGTCACAGAAACCTTTCCAGTGCCGGATTTGTATGAGAAACTTTA |
| | | | GCCGGTCCGACCACCTGTCCGAGCACATCAGAACACATACTGGGCTGAGAGAGAAG |
| | | | ATCATCGCCCACTTCCTGATCCAGCAGTACCTGAAGGAGGACTACTCCTTCACCGC |
| | | | CTACGCCACCATCTCCTACCTGAAGATCGGCTGAGCGGCCGCTCGAGTCTAGAGGG |
| | | | CCCGTTTAAACCCGCTGATCAGCCTCGACTGTGCCTTCTAGTTGCCAGCCATCTGT |
| | | | TGTTTGCCCCTCCCCCGTGCCTTCCTTGACCCTGGAAGGTGCCACTCCCACTGTCC |
| | | | TTTCCTAATAAAATGAGGAAATTGCATCGCATTGTCTGAGTAGGTGTCATTCTATT |
| | | | CTGGGGGGTGGGGTGGGGCAGGACAGCAAGGGGGAGGATTGGGAAGACAATAGCAG |
| | | | GCATGCTGGGGATGCGGTGGGCTCTATGGCTTCTACTGGGCGGTTTTATGGACAGC |
| | | | AAGCGAACCGGAATTGCCAGCTGGGGCGCCCTCTGGTAAGGTTGGGAAGCCCTGCA |
| | | | AAGTAAACTGGATGGCTTTCTCGCCGCCAAGGATCTGATGGCGCAGGGGATCAAGC |
| | | | TCTGATCAAGAGACAGGATGAGGATCGTTTCGCATGATTGAACAAGATGGATTGCA |
| | | | CGCAGGTTCTCCGGCCGCTTGGGTGGAGAGGCTATTCGGCTATGACTGGGCACAAC |
| | | | AGACAATCGGCTGCTCTGATGCCGCCGTGTTCCGGCTGTCAGCGCAGGGGCGCCCG |
| | | | GTTCTTTTTGTCAAGACCGACCTGTCCGGTGCCCTGAATGAACTGCAAGACGAGGC |
| | | | AGCGCGGCTATCGTGGCTGGCCACGACGGGCGTTCCTTGCGCAGCTGTGCTCGACG |
| | | | TTGTCACTGAAGCGGGAAGGGACTGGCTGCTATTGGGCGAAGTGCCGGGGCAGGAT |
| | | | CTCCTGTCATCTCACCTTGCTCCTGCCGAGAAAGTATCCATCATGGCTGATGCAAT |
| | | | GCGGCGGCTGCATACGCTTGATCCGGCTACCTGCCCATTCGACCACCAAGCGAAAC |
| | | | ATCGCATCGAGCGAGCACGTACTCGGATGGAAGCCGGTCTTGTCGATCAGGATGAT |
| | | | CTGGACGAAGAGCATCAGGGGCTCGCGCCAGCCGAACTGTTCGCCAGGCTCAAGGC |
| | | | GAGCATGCCCGACGGCGAGGATCTCGTCGTGACCCATGGCGATGCCTGCTTGCCGA |
| | | | ATATCATGGTGGAAAATGGCCGCTTTTCTGGATTCATCGACTGTGGCCGGCTGGGT |
| | | | GTGGCGGACCGCTATCAGGACATAGCGTTGGCTACCCGTGATATTGCTGAAGAGCT |
| | | | TGGCGGCGAATGGGCTGACCGCTTCCTCGTGCTTTACGGTATCGCCGCTCCCGATT |
| | | | CGCAGCGCATCGCCTTCTATCGCCTTCTTGACGAGTTCTTCTGAATTATTAACGCT |
| | | | TACAATTTCCTGATGCGGTATTTTCTCCTTACGCATCTGTGCGGTATTTCACACCG |
| | | | CATACAGGTGGCACTTTTCGGGGAAATGTGCGCGGAACCCCTATTTGTTTATTTTT |
| | | | CTAAATACATTCAAATATGTATCCGCTCATGAGACAATAACCCTGATAAATGCTTC |
| | | | AATAATAGCACGTGCTAAAACTTCATTTTTAATTTAAAAGGATCTAGGTGAAGATC |
| | | | CTTTTTGATAATCTCATGACCAAAATCCCTTAACGTGAGTTTTCGTTCCACTGAGC |
| | | | GTCAGACCCCGTAGAAAAGATCAAAGGATCTTCTTGAGATCCTTTTTTTCTGCGCG |
| | | | TAATCTGCTGCTTGCAAACAAAAAAACCACCGCTACCAGCGGTGGTTTGTTTGCCG |
| | | | GATCAAGAGCTACCAACTCTTTTTCCGAAGGTAACTGGCTTCAGCAGAGCGCAGAT |
| | | | ACCAAATACTGTCCTTCTAGTGTAGCCGTAGTTAGGCCACCACTTCAAGAACTCTG |
| | | | TAGCACCGCCTACATACCTCGCTCTGCTAATCCTGTTACCAGTGGCTGCTGCCAGT |
| | | | GGCGATAAGTCGTGTCTTACCGGGTTGGACTCAAGACGATAGTTACCGGATAAGGC |
| | | | GCAGCGGTCGGGCTGAACGGGGGGTTCGTGCACACAGCCCAGCTTGGAGCGAACGA |
| | | | CCTACACCGAACTGAGATACCTACAGCGTGAGCTATGAGAAAGCGCCACGCTTCCC |
| | | | GAAGGGAGAAAGGCGGACAGGTATCCGGTAAGCGGCAGGGTCGGAACAGGAGAGCG |
| | | | CACGAGGGAGCTTCCAGGGGGAAACGCCTGGTATCTTTATAGTCCTGTCGGGTTTC |
| | | | GCCACCTCTGACTTGAGCGTCGATTTTTGTGATGCTCGTCAGGGGGGCGGAGCCTA |
| | | | TGGAAAAACGCCAGCAACGCGGCCTTTTTACGGTTCCTGGGCTTTTGCTGGCCTTT |
| | | | TGCTCACATGTTCTT |
| 74 | pb1125 | cDNA | ATGGCGGCGATGGCCGAGCGGCCCTTCCAGTGCAGGATCTGTATGCGCAACTTTTC |
| | | | TACCTCCGGCAACCTGACCCGGCACATCAGAACCCATACAGGCGAAAAGCCTTTCG |
| | | | CCTGCGACATTTGTGGGAGAAAATTTGCTCGGTCCGACGCCCTGTCCCAGCATACC |
| | | | AAGATCCACACCGGCTCTCAGAAACCATTCCAGTGCCGCATTTGTATGCGGAATTT |
| | | | TTCCCGGTCCTCCGCCCTGACCACCCATATCCGCACTCACACCGGAGAGAAGCCCT |
| | | | TTGCTTGCGACATTTGTGGCAGGAAATTTGCTCGGTCCTCCGACCTGACCACCCAC |
| | | | ACTAAGATCCATACTGGGTCACAGAAACCTTTCCAGTGCCGGATTTGTATGAGAAA |
| | | | CTTTAGCCGGTCCTCCAACCTGTCCCAGCACATCAGAACCCATACAGGCGAAAAGC |
| | | | CTTTCGCCTGCGACATTTGTGGGAGAAAATTTGCTCGGTCCTCCAACCTGTCCCAG |
| | | | CATACCAAGATCCACACCGGCTCTCAGAAACCATTCCAGTGCCGCATTTGTATGCG |
| | | | GAATTTTTCCCGGTCCTCCGCCCTGTCCCAGCATATCCGCACTCACACCGGAGAGA |
| | | | AGCCCTTTGCTTGCGACATTTGTGGCAGGAAATTTGCTCGGTCCTCCAACCTGACC |
| | | | CGGCACACTAAGATCCATACTGGGTCACAGAAACCTTTCCAGTGCCGGATTTGTAT |
| | | | GAGAAACTTTAGCCGGTCCGACCACCTGTCCGAGCACATCAGAACACATACTGGGC |
| | | | TGAGAGAGAAGATCATCGCCCACTTCCTGATCCAGCAGTACCTGAAGGAGGACTAC |
| | | | TCCTTCACCGCCTACGCCACCATCTCCTACCTGAAGATCGGCTGA |
| 75 | pb1125 | AA | MAAMAERPFQCRICMRNFSTSGNLTRHIRTHTGEKPFACDICGRKFARSDALSQHT |
| | | | KIHTGSQKPFQCRICMRNFSRSSALTTHIRTHTGEKPFACDICGRKFARSSDLTTH |
| | | | TKIHTGSQKPFQCRICMRNFSRSSNLSQHIRTHTGEKPFACDICGRKFARSSNLSQ |
| | | | HTKIHTGSQKPFQCRICMRNFSRSSALSQHIRTHTGEKPFACDICGRKFARSSNLT |
| | | | RHTKIHTGSQKPFQCRICMRNFSRSDHLSEHIRTHTGLREKIIAHFLIQQYLKEDY |
| | | | SFTAYATISYLKIG* |

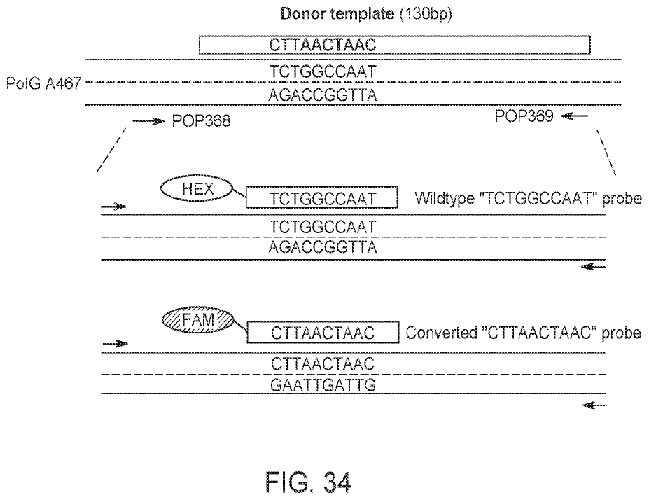

Detections of genetic “tcTggCcAAt-to-CTTAACTAAC” conversion after SSB-HbW-based gene edition were performed by droplet digital PCR (ddPCR). Relative positions of a correction ssODN (i.e., sequence modification polynucleotide) and position of an exemplary primer pair: POP368 and POP369 are also indicated in FIG. 34. One common primer, POP368 was located outside this ssODN template (i.e., sequence modification polynucleotide) sequence, while POP369, located inside. Allele-specific probes conjugated with fluorophores FAM and HEX were designed to distinguish between “CTTAACTAAC” and “tcTggCcAAt” respectively.

POP368 PolG a forward primer has a sequence of
(SEQ ID NO.: 76)
AACCAGAACTGGGAGCGTTA.
POP369 PolG reverse primer has a sequence of
(SEQ ID NO.: 77)
CTAGATCCTGCCCACCCAAG.

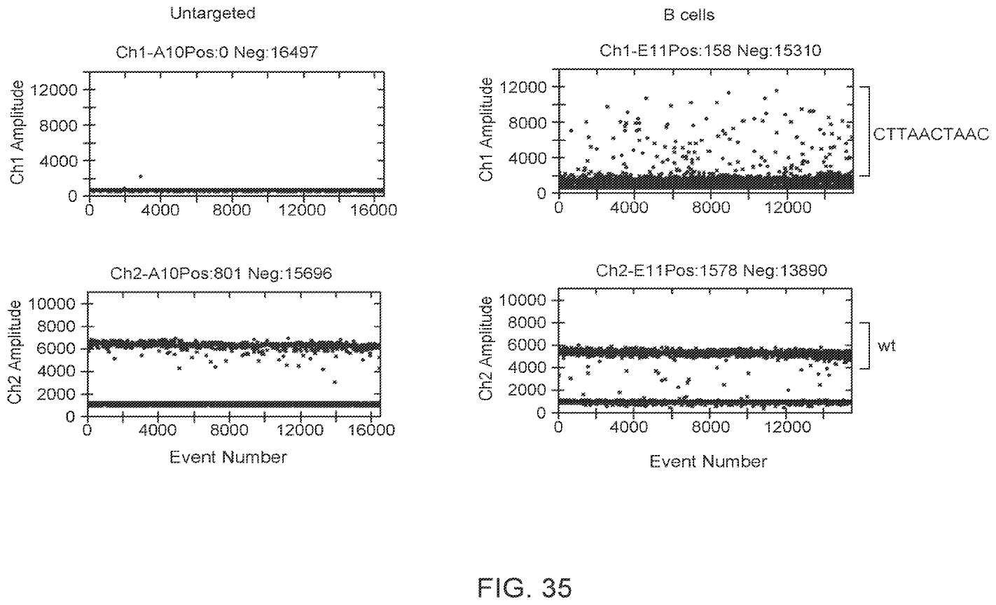

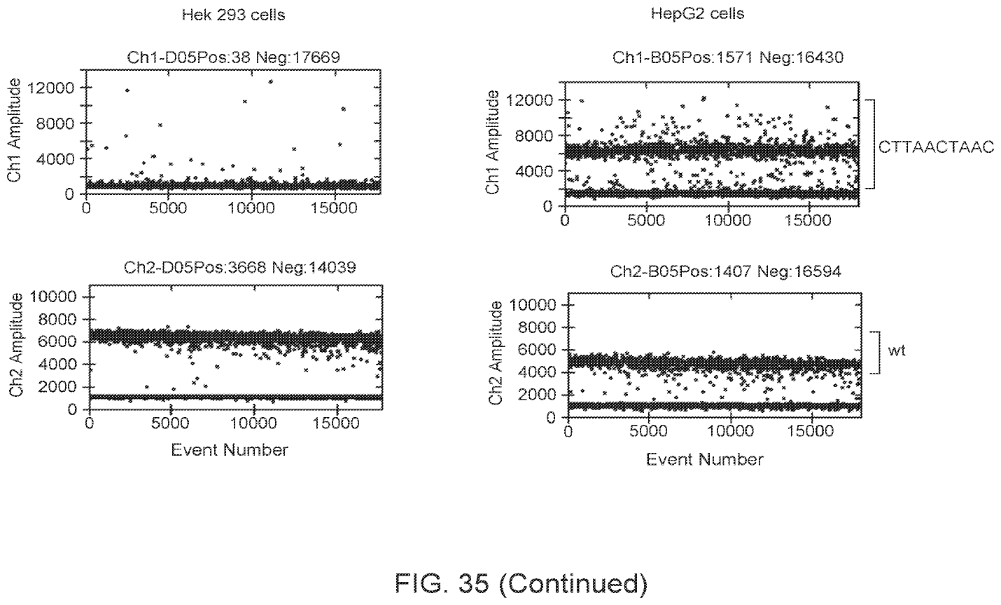

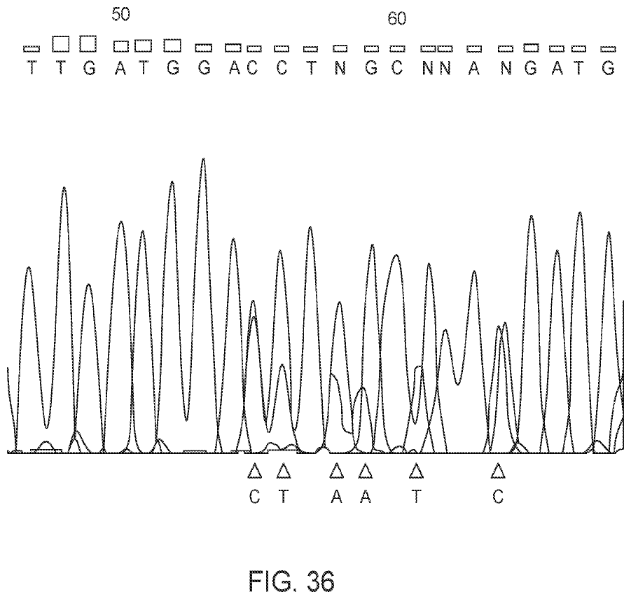

FIG. 35 demonstrates successful “tcTggCcAAt-to-CTTAACTAAC” genetic conversion at and close to codon 467 of human PolG as measured by ddPCR. In this example, after transfection of B cells, HEK293 and HepG2 cell lines with plasmid pb 125 and this 130-nucleotide correction template, cells were allowed to recover and grow on complete culture medium, containing 15% FBS, for five days. After five days genomic DNA was isolated and used in ddPCR analysis. Raw droplet data are shown as in FIG. 35 where these “CTTAACTAAC” droplets are displayed in the top panel in each set, while “tcTggCcAAt” droplets were in the lower one. Untargeted B-cells were used as a negative control, showing only “tcTggCcAAt” droplets, but no “CTTAACTAAC” droplets. Untargeted B cells only had “tcTggCcAAt” droplets, demonstrating untargeted wildtype genotype. After human B, Hek293, and HepG2 cells were transfected with pb 125 and ssODN template (i.e., sequence modification polynucleotide), “CTTAACTAAC” droplets appeared after being targeted and edited by this polynucleotide modification agent in combination with a correcting template, demonstrating successful “tcTggCcAAt-to-CTTAACTAAC” genetic conversion at codon A467 site of human PolG.

FIG. 36 shows exemplary Sanger sequencing results used to further confirm successful targeting and editing of codon A467 site of the human PolG gene. Genomic DNA was isolated and used as a template on which a 152-bp PCR amplicon surrounding PolG codon A467 was generated by using a primer set of POP368 and POP369. Amplified PCR products from targeted B cells were analyzed. It shows an exemplary chromatogram of a pb125 edited B-cell population, showing a “tcTggCcAAt-to-CTTAACTAAC” sequence conversion by Sanger sequencing. These results confirm that a polynucleotide modification agent in combination with a sequence modification polynucleotide can successfully target and edit a sequence in an endogenous mammalian gene in mammalian cells to successfully modify a disease-causing genotype.

This example further demonstrates use of SSB-HbW-based gene editing to modify disease-relevant nucleotide targets in mammalian cells by using a SSB-HbW-based gene editing approach and system to genetically modify sequences within various human genes. Thus, provided polynucleotide modification agents and systems can be used to successfully modify a variety of different disease relevant genetic targets.

### Example 6: Modification of an Endogenous Genomic Target: MMACHC by SSB-HbW-Based Gene Editing in Human B-Cells

The present example describes sequence specific genetic modification of MMACHC in exemplary cells using technologies described herein. In this example, human MMACHC at and close to codon R271 was targeted and edited by SSB-HbW-based gene editing with a polynucleotide modification agent and a single stranded oligonucleotide template (i.e., a sequence modification polynucleotide). Cobalamin C (cblC) deficiency, the most common inborn error of intracellular cobalamin metabolism, is caused by mutations in MMACHC, a gene responsible for the processing and intracellular trafficking of vitamin B12. It is a recessive disorder characterized by a failure to metabolize cobalamin resulting in the biochemical perturbations of methylmalonic acidemia, hyperhomocysteinemia and hypomethioninemia caused by the impaired activity of the downstream enzymes, methylmalonyl-CoA mutase and methionine synthase. Mutation of MMACHC gene causes the combination of acidemia and homocystinuria. It affects 1 in every 5000 live births. The most common mutation is a duplication of A at the codon 271 (c.271dupA), which causes a frameshift truncation, accounting for 42% of pathogenic alleles. Clinically, the presentation of mutations of MMACHC are wide spectrum, ranging from mild symptoms to very severe. Most severe cases feature psychomotor retardation and blindness. (Lerner-Ellis, J. P. et al. Human Mutation, 2009, Vol. 30:1072-1081, which is herein incorporated by reference in its entirety).

The present disclosure contemplates that, in some embodiments, a polynucleotide modification system as described herein can be used for conversion of nucleotides at or around position of codon 271 of human MMACHC gene. This example describes development of a specifically designed polynucleotide modification agent (e.g., a SSB-HbW agent) with in combination with a sequence modification polynucleotide (e.g., a specific single stranded oligonucleotide template) to convert a DNA sequence at or around codon 271 site to create amino acid substitution(s).

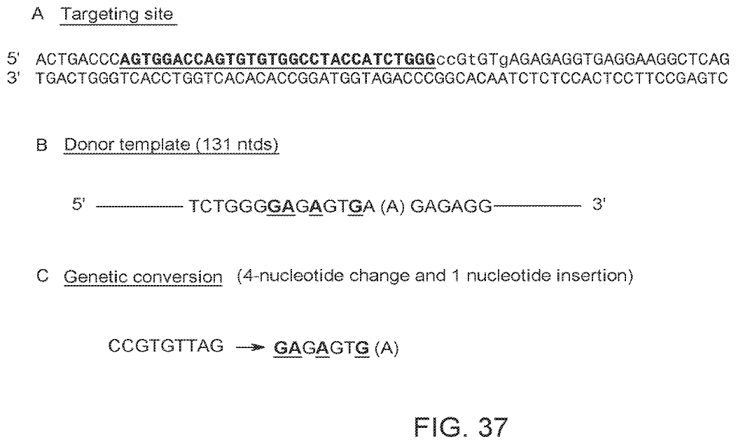

FIG. 37 depicts a schematic gene editing approach taken for gene editing of an endogenous genomic target around codon 271 of human MMACHC gene in B cells. In this example, an exemplary polynucleotide modification agent, encoded on plasmid pb 142, includes a DNA recognition domain which was an array of 10 zinc-fingers, specifically designed to recognize 5′-GTGGACCAGTGTGTGGCCTACCATCTGGGC-3′ (SEQ ID NO.: 79), a 30-nucleotide sequence on the leading strand of human MMACHC, displayed with underlined and bold letters. As depicted in FIG. 37, panel A, targeted nucleotides were displayed as a lowercase letters “ccGtGTgAG”, 3′ upstream of this binding site. In this embodiment, a donor template was used: a 131-nucleotide single stranded DNA oligonucleotide with a desired ccGtGTgAG→GAGAGTG(A) conversion roughly located in the middle of this oligonucleotide, as depicted in FIG. 37 panel B. The sequence modification polynucleotide used is provided as SEQ ID NO.: 80 (below) with an underlined and bold “GAGAGTG(A)” to indicate “ccGtGTgAG→GAGAGTG(A)” conversion. Nucleotide “A” in parentheses indicates insertion. FIG. 37 panel C depicts a 4-nucleotide change and 1 nucleotide insertion resulting from successful genetic conversion by SSB-HbW-based gene editing.

(SEQ ID NO.: 80)
5′-GCTGCCACCTCCGAATGCTGACTGACCCAGTGGACCAGTGTGTGGC
CTACCATCTGGGGAGAGTGA(A)GAGAGGTGAGGAAGGCTCAGTTTTCC
CCCAGCTCCCAAACCTACAGCTGCCTCCAGTTCCTCCA-3′

Here, a polynucleotide modification agent (encoded on plasmid pb 142 (full length DNA (SEQ ID NO.: 81); cDNA (SEQ ID. NO.: 82); amino acid sequence (SEQ ID. NO.: 83)), which has a DNA recognition domain comprised in an array of 10 zinc-fingers, was designed to specifically recognize 5′-GTGGACCAGTGTGTGGCCTACCATCTGGGC-3′ (SEQ ID NO.: 79), a 30-nucleotide sequence on the leading DNA strand (top row of nucleotides) of human MMACHC. Exemplary sequences are provided in Table 7 below.

TABLE 7
Exemplary polynucleotide modification agents targeting MMACHC

| SEQ | | AA/ | |
|---|---|---|---|
| ID | | DNA/ | |
| NO: | Feature | cDNA | Sequence |
| 81 | pb142 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAAGGGGAGACCCAAGCTGGCTAGCGTTTAAACTTAAGCTTATGGCGGCG |
| | | | ATGGCCGAGCGGCCCTTCGCCTGCGACATTTGTGGGAGAAAATTTGCTGATGACCG |
| | | | GTCCCACCTGACCCGGCATACCAAGATCCACACCGGCTCTCAGAAACCATTCCAGT |
| | | | GCAGGATCTGTATGCGCAACTTTTCTCGGTCCGACGCCCTGTCCGAGCACATCAGA |
| | | | ACCCATACAGGCGAAAAGCCTTTTGCTTGCGACATTTGTGGCAGGAAATTTGCTAC |
| | | | CTCCGGCAACCTGTCCGAGCACACTAAGATCCATACTGGGTCACAGAAACCTTTCC |
| | | | AGTGCCGCATTTGTATGCGGAATTTTTCCGACTCCTCCAACCTGACCACCCATATC |
| | | | CGCACTCACACCGGAGAGAAGCCCTTTGCATGCGACATTTGTGGACGGAAATTTGC |
| | | | TGACCGGTCCGACCTGACCCGGCATACCAAGATTCACACTGGGTCTCAGAAACCTT |
| | | | TCCAGTGCAGGATTTGTATGAGAAATTTTTCCCGGTCCGACGCCCTGACCCGGCAC |
| | | | ATCAGAACCCATACAGGCGAAAAGCCTTTTGCTTGCGACATTTGTGGCAGGAAATT |
| | | | TGCTACCTCCGGCCACCTGACCACCCACACTAAGATCCATACTGGGTCACAGAAAC |
| | | | CTTTCCAGTGCCGCATTTGTATGCGGAATTTTTCCCGGTCCTCCAACCTGTCCGAG |
| | | | CACATCAGAACCCATACAGGCGAAAAGCCTTTTGCTTGCGACATTTGTGGCAGGAA |
| | | | ATTTGCTGACCGGTCCAACCTGACCCGGCACACTAAGATCCATACTGGGTCACAGA |
| | | | AACCTTTCCAGTGCCGCATTTGTATGCGGAATTTTTCCCGGTCCGACGCCCTGACC |
| | | | CGGCACATCAGAACACATACTGGGCTGAGAGAGAAGATCATCGCCCACTTCCTGAT |
| | | | CCAGCAGTACCTGAAGGAGGACTACTCCTTCACCGCCTACGCCACCATCTCCTACC |
| | | | TGAAGATCGGCTGAGCGGCCGCTCGAGTCTAGAGGGCCCGTTTAAACCCGCTGATC |
| | | | AGCCTCGACTGTGCCTTCTAGTTGCCAGCCATCTGTTGTTTGCCCCTCCCCCGTGC |
| | | | CTTCCTTGACCCTGGAAGGTGCCACTCCCACTGTCCTTTCCTAATAAAATGAGGAA |
| | | | ATTGCATCGCATTGTCTGAGTAGGTGTCATTCTATTCTGGGGGGTGGGGTGGGGCA |
| | | | GGACAGCAAGGGGGAGGATTGGGAAGACAATAGCAGGCATGCTGGGGATGCGGTGG |
| | | | GCTCTATGGCTGCAGCTTCTACTGGGCGGTTTTATGGACAGCAAGCGAACCGGAAT |
| | | | TGCCAGCTGGGGCGCCCTCTGGTAAGGTTGGGAAGCCCTGCAAAGTAAACTGGATG |
| | | | GCTTTCTCGCCGCCAAGGATCTGATGGCGCAGGGGATCAAGCTCTGATCAAGAGAC |
| | | | AGGATGAGGATCGTTTCGCATGATTGAACAAGATGGATTGCACGCAGGTTCTCCGG |
| | | | CCGCTTGGGTGGAGAGGCTATTCGGCTATGACTGGGCACAACAGACAATCGGCTGC |
| | | | TCTGATGCCGCCGTGTTCCGGCTGTCAGCGCAGGGGCGCCCGGTTCTTTTTGTCAA |
| | | | GACCGACCTGTCCGGTGCCCTGAATGAACTGCAAGACGAGGCAGCGCGGCTATCGT |
| | | | GGCTGGCCACGACGGGCGTTCCTTGCGCAGCTGTGCTCGACGTTGTCACTGAAGCG |
| | | | GGAAGGGACTGGCTGCTATTGGGCGAAGTGCCGGGGCAGGATCTCCTGTCATCTCA |
| | | | CCTTGCTCCTGCCGAGAAAGTATCCATCATGGCTGATGCAATGCGGCGGCTGCATA |
| | | | CGCTTGATCCGGCTACCTGCCCATTCGACCACCAAGCGAAACATCGCATCGAGCGA |
| | | | GCACGTACTCGGATGGAAGCCGGTCTTGTCGATCAGGATGATCTGGACGAAGAGCA |
| | | | TCAGGGGCTCGCGCCAGCCGAACTGTTCGCCAGGCTCAAGGCGAGCATGCCCGACG |
| | | | GCGAGGATCTCGTCGTGACCCATGGCGATGCCTGCTTGCCGAATATCATGGTGGAA |
| | | | AATGGCCGCTTTTCTGGATTCATCGACTGTGGCCGGCTGGGTGTGGCGGACCGCTA |
| | | | TCAGGACATAGCGTTGGCTACCCGTGATATTGCTGAAGAGCTTGGCGGCGAATGGG |
| | | | CTGACCGCTTCCTCGTGCTTTACGGTATCGCCGCTCCCGATTCGCAGCGCATCGCC |
| | | | TTCTATCGCCTTCTTGACGAGTTCTTCTGAATTATTAACGCTTACAATTTCCTGAT |
| | | | GCGGTATTTTCTCCTTACGCATCTGTGCGGTATTTCACACCGCATACAGGTGGCAC |
| | | | TTTTCGGGGAAATGTGCGCGGAACCCCTATTTGTTTATTTTTCTAAATACATTCAA |
| | | | ATATGTATCCGCTCATGAGACAATAACCCTGATAAATGCTTCAATAATAGCACGTG |
| | | | CTAAAACTTCATTTTTAATTTAAAAGGATCTAGGTGAAGATCCTTTTTGATAATCT |
| | | | CATGACCAAAATCCCTTAACGTGAGTTTTCGTTCCACTGAGCGTCAGACCCCGTAG |
| | | | AAAAGATCAAAGGATCTTCTTGAGATCCTTTTTTTCTGCGCGTAATCTGCTGCTTG |
| | | | CAAACAAAAAAACCACCGCTACCAGCGGTGGTTTGTTTGCCGGATCAAGAGCTACC |
| | | | AACTCTTTTTCCGAAGGTAACTGGCTTCAGCAGAGCGCAGATACCAAATACTGTCC |
| | | | TTCTAGTGTAGCCGTAGTTAGGCCACCACTTCAAGAACTCTGTAGCACCGCCTACA |
| | | | TACCTCGCTCTGCTAATCCTGTTACCAGTGGCTGCTGCCAGTGGCGATAAGTCGTG |
| | | | TCTTACCGGGTTGGACTCAAGACGATAGTTACCGGATAAGGCGCAGCGGTCGGGCT |
| | | | GAACGGGGGGTTCGTGCACACAGCCCAGCTTGGAGCGAACGACCTACACCGAACTG |
| | | | AGATACCTACAGCGTGAGCTATGAGAAAGCGCCACGCTTCCCGAAGGGAGAAAGGC |
| | | | GGACAGGTATCCGGTAAGCGGCAGGGTCGGAACAGGAGAGCGCACGAGGGAGCTTC |
| | | | CAGGGGGAAACGCCTGGTATCTTTATAGTCCTGTCGGGTTTCGCCACCTCTGACTT |
| | | | GAGCGTCGATTTTTGTGATGCTCGTCAGGGGGGCGGAGCCTATGGAAAAACGCCAG |
| | | | CAACGCGGCCTTTTTACGGTTCCTGGGCTTTTGCTGGCCTTTTGCTCACATGTTCT |
| | | | T |
| 82 | pb142 | cDNA | ATGGCGGCGATGGCCGAGCGGCCCTTCGCCTGCGACATTTGTGGGAGAAAATTTGC |
| | | | TGATGACCGGTCCCACCTGACCCGGCATACCAAGATCCACACCGGCTCTCAGAAAC |
| | | | CATTCCAGTGCAGGATCTGTATGCGCAACTTTTCTCGGTCCGACGCCCTGTCCGAG |
| | | | CACATCAGAACCCATACAGGCGAAAAGCCTTTTGCTTGCGACATTTGTGGCAGGAA |
| | | | ATTTGCTACCTCCGGCAACCTGTCCGAGCACACTAAGATCCATACTGGGTCACAGA |
| | | | AACCTTTCCAGTGCCGCATTTGTATGCGGAATTTTTCCGACTCCTCCAACCTGACC |
| | | | ACCCATATCCGCACTCACACCGGAGAGAAGCCCTTTGCATGCGACATTTGTGGACG |
| | | | GAAATTTGCTGACCGGTCCGACCTGACCCGGCATACCAAGATTCACACTGGGTCTC |
| | | | AGAAACCTTTCCAGTGCAGGATTTGTATGAGAAATTTTTCCCGGTCCGACGCCCTG |
| | | | ACCCGGCACATCAGAACCCATACAGGCGAAAAGCCTTTTGCTTGCGACATTTGTGG |
| | | | CAGGAAATTTGCTACCTCCGGCCACCTGACCACCCACACTAAGATCCATACTGGGT |
| | | | CACAGAAACCTTTCCAGTGCCGCATTTGTATGCGGAATTTTTCCCGGTCCTCCAAC |
| | | | CTGTCCGAGCACATCAGAACCCATACAGGCGAAAAGCCTTTTGCTTGCGACATTTG |
| | | | TGGCAGGAAATTTGCTGACCGGTCCAACCTGACCCGGCACACTAAGATCCATACTG |
| | | | GGTCACAGAAACCTTTCCAGTGCCGCATTTGTATGCGGAATTTTTCCCGGTCCGAC |
| | | | GCCCTGACCCGGCACATCAGAACACATACTGGGCTGAGAGAGAAGATCATCGCCCA |
| | | | CTTCCTGATCCAGCAGTACCTGAAGGAGGACTACTCCTTCACCGCCTACGCCACCA |
| | | | TCTCCTACCTGAAGATCGGCTGA |
| 83 | pb142 | AA | MAAMAERPFACDICGRKFADDRSHLTRHTKIHTGSQKPFQCRICMRNFSRSDALSE |
| | | | HIRTHTGEKPFACDICGRKFATSGNLSEHTKIHTGSQKPFQCRICMRNFSDSSNLT |
| | | | THIRTHTGEKPFACDICGRKFADRSDLTRHTKIHTGSQKPFQCRICMRNFSRSDAL |
| | | | TRHIRTHTGEKPFACDICGRKFATSGHLTTHTKIHTGSQKPFQCRICMRNFSRSSN |
| | | | LSEHIRTHTGEKPFACDICGRKFADRSNLTRHTKIHTGSQKPFQCRICMRNFSRSD |
| | | | ALTRHIRTHTGLREKIIAHFLIQQYLKEDYSFTAYATISYLKIG* |

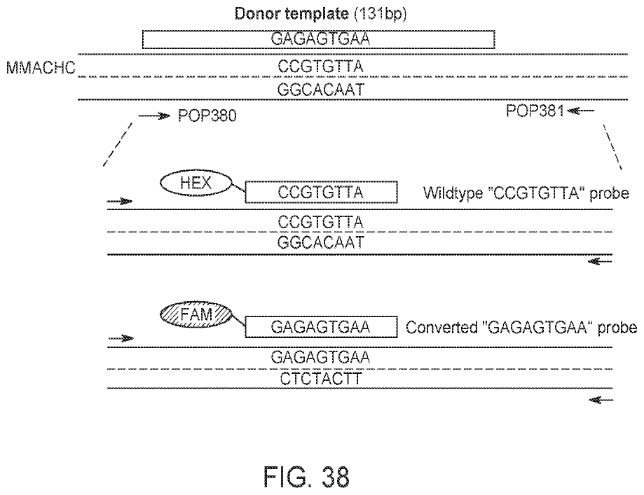

Detections of genetic “ccGtGTgAG→GAGAGTG(A)” conversion after SSB-HbW-based gene edition were performed by droplet digital PCR (ddPCR). Relative positions of a correction ssODN (i.e., sequence modification polynucleotide) and position of an exemplary primer pair: POP380 and POP381 are also indicated in FIG. 38. One common primer, POP380 was located inside this ssODN template (i.e., sequence modification polynucleotide) sequence, while POP381, located outside. Allele-specific probes conjugated with fluorophores FAM and HEX were designed to distinguish between “GAGAGTG(A)” and “ccGtGTgAG” respectively.

POP380 PolG a forward primer has a sequence of
(SEQ ID NO.: 84)
ACCTCCGAATGCTGACTGAC.
POP381 PolG reverse primer has a sequence of
(SEQ ID NO.: 85)
TCACCTTTGAAGTGGCTCCT.

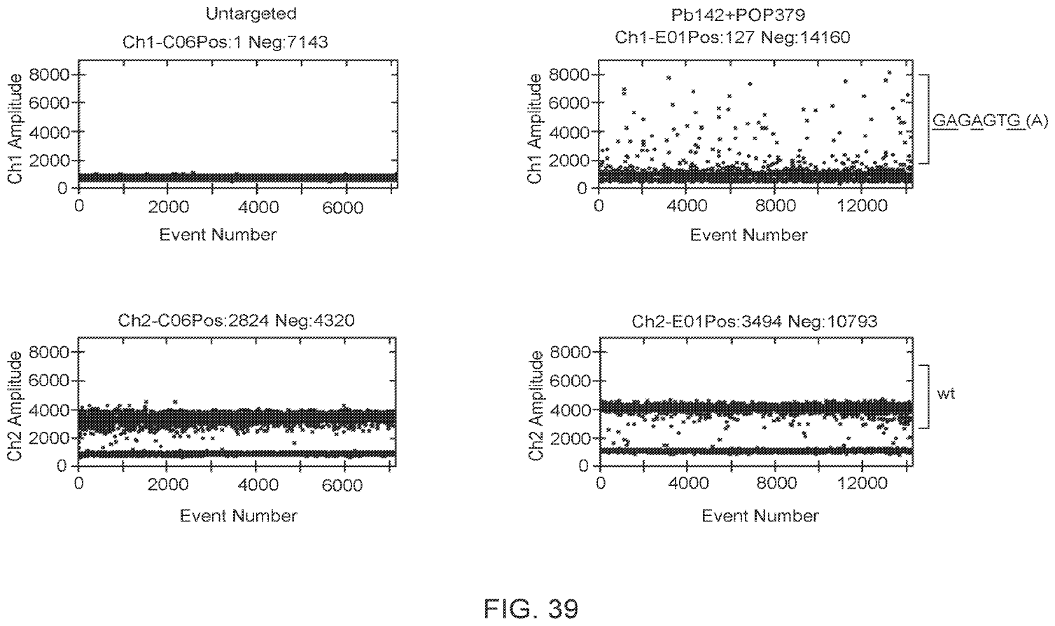

FIG. 39 demonstrates successful “ccGtGTgAG→GAGAGTG(A)” genetic conversion at and close to codon 271 of human MMACHC as measured by ddPCR. In this example, after transfection of B cells with plasmid pb 142 and this 131-nucleotide modification template, cells were allowed to recover and grow on complete culture medium, containing 15% FBS IN RPMI1640 medium, for five days. After five days genomic DNA was isolated and used in ddPCR analysis. Raw droplet data are shown as in FIG. 39 where these “GAGAGTG(A)” droplets are displayed in the top panel in each set, while “ccGtGTgAG” droplets were in the lower one. Untargeted B-cells were used as a negative control, showing only “ccGtGTgAG” droplets, but no “GAGAGTG(A)” droplets. Untargeted B cells only had “ccGtGTgAG” droplets, demonstrating untargeted wildtype genotype. After human B cells were transfected with pb 142 and ssODN template (i.e., sequence modification polynucleotide), “GAGAGTG(A)” droplets appeared after being targeted and edited by this polynucleotide modification agent in combination with a correcting template, demonstrating successful “ccGtGTgAG-to-GAGAGTG(A)” genetic conversion at codon 271 site of human MMACHC gene.

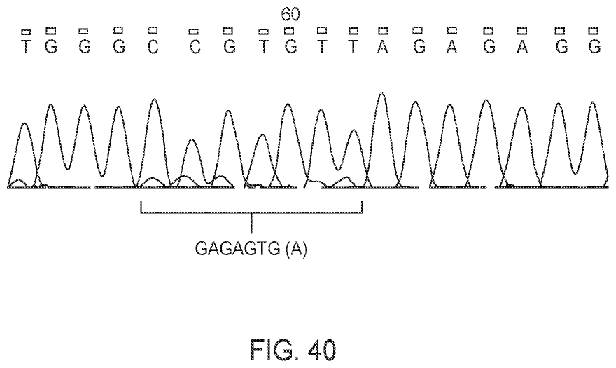

FIG. 40 shows exemplary Sanger sequencing results used to further confirm successful targeting and editing of codon 271 site of the human MMACHC gene. Genomic DNA was isolated and used as a template on which a 173-bp PCR amplicon surrounding codon 271 of human MMACHC was generated by using a primer set of POP380 and POP381. Amplified PCR products from targeted B cells were analyzed. It shows an exemplary chromatogram of a pb 142 edited B-cell population, showing a “ccGtGTgAG-to-GAGAGTG(A)” sequence conversion by Sanger sequencing. These results confirm that a polynucleotide modification agent in combination with a sequence modification polynucleotide can successfully target and edit a sequence in an endogenous mammalian gene in mammalian cells to successfully modify a disease-causing genotype.

This example further demonstrates use of SSB-HbW-based gene editing to modify disease-relevant nucleotide targets in mammalian cells by using a SSB-HbW-based gene editing approach and system to genetically modify sequences within various human genes. Thus, provided polynucleotide modification agents and systems can be used to successfully modify a variety of different disease relevant genetic targets.

### Example 7: Modification of an Endogenous Genomic Target: MMUT by SSB-HbW-Based Gene Editing in Human B-Cells

The present example describes sequence specific genetic modification of MMUT in exemplary cells using technologies described herein. In this example, human MMUT was targeted and edited by SSB-HbW-based gene editing with a polynucleotide modification agent and a single stranded oligonucleotide template (i.e., a sequence modification polynucleotide). Mut methylmalonic acidemia (MMA) is heterogenous genetic disorder most commonly caused by mutations in the MMUT gene encoding the enzyme methylmalonyl CoA mutase. MMUT, with its cofactor 5′deoxyadenosylcobalamin (a form of vitamin B12), plays a key role in catabolism of branched-chain amino acids, odd-chain fatty acids, propionate, and cholesterol. Loss of function of MMUT results in accumulation of metabolic intermediates that cause a range of phenotypes, with the most severe subtype associated with a neonatal presentation of encephalopathy, metabolic acidosis, and death, if not promptly treated. (Francis J. May. et al. Molecular Therapy Methods & Clinical Development, 2021, 21:765-776, which is herein incorporated by reference in its entirety).

The present disclosure contemplates that, in some embodiments, a polynucleotide modification system as described herein can be used for conversion of nucleotides in the exon 12 of human MMUT gene. This example describes development of a specifically designed polynucleotide modification agent (e.g., a SSB-HbW agent) with in combination with a sequence modification polynucleotide (e.g., a specific single stranded oligonucleotide template) to convert a DNA sequence in exon 12 of MMACHC to create amino acid substitution(s).

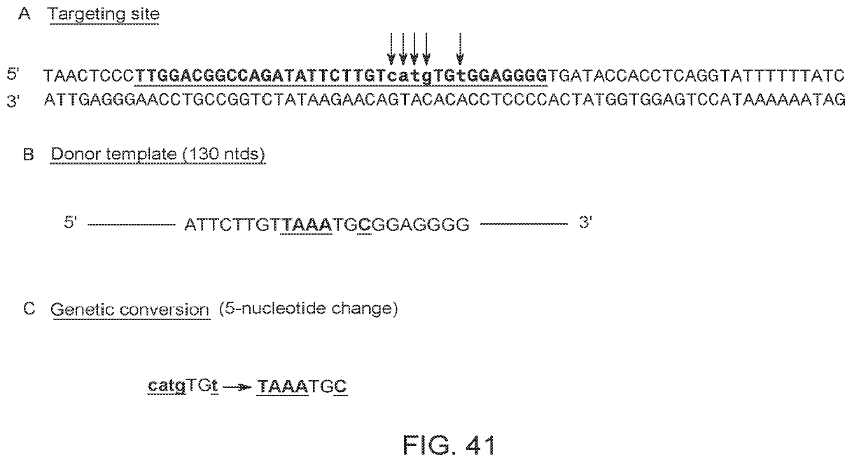

FIG. 41 depicts a schematic of the approach taken for gene editing of an endogenous genomic target human MMUT gene in B cells. In this example, an exemplary polynucleotide modification agent, encoded on plasmids pb 130, include a DNA recognition domain which was an array of 12 zinc-fingers, specifically designed to recognize 5′-TTGGACGGCCAGATATTCTTGTCATGTGTGGAGGGG-3′ (SEQ ID NO.: 87), a 36-nucleotide sequence on the leading strand of human MMUT, displayed with underlined and bold letters. As depicted in FIG. 41, panel A, targeted nucleotides were displayed as a lowercase letters “catgTGt”, overlapping this binding site. In this embodiment, a donor template was used: a 130-nucleotide single stranded DNA oligonucleotide with a desired catgTGt-to-TAAATGC conversion roughly located in the middle of this oligonucleotide, as depicted in FIG. 41, panel B. The sequence of this sequence modification polynucleotide used is provided as SEQ ID NO.: 88 (below) with an underlined and bold “TAAATGC” to indicate “catgTGt-to-TAAATGC” conversion. FIG. 41, panel C depicts 5-nucleotide substitutions resulted from successful genetic conversion by SSB-HbW-based gene editing.

(SEQ ID NO.: 88)
5′-AAAACCCTAGTTCCTGAACTCATCAAAGAACTTAACTCCCTTGGAC
GGCCAGATATTCTTGTTAAATGCGGAGGGGTGATACCACCTCAGGTATT
TTTTATCTCTATTTTTCTAGTACTGTGATGGGAAT-3′

Here, a polynucleotide modification agent (encoded on plasmid pb 130 (full length DNA (SEQ ID NO.: 89); cDNA (SEQ ID. NO.: 90); amino acid sequence (SEQ ID. NO.: 91)), which has a DNA recognition domain comprised in an array of 10 zinc-fingers, was designed to specifically recognize 5′-TTGGACGGCCAGATATTCTTGTCATGTGTGGAGGG-3′ (SEQ ID NO.: 87), a 36-nucleotide sequence on the leading DNA strand (top row of nucleotides) of human MMUT gene. Exemplary sequences are provided in Table 8 below.

TABLE 8
Exemplary polynucleotide modification agents targeting MMUT

| SEQ | | AA/ | |
|---|---|---|---|
| ID | | DNA/ | |
| NO: | Feature | cDNA | Sequence |
| 89 | pb130 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCGCCTGCGACATTTGTGGGAGAAAATTTGCTGATCGGTCCT |
| | | | CCCACCTGACCCGGCATACCAAGATCCACACCGGCTCTCAGAAACCATTCCAGTGC |
| | | | AGGATCTGTATGCGCAACTTTTCTCGGTCCGACAACCTGACCCGGCACATCAGAAC |
| | | | CCATACAGGCGAAAAGCCTTTTGCTTGCGACATTTGTGGCAGGAAATTTGCTCGGT |
| | | | CCGACGCCCTGACCCGGCACACTAAGATCCATACTGGGTCACAGAAACCTTTCCAG |
| | | | TGCCGCATTTGTATGCGGAATTTTTCCACCTCCGGCCACCTGACCACCCATATCCG |
| | | | CACTCACACCGGAGAGAAGCCCTTTGCATGCGACATTTGTGGACGGAAATTTGCTC |
| | | | AGTCCGGCGACCTGACCACCCATACCAAGATTCACACTGGGTCTCAGAAACCTTTC |
| | | | CAGTGCAGGATTTGTATGAGAAATTTTTCCCGGTCCTCCGCCCTGACCACCCACAT |
| | | | CAGAACCCATACAGGCGAAAAGCCTTTTGCTTGCGACATTTGTGGCAGGAAATTTG |
| | | | CTGACTCCTCCGCCCTGACCACCCACACTAAGATCCATACTGGGTCACAGAAACCT |
| | | | TTCCAGTGCCGCATTTGTATGCGGAATTTTTCCCAGTCCGGCTCCCTGTCCCAGCA |
| | | | CATCAGAACCCATACAGGCGAAAAGCCTTTTGCTTGCGACATTTGTGGCAGGAAAT |
| | | | TTGCTCGGTCCTCCAACCTGTCCGAGCACACTAAGATCCATACTGGGTCACAGAAA |
| | | | CCTTTCCAGTGCCGCATTTGTATGCGGAATTTTTCCGACTCCTCCCACCTGACCCG |
| | | | GCACATCAGAACCCATACAGGCGAAAAGCCTTTTGCTTGCGACATTTGTGGCAGGA |
| | | | AATTTGCTGACCGGTCCAACCTGACCCGGCACACTAAGATCCATACTGGGTCACAG |
| | | | AAACCTTTCCAGTGCCGCATTTGTATGCGGAATTTTTCCCGGTCCGACGCCCTGAC |
| | | | CACCCACATCAGAACACATACTGGGCTGAGAGAGAAGATCATCGCCCACTTCCTGA |
| | | | TCCAGCAGTACCTGAAGGAGGACTACTCCTTCACCGCCTACGCCACCATCTCCTAC |
| | | | CTGAAGATCGGCTGAGCGGCCGCTCGAGTCTAGAGGGCCCGTTTAAACCCGCTGAT |
| | | | CAGCCTCGACTGTGCCTTCTAGTTGCCAGCCATCTGTTGTTTGCCCCTCCCCCGTG |
| | | | CCTTCCTTGACCCTGGAAGGTGCCACTCCCACTGTCCTTTCCTAATAAAATGAGGA |
| | | | AATTGCATCGCATTGTCTGAGTAGGTGTCATTCTATTCTGGGGGGTGGGGTGGGGC |
| | | | AGGACAGCAAGGGGGAGGATTGGGAAGACAATAGCAGGCATGCTGGGGATGCGGTG |
| | | | GGCTCTATGGCTTCTACTGGGCGGTTTTATGGACAGCAAGCGAACCGGAATTGCCA |
| | | | GCTGGGGCGCCCTCTGGTAAGGTTGGGAAGCCCTGCAAAGTAAACTGGATGGCTTT |
| | | | CTCGCCGCCAAGGATCTGATGGCGCAGGGGATCAAGCTCTGATCAAGAGACAGGAT |
| | | | GAGGATCGTTTCGCATGATTGAACAAGATGGATTGCACGCAGGTTCTCCGGCCGCT |
| | | | TGGGTGGAGAGGCTATTCGGCTATGACTGGGCACAACAGACAATCGGCTGCTCTGA |
| | | | TGCCGCCGTGTTCCGGCTGTCAGCGCAGGGGCGCCCGGTTCTTTTTGTCAAGACCG |
| | | | ACCTGTCCGGTGCCCTGAATGAACTGCAAGACGAGGCAGCGCGGCTATCGTGGCTG |
| | | | GCCACGACGGGCGTTCCTTGCGCAGCTGTGCTCGACGTTGTCACTGAAGCGGGAAG |
| | | | GGACTGGCTGCTATTGGGCGAAGTGCCGGGGCAGGATCTCCTGTCATCTCACCTTG |
| | | | CTCCTGCCGAGAAAGTATCCATCATGGCTGATGCAATGCGGCGGCTGCATACGCTT |
| | | | GATCCGGCTACCTGCCCATTCGACCACCAAGCGAAACATCGCATCGAGCGAGCACG |
| | | | TACTCGGATGGAAGCCGGTCTTGTCGATCAGGATGATCTGGACGAAGAGCATCAGG |
| | | | GGCTCGCGCCAGCCGAACTGTTCGCCAGGCTCAAGGCGAGCATGCCCGACGGCGAG |
| | | | GATCTCGTCGTGACCCATGGCGATGCCTGCTTGCCGAATATCATGGTGGAAAATGG |
| | | | CCGCTTTTCTGGATTCATCGACTGTGGCCGGCTGGGTGTGGCGGACCGCTATCAGG |
| | | | ACATAGCGTTGGCTACCCGTGATATTGCTGAAGAGCTTGGCGGCGAATGGGCTGAC |
| | | | CGCTTCCTCGTGCTTTACGGTATCGCCGCTCCCGATTCGCAGCGCATCGCCTTCTA |
| | | | TCGCCTTCTTGACGAGTTCTTCTGAATTATTAACGCTTACAATTTCCTGATGCGGT |
| | | | ATTTTCTCCTTACGCATCTGTGCGGTATTTCACACCGCATACAGGTGGCACTTTTC |
| | | | GGGGAAATGTGCGCGGAACCCCTATTTGTTTATTTTTCTAAATACATTCAAATATG |
| | | | TATCCGCTCATGAGACAATAACCCTGATAAATGCTTCAATAATAGCACGTGCTAAA |
| | | | ACTTCATTTTTAATTTAAAAGGATCTAGGTGAAGATCCTTTTTGATAATCTCATGA |
| | | | CCAAAATCCCTTAACGTGAGTTTTCGTTCCACTGAGCGTCAGACCCCGTAGAAAAG |
| | | | ATCAAAGGATCTTCTTGAGATCCTTTTTTTCTGCGCGTAATCTGCTGCTTGCAAAC |
| | | | AAAAAAACCACCGCTACCAGCGGTGGTTTGTTTGCCGGATCAAGAGCTACCAACTC |
| | | | TTTTTCCGAAGGTAACTGGCTTCAGCAGAGCGCAGATACCAAATACTGTCCTTCTA |
| | | | GTGTAGCCGTAGTTAGGCCACCACTTCAAGAACTCTGTAGCACCGCCTACATACCT |
| | | | CGCTCTGCTAATCCTGTTACCAGTGGCTGCTGCCAGTGGCGATAAGTCGTGTCTTA |
| | | | CCGGGTTGGACTCAAGACGATAGTTACCGGATAAGGCGCAGCGGTCGGGCTGAACG |
| | | | GGGGGTTCGTGCACACAGCCCAGCTTGGAGCGAACGACCTACACCGAACTGAGATA |
| | | | CCTACAGCGTGAGCTATGAGAAAGCGCCACGCTTCCCGAAGGGAGAAAGGCGGACA |
| | | | GGTATCCGGTAAGCGGCAGGGTCGGAACAGGAGAGCGCACGAGGGAGCTTCCAGGG |
| | | | GGAAACGCCTGGTATCTTTATAGTCCTGTCGGGTTTCGCCACCTCTGACTTGAGCG |
| | | | TCGATTTTTGTGATGCTCGTCAGGGGGGCGGAGCCTATGGAAAAACGCCAGCAACG |
| | | | CGGCCTTTTTACGGTTCCTGGGCTTTTGCTGGCCTTTTGCTCACATGTTCTT |
| 90 | pb130 | cDNA | ATGGCGGCGATGGCCGAGCGGCCCTTCGCCTGCGACATTTGTGGGAGAAAATTTGC |
| | | | TGATCGGTCCTCCCACCTGACCCGGCATACCAAGATCCACACCGGCTCTCAGAAAC |
| | | | CATTCCAGTGCAGGATCTGTATGCGCAACTTTTCTCGGTCCGACAACCTGACCCGG |
| | | | CACATCAGAACCCATACAGGCGAAAAGCCTTTTGCTTGCGACATTTGTGGCAGGAA |
| | | | ATTTGCTCGGTCCGACGCCCTGACCCGGCACACTAAGATCCATACTGGGTCACAGA |
| | | | AACCTTTCCAGTGCCGCATTTGTATGCGGAATTTTTCCACCTCCGGCCACCTGACC |
| | | | ACCCATATCCGCACTCACACCGGAGAGAAGCCCTTTGCATGCGACATTTGTGGACG |
| | | | GAAATTTGCTCAGTCCGGCGACCTGACCACCCATACCAAGATTCACACTGGGTCTC |
| | | | AGAAACCTTTCCAGTGCAGGATTTGTATGAGAAATTTTTCCCGGTCCTCCGCCCTG |
| | | | ACCACCCACATCAGAACCCATACAGGCGAAAAGCCTTTTGCTTGCGACATTTGTGG |
| | | | CAGGAAATTTGCTGACTCCTCCGCCCTGACCACCCACACTAAGATCCATACTGGGT |
| | | | CACAGAAACCTTTCCAGTGCCGCATTTGTATGCGGAATTTTTCCCAGTCCGGCTCC |
| | | | CTGTCCCAGCACATCAGAACCCATACAGGCGAAAAGCCTTTTGCTTGCGACATTTG |
| | | | TGGCAGGAAATTTGCTCGGTCCTCCAACCTGTCCGAGCACACTAAGATCCATACTG |
| | | | GGTCACAGAAACCTTTCCAGTGCCGCATTTGTATGCGGAATTTTTCCGACTCCTCC |
| | | | CACCTGACCCGGCACATCAGAACCCATACAGGCGAAAAGCCTTTTGCTTGCGACAT |
| | | | TTGTGGCAGGAAATTTGCTGACCGGTCCAACCTGACCCGGCACACTAAGATCCATA |
| | | | CTGGGTCACAGAAACCTTTCCAGTGCCGCATTTGTATGCGGAATTTTTCCCGGTCC |
| | | | GACGCCCTGACCACCCACATCAGAACACATACTGGGCTGAGAGAGAAGATCATCGC |
| | | | CCACTTCCTGATCCAGCAGTACCTGAAGGAGGACTACTCCTTCACCGCCTACGCCA |
| | | | CCATCTCCTACCTGAAGATCGGCTGA |
| 91 | pb130 | AA | MAAMAERPFACDICGRKFADRSSHLTRHTKIHTGSQKPFQCRICMRNFSRSDNLTR |
| | | | HIRTHTGEKPFACDICGRKFARSDALTRHTKIHTGSQKPFQCRICMRNFSTSGHLT |
| | | | THIRTHTGEKPFACDICGRKFAQSGDLTTHTKIHTGSQKPFQCRICMRNFSRSSAL |
| | | | TTHIRTHTGEKPFACDICGRKFADSSALTTHTKIHTGSQKPFQCRICMRNFSQSGS |
| | | | LSQHIRTHTGEKPFACDICGRKFARSSNLSEHTKIHTGSQKPFQCRICMRNFSDSS |
| | | | HLTRHIRTHTGEKPFACDICGRKFADRSNLTRHTKIHTGSQKPFQCRICMRNFSRS |
| | | | DALTTHIRTHTGLREKIIAHFLIQQYLKEDYSFTAYATISYLKIG* |

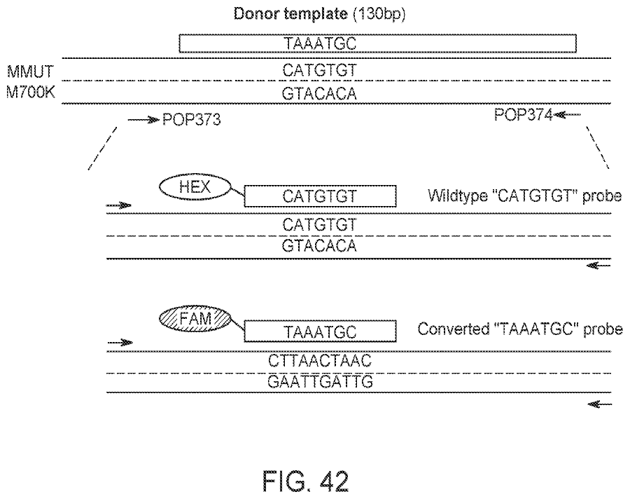

Detections of genetic “catgTGt-to-TAAATGC” conversion after SSB-HbW-based gene edition were performed by droplet digital PCR (ddPCR). Relative positions of a correction ssODN (i.e., sequence modification polynucleotide) and position of an exemplary primer pair: POP373 and POP374 are also indicated in FIG. 42. One common primer, POP373 was located outside this ssODN template (i.e., sequence modification polynucleotide) sequence, while POP374, located inside. Allele-specific probes conjugated with fluorophores FAM and HEX were designed to distinguish between “catgTGt-to-TAAATGC” respectively.

```
POP373 MMUTa forward primer has a sequence of
  (SEQ ID NO.: 92)
GTGCATGCTGTGGGCATA.
POP374 MMUT reverse primer has a sequence of
  (SEQ ID NO.: 93)
TTCCCATCACAGTACTAGAAAAATAGA.
```

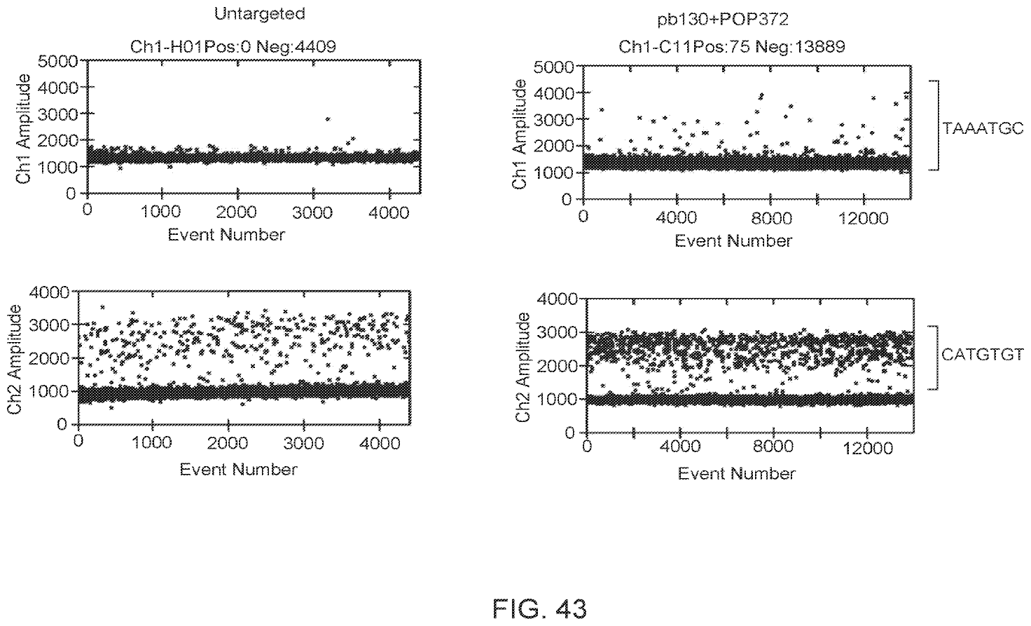

FIG. 43 demonstrates successful “catgTGt-to-TAAATGC” genetic conversion at exon 12 of human MMUT as measured by ddPCR. In this example, after transfection of B cells with plasmid pb 130 and this 130-nucleotide correction template, cells were allowed to recover and grow on complete culture medium, containing 15% FBS IN RPMI1640 medium, for five days. After five days genomic DNA was isolated and used in ddPCR analysis. Raw droplet data are shown as in FIG. 43 where these “TAAATGC” droplets are displayed in the top panel in each set, while “catgTGt” droplets were in the lower one. Untargeted B-cells were used as a negative control, showing only “catgTGt” droplets, but no “TAAATGC” droplets. Untargeted B cells only had “catgTGt” droplets, demonstrating untargeted wildtype genotype. After human B cells were transfected with pb 130 and ssODN template (i.e., sequence modification polynucleotide), “TAAATGC” droplets appeared after being targeted and edited by this polynucleotide modification agent in combination with a correcting template, demonstrating successful “catgTGt-to-TAAATGC” genetic conversion at exon 12 site of human MMUT gene. These results confirm that a polynucleotide modification agent in combination with a sequence modification polynucleotide can successfully target and edit a sequence in an endogenous mammalian gene in mammalian cells to successfully modify a disease-causing genotype.

This example further demonstrates use of SSB-HbW-based gene editing to modify disease-relevant nucleotide targets in mammalian cells by using a SSB-HbW-based gene editing approach and system to genetically modify sequences within various human genes. Thus, provided polynucleotide modification agents and systems can be used to successfully modify a variety of different disease relevant genetic targets.

### Example 8: Modification of an Endogenous Genomic Target: PAH by SSB-HbW-Based Gene Editing in Human B-Cells

The present example describes sequence specific genetic modification of PAH exemplary cells using technologies described herein. In this example, human PAH was targeted and edited by SSB-HbW-based gene editing with a polynucleotide modification agent and a single stranded oligonucleotide template (i.e., a sequence modification polynucleotide). The human PAH gene encodes the enzyme phenylalanine hydroxylase. Mutations of PAH gene are associated with Phenylalanine hydroxylase deficiency, usually leading to a loss in enzyme activity and an increase in serum concentrations of phenylalanine, clinically diagnosed as phenylketonuria (PKU). Abnormal accumulation of phenylalanine in serum can damage the peripheral and central nervous systems, resulting in mental retardation, seizures, and cerebral palsy to varying degrees, if left untreated. PKU is an autosomal recessive metabolic disorder caused by mutations in PAH gene and it is the most common inborn genetic defect of the amino acid metabolism. (Alicia Hillert. et al. Am J Hum Genet. 2020, 107(2):234-250, which is herein incorporated by reference in its entirety). In this example, human PAH gene was targeted and edited by SSB-HbW-based gene editing with a polynucleotide modification agent and a single stranded oligonucleotide template (i.e., a sequence modification polynucleotide).

The present disclosure contemplates that, in some embodiments, a polynucleotide modification system as described herein can be used for conversion of nucleotides in exon 12 of human PAH gene. This example describes development of a specifically designed polynucleotide modification agent (e.g., a SSB-HbW agent) with in combination with a sequence modification polynucleotide (e.g., a specific single stranded oligonucleotide template) to convert a DNA sequence in exon 12 to create amino acid substitution(s).

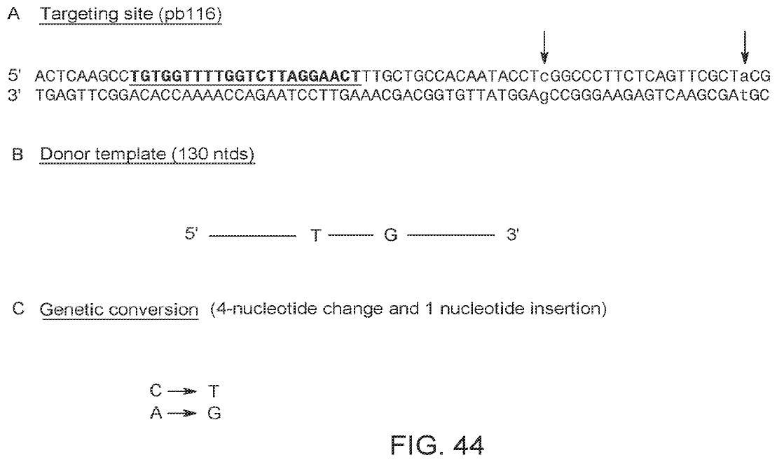

FIG. 44 depicts a schematic of the approach taken for gene editing of an endogenous genomic target human PAH gene in B cells. In this example, an exemplary polynucleotide modification agent, encoded on plasmids pb 116, include a DNA recognition domain which was an array of 7 zinc-fingers, specifically designed to recognize 5′-GTGGTTTTGGTTTAGGAACT-3′ (SEQ ID NO.: 95), a 21-nucleotide sequence on the leading strand of human PAH, displayed with underlined and bold letters. As depicted in FIG. 44, panel A, two targeted nucleotides are displayed as lowercase letters, and are also indicated by two arrows. In this example, there is an 18-nucleotide distance between these two nucleotides. These two specific targeted nucleotides are at positions that can be involved in mutation R408W or Y414C, which are associated with PKU. In this embodiment, a donor template was used: a 130-nucleotide single stranded DNA oligonucleotide with desired C-to-T and A-to-G conversions roughly located in the middle of this oligonucleotide, as depicted in FIG. 44, panel B. This sequence of the sequence modification polynucleotide used is provided as SEQ ID NO.: 96 (below) with an underlined and bold “T” and “G” to indicate both C-to-T and A-to-G conversions. FIG. 44, panel C depicts both single nucleotide substitutions resulting from successful genetic conversion SSB-HbW-based gene editing.

(SEQ ID NO.: 96)
5′-CCCTTCACTCAAGCCTGTGGTTTTGGTCTTAGGAACTTTGC
TGCCACAATACCTTGGCCCTTCTCAGTTCGCTGCGACCCATACA
CCCAAAGGATTGAGGTCTTGGACAATACCCAGCAGCTTAAGATT
T-3′.

Here, a polynucleotide modification agent (encoded on plasmid pb 116 (full length DNA (SEQ ID NO.: 97); cDNA (SEQ ID. NO.: 98); amino acid sequence (SEQ ID. NO.: 99)), which has a DNA recognition domain comprised in an array of 7 zinc-fingers, was designed to specifically recognize 5′-GTGGTTTTGGTTTAGGAACT-3′ (SEQ ID NO.: 95), a 21-nucleotide sequence on the leading DNA strand (top row of nucleotides) of human PAH gene. Exemplary sequences are provided in Table 9 below.

TABLE 9
Exemplary polynucleotide modification agents targeting PAH

| SEQ | | AA/ | |
|---|---|---|---|
| ID | | DNA/ | |
| NO: | Feature | CDNA | Sequence |
| 97 | pb116 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCCAGTGCAGGATCTGTATGCGCAACTTTTCTACCTCCGGCG |
| | | | ACCTGTCCCAGCACATCAGAACCCATACAGGCGAAAAGCCTTTCGCCTGCGACATT |
| | | | TGTGGGAGAAAATTTGCTCAGTCCGGCCACCTGACCCGGCATACCAAGATCCACAC |
| | | | CGGCTCTCAGAAACCATTCCAGTGCCGCATTTGTATGCGGAATTTTTCCCAGTCCG |
| | | | GCTCCCTGACCACCCATATCCGCACTCACACCGGAGAGAAGCCCTTTGCTTGCGAC |
| | | | ATTTGTGGCAGGAAATTTGCTGACCGCTCCGCCCTGACCCGGCACACTAAGATCCA |
| | | | TACTGGGTCACAGAAACCTTTCCAGTGCCGGATTTGTATGAGAAACTTTAGCCGGT |
| | | | CCGACTCCCTGACCACCCACATCAGAACCCATACAGGCGAAAAGCCTTTCGCCTGC |
| | | | GACATTTGTGGGAGAAAATTTGCTACCTCCGGCGCCCTGACCCGGCATACCAAGAT |
| | | | CCACACCGGCTCTCAGAAACCATTCCAGTGCCGCATTTGTATGCGGAATTTTTCCC |
| | | | GGTCCGACTCCCTGACCCGGCACATCAGAACACATACCGGTGAGAAGATCATCGCC |
| | | | CACTTCCTGATCCAGCAGTACCTGAAGGAGGACTACTCCTTCACCGCCTACGCCAC |
| | | | CATCTCCTACCTGAAGATCGGCTGAGCGGCCGCTCGAGTCTAGAGGGCCCGTTTAA |
| | | | ACCCGCTGATCAGCCTCGACTGTGCCTTCTAGTTGCCAGCCATCTGTTGTTTGCCC |
| | | | CTCCCCCGTGCCTTCCTTGACCCTGGAAGGTGCCACTCCCACTGTCCTTTCCTAAT |
| | | | AAAATGAGGAAATTGCATCGCATTGTCTGAGTAGGTGTCATTCTATTCTGGGGGGT |
| | | | GGGGTGGGGCAGGACAGCAAGGGGGAGGATTGGGAAGACAATAGCAGGCATGCTGG |
| | | | GGATGCGGTGGGCTCTATGGCTTCTACTGGGCGGTTTTATGGACAGCAAGCGAACC |
| | | | GGAATTGCCAGCTGGGGCGCCCTCTGGTAAGGTTGGGAAGCCCTGCAAAGTAAACT |
| | | | GGATGGCTTTCTCGCCGCCAAGGATCTGATGGCGCAGGGGATCAAGCTCTGATCAA |
| | | | GAGACAGGATGAGGATCGTTTCGCATGATTGAACAAGATGGATTGCACGCAGGTTC |
| | | | TCCGGCCGCTTGGGTGGAGAGGCTATTCGGCTATGACTGGGCACAACAGACAATCG |
| | | | GCTGCTCTGATGCCGCCGTGTTCCGGCTGTCAGCGCAGGGGCGCCCGGTTCTTTTT |
| | | | GTCAAGACCGACCTGTCCGGTGCCCTGAATGAACTGCAAGACGAGGCAGCGCGGCT |
| | | | ATCGTGGCTGGCCACGACGGGCGTTCCTTGCGCAGCTGTGCTCGACGTTGTCACTG |
| | | | AAGCGGGAAGGGACTGGCTGCTATTGGGCGAAGTGCCGGGGCAGGATCTCCTGTCA |
| | | | TCTCACCTTGCTCCTGCCGAGAAAGTATCCATCATGGCTGATGCAATGCGGCGGCT |
| | | | GCATACGCTTGATCCGGCTACCTGCCCATTCGACCACCAAGCGAAACATCGCATCG |
| | | | AGCGAGCACGTACTCGGATGGAAGCCGGTCTTGTCGATCAGGATGATCTGGACGAA |
| | | | GAGCATCAGGGGCTCGCGCCAGCCGAACTGTTCGCCAGGCTCAAGGCGAGCATGCC |
| | | | CGACGGCGAGGATCTCGTCGTGACCCATGGCGATGCCTGCTTGCCGAATATCATGG |
| | | | TGGAAAATGGCCGCTTTTCTGGATTCATCGACTGTGGCCGGCTGGGTGTGGCGGAC |
| | | | CGCTATCAGGACATAGCGTTGGCTACCCGTGATATTGCTGAAGAGCTTGGCGGCGA |
| | | | ATGGGCTGACCGCTTCCTCGTGCTTTACGGTATCGCCGCTCCCGATTCGCAGCGCA |
| | | | TCGCCTTCTATCGCCTTCTTGACGAGTTCTTCTGAATTATTAACGCTTACAATTTC |
| | | | CTGATGCGGTATTTTCTCCTTACGCATCTGTGCGGTATTTCACACCGCATACAGGT |
| | | | GGCACTTTTCGGGGAAATGTGCGCGGAACCCCTATTTGTTTATTTTTCTAAATACA |
| | | | TTCAAATATGTATCCGCTCATGAGACAATAACCCTGATAAATGCTTCAATAATAGC |
| | | | ACGTGCTAAAACTTCATTTTTAATTTAAAAGGATCTAGGTGAAGATCCTTTTTGAT |
| | | | AATCTCATGACCAAAATCCCTTAACGTGAGTTTTCGTTCCACTGAGCGTCAGACCC |
| | | | CGTAGAAAAGATCAAAGGATCTTCTTGAGATCCTTTTTTTCTGCGCGTAATCTGCT |
| | | | GCTTGCAAACAAAAAAACCACCGCTACCAGCGGTGGTTTGTTTGCCGGATCAAGAG |
| | | | CTACCAACTCTTTTTCCGAAGGTAACTGGCTTCAGCAGAGCGCAGATACCAAATAC |
| | | | TGTCCTTCTAGTGTAGCCGTAGTTAGGCCACCACTTCAAGAACTCTGTAGCACCGC |
| | | | CTACATACCTCGCTCTGCTAATCCTGTTACCAGTGGCTGCTGCCAGTGGCGATAAG |
| | | | TCGTGTCTTACCGGGTTGGACTCAAGACGATAGTTACCGGATAAGGCGCAGCGGTC |
| | | | GGGCTGAACGGGGGGTTCGTGCACACAGCCCAGCTTGGAGCGAACGACCTACACCG |
| | | | AACTGAGATACCTACAGCGTGAGCTATGAGAAAGCGCCACGCTTCCCGAAGGGAGA |
| | | | AAGGCGGACAGGTATCCGGTAAGCGGCAGGGTCGGAACAGGAGAGCGCACGAGGGA |
| | | | GCTTCCAGGGGGAAACGCCTGGTATCTTTATAGTCCTGTCGGGTTTCGCCACCTCT |
| | | | GACTTGAGCGTCGATTTTTGTGATGCTCGTCAGGGGGGCGGAGCCTATGGAAAAAC |
| | | | GCCAGCAACGCGGCCTTTTTACGGTTCCTGGGCTTTTGCTGGCCTTTTGCTCACAT |
| | | | GTTCTT |
| 98 | pb116 | CDNA | ATGGCGGCGATGGCCGAGCGGCCCTTCCAGTGCAGGATCTGTATGCGCAACTTTTC |
| | | | TACCTCCGGCGACCTGTCCCAGCACATCAGAACCCATACAGGCGAAAAGCCTTTCG |
| | | | CCTGCGACATTTGTGGGAGAAAATTTGCTCAGTCCGGCCACCTGACCCGGCATACC |
| | | | AAGATCCACACCGGCTCTCAGAAACCATTCCAGTGCCGCATTTGTATGCGGAATTT |
| | | | TTCCCAGTCCGGCTCCCTGACCACCCATATCCGCACTCACACCGGAGAGAAGCCCT |
| | | | TTGCTTGCGACATTTGTGGCAGGAAATTTGCTGACCGCTCCGCCCTGACCCGGCAC |
| | | | ACTAAGATCCATACTGGGTCACAGAAACCTTTCCAGTGCCGGATTTGTATGAGAAA |
| | | | CTTTAGCCGGTCCGACTCCCTGACCACCCACATCAGAACCCATACAGGCGAAAAGC |
| | | | CTTTCGCCTGCGACATTTGTGGGAGAAAATTTGCTACCTCCGGCGCCCTGACCCGG |
| | | | CATACCAAGATCCACACCGGCTCTCAGAAACCATTCCAGTGCCGCATTTGTATGCG |
| | | | GAATTTTTCCCGGTCCGACTCCCTGACCCGGCACATCAGAACACATACCGGTGAGA |
| | | | AGATCATCGCCCACTTCCTGATCCAGCAGTACCTGAAGGAGGACTACTCCTTCACC |
| | | | GCCTACGCCACCATCTCCTACCTGAAGATCGGCTGA |
| 99 | pb116 | AA | MAAMAERPFQCRICMRNFSTSGDLSQHIRTHTGEKPFACDICGRKFAQSGHLTRHT |
| | | | KIHTGSQKPFQCRICMRNFSQSGSLTTHIRTHTGEKPFACDICGRKFADRSALTRH |
| | | | TKIHTGSQKPFQCRICMRNFSRSDSLTTHIRTHTGEKPFACDICGRKFATSGALTR |
| | | | HTKIHTGSQKPFQCRICMRNFSRSDSLTRHIRTHTGEKIIAHFLIQQYLKEDYSFT |
| | | | AYATISYLKIG* |

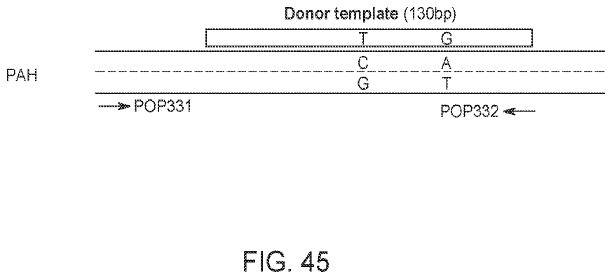

Detections of genetic “C-to-T” and “A-to-G” conversions after SSB-HbW-based gene edition were performed by Sanger Sequencing and next generation sequencing. Relative positions of a correction ssODN (i.e., sequence modification polynucleotide) and position of an exemplary primer pair: POP331 and POP332 are also indicated in FIG. 45. One common primer, POP331 was located outside this ssODN template (i.e., sequence modification polynucleotide) sequence, while POP332, located inside.

POP331 PAH a forward primer has a sequence of
(SEQ ID NO.: 100)
GGAGGTGTCCGTGTTCCTAA.
POP332 PAH reverse primer has a sequence of
(SEQ ID NO.: 101)
GCTGCTGGGTATTGTCCAAG.

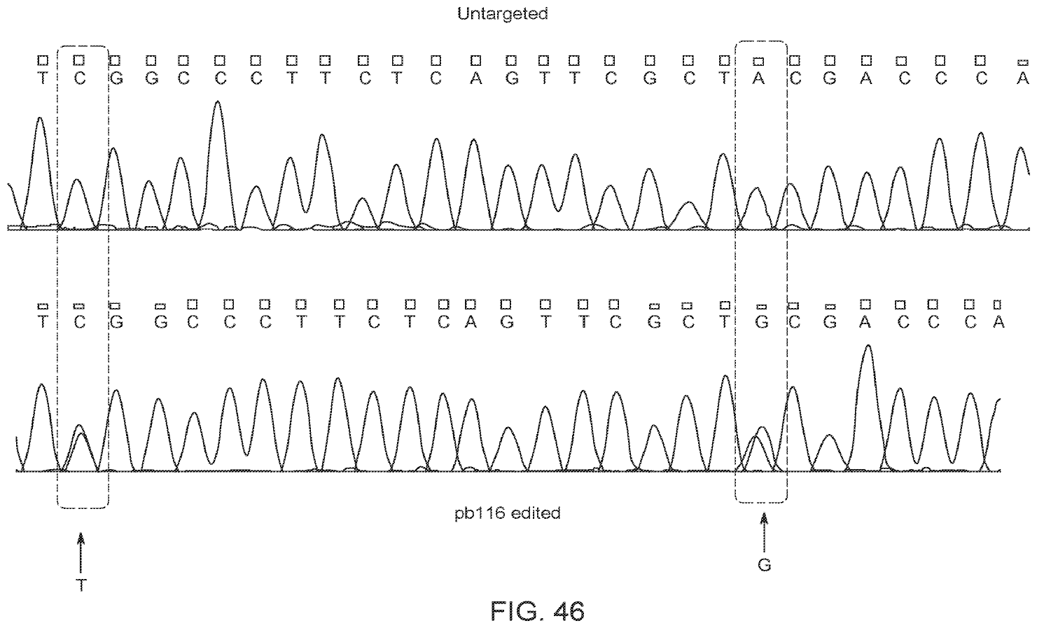

FIG. 46 shows exemplary Sanger sequencing results used to further confirm successful targeting and editing in exon 12 of human PAH gene. Genomic DNA was isolated and used as a template on which a 197-bp PCR amplicon surrounding double conversion sites in exon 12 of human PAH gene was generated by using a primer set of POP331 and POP332. Amplified PCR products from targeted B cells were analyzed. Upper panel of FIG. 46 shows an exemplary chromatogram from untargeted B-cell population, showing “C” and “A” in wild type sequence by Sanger sequencing. Lower panel of FIG. 46 shows an exemplary chromatogram of a pb 116 edited B-cell population, showing double genetic “C-to-T” and “A-to-G” conversions sequence by Sanger sequencing. These results confirm that a polynucleotide modification agent in combination with a sequence modification polynucleotide can successfully target and edit a sequence in an endogenous mammalian gene in mammalian cells to successfully modify a disease-causing genotype.

In the present Example, next generation sequencing was performed to determine, in more detail, gene conversion frequencies and patterns and also potential generation of insertions, deletions, and unintended single nucleotide polymorphisms after SSB-HbW-based gene editing. In order to do so, next generation sequencing of targeted pooled B cells (and untransfected B cells as control) was performed. Genomic DNA was isolated and used as a template on which a 197-bp PCR amplicon of sequences in exon 12 of human PAH was generated by using a primer set of POP331 and POP332. Amplified PCR products from targeted B cells and control B cells were analyzed for indels and SNPs on an Illumina next generation sequencing platform (GENEWIZ, South Plainfield, NJ).

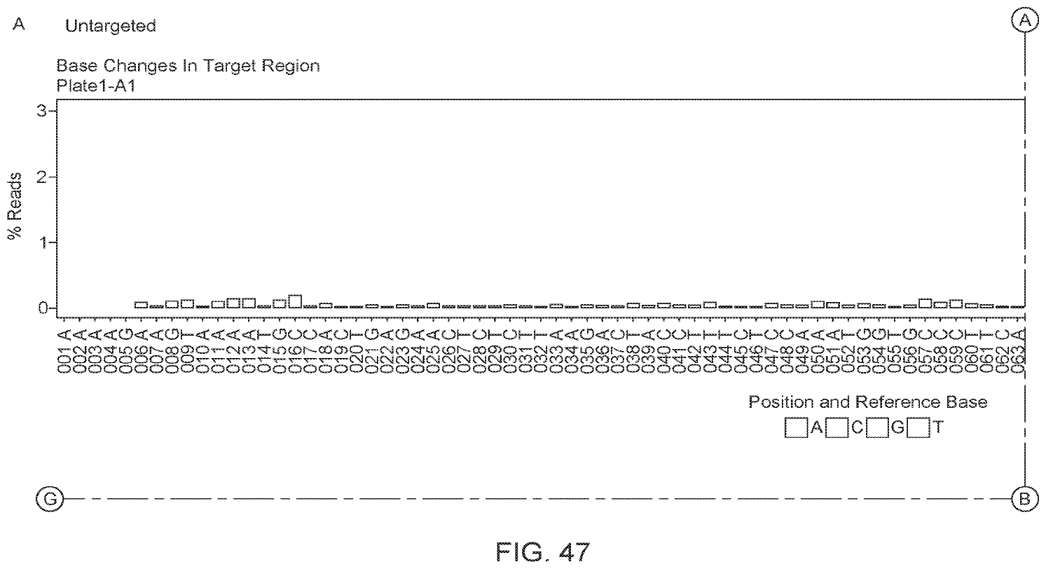

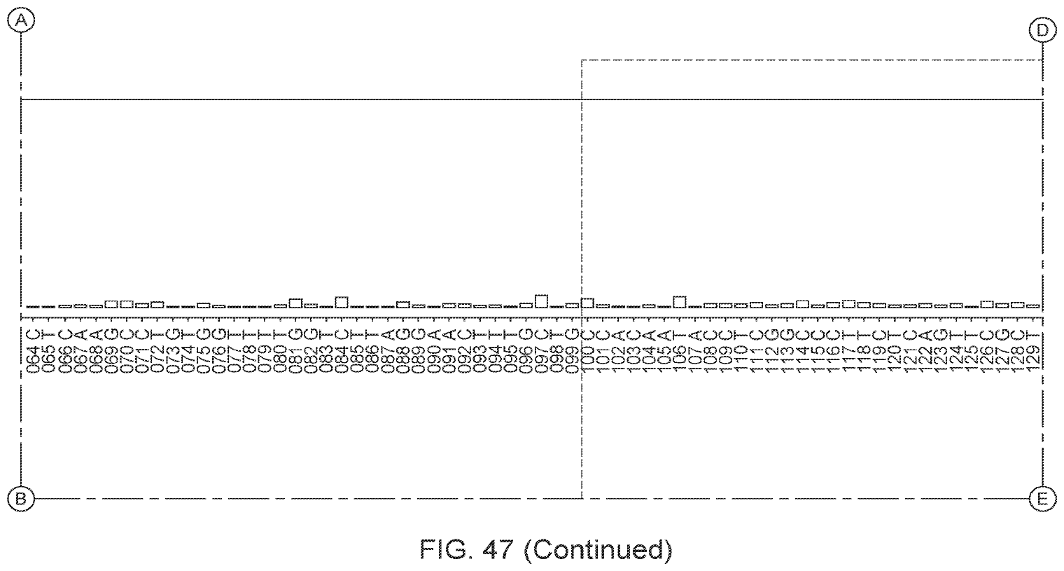

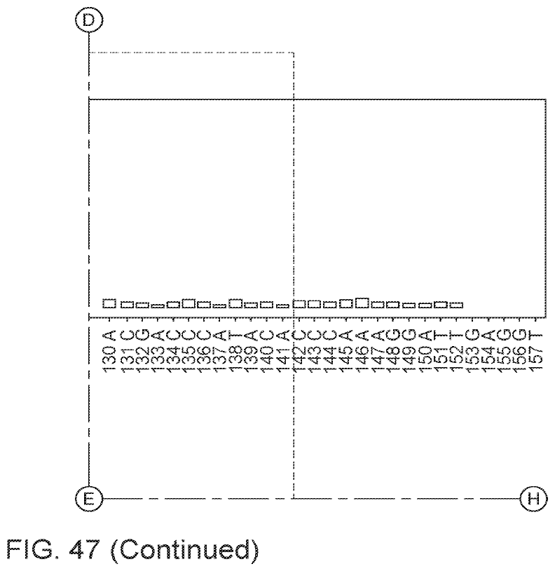

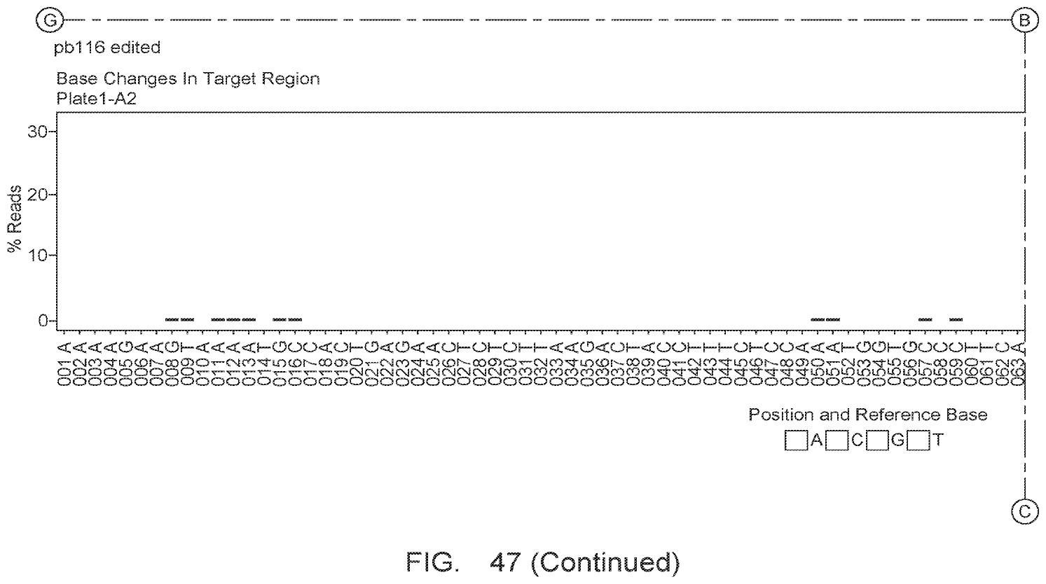

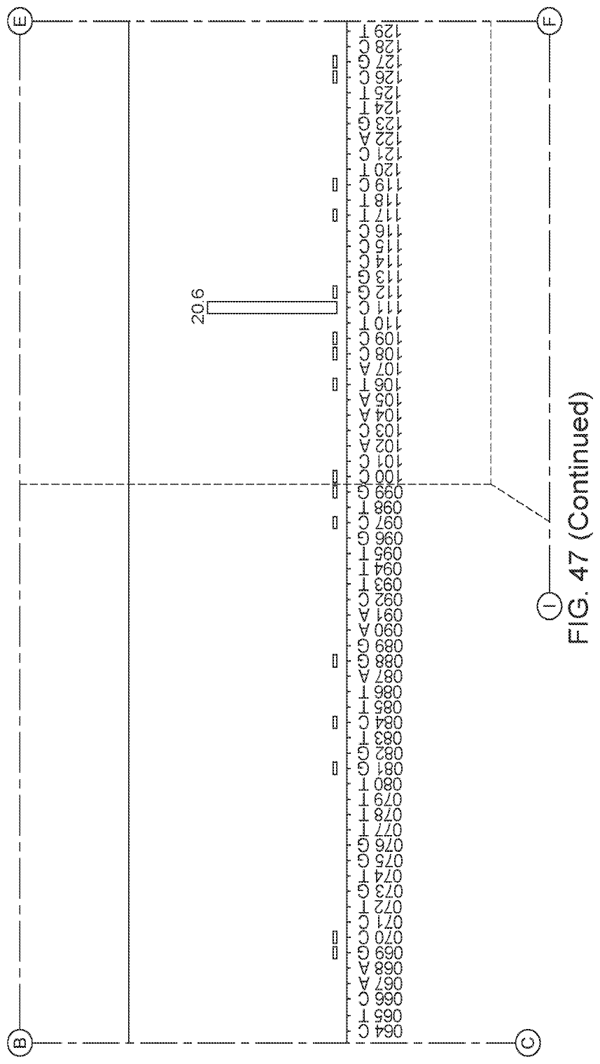

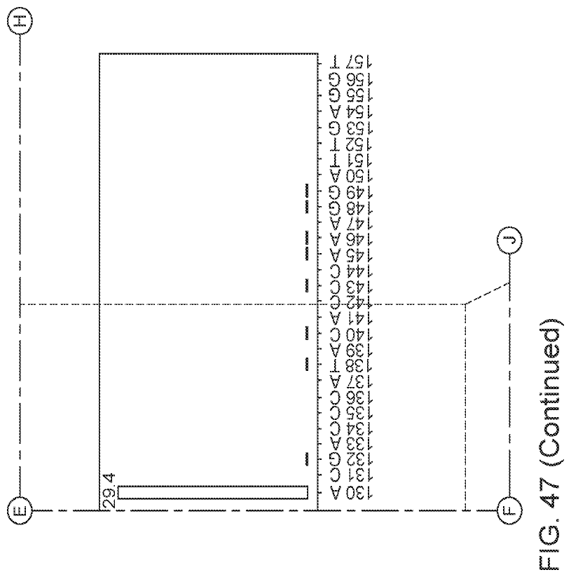

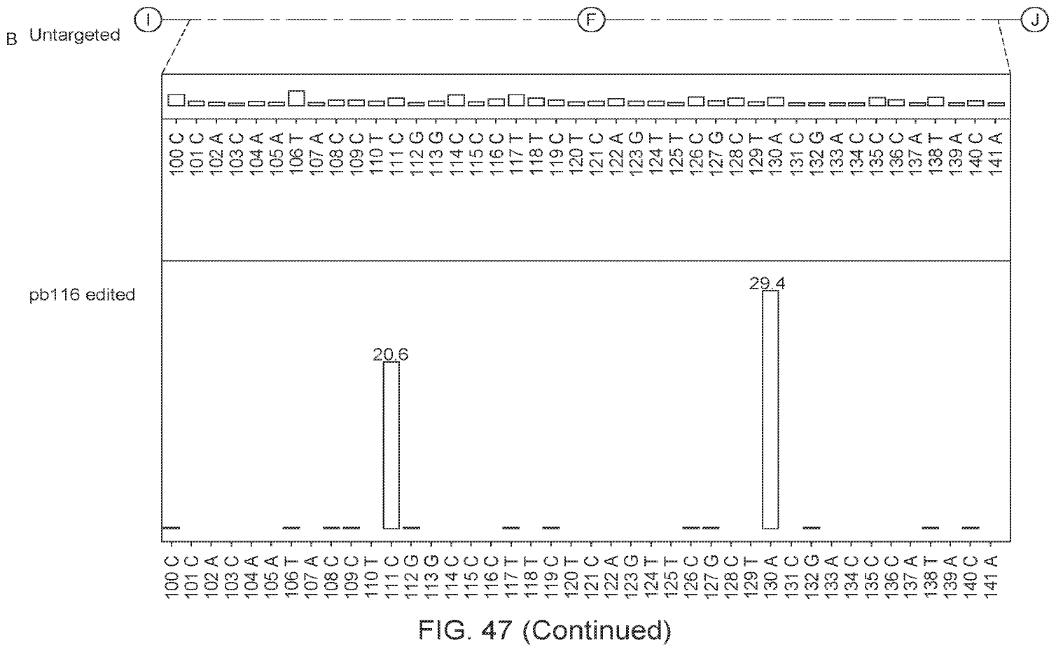

FIG. 47 shows confirmation of detection of both “C-to-T” and “A-to-G” conversions at this target site as well as single nucleotide polymorphisms (SNPs) analysis within a target region of in exon 12 of this PAH locus. Panel A shows overall views of SNPs analysis at these target sites obtained with untargeted B cells, and targeted pooled B cells. Bar graphs plot frequencies of SNPs at each nucleotide position in this 197 bp PCR amplification region. Panel A shows overall views of SNPs analysis at these target sites obtained with untargeted B cells, and targeted pooled B cells. Bar graphs plot frequencies of SNPs at each nucleotide position in this 197 bp PCR amplification region. Panel B is a magnified view of the portion close to this gene repair site. In this example cells transfected with pb 116 and a correction template showed both “C-to-T” and “A-to-G” conversions at expected nucleotide positions with a frequency of 20.5% and 29.4%, respectively. Compared to non-transfected B cells, no other nucleotide conversions had occurred at a level significantly above background. Comparing to untransfected cells, no obvious unwanted SNPs were detected.

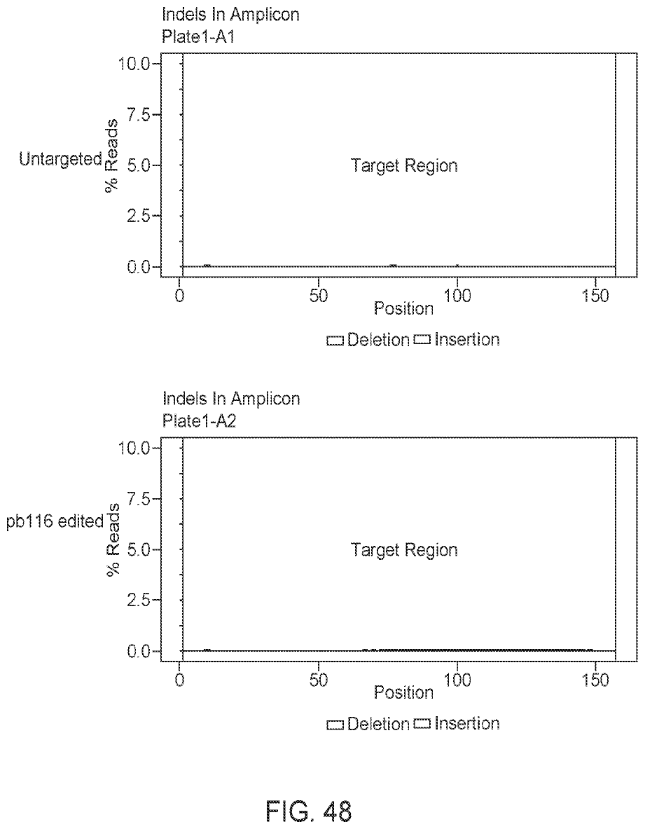

FIG. 48 shows overall insertion and deletion analysis within this amplification region of PAH, displayed as a frequency plot of insertions and deletions analysis in targeted pooled B cells. Distribution plot percentage of reads with insertions and deletions at each nucleotide position of this 197 bp PCR amplification region is indicated. This indel analysis showed, in general, a very low frequency of insertions and/or deletions, both in untargeted B cells (panel A) and pb116 edited B cells (panel B). In addition, patterns and frequencies of indels at each position from both targeted and untransfected B cells were not statistically significantly different and considered to be within an error range and within detection limitations typical for the PCR and next generation sequencing methods used.

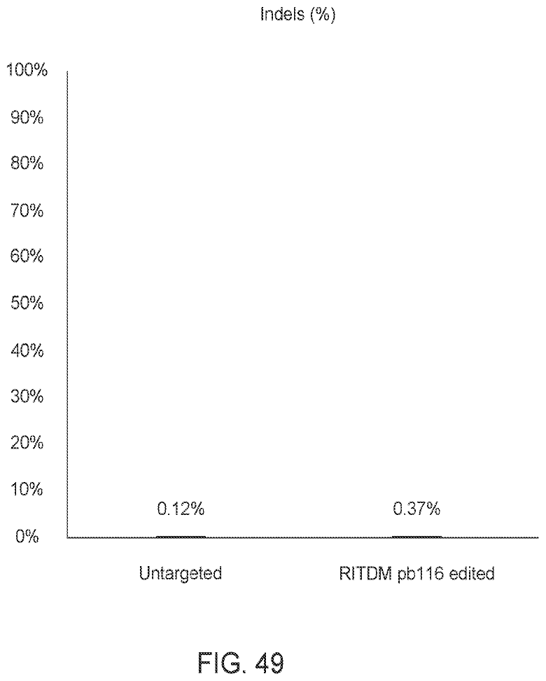

FIG. 49 shows overall indels frequencies using zinc finger helicase beta wing mediated gene editing targeting with pb116 and a sequence modification polynucleotide in comparison of untargeted B cells. Overall indel frequencies after zinc finger helicase beta wing mediated gene editing is only 0.37%, while untargeted control levels are at 0.12%. This difference is considered to be within detection limitations of technologies used. Taken together, zinc finger helicase beta wing mediated gene editing is able to achieve relatively high gene editing efficiencies with low indel frequencies. These results confirm that a polynucleotide modification agent in combination with a sequence modification polynucleotide can successfully target and edit a sequence in an endogenous mammalian gene in mammalian cells to successfully modify a disease-causing genotype. Notably, this example also demonstrates that provided technologies are capable of genetic modification with low levels of insertions and deletions. Thus, technologies of the present disclosure are capable of targeted gene conversion without potentially detrimental generation of insertions, deletions and/or undesired single nucleotide polymorphisms.

### Example 9: Modification of an Endogenous Genomic Target: CFTR by SSB-HbW-Based Gene Editing in Human B-Cells

The present example describes sequence specific genetic modification of CFTR in exemplary cells using technologies described herein. In this example, human CFTR was targeted and edited by SSB-HbW-based gene editing with a polynucleotide modification agent and a single stranded oligonucleotide template (i.e., a sequence modification polynucleotide). The human CFTR gene encodes cystic fibrosis transmembrane conductance regulator (CFTR) protein. Mutations in CFTR gene can result in dysfunctional CFTR, causing cystic fibrosis clinical symptoms. Dysfunctional CFTR protein is unable to move chloride from inside a cell to the cell surface in impacted cell types. As a consequence of a dysfunctional chloride transport, a reduced concentration of chloride to attract water to the cell surface, the mucus in various organs becomes thick and sticky. In the lungs, the thickened mucus clogs the airways and traps germs, like bacteria, leading to infections, inflammation, respiratory failure, and other complications. The most common mutation in CFTR gene causes deletion of phenylalanine codon 508 (delta F508). This mutation results in the synthesis of a variant CFTR protein that is defective in its ability to traffic to the plasma membrane. (S. H. Cheng. et al. Am J Physiol. 1995 April; 268 (4 Pt 1): L615-24, which is herein incorporated by reference in its entirety). In this example, codon 508 site of human CFTR gene was targeted and edited by SSB-HbW-based gene editing with a polynucleotide modification agent and a single stranded oligonucleotide template (i.e., a sequence modification polynucleotide).

The present disclosure contemplates that, in some embodiments, a polynucleotide modification system as described herein can be used for conversion of nucleotides in and close to codon F508 of human CFTR gene. This example describes development of a specifically designed polynucleotide modification agent (e.g., a SSB-HbW agent) with in combination with a sequence modification polynucleotide (e.g., a specific single stranded oligonucleotide template) to convert a DNA sequence in codon F508 site to create amino acid substitution(s).

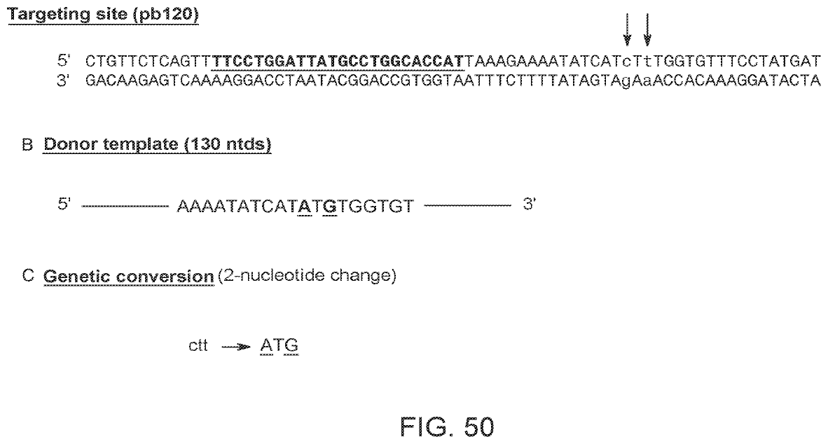

FIG. 50 provides a schematic that depicts gene editing of an endogenous genomic target human CFTR gene in B cells. In this example, an exemplary polynucleotide modification agent, encoded on plasmids pb 120, include a DNA recognition domain which was an array of 8 zinc-fingers, specifically designed to recognize 5′-ATGGTGCCAGGCATAATCCAGGAA-3′ (SEQ ID NO.: 103), a 24-nucleotide sequence on the leading strand of human CFTR, displayed with underlined and bold letters. As depicted in FIG. 50 panel A, a targeted nucleotide on leading strand of target site was displayed as a lowercase letter “cTt”, 3′ downstream of this binding site. Targeted nucleotides are displayed as lowercase letters, also indicated by two arrows. In this embodiment, a donor template was used: a 130-nucleotide single stranded DNA oligonucleotide with a desired “cTt-to-ATG” conversions roughly located in the middle of this oligonucleotide, as depicted in FIG. 50 panel B. The sequence of this sequence modification polynucleotide used is provided as SEQ ID NO.: 104 (below) with an underlined and bold “A” and “G” to indicate “cTt-to ATG” conversions. FIG. 50 panel C depicts 2-nucleotide genetic conversion resulted from successful genetic conversion SSB-HbW-based gene editing.

(SEQ ID NO.: 104)
5′-GAATTTCATTCTGTTCTCAGTTTTCCTGGATTATGCCTGGCACCATT
AAAGAAAATATCATATGTGGTGTTTCCTATGATGAATATAGATACAGAAG
CGTCATCAAAGCATGCCAACTAGAAGAGGTAAG-3′.

Here, a polynucleotide modification agent (encoded on plasmid pb 120 (full length DNA (SEQ ID NO.: 105); cDNA (SEQ ID. NO.: 106); amino acid sequence (SEQ ID. NO.: 107)), which has a DNA recognition domain comprised in an array of 8 zinc-fingers, was designed to specifically recognize 5′-ATGGTGCCAGGCATAATCCAGGAA-3′ (SEQ ID NO.: 103), a 24-nucleotide sequence on the leading DNA strand (top row of nucleotides) of human CFTR gene. Exemplary sequences are provided in Table 10 below.

TABLE 10
Exemplary polynucleotide modification agents targeting CFTR

| SEQ | | AA/ | |
|---|---|---|---|
| ID | | DNA/ | |
| NO: | Feature | CDNA | Sequence |
| 105 | pb120 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTATGGCGGCGAT |
| | | | GGCCGAGCGGCCCTTCGCCTGCGACATTTGTGGGAGAAAATTTGCTGATCAGTCCG |
| | | | GCAACCTGACCCGGCATACCAAGATCCACACCGGCTCTCAGAAACCATTCCAGTGC |
| | | | AGGATCTGTATGCGCAACTTTTCTCGGTCCGACAACCTGTCCGAGCACATCAGAAC |
| | | | CCATACAGGCGAAAAGCCTTTTGCTTGCGACATTTGTGGCAGGAAATTTGCTGACT |
| | | | CCTCCGCCCTGTCCCAGCACACTAAGATCCATACTGGGTCACAGAAACCTTTCCAG |
| | | | TGCCGCATTTGTATGCGGAATTTTTCCCAGTCCGGCTCCCTGTCCCAGCATATCCG |
| | | | CACTCACACCGGAGAGAAGCCCTTTGCATGCGACATTTGTGGACGGAAATTTGCTG |
| | | | ACCGGTCCCACCTGACCCGGCATACCAAGATTCACACTGGGTCTCAGAAACCTTTC |
| | | | CAGTGCAGGATTTGTATGAGAAATTTTTCCCAGTCCGGCGACCTGTCCGAGCACAT |
| | | | CAGAACCCATACAGGCGAAAAGCCTTTTGCTTGCGACATTTGTGGCAGGAAATTTG |
| | | | CTCGGTCCTCCGCCCTGACCCGGCACACTAAGATCCATACTGGGTCACAGAAACCT |
| | | | TTCCAGTGCCGCATTTGTATGCGGAATTTTTCCCGGTCCGACTCCCTGTCCCAGCA |
| | | | CATCAGAACACATACTGGGGAGAAGATCATCGCCCACTTCCTGATCCAGCAGTACC |
| | | | TGAAGGAGGACTACTCCTTCACCGCCTACGCCACCATCTCCTACCTGAAGATCGGC |
| | | | TGAGCGGCCGCTCGAGTCTAGAGGGCCCGTTTAAACCCGCTGATCAGCCTCGACTG |
| | | | TGCCTTCTAGTTGCCAGCCATCTGTTGTTTGCCCCTCCCCCGTGCCTTCCTTGACC |
| | | | CTGGAAGGTGCCACTCCCACTGTCCTTTCCTAATAAAATGAGGAAATTGCATCGCA |
| | | | TTGTCTGAGTAGGTGTCATTCTATTCTGGGGGGTGGGGTGGGGCAGGACAGCAAGG |
| | | | GGGAGGATTGGGAAGACAATAGCAGGCATGCTGGGGATGCGGTGGGCTCTATGGCT |
| | | | TCTACTGGGCGGTTTTATGGACAGCAAGCGAACCGGAATTGCCAGCTGGGGCGCCC |
| | | | TCTGGTAAGGTTGGGAAGCCCTGCAAAGTAAACTGGATGGCTTTCTCGCCGCCAAG |
| | | | GATCTGATGGCGCAGGGGATCAAGCTCTGATCAAGAGACAGGATGAGGATCGTTTC |
| | | | GCATGATTGAACAAGATGGATTGCACGCAGGTTCTCCGGCCGCTTGGGTGGAGAGG |
| | | | CTATTCGGCTATGACTGGGCACAACAGACAATCGGCTGCTCTGATGCCGCCGTGTT |
| | | | CCGGCTGTCAGCGCAGGGGCGCCCGGTTCTTTTTGTCAAGACCGACCTGTCCGGTG |
| | | | CCCTGAATGAACTGCAAGACGAGGCAGCGCGGCTATCGTGGCTGGCCACGACGGGC |
| | | | GTTCCTTGCGCAGCTGTGCTCGACGTTGTCACTGAAGCGGGAAGGGACTGGCTGCT |
| | | | ATTGGGCGAAGTGCCGGGGCAGGATCTCCTGTCATCTCACCTTGCTCCTGCCGAGA |
| | | | AAGTATCCATCATGGCTGATGCAATGCGGCGGCTGCATACGCTTGATCCGGCTACC |
| | | | TGCCCATTCGACCACCAAGCGAAACATCGCATCGAGCGAGCACGTACTCGGATGGA |
| | | | AGCCGGTCTTGTCGATCAGGATGATCTGGACGAAGAGCATCAGGGGCTCGCGCCAG |
| | | | CCGAACTGTTCGCCAGGCTCAAGGCGAGCATGCCCGACGGCGAGGATCTCGTCGTG |
| | | | ACCCATGGCGATGCCTGCTTGCCGAATATCATGGTGGAAAATGGCCGCTTTTCTGG |
| | | | ATTCATCGACTGTGGCCGGCTGGGTGTGGCGGACCGCTATCAGGACATAGCGTTGG |
| | | | CTACCCGTGATATTGCTGAAGAGCTTGGCGGCGAATGGGCTGACCGCTTCCTCGTG |
| | | | CTTTACGGTATCGCCGCTCCCGATTCGCAGCGCATCGCCTTCTATCGCCTTCTTGA |
| | | | CGAGTTCTTCTGAATTATTAACGCTTACAATTTCCTGATGCGGTATTTTCTCCTTA |
| | | | CGCATCTGTGCGGTATTTCACACCGCATACAGGTGGCACTTTTCGGGGAAATGTGC |
| | | | GCGGAACCCCTATTTGTTTATTTTTCTAAATACATTCAAATATGTATCCGCTCATG |
| | | | AGACAATAACCCTGATAAATGCTTCAATAATAGCACGTGCTAAAACTTCATTTTTA |
| | | | ATTTAAAAGGATCTAGGTGAAGATCCTTTTTGATAATCTCATGACCAAAATCCCTT |
| | | | AACGTGAGTTTTCGTTCCACTGAGCGTCAGACCCCGTAGAAAAGATCAAAGGATCT |
| | | | TCTTGAGATCCTTTTTTTCTGCGCGTAATCTGCTGCTTGCAAACAAAAAAACCACC |
| | | | GCTACCAGCGGTGGTTTGTTTGCCGGATCAAGAGCTACCAACTCTTTTTCCGAAGG |
| | | | TAACTGGCTTCAGCAGAGCGCAGATACCAAATACTGTCCTTCTAGTGTAGCCGTAG |
| | | | TTAGGCCACCACTTCAAGAACTCTGTAGCACCGCCTACATACCTCGCTCTGCTAAT |
| | | | CCTGTTACCAGTGGCTGCTGCCAGTGGCGATAAGTCGTGTCTTACCGGGTTGGACT |
| | | | CAAGACGATAGTTACCGGATAAGGCGCAGCGGTCGGGCTGAACGGGGGGTTCGTGC |
| | | | ACACAGCCCAGCTTGGAGCGAACGACCTACACCGAACTGAGATACCTACAGCGTGA |
| | | | GCTATGAGAAAGCGCCACGCTTCCCGAAGGGAGAAAGGCGGACAGGTATCCGGTAA |
| | | | GCGGCAGGGTCGGAACAGGAGAGCGCACGAGGGAGCTTCCAGGGGGAAACGCCTGG |
| | | | TATCTTTATAGTCCTGTCGGGTTTCGCCACCTCTGACTTGAGCGTCGATTTTTGTG |
| | | | ATGCTCGTCAGGGGGGCGGAGCCTATGGAAAAACGCCAGCAACGCGGCCTTTTTAC |
| | | | GGTTCCTGGGCTTTTGCTGGCCTTTTGCTCACATGTTCTT |
| 106 | pb120 | CDNA | ATGGCGGCGATGGCCGAGCGGCCCTTCGCCTGCGACATTTGTGGGAGAAAATTTGC |
| | | | TGATCAGTCCGGCAACCTGACCCGGCATACCAAGATCCACACCGGCTCTCAGAAAC |
| | | | CATTCCAGTGCAGGATCTGTATGCGCAACTTTTCTCGGTCCGACAACCTGTCCGAG |
| | | | CACATCAGAACCCATACAGGCGAAAAGCCTTTTGCTTGCGACATTTGTGGCAGGAA |
| | | | ATTTGCTGACTCCTCCGCCCTGTCCCAGCACACTAAGATCCATACTGGGTCACAGA |
| | | | AACCTTTCCAGTGCCGCATTTGTATGCGGAATTTTTCCCAGTCCGGCTCCCTGTCC |
| | | | CAGCATATCCGCACTCACACCGGAGAGAAGCCCTTTGCATGCGACATTTGTGGACG |
| | | | GAAATTTGCTGACCGGTCCCACCTGACCCGGCATACCAAGATTCACACTGGGTCTC |
| | | | AGAAACCTTTCCAGTGCAGGATTTGTATGAGAAATTTTTCCCAGTCCGGCGACCTG |
| | | | TCCGAGCACATCAGAACCCATACAGGCGAAAAGCCTTTTGCTTGCGACATTTGTGG |
| | | | CAGGAAATTTGCTCGGTCCTCCGCCCTGACCCGGCACACTAAGATCCATACTGGGT |
| | | | CACAGAAACCTTTCCAGTGCCGCATTTGTATGCGGAATTTTTCCCGGTCCGACTCC |
| | | | CTGTCCCAGCACATCAGAACACATACTGGGGAGAAGATCATCGCCCACTTCCTGAT |
| | | | CCAGCAGTACCTGAAGGAGGACTACTCCTTCACCGCCTACGCCACCATCTCCTACC |
| | | | TGAAGATCGGC |
| 107 | pb120 | AA | MAAMAERPFACDICGRKFADQSGNLTRHTKIHTGSQKPFQCRICMRNFSRSDNLSE |
| | | | HIRTHTGEKPFACDICGRKFADSSALSQHTKIHTGSQKPFQCRICMRNFSQSGSLS |
| | | | QHIRTHTGEKPFACDICGRKFADRSHLTRHTKIHTGSQKPFQCRICMRNFSQSGDL |
| | | | SEHIRTHTGEKPFACDICGRKFARSSALTRHTKIHTGSQKPFQCRICMRNFSRSDS |
| | | | LSQHIRTHTGEKIIAHFLIQQYLKEDYSFTAYATISYLKIG* |

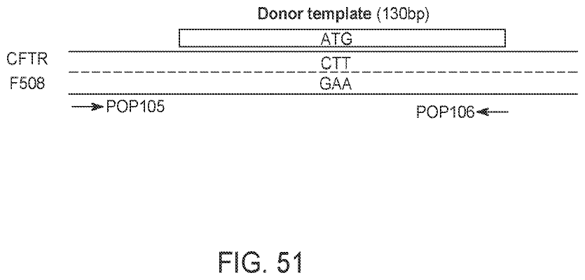

Detections of genetic “cTt-to-ATG” conversions after SSB-HbW-based gene edition were performed by next generation sequencing. Relative positions of a correction ssODN (i.e., sequence modification polynucleotide) and position of an exemplary primer pair: POP105 and POP106 are also indicated in FIG. 51. One common primer, POP105 was located outside this ssODN template (i.e., sequence modification polynucleotide) sequence, while POP106, located inside.

```
POP105 CFTR a forward primer has a sequence of
  (SEQ ID NO.: 108)
TGGAGCCTTCAGAGGGTAAA.
POP106 CFTR reverse primer has a sequence of
  (SEQ ID NO.: 109)
AGTTGGCATGCTTTGATGAC.
```

In the present Example, next generation sequencing was performed to determine, in more detail, gene conversion frequencies and patterns and also assess potential generation of insertions, deletions, and unintended single nucleotide polymorphisms after SSB-HbW-based gene editing. Next generation sequencing was performed on both targeted pooled B cells and untransfected B cells as a control. Genomic DNA was isolated and used as a template on which a 154-bp PCR amplicon surrounding codon 508 of human CFTR was generated by using a primer set of POP105 and POP106. Amplified PCR products from targeted B cells and control B cells were analyzed for indels and SNPs on an Illumina next generation sequencing platform (GENEWIZ, South Plainfield, NJ).

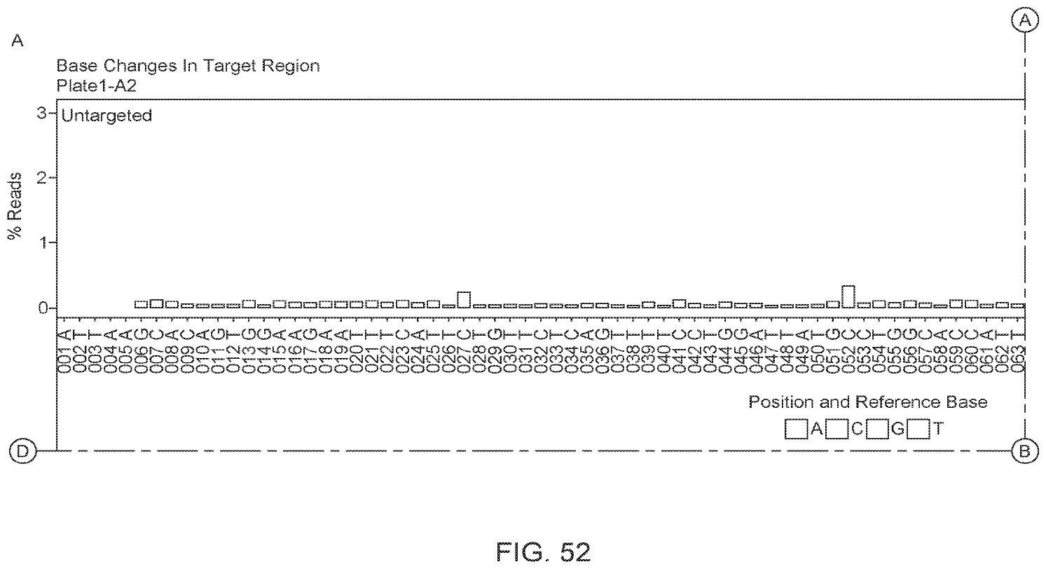

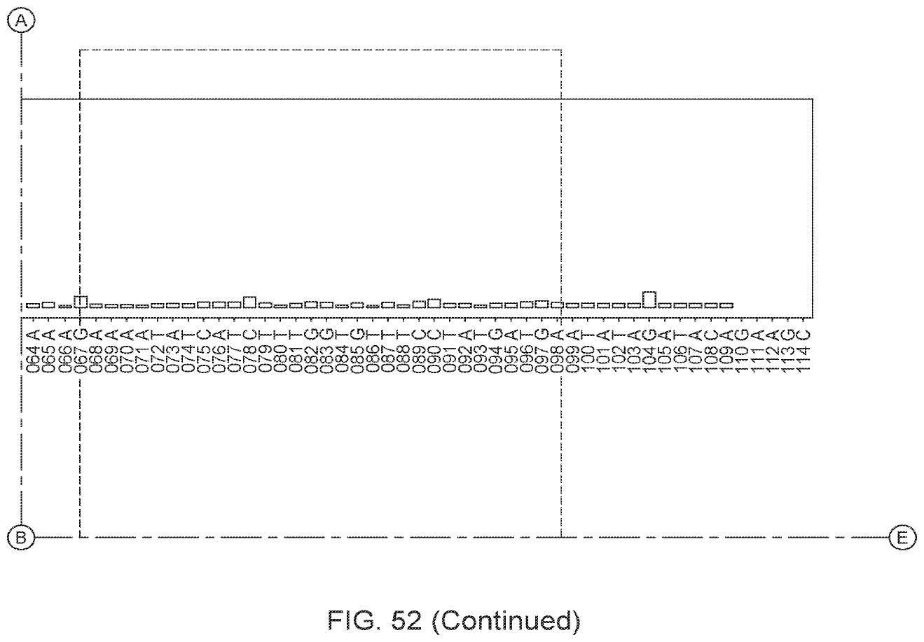

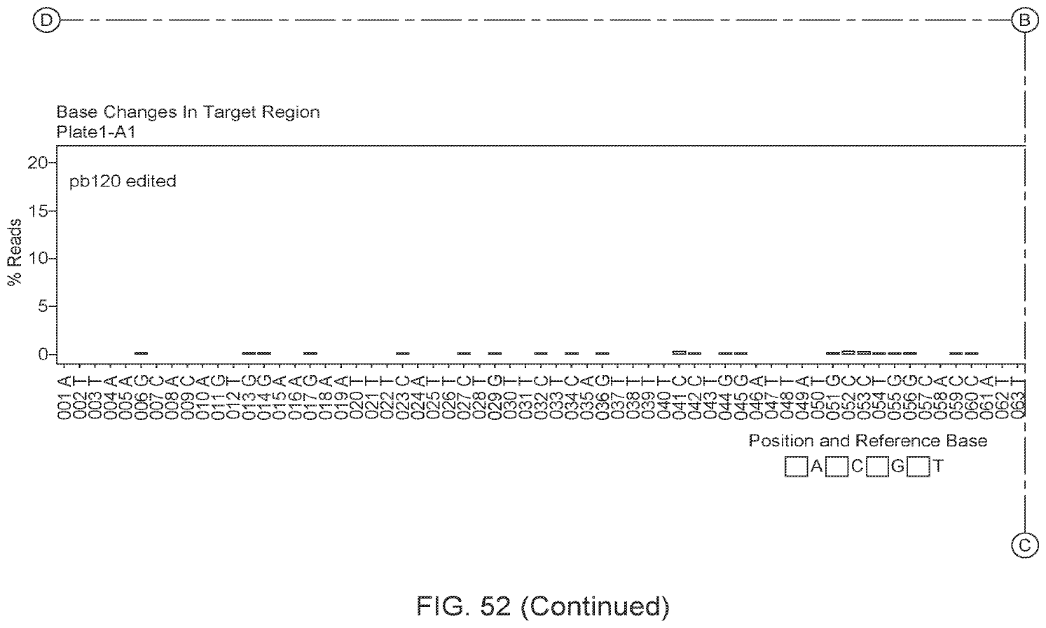

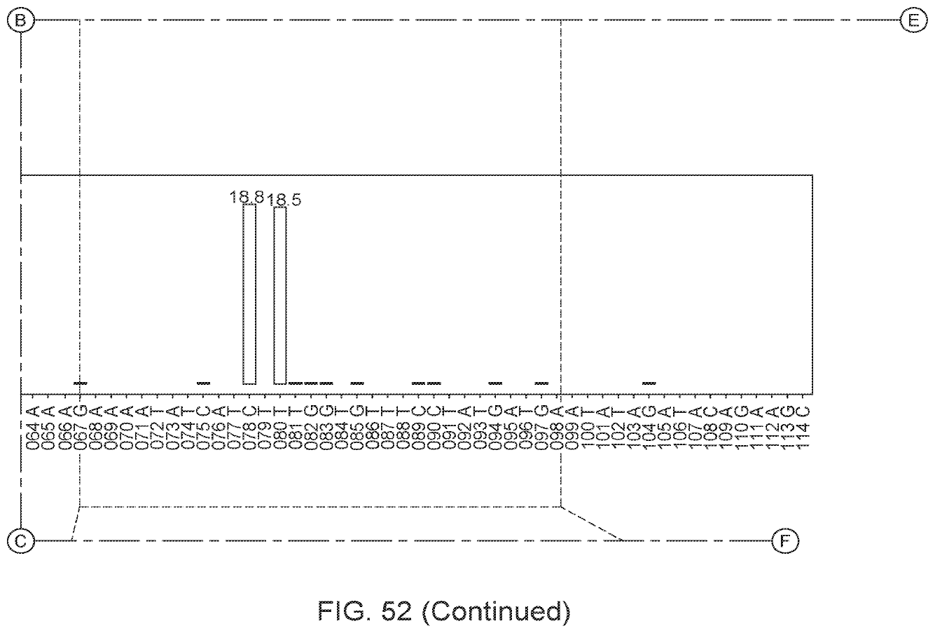

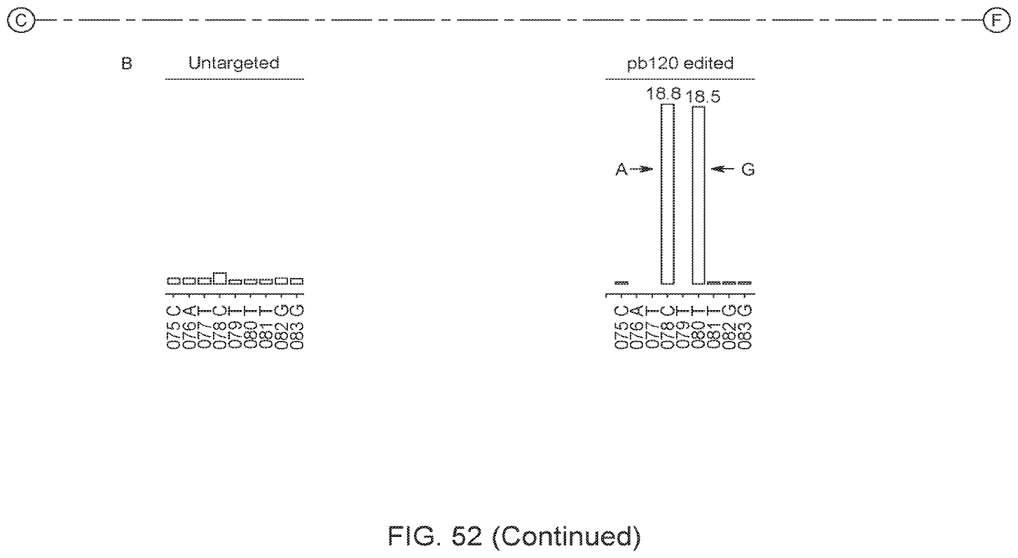

FIG. 52 shows confirmation of detection of a 2-nucleotide cTt-to ATG conversion at this target site as well as single nucleotide polymorphisms (SNPs) analysis within a target region of surrounding codon 508 of this CFTR locus. Panel A shows overall views of SNPs analysis at these target sites obtained with untargeted B cells, and targeted pooled B cells. Bar graphs plot frequencies of SNPs at each nucleotide position in this 154 bp PCR amplification region. Panel B is a magnified view of the portion close to this gene repair site. In this example cells transfected with pb 120 and a correction template showed a cTt-to ATG conversion at this expected nucleotide position with a frequency of 18.5%. Compared to non-transfected B cells, no other nucleotide conversions had occurred at a level significantly above background. Comparing to untransfected cells, no obvious unwanted SNPs were detected.

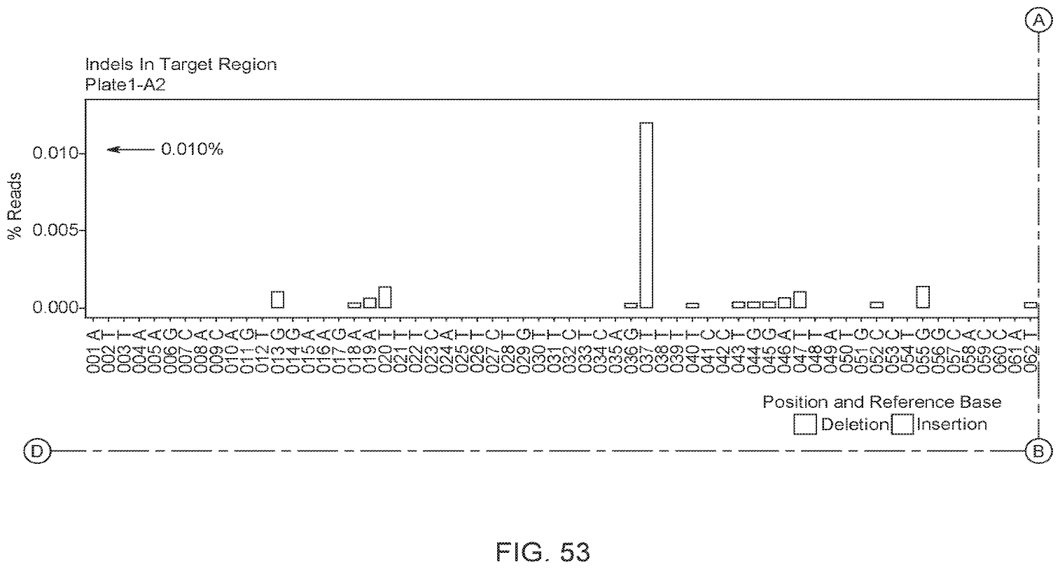

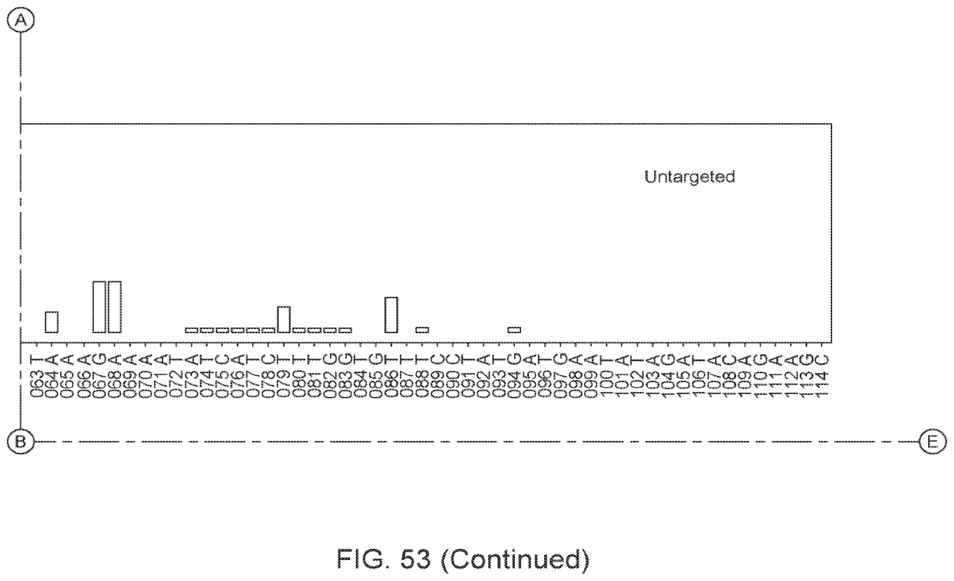

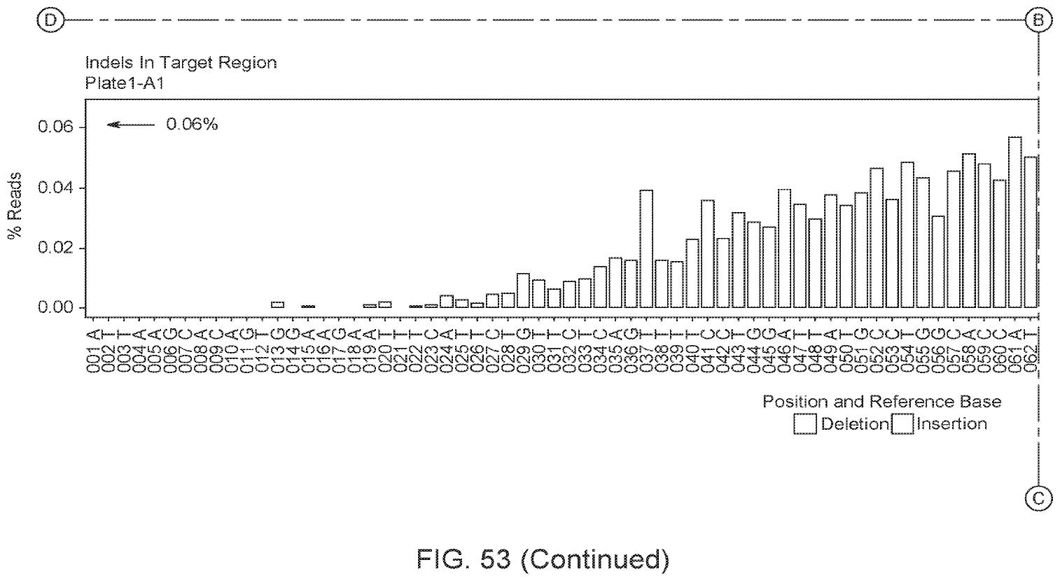

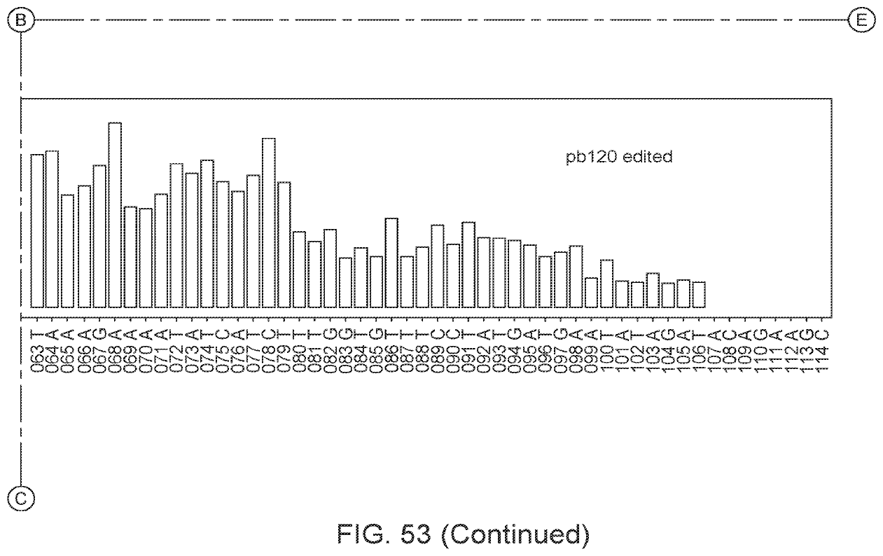

FIG. 53 shows insertion and deletion analysis around codon 508 of CFTR in an example using pb 120, displayed as a frequency plot of insertions and deletions analysis for targeted pooled B cells. Bar graphs plot frequencies of insertions and deletions at each nucleotide position of this 154 bp PCR amplification region. This indels analysis showed, in general, a very low frequency (<0.06%) of insertions and/or deletions. These results confirm that a polynucleotide modification agent in combination with a sequence modification polynucleotide can successfully target and edit a sequence in an endogenous mammalian gene in mammalian cells to successfully modify a disease-causing genotype. Notably, this example also demonstrates that provided technologies are capable of genetic modification with very low levels of insertions and deletions. Thus, technologies of the present disclosure are capable of targeted gene conversion without potentially detrimental generation of insertions, deletions and/or undesired single nucleotide polymorphisms.

### Example 10: Modification of an Endogenous Genomic Targets by Using mRNA-Based SSB-HbW-Based Gene Editing in Human B-Cells

The above examples show, inter alia, that plasmid-based delivery of a polynucleotide modification agent, e.g., SSB-HbW, can achieve genetic conversion at target loci. The present disclosure encompasses a recognition that it would be useful to have additional technologies to deliver polynucleotide modification agents (e.g., SSB-HbW compounds) into a cell or cells of interest. The present example describes using mRNA (messengerRNA) as an exemplary additional delivery system. In an eukaryotic cell, RNA, such as mRNA, is transcribed from a DNA template, such as endogenous gene or exogenous DNA, for example, a DNA plasmid. A transcribed mRNA can subsequently be translated into e.g., peptides, polypeptides, or proteins. In this example, an RNA encoding a protein described herein can be prepared by various methods, known to those skilled in the art. For example, an in vitro transcription system can be used, in which a DNA-vector or DNA-plasmid encoding the above-mentioned a polynucleotide modification agent, can be used to generate mRNA molecules encoding said protein. This example illustrates mRNA-mediated delivery of SSB-HbW-based polynucleotide modification agents.

The present disclosure provides a recognition that, in addition to a use as delivery method, mRNA delivery may provide additional or different benefits over plasmid-based delivery. As illustration, without being bound to a particular theory plasmid-based delivery may be deemed to carry a risk of transgene integration. A useful advantage of mRNA-based delivery of an editing agent may be in reducing risks of transgene integration within a genome. Thus, this Example confirms that a SSB-HbW-based polynucleotide modification agent can be delivered as mRNA.

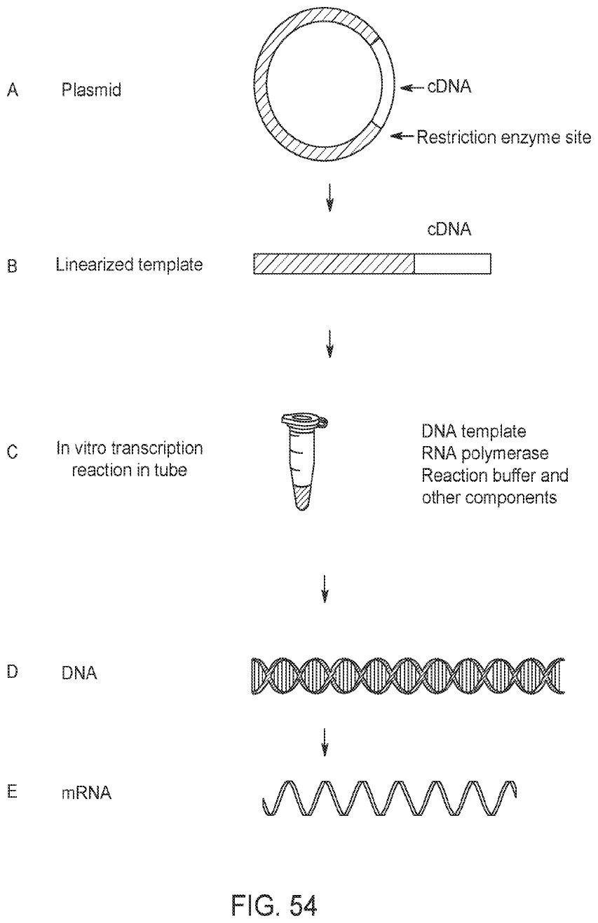

FIG. 54 shows a schematic process of in vitro transcription of mRNA from a DNA plasmid serving as a DNA template. FIG. 54 panel A depicts that a DNA plasmid can be displayed in a circular form. In this figure a black region indicates DNA plasmid backbone and a bright region indicates cDNA (copyDNA). In this disclosure, a cDNA sequence is encoding a polynucleotide modification agent. In this example, at one end of a cDNA coding region in this circular DNA plasmid (usually 3′ end of cDNA sequence), a restriction enzyme site is indicated. FIG. 54 panel B depicts that such a circular DNA plasmid can be linearized using a restriction enzyme, in this example at the 3′ end of cDNA coding region. FIG. 54 panel C depicts that a mRNA can be transcribed in vitro by mixing a linearized DNA template, RNA polymerase, reaction buffer and other components, typically from a commercial available in vitro transcription kit, in this example, T7 HiScribe T7 ARCA mRNA Kit from New England Biolabs (Ipswich, MA, USA). Upon successful completion of such a in vitro transcription procedure, a double stranded DNA template, illustrated in FIG. 54 panel D, is transcribed into single stranded mRNA, as illustrated in FIG. 54 panel E.

In this example, as illustration, mRNA is used to perform genomic editing. An approach is taken as depicted in FIG. 6 described in Example 2 above, using mRNA instead of plasmid DNA. This specific example aimed at gene editing of an endogenous genomic target around codon 112 of human ApoE in B cells. In this example, mRNA is transcribed from plasmid pb 121 (SEQ ID NO.: 111), including a DNA recognition domain which was an array of 9 zinc-fingers, specifically designed to recognize 5′-GCGGCCGCCTGGTGCAGTACCGCGGCG-3′ (SEQ ID NO.: 22), a 27-nucleotide sequence on the leading strand of human ApoE. Exemplary sequences are provided in Table 11 below. A targeted nucleotide “T” was displayed as a lowercase letter “t”, 5′ upstream of this binding site. In this embodiment, a donor template was used: a 129-nucleotide single stranded DNA oligonucleotide with a desired T→C substitution roughly located in the middle of this oligonucleotide. This single stranded donor template used herein is provided below as a sequence with an underlined and bold “C” to for T→C conversion.

(SEQ ID NO: 11)
5′-CCCCGGTGGCGGAGGAGACGCGGGCACGGCTGTCCAAGGAGCTGC
AGGCGGCGCAGGCCCGGCTGGGCGCGGACATGGAGGACGTGCGCGGCC
GCCTGGTGCAGTACCGCGGCGAGGTGCAGGCCATGC-3′

TABLE 11
Exemplary polynucleotide modification agent sequences

| SEQ | | DNA | |
|---|---|---|---|
| ID | | CDNA | |
| NO: | Feature | mRNA | Sequence |
| 111 | pb121 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAAGGGGAGACCCAAGCTGGCTAGCGTTTAAACTTAAGCTTATGGCGGCG |
| | | | ATGGCCGAGCGGCCCTTCCAGTGCAGGATCTGTATGCGCAACTTTTCTCGGTCCTC |
| | | | CGACCTGACCCGGCACATCAGAACCCATACAGGCGAAAAGCCTTTCGCCTGCGACA |
| | | | TTTGTGGGAGAAAATTTGCTCGGTCCGACACCCTGACCCGGCATACCAAGATCCAC |
| | | | ACCGGCTCTCAGAAACCATTCCAGTGCCGCATTTGTATGCGGAATTTTTCCCAGTC |
| | | | CGGCGACCTGTCCGAGCATATCCGCACTCACACCGGAGAGAAGCCCTTTGCTTGCG |
| | | | ACATTTGTGGCAGGAAATTTGCTACCTCCGGCCACCTGACCACCCACACTAAGATC |
| | | | CATACTGGGTCACAGAAACCTTTCCAGTGCCGGATTTGTATGAGAAACTTTAGCGA |
| | | | CTCCTCCCACCTGACCACCCACATCAGAACCCATACAGGCGAAAAGCCTTTCGCCT |
| | | | GCGACATTTGTGGGAGAAAATTTGCTCGGTCCTCCCACCTGACCACCCATACCAAG |
| | | | ATCCACACCGGCTCTCAGAAACCATTCCAGTGCCGCATTTGTATGCGGAATTTTTC |
| | | | CGACCGGTCCGACCTGACCCGGCATATCCGCACTCACACCGGAGAGAAGCCCTTTG |
| | | | CTTGCGACATTTGTGGCAGGAAATTTGCTGACCGGTCCGACCTGACCCGGCACACT |
| | | | AAGATCCATACTGGGTCACAGAAACCTTTCCAGTGCCGGATTTGTATGAGAAACTT |
| | | | TAGCCGGTCCGACACCCTGACCCGGCACATCAGAACACATACTGGGGAGAAGATCA |
| | | | TCGCCCACTTCCTGATCCAGCAGTACCTGAAGGAGGACTACTCCTTCACCGCCTAC |
| | | | GCCACCATCTCCTACCTGAAGATCGGCTGAGCGGCCGCTCGAGTCTAGAGGGCCCG |
| | | | TTTAAACCCGCTGATCAGCCTCGACTGTGCCTTCTAGTTGCCAGCCATCTGTTGTT |
| | | | TGCCCCTCCCCCGTGCCTTCCTTGACCCTGGAAGGTGCCACTCCCACTGTCCTTTC |
| | | | CTAATAAAATGAGGAAATTGCATCGCATTGTCTGAGTAGGTGTCATTCTATTCTGG |
| | | | GGGGTGGGGTGGGGCAGGACAGCAAGGGGGAGGATTGGGAAGACAATAGCAGGCAT |
| | | | GCTGGGGATGCGGTGGGCTCTATGGCTGCAGCTTCTACTGGGCGGTTTTATGGACA |
| | | | GCAAGCGAACCGGAATTGCCAGCTGGGGCGCCCTCTGGTAAGGTTGGGAAGCCCTG |
| | | | CAAAGTAAACTGGATGGCTTTCTTGCCGCCAAGGATCTGATGGCGCAGGGGATCAA |
| | | | GCTCTGATCAAGAGACAGGATGAGGATCGTTTCGCATGATTGAACAAGATGGATTG |
| | | | CACGCAGGTTCTCCGGCCGCTTGGGTGGAGAGGCTATTCGGCTATGACTGGGCACA |
| | | | ACAGACAATCGGCTGCTCTGATGCCGCCGTGTTCCGGCTGTCAGCGCAGGGGCGCC |
| | | | CGGTTCTTTTTGTCAAGACCGACCTGTCCGGTGCCCTGAATGAACTGCAAGACGAG |
| | | | GCAGCGCGGCTATCGTGGCTGGCCACGACGGGCGTTCCTTGCGCAGCTGTGCTCGA |
| | | | CGTTGTCACTGAAGCGGGAAGGGACTGGCTGCTATTGGGCGAAGTGCCGGGGCAGG |
| | | | ATCTCCTGTCATCTCACCTTGCTCCTGCCGAGAAAGTATCCATCATGGCTGATGCA |
| | | | ATGCGGCGGCTGCATACGCTTGATCCGGCTACCTGCCCATTCGACCACCAAGCGAA |
| | | | ACATCGCATCGAGCGAGCACGTACTCGGATGGAAGCCGGTCTTGTCGATCAGGATG |
| | | | ATCTGGACGAAGAGCATCAGGGGCTCGCGCCAGCCGAACTGTTCGCCAGGCTCAAG |
| | | | GCGAGCATGCCCGACGGCGAGGATCTCGTCGTGACCCATGGCGATGCCTGCTTGCC |
| | | | GAATATCATGGTGGAAAATGGCCGCTTTTCTGGATTCATCGACTGTGGCCGGCTGG |
| | | | GTGTGGCGGACCGCTATCAGGACATAGCGTTGGCTACCCGTGATATTGCTGAAGAG |
| | | | CTTGGCGGCGAATGGGCTGACCGCTTCCTCGTGCTTTACGGTATCGCCGCTCCCGA |
| | | | TTCGCAGCGCATCGCCTTCTATCGCCTTCTTGACGAGTTCTTCTGAATTATTAACG |
| | | | CTTACAATTTCCTGATGCGGTATTTTCTCCTTACGCATCTGTGCGGTATTTCACAC |
| | | | CGCATACAGGTGGCACTTTTCGGGGAAATGTGCGCGGAACCCCTATTTGTTTATTT |
| | | | TTCTAAATACATTCAAATATGTATCCGCTCATGAGACAATAACCCTGATAAATGCT |
| | | | TCAATAATAGCACGTGCTAAAACTTCATTTTTAATTTAAAAGGATCTAGGTGAAGA |
| | | | TCCTTTTTGATAATCTCATGACCAAAATCCCTTAACGTGAGTTTTCGTTCCACTGA |
| | | | GCGTCAGACCCCGTAGAAAAGATCAAAGGATCTTCTTGAGATCCTTTTTTTCTGCG |
| | | | CGTAATCTGCTGCTTGCAAACAAAAAAACCACCGCTACCAGCGGTGGTTTGTTTGC |
| | | | CGGATCAAGAGCTACCAACTCTTTTTCCGAAGGTAACTGGCTTCAGCAGAGCGCAG |
| | | | ATACCAAATACTGTCCTTCTAGTGTAGCCGTAGTTAGGCCACCACTTCAAGAACTC |
| | | | TGTAGCACCGCCTACATACCTCGCTCTGCTAATCCTGTTACCAGTGGCTGCTGCCA |
| | | | GTGGCGATAAGTCGTGTCTTACCGGGTTGGACTCAAGACGATAGTTACCGGATAAG |
| | | | GCGCAGCGGTCGGGCTGAACGGGGGGTTCGTGCACACAGCCCAGCTTGGAGCGAAC |
| | | | GACCTACACCGAACTGAGATACCTACAGCGTGAGCTATGAGAAAGCGCCACGCTTC |
| | | | CCGAAGGGAGAAAGGCGGACAGGTATCCGGTAAGCGGCAGGGTCGGAACAGGAGAG |
| | | | CGCACGAGGGAGCTTCCAGGGGGAAACGCCTGGTATCTTTATAGTCCTGTCGGGTT |
| | | | TCGCCACCTCTGACTTGAGCGTCGATTTTTGTGATGCTCGTCAGGGGGGCGGAGCC |
| | | | TATGGAAAAACGCCAGCAACGCGGCCTTTTTACGGTTCCTGGGCTTTTGCTGGCCT |
| | | | TTTGCTCACATGTTCTT |
| 112 | pb121 | CDNA | ATGGCGGCGATGGCCGAGCGGCCCTTCCAGTGCAGGATCTGTATGCGCAACTTTTC |
| | | | TCGGTCCTCCGACCTGACCCGGCACATCAGAACCCATACAGGCGAAAAGCCTTTCG |
| | | | CCTGCGACATTTGTGGGAGAAAATTTGCTCGGTCCGACACCCTGACCCGGCATACC |
| | | | AAGATCCACACCGGCTCTCAGAAACCATTCCAGTGCCGCATTTGTATGCGGAATTT |
| | | | TTCCCAGTCCGGCGACCTGTCCGAGCATATCCGCACTCACACCGGAGAGAAGCCCT |
| | | | TTGCTTGCGACATTTGTGGCAGGAAATTTGCTACCTCCGGCCACCTGACCACCCAC |
| | | | ACTAAGATCCATACTGGGTCACAGAAACCTTTCCAGTGCCGGATTTGTATGAGAAA |
| | | | CTTTAGCGACTCCTCCCACCTGACCACCCACATCAGAACCCATACAGGCGAAAAGC |
| | | | CTTTCGCCTGCGACATTTGTGGGAGAAAATTTGCTCGGTCCTCCCACCTGACCACC |
| | | | CATACCAAGATCCACACCGGCTCTCAGAAACCATTCCAGTGCCGCATTTGTATGCG |
| | | | GAATTTTTCCGACCGGTCCGACCTGACCCGGCATATCCGCACTCACACCGGAGAGA |
| | | | AGCCCTTTGCTTGCGACATTTGTGGCAGGAAATTTGCTGACCGGTCCGACCTGACC |
| | | | CGGCACACTAAGATCCATACTGGGTCACAGAAACCTTTCCAGTGCCGGATTTGTAT |
| | | | GAGAAACTTTAGCCGGTCCGACACCCTGACCCGGCACATCAGAACACATACTGGGG |
| | | | AGAAGATCATCGCCCACTTCCTGATCCAGCAGTACCTGAAGGAGGACTACTCCTTC |
| | | | ACCGCCTACGCCACCATCTCCTACCTGAAGATCGGCTGA |
| 113 | pb121 | mRNA | ATGGCGGCGATGGCCGAGCGGCCCTTCCAGTGCAGGATCTGTATGCGCAACTTTTC |
| | | | TCGGTCCTCCGACCTGACCCGGCACATCAGAACCCATACAGGCGAAAAGCCTTTCG |
| | | | CCTGCGACATTTGTGGGAGAAAATTTGCTCGGTCCGACACCCTGACCCGGCATACC |
| | | | AAGATCCACACCGGCTCTCAGAAACCATTCCAGTGCCGCATTTGTATGCGGAATTT |
| | | | TTCCCAGTCCGGCGACCTGTCCGAGCATATCCGCACTCACACCGGAGAGAAGCCCT |
| | | | TTGCTTGCGACATTTGTGGCAGGAAATTTGCTACCTCCGGCCACCTGACCACCCAC |
| | | | ACTAAGATCCATACTGGGTCACAGAAACCTTTCCAGTGCCGGATTTGTATGAGAAA |
| | | | CTTTAGCGACTCCTCCCACCTGACCACCCACATCAGAACCCATACAGGCGAAAAGC |
| | | | CTTTCGCCTGCGACATTTGTGGGAGAAAATTTGCTCGGTCCTCCCACCTGACCACC |
| | | | CATACCAAGATCCACACCGGCTCTCAGAAACCATTCCAGTGCCGCATTTGTATGCG |
| | | | GAATTTTTCCGACCGGTCCGACCTGACCCGGCATATCCGCACTCACACCGGAGAGA |
| | | | AGCCCTTTGCTTGCGACATTTGTGGCAGGAAATTTGCTGACCGGTCCGACCTGACC |
| | | | CGGCACACTAAGATCCATACTGGGTCACAGAAACCTTTCCAGTGCCGGATTTGTAT |
| | | | GAGAAACTTTAGCCGGTCCGACACCCTGACCCGGCACATCAGAACACATACTGGGG |
| | | | AGAAGATCATCGCCCACTTCCTGATCCAGCAGTACCTGAAGGAGGACTACTCCTTC |
| | | | ACCGCCTACGCCACCATCTCCTACCTGAAGATCGGCTGA |

Detection of genetic T→C conversion after SSB-HbW-based gene edition was performed by droplet digital PCR (ddPCR). Relative positions of a correction ssODN (i.e., sequence modification polynucleotide) and position of an exemplary primer pair: POP46 and POP37 are also indicated in FIG. 6 described in Example 2. One common primer, POP46 was located inside this ssODN template (i.e., sequence modification polynucleotide) sequence, while POP37, located outside. Allele-specific probes conjugated with fluorophores FAM and HEX were designed to distinguish between “C” and “T” respectively. PstI restriction enzyme sites indicated were used in preparations for ddPCR reactions.

POP46-511-Alu-apoE-f forward primer
has a sequence of
(SEQ ID NO: 62)
CTGCAGGCGGCGCAGGC
POP37 ApoE reverse primer has a sequence of
(SEQ ID NO: 63)
GGTCATCGGCATCGCGGAGGAG

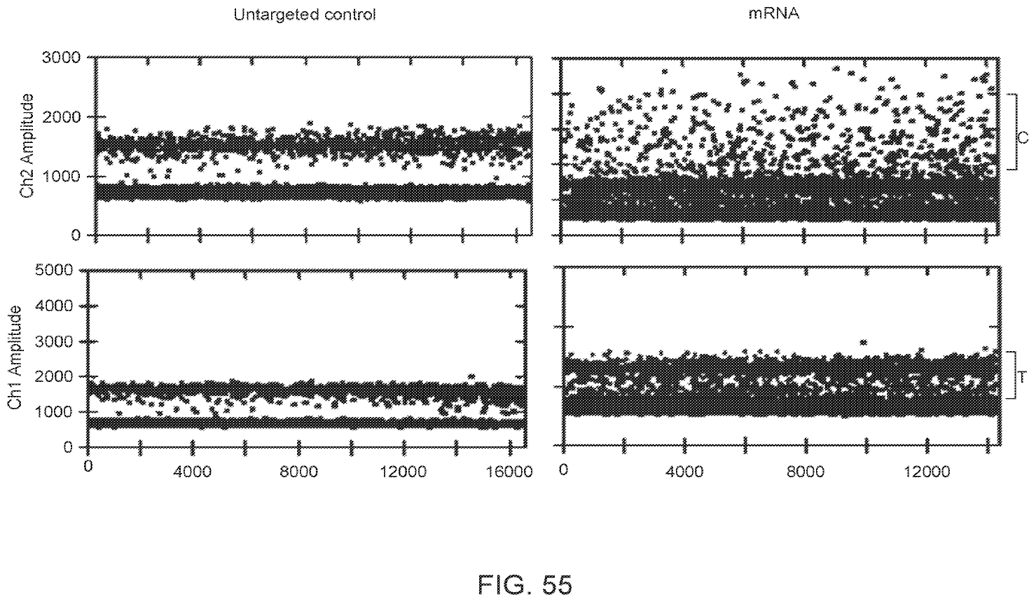

FIG. 55 demonstrates successful T→C genetic conversion at codon 112 of human ApoE as measured by ddPCR. In this example, after transfection of B cells with mRNA generated from pb121 and this 129-nucleotide correction template, cells were allowed to recover and grow on complete culture medium, containing 15% FBS in DMEM, for seven days. After seven days genomic DNA was isolated and used in ddPCR analysis. Raw droplet data are shown in FIG. 55 where these “C” droplets are displayed in the top panel, while “T” droplets were in the lower one. After B cells were transfected with mRNA derived from pb121 and ssODN template (i.e., sequence modification polynucleotide), “C” droplets appeared after being targeted and edited by this polynucleotide modification agent in combination with a correcting template, demonstrating successful T→C genetic conversion at codon 112 of human ApoE.

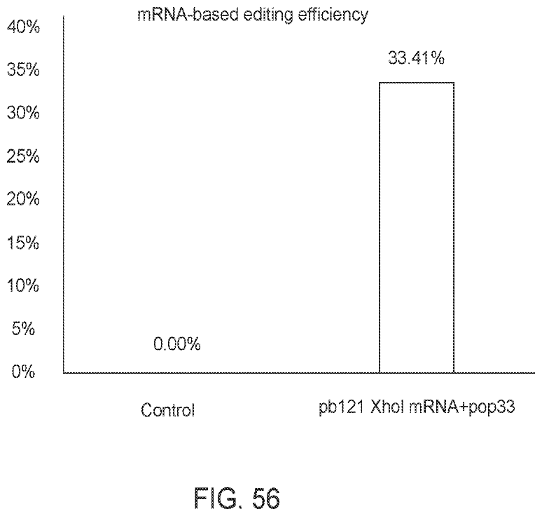

FIG. 56 shows genetic “T-to-C” conversion frequencies as measured by ddPCR after mRNA-based HbW-based gene editing. It shows editing frequencies corresponding to cellular “T-to-C” conversion percentages, defined as a percentage of “C” droplet events divided by the sum of “T” and “C” droplet events. Here, this mRNA-HbW-based gene editing achieved a 33.41% genetic conversion frequency compared to a background level of 0.00% of “T-to-C” conversion.

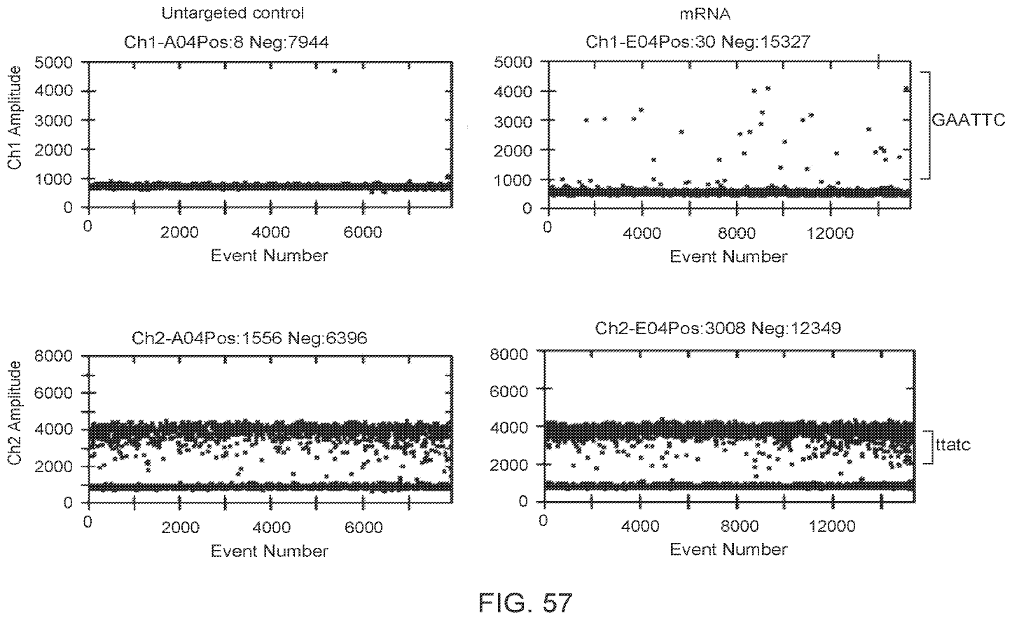

FIG. 57 shows mRNA-based HbW-based gene editing of an endogenous genomic target in a “GATAA” motif in an enhancer in intron 2 of human Bcl11A in human B cells, described in Example 1 (note in this provisional draft) above. Successful “ttatc-to-GAATC” genetic conversion at a GATTA motif of human Bcl11a as measured by ddPCR. After transfection of B cells with mRNA transcribed from plasmid pb112 (SEQ ID NO.: 65) and 141-nucleotide correction template (SEQ ID NO.: 12), cells were allowed to recover and grow on complete culture medium, containing 15% FBS in RPMI, for five days. After five days genomic DNA was isolated and used in ddPCR analysis. Raw droplet data are shown as in FIG. 57 where these “GAATTC” droplets are displayed in the top panel in each set, while “ttatc” droplets were in the lower one. Untargeted B-cells were used as a negative control, showing only “ttatc” droplets, but no “GAATTC” droplets. Untargeted B cells only had “ttatc” droplets, demonstrating untargeted wildtype genotype. After B cells were transfected with mRNA transcribed from pb112 and ssODN template (i.e., sequence modification polynucleotide), “GAATTC” droplets appeared after being targeted and edited by this polynucleotide modification agent in combination with a correcting template, demonstrating successful “ttatc-to-GAATC” genetic conversion at this GATAA motif of human Bcl11a using mRNA.

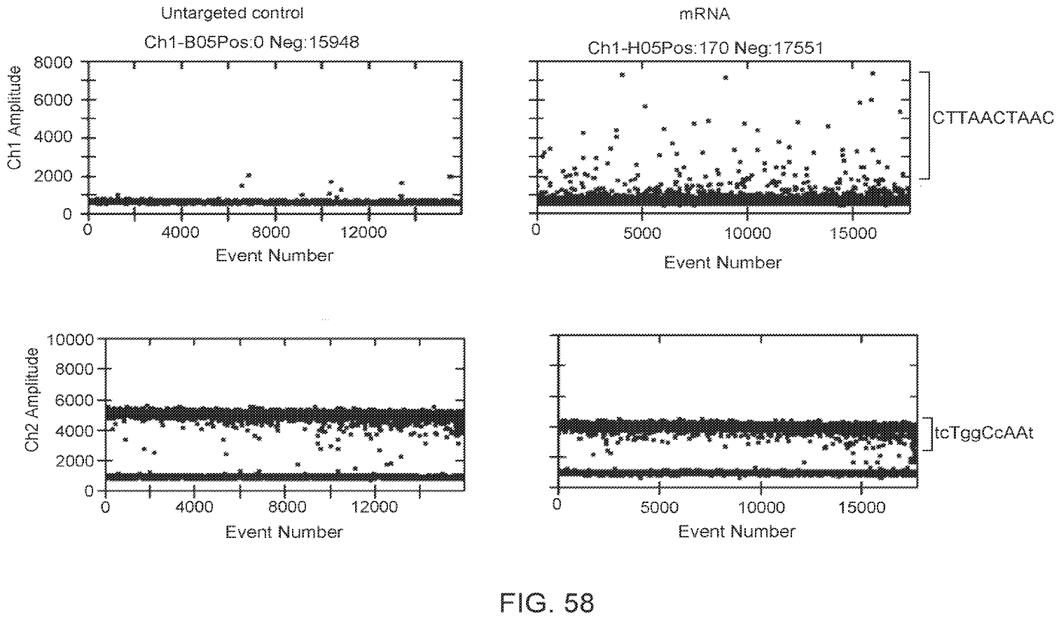

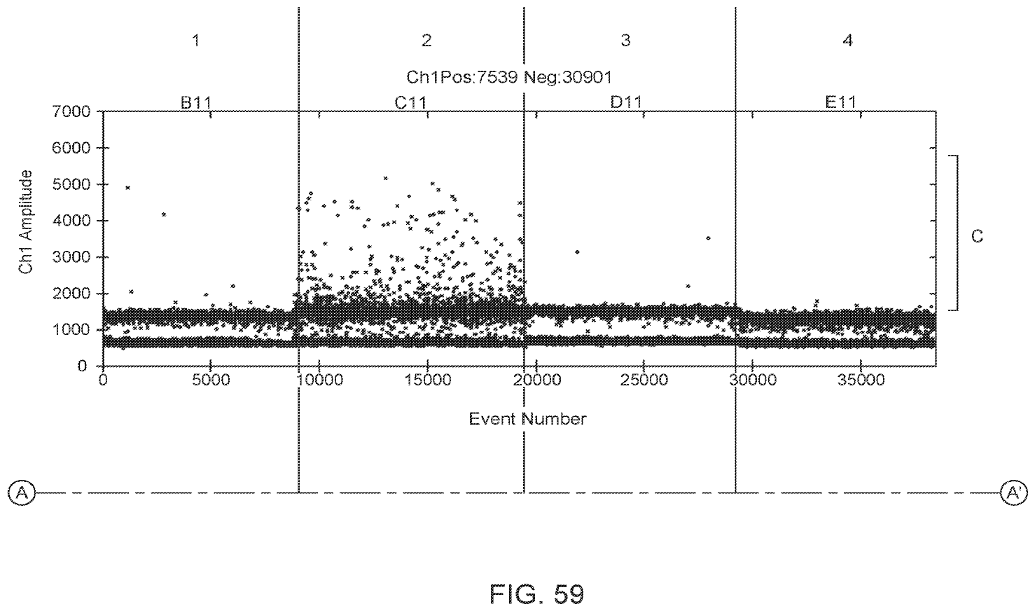

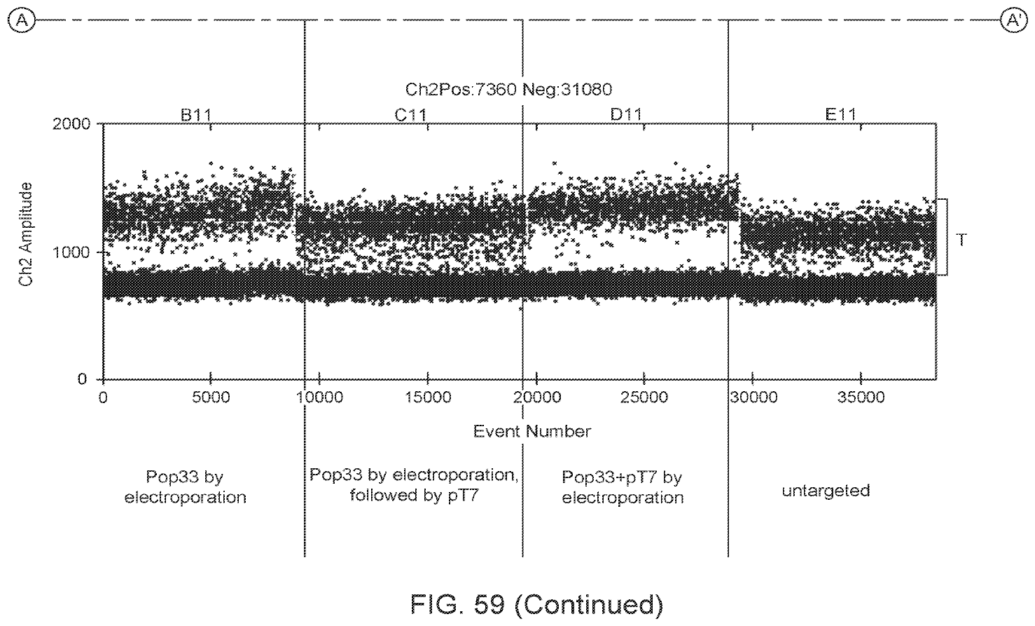

FIG. 58 shows mRNA-based HbW-based gene editing of an endogenous genomic target in region around codon A467 of human PolG in B cells, described in Example 5 above. Successful “tcTggCcAAt-to-CTTAACTAAC” genetic conversion at and close to codon 467 of human PolG as measured by ddPCR. After transfection of B cells with mRNA transcribed from plasmid pb 125 (SEQ ID NO.: 73) and 130-nucleotide correction template ((SEQ ID NO.: 72), cells were allowed to recover and grow on complete culture medium, containing 15% FBS, for five days. After five days genomic DNA was isolated and used in ddPCR analysis. Raw droplet data are shown as in FIG. 59 where these “CTTAACTAAC” droplets are displayed in the top panel in each set, while “tcTggCcAAt” droplets were in the lower one. Untargeted B-cells were used as a negative control, showing only “tcTggCcAAt” droplets, but no “CTTAACTAAC” droplets. Untargeted B cells only had “tcTggCcAAt” droplets, demonstrating untargeted wildtype genotype. After human B cells were transfected with mRNA transcribed from pb 125 and ssODN template (i.e., sequence modification polynucleotide), “CTTAACTAAC” droplets appeared after being targeted and edited by this polynucleotide modification agent in combination with a correcting template, demonstrating successful “tcTggCcAAt-to-CTTAACTAAC” genetic conversion at codon A467 site of human PolG using mRNA. Collectively, this example demonstrates that a SSB-HbW-based polynucleotide modification agent can be delivered as mRNA together with a single stranded DNA donor template for genetic conversion at various target sites within a genome.

### Example 11: Modification of an Endogenous Genomic Target: Codon 112 of ApoE Gene by SSB-HbW-Based Gene Editing Through Protein Delivery

SSB-HbW-based gene editing involves delivery of both a polynucleotide modification agent and a single stranded oligonucleotide template into cells. In addition to plasmid/DNA based delivery or mRNA based delivery, this disclosure demonstrates SSB-HbW-based gene editing can also be delivered in forms of a protein-single stranded oligonucleotide template complex.

The present disclosure provides a recognition that additional delivery methods may provide features and benefits relevant for targeted genome editing and that having an opportunity to select from various delivery methods can be useful for development of therapies and medicines. For example, unlike methods that rely on expression from nucleic acids, protein delivery has advantage a reduced risk of chromosomal insertion, thus minimizing tumorigenic or oncogenic side effects.

In this example, polynucleotide modification agents for SSB-HbW-based gene editing are produced as proteins, using protein production methods known to those skilled in the art, such as protein production in a prokaryotic or eukaryotic host from a suitable expression plasmid. After (partial) purification using protein purification methods known to those skilled in the art, such as ion exchange chromatography, size exclusion chromatography, precipitation technologies, a protein can be obtained that can be used as a SSB-HbW component. In this example a partially purified protein is incubated with a single-stranded oligonucleotide template in a buffer system of appropriate pH, ionic strength and ionic composition. After incubation, in this example, such a protein-oligonucleotide complex can be directly added to a cell culture, in single or multiple doses, and subsequently genome editing can be achieved.

FIG. 59 illustrates results from an experiment in which a sequence-specific SSB-HbW protein can be used to enter human B-cells to enable gene editing. In this example one aim is to genetically convert “T” into “C” at codon 112 of human ApoE in B cells using a protein as a polynucleotide modification agent. Protein pT7 (SEQ ID NO.: 114), encoding a polynucleotide modification agent, contains a DNA recognition domain which was an array of 5 zinc-fingers, specifically designed to recognize 5′-GCGGACATGGAGGAC-3′ (SEQ ID NO.: 115), a 15-nucleotide sequence on the leading strand of human ApoE. Single-stranded oligonucleotide template, POP 33 (SEQ ID NO.: 11), and detection of genetic T-to-C conversion methods are indicated (equivalent to FIG. 6 described in Example 2). Detection of genetic T→C conversion after SSB-HbW-based gene edition was performed by droplet digital PCR (ddPCR). Relative positions of a correction ssODN (i.e., sequence modification polynucleotide) and position of an exemplary primer pair: POP 46 and POP 37 are also indicated in FIG. 6 described in Example 2. Illustrated in FIG. 59, positive T-to-C conversions are detected by ddPCR. Positive “C” droplets are displayed in upper panels, while wildtype “T” droplets in lower panel. Lane 1 shows that electroporation of a donor template, POP 33 (SEQ ID NO.: 11), by itself, does not enable genetic conversion in human B cells. Lane 3 shows no T-to-C conversions after electroporation both pT7 protein (SEQ ID NO.: 114) and POP 33, without pre-incubation of this protein and oligonucleotide. Lane 4 shows a negative control. Lane 2 shows successful genetic T-to-C conversion when a single-stranded correction template, POP 33, is introduced into B cells by electroporation, followed by adding pT7 protein to cell culture. This example demonstrates that a polynucleotide modification agent in form of a protein, is able to enter into a cell, migrate to its nucleus, and enable genetic conversion at a target site, when a modification template is present (in this example such a modification template was pre-delivered by electroporation).

pT7 protein sequence
(SEQ ID NO.: 114)
MDYKDDDDKAAMAERPFQCRICMRNFSDRSNLTRHIRTHTGEKPFACDI
CGRKFARSDNLTRHTKIHTGSQKPFQCRICMRNFSRSDSLSQHIRTHTG
EKPFACDICGRKFADRSNLTRHTKIHTGSQKPFQCRICMRNFSRSDTLT
RHIRTHTGLRGSNSGDPRRHSLGGSRKPDLIAYKNFDLLVIELKP
pT7 DNA binding sequence for ApoE:
(SEQ ID NO: 115)
GCGGACATGGAGGAC

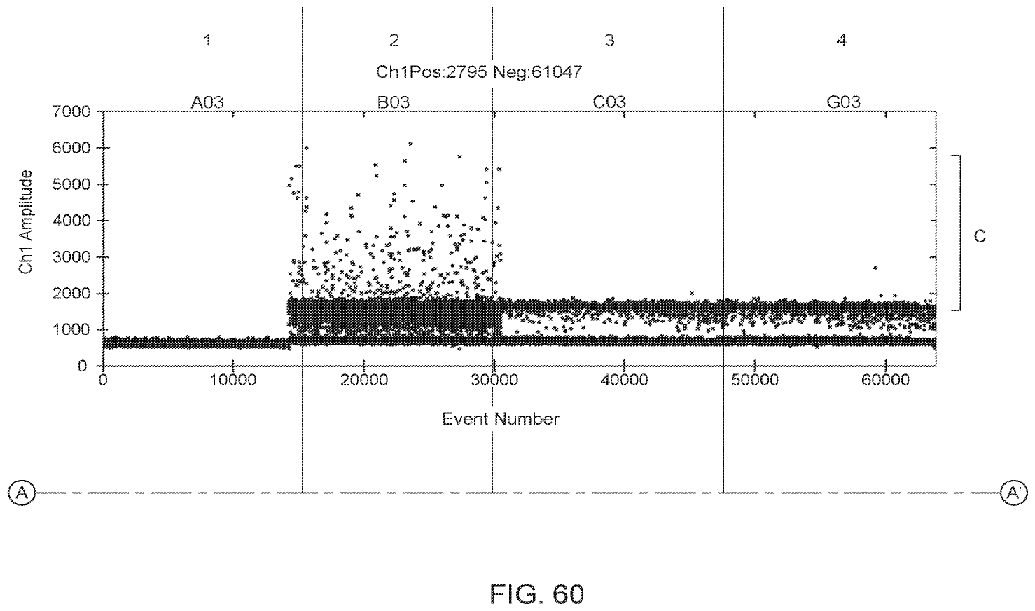

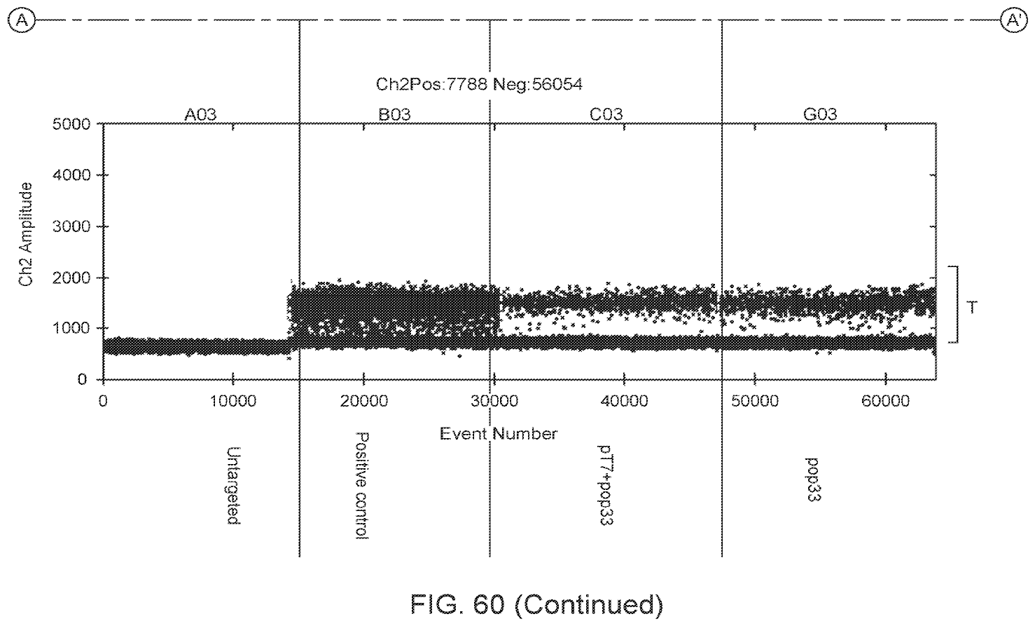

FIG. 60 illustrates an example that shows that single-stranded oligonucleotide is not able to go into cells by itself, when it is added in cell culture. As detection for genetic conversion ddPCR is used. Positive “C” droplets are displayed in upper panels, while wildtype “T” droplets are shown in the lower panel. Lane 1 shows a negative control with no positive “C” droplets detected. Lane 2 shows a positive control of a successful “T-to-C conversion using plasmid DNA and correction template in human B cells. Lane 3 illustrates that directly adding pT7 protein and POP 33 modification oligonucleotide template to a cell culture does not result in successful genetic conversion. Lane 4 shows that adding POP 33 single stranded oligonucleotide template does not induce gene editing at its target locus in human B cells.

Results depicted in FIG. 59 demonstrated that a protein-based polynucleotide modification agent for SSB-HbW-based gene editing is able to enter into cells. Results shown in FIG. 59 and FIG. 60 support that addition of a SSB-HbW modification donor template to cells do not result in genetic conversion. Without being bound to any particular theory, this disclosure contemplates that, in order to achieve genetic conversion a at target locus in cells via protein-based delivery, it will be useful for a single stranded oligonucleotide correction template to be able to enter cells as part of a protein-oligonucleotide complex or mixture. In particular, it will be useful not to have make use of electroporation or other transfection methods.

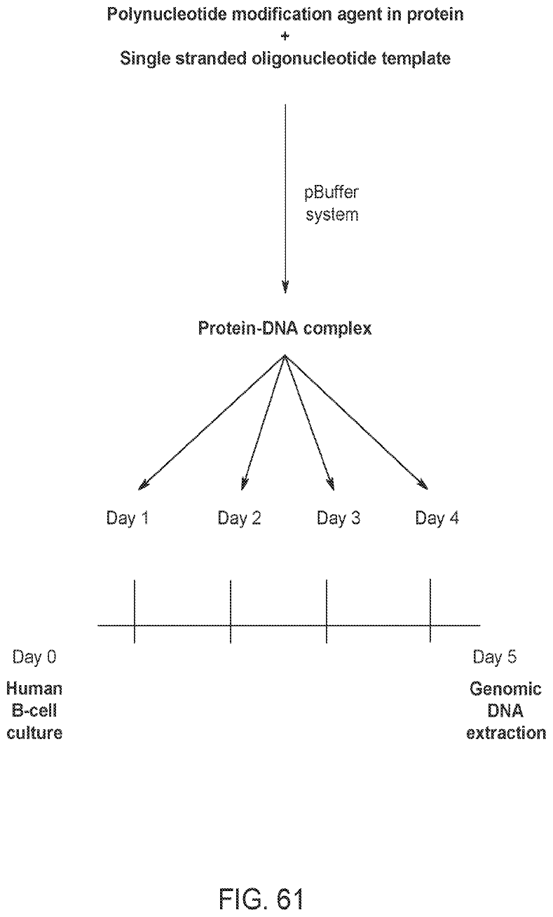

FIG. 61 depicts a scheme for an approach used in this example for protein-based delivery for SSB-HbW-based gene editing. Prior to addition to cells for genomic conversion at target loci, a (partially) purified protein-based polynucleotide modification agent and a single stranded oligonucleotide are incubated in a buffer to form a complex. Following this incubation step, such a protein-oligo complex or mixture can be added to cells, either as a single dose or as multiple doses. In this example, genomic DNA is extracted for genomic editing validation after four repeated dosings to human B cells.

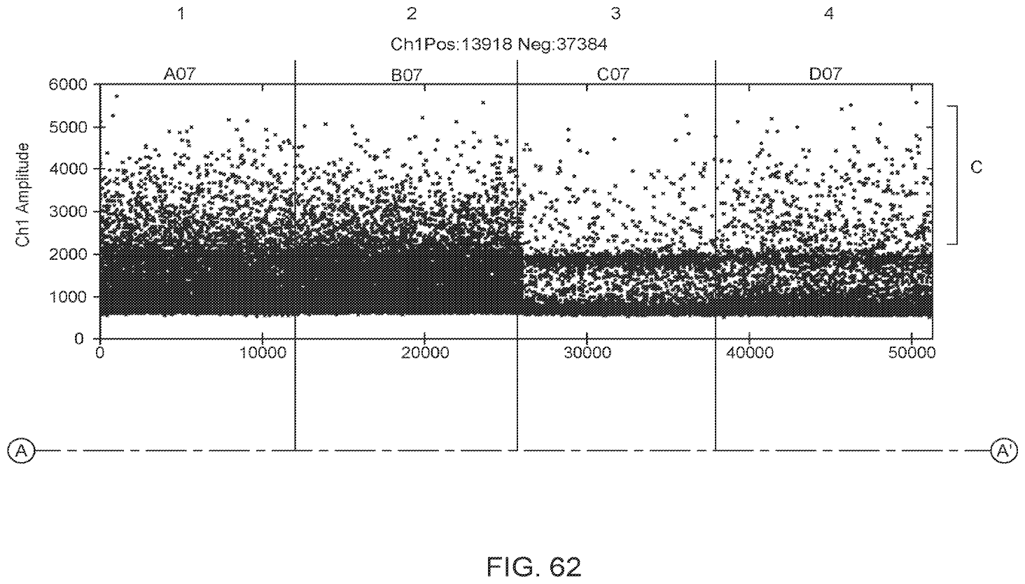

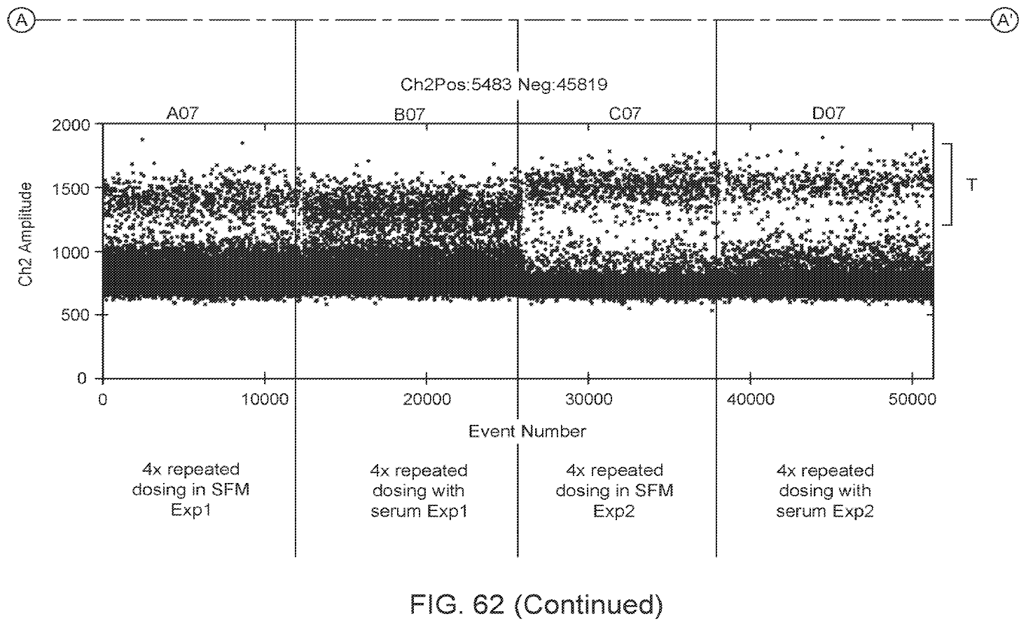

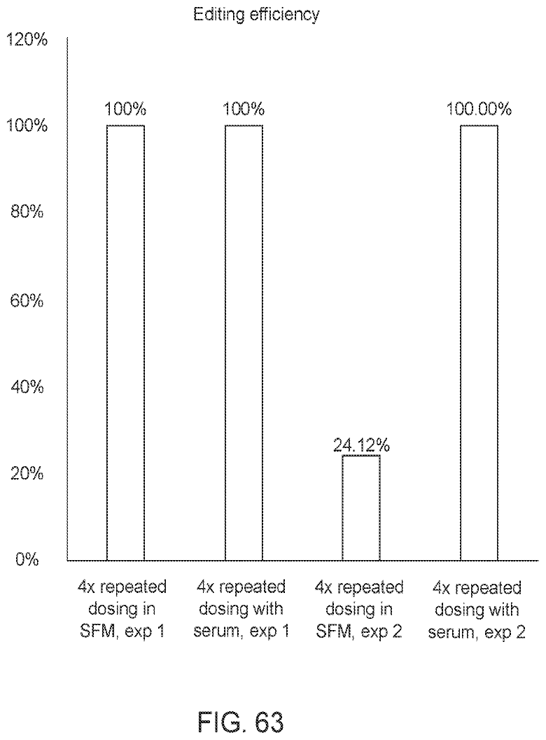

FIG. 62 shows gene editing results from a protein-based delivery system. Herein, a protein-based polynucleotide modification agent is derived from DNA plasmid template pb 121 (SEQ ID NO.: 111 and 112), wherein a 9-zinc finger domain is designed to recognize a DNA sequence of 5′-GCGGCCGCCTGGTGCAGTACCGCGGCG-3′ (SEQ ID NO.: 22), a 27-nucleotide sequence on the leading strand of human ApoE. A detection method is illustrated in FIG. 6 described in Example 2. Results of successful positive T-to-C conversion after four repeated dosings of protein-oligonucleotides in the presence of serum (Lane 1 and 3) and serum-free conditions (Lane 2 and 4) are shown. Results were obtained in two independent experiments. FIG. 62 shows genetic “T-to-C” conversion frequencies as measured by ddPCR after HbW-based gene editing. It shows editing frequencies corresponding to cellular “T-to-C” conversion percentages, defined as a percentage of “C” droplet events divided by the sum of “T” and “C” droplet events. Here, in this HbW-based gene editing example almost 100% genetic conversion frequencies was achieved, in two independent conditions with serum and one experiment using serum free conditions. In one experiment, using serum added to this cell culture, a 24.12% editing efficiency was achieved, as shown in FIG. 63.

In this present Example, next generation sequencing was also performed to determine, in more detail, gene conversion frequencies and patterns and also to detect potential generation of insertions, deletions, and unintended single nucleotide polymorphisms after SSB-HbW-based gene editing. Next generation sequencing of both targeted pooled B cells and untransfected B cells as control was performed. Genomic DNA was isolated and used as a template on which a 175-bp PCR amplicon surrounding ApoE codon 112 was generated by using a primer set of POP 46 and POP 37. Amplified PCR products from targeted B cells via protein-based delivery was analyzed for indels and SNPs on an Illumina next generation sequencing platform (GENEWIZ, South Plainfield, NJ).

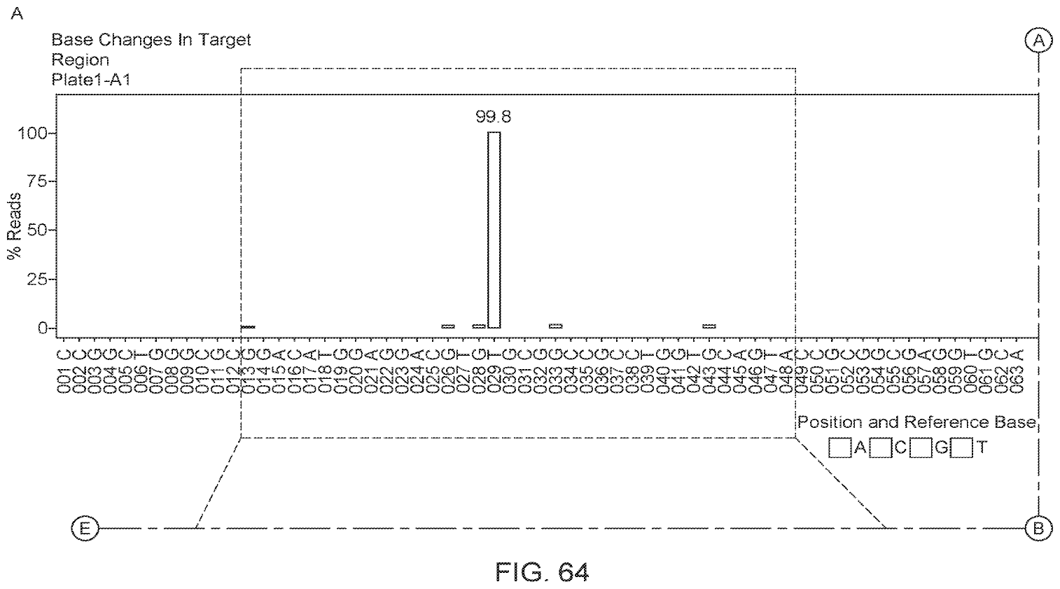

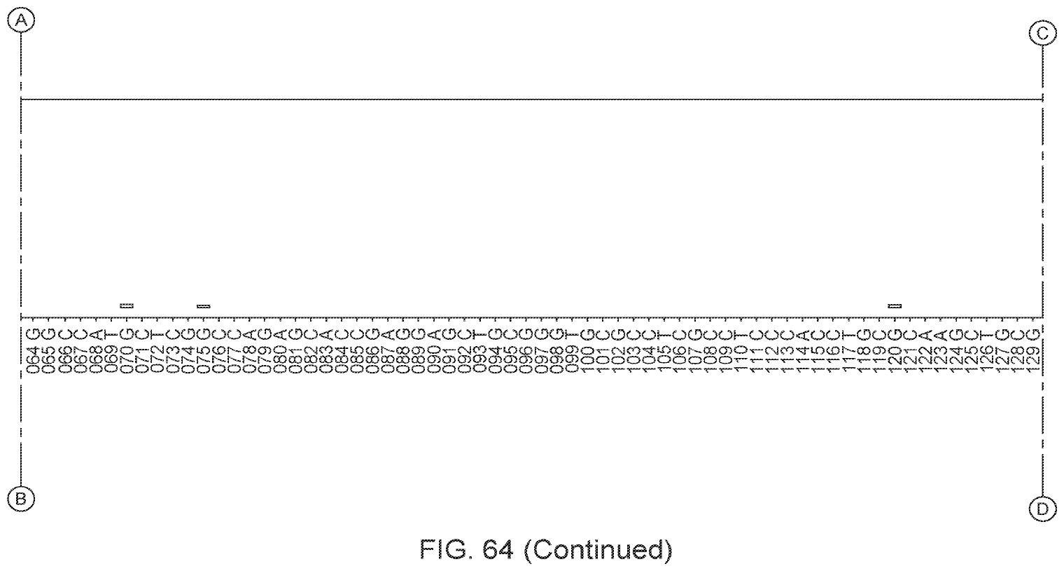

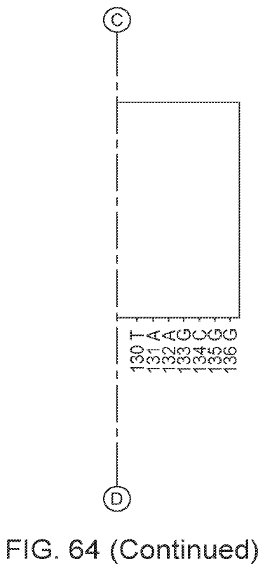

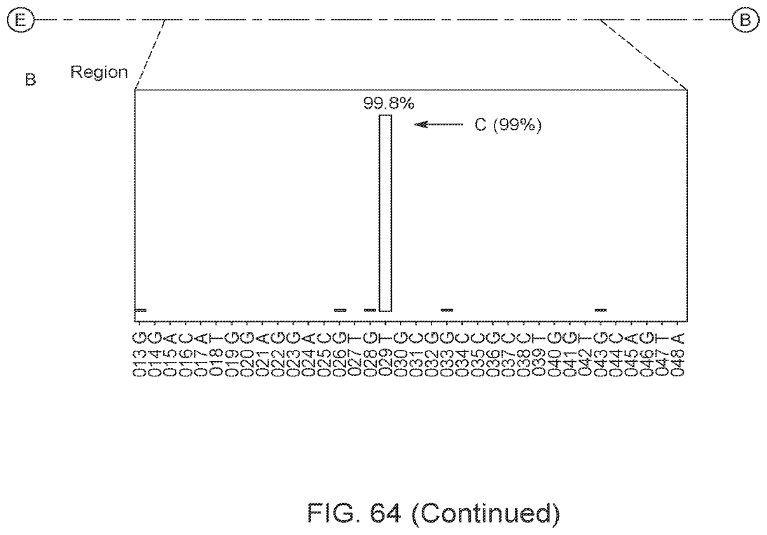

FIG. 64 shows confirmation of detection of single nucleotide T→C conversion at this target site as well as single nucleotide polymorphisms (SNPs) analysis within a target region surrounding codon 112 of this ApoE locus using 4 repeated dosing in serum free condition in human B cells. Panel A shows overall views of SNPs analysis at these target sites obtained with untargeted B cells, and targeted pooled B cells. Bar graphs plot frequencies of SNPs at each nucleotide position in this 175 bp PCR amplification region. Panel B is a magnified view of a portion close to this gene conversion site. In this example cells treated four times with protein derived from plasmid DNA template of with pb 121 (SEQ ID NO.: 111 and 112) and a correction template showed a T-to-C conversion at this expected nucleotide position with a frequency of 99.7%.

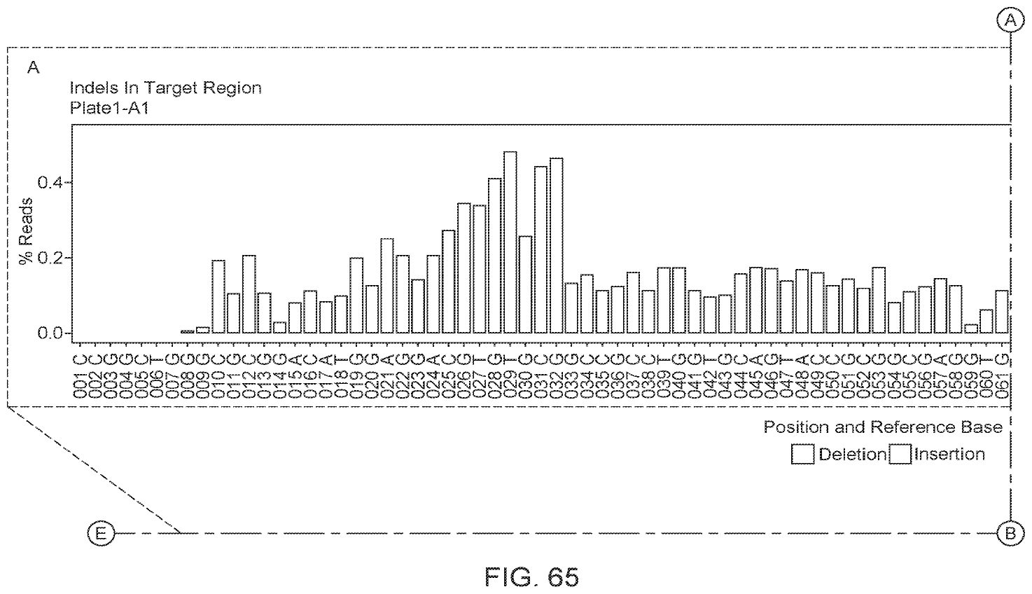

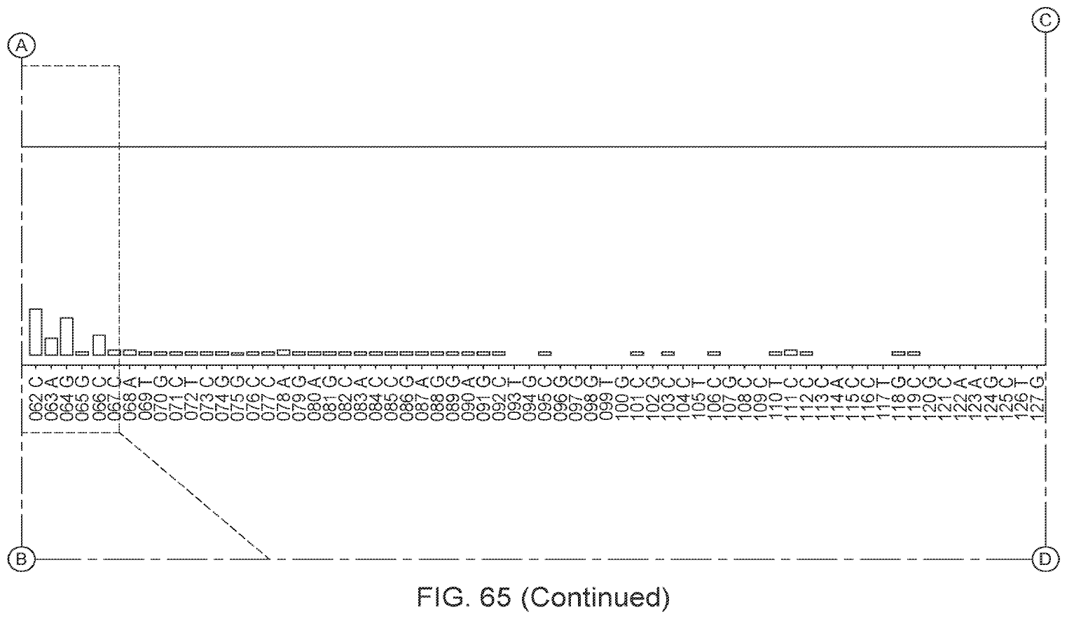

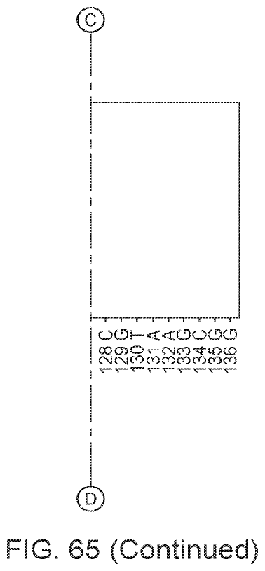

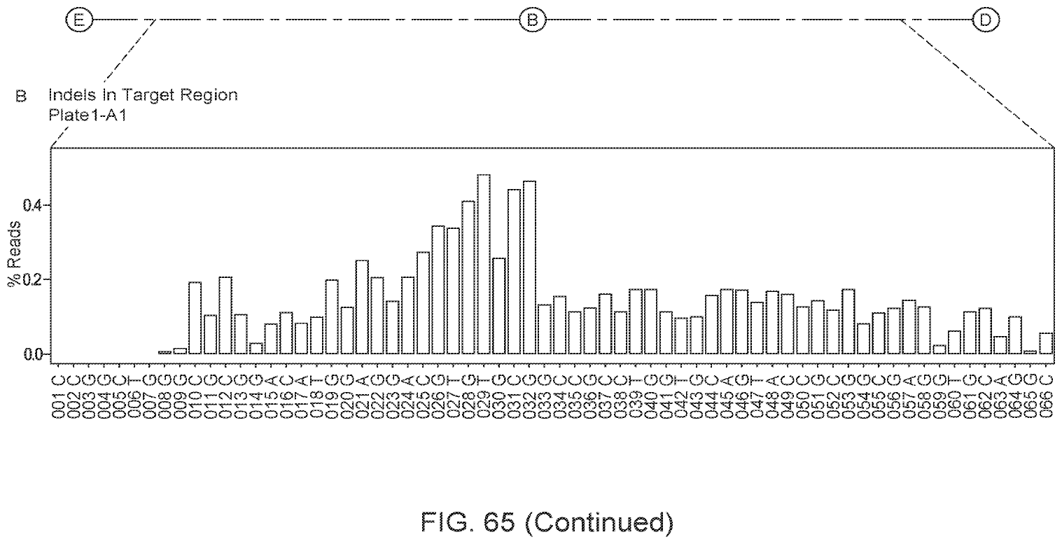

FIG. 65 shows insertion and deletion analysis around codon 112 of ApoE in an example using protein-based delivery for SSB-HbW-based gene editing, displayed as a frequency plot of insertions and deletions analysis for targeted pooled B cells. Bar graphs plot frequencies of insertions and deletions at each nucleotide position of this 175 bp PCR amplification region. Panel A shows overall views of insertion and deletions analysis at this target site obtained with edited B cells; while Panel B is a magnified view of a portion close to y-axis where shows frequencies of insertions and deletions. This indels analysis showed, in general, a very low frequency (<0.5%) of insertions and/or deletions in each given position in the region of 175 bp PCR amplification.

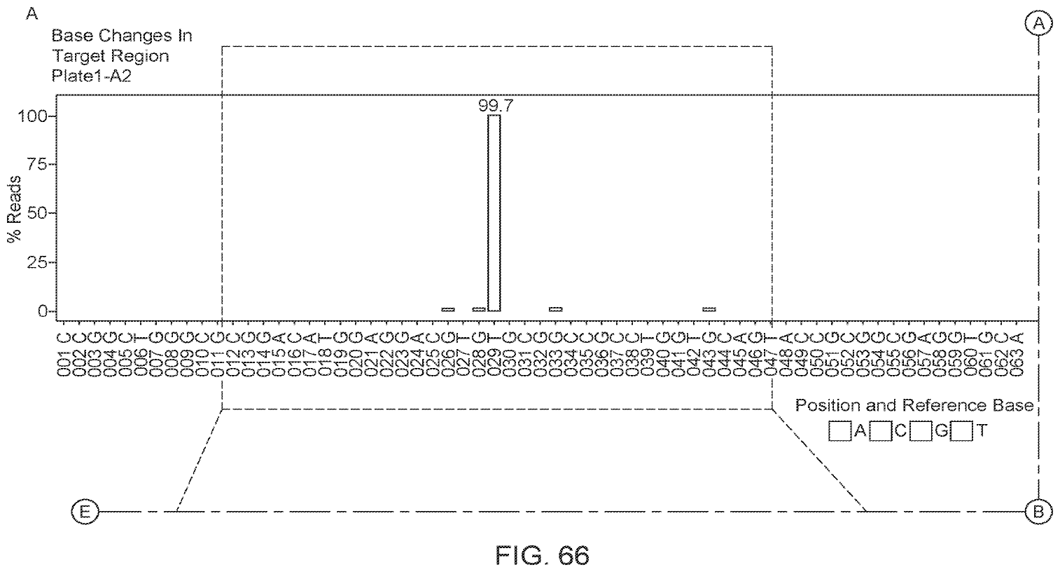

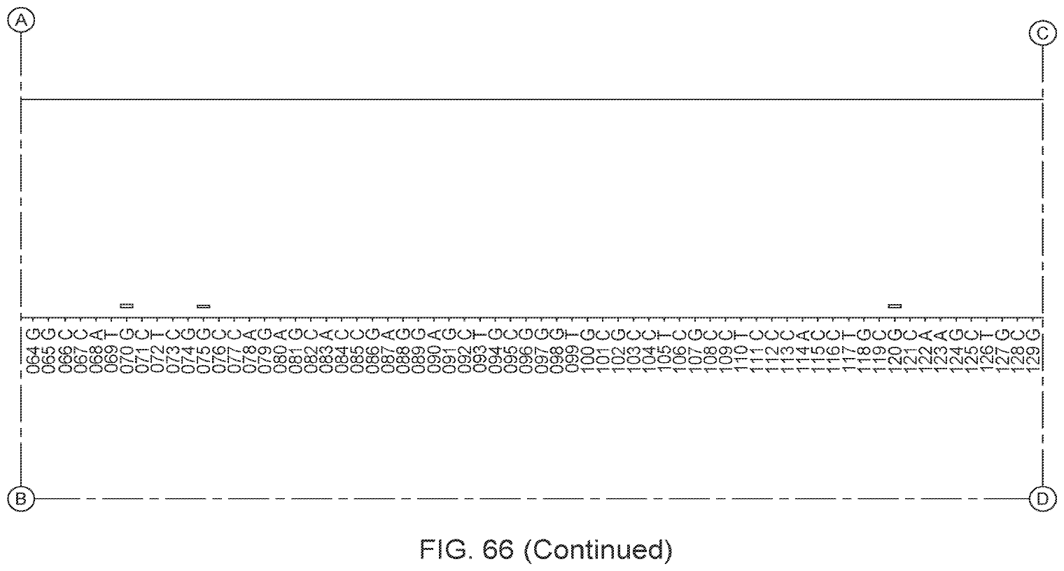

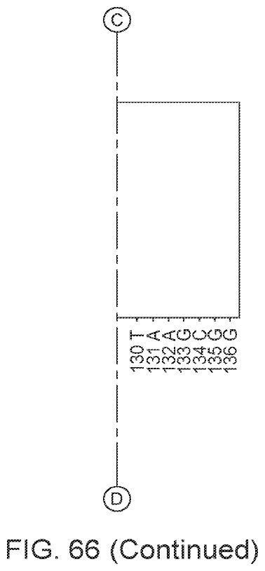

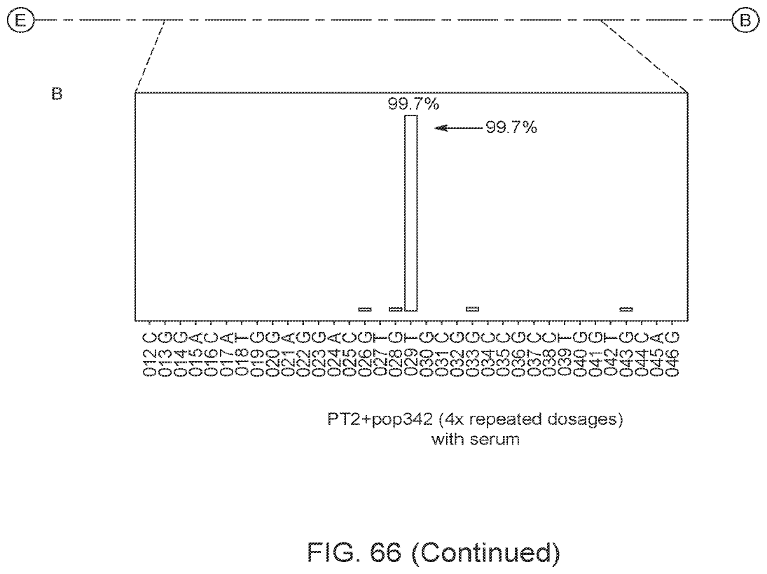

FIG. 66 shows confirmation of detection of single nucleotide T→C conversion at this target site as well as single nucleotide polymorphisms (SNPs) analysis within a target region of surrounding codon 112 of this ApoE locus using 4 repeated dosing in presence of serum in human B cells. Panel A shows overall views of SNPs analysis at these target sites obtained with untargeted B cells, and targeted pooled B cells. Bar graphs plot frequencies of SNPs at each nucleotide position in this 175 bp PCR amplification region. Panel B is a magnified view of a portion close to this gene repair site. In this example cells treated four times with protein derived from plasmid DNA template of with pb 121 pb 121 (SEQ ID NO.: PF-43 and PF-44) and a correction template showed a T-to-C conversion at this expected nucleotide position with a frequency of 99.8%.

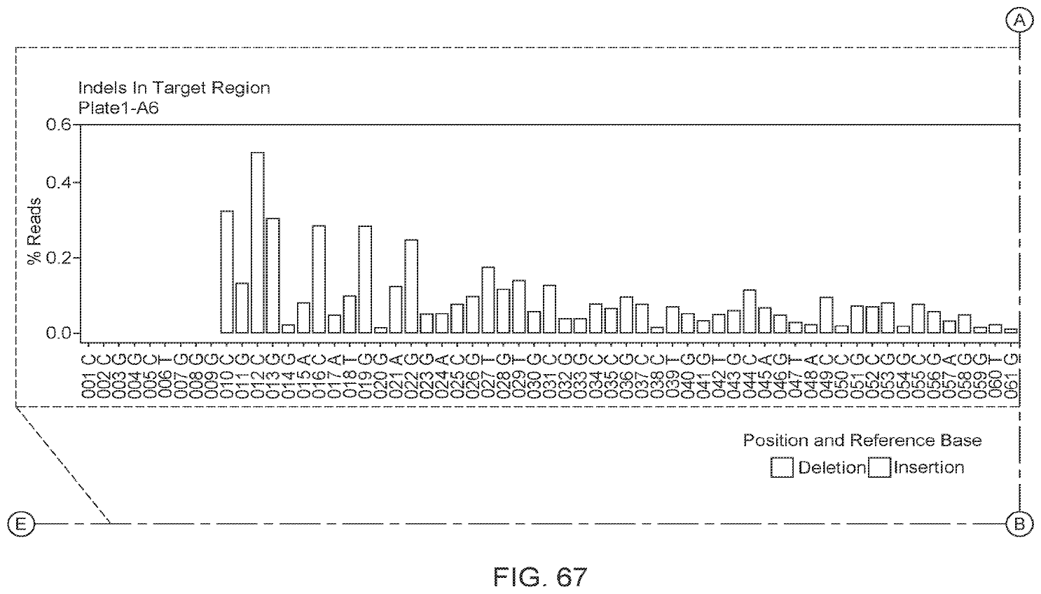

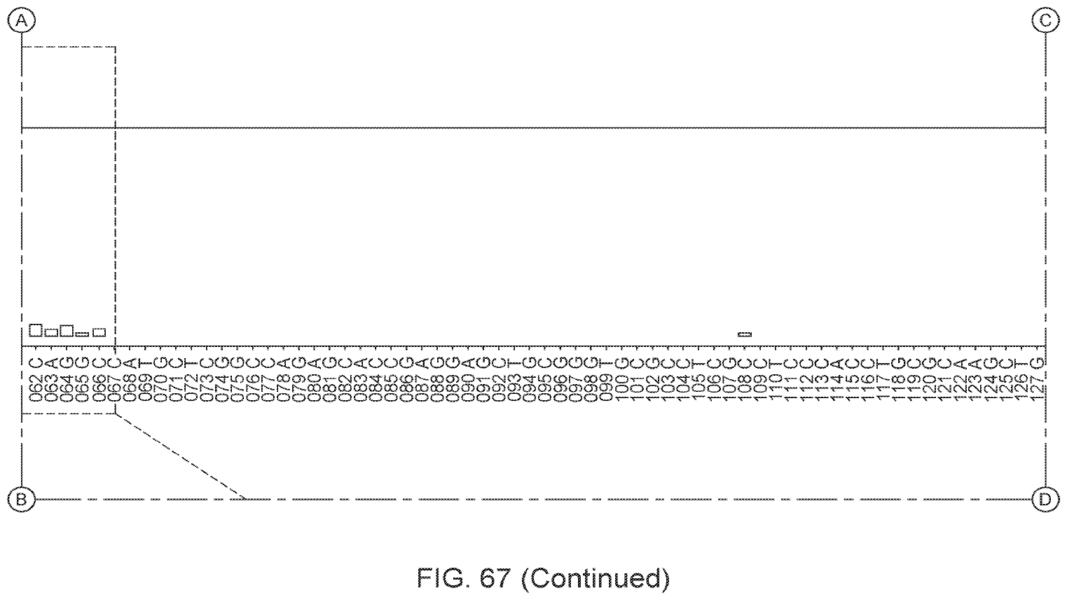

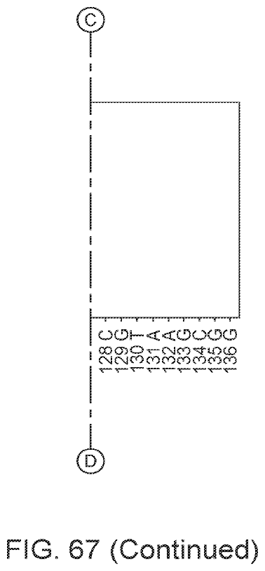

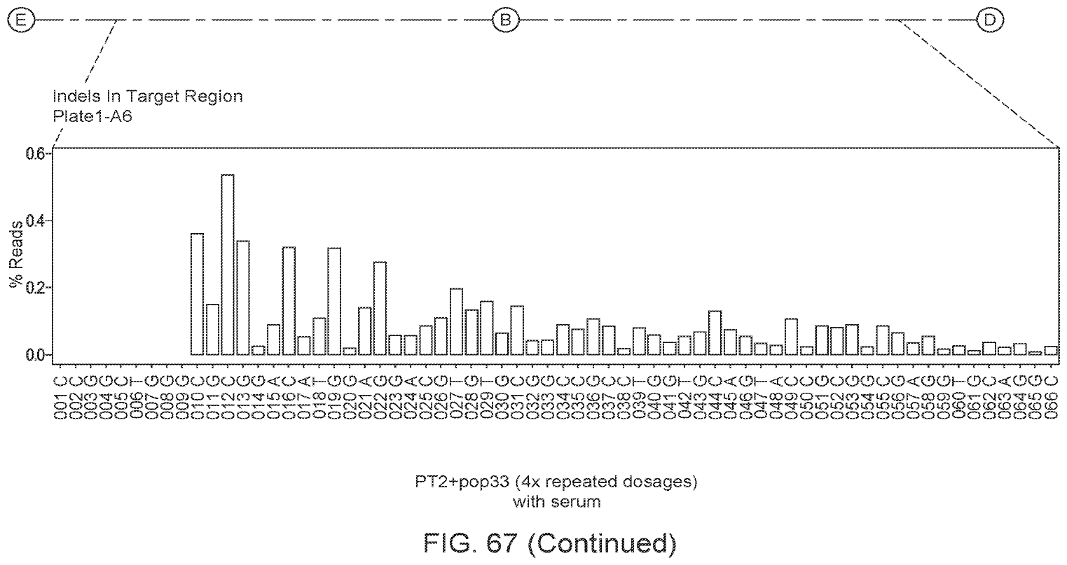

FIG. 67 shows insertion and deletion analysis around codon 112 of ApoE in an example using protein-based delivery for SSB-HbW-based gene editing, displayed as a frequency plot of insertions and deletions analysis for targeted pooled B cells. Bar graphs plot frequencies of insertions and deletions at each nucleotide position of this 175 bp PCR amplification region. Panel A shows overall views of insertion and deletions analysis at this target site obtained with edited B cells; while Panel B is a magnified view of the portion close to y-axis which shows frequencies of insertions and deletions. This indels analysis showed, in general, a very low frequency (<0.6%) of insertions and/or deletions in each given position in the region of 175 bp PCR amplification. These results confirm that a protein-based polynucleotide modification agent in combination with a sequence modification polynucleotide can successfully target and edit a sequence in an endogenous mammalian gene in mammalian cells to successfully modify a disease-causing genotype using protein-based delivery. Notably, this example also demonstrates that provided technologies are capable of genetic modification with low levels of insertions and deletions. Thus, technologies of the present disclosure are capable of targeted gene conversion without potentially detrimental generation of insertions, deletions and/or undesired single nucleotide polymorphisms.

### Example 12: Modification of an Endogenous Genomic Target: Exon 51 of Dystrophin Gene by SSB-HbW-Based Gene Editing Through Protein Delivery

In this example, exon 51 of human dystrophin (DMD), was targeted and edited using a SSB-HbW approach to change a dystrophin reading frame via two-nucleotide insertion by SSB-HbW, using specifically designed SSB-HbW molecules and a single stranded oligonucleotide template (i.e., a sequence modification polynucleotide). Duchenne muscular dystrophy (DMD) is an X-linked disease caused by mutations in the dystrophin and presents, clinically, throughout the entire body, a progressive muscle wasting disease. One commonly occurring DMD-causing mutation is a deletion of exon 50 of the human dystrophin, which causes a frame shift and distorts dystrophin translation such that little to no functional dystrophin protein is produced. One known manner in which any detrimental impact of such mutations (e.g., deletion of exon 50) can be overcome is by skipping exon 51 using antisense oligonucleotides to “mask” exon 51, thereby restoring the dystrophin reading frame and resulting in functional (albeit shorter) dystrophin protein which results in a milder clinical phenotype as compared to DMD; however as masking techniques do not change the underlying genetic code, they still requires continuous treatment to mask genetic mutations in order to make dystrophin (Falzarano et al., Molecules. 2015 October; 20(10):18168-18184, which is herein incorporated by reference in its entirety). As described in the present Example, a SSB-HbW system with a specifically-designed SSB-HbW molecule and sequence modification polynucleotide can successfully edit the dystrophin gene by inserting two nucleotides into exon 51 such that a normal reading frame is achieved.

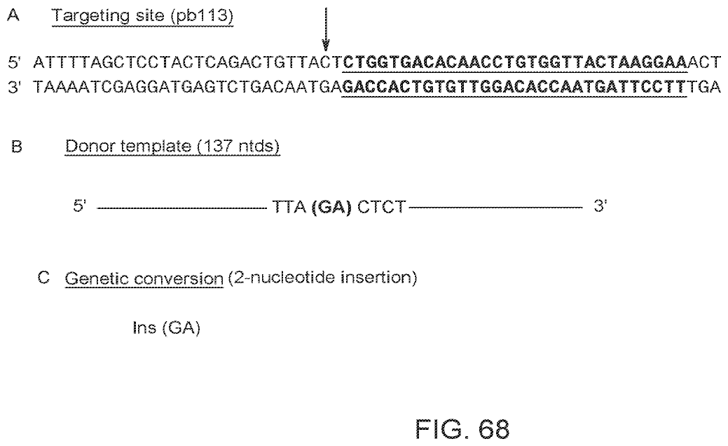

FIG. 68 is a schematic illustrating an editing strategy used in this Example. Human B cells were used and a polynucleotide modification agent, encoded on plasmid pb113 (full length DNA (SEQ ID NO.: 116); cDNA (SEQ ID NO.: 117); its amino acid sequence (SEQ ID NO.: 118)), has a DNA recognition domain which was an array of 10 zinc-fingers, specifically designed to recognize 5′-CTGGTGACACAACCTGTGGTTACTAAGGAA-3′ (SEQ ID NO.: 119), a 30-nucleotide sequence on the leading strand of human dystrophin, as underlined. Exemplary sequences are provided in Table 12 below. FIG. 68 panel B shows a 137-nucleotide single stranded DNA oligonucleotide with a desired TTACTCT→TTAGACTCT substitution roughly located in the middle of the length of this oligonucleotide served as the sequence modification polynucleotide. A two-nucleotide sequence “GA” was inserted between “a” and “c” of sequence “TTacTCT” in exon 51 of a dystrophin gene and resulted in an altered reading frame in exons downstream of the insertion The sequence of the sequence modification polynucleotide used in this Example is provided below with the “GA” insertion indicated in “ins(GA)”, as showed in FIG. 68 panel C.

(SEQ ID NO.: 13)
5′TAATTTTTCTTTTTCTTCTTTTTTCCTTTTTGCAAAAACCCAAAATA
TTTTAGCTCCTACTCAGACTGTTAGACTCTGGTGACACAACCTGTGGTT
ACTAAGGAAACTGCCATCTCCAAACTAGAAATGCCATCTTCC 3′

TABLE 12
Exemplary polynucleotide modification agents targeting DMD

| SEQ | | AA/ | |
|---|---|---|---|
| ID | | DNA/ | |
| NO: | Feature | CDNA | Sequence |
| 116 | pb113 | DNA | GACTCTTCGCGATGTACGGGCCAGATATACGCGTTGACATTGATTATTGACTAGTT |
| | | | ATTAATAGTAATCAATTACGGGGTCATTAGTTCATAGCCCATATATGGAGTTCCGC |
| | | | GTTACATAACTTACGGTAAATGGCCCGCCTGGCTGACCGCCCAACGACCCCCGCCC |
| | | | ATTGACGTCAATAATGACGTATGTTCCCATAGTAACGCCAATAGGGACTTTCCATT |
| | | | GACGTCAATGGGTGGACTATTTACGGTAAACTGCCCACTTGGCAGTACATCAAGTG |
| | | | TATCATATGCCAAGTACGCCCCCTATTGACGTCAATGACGGTAAATGGCCCGCCTG |
| | | | GCATTATGCCCAGTACATGACCTTATGGGACTTTCCTACTTGGCAGTACATCTACG |
| | | | TATTAGTCATCGCTATTACCATGGTGATGCGGTTTTGGCAGTACATCAATGGGCGT |
| | | | GGATAGCGGTTTGACTCACGGGGATTTCCAAGTCTCCACCCCATTGACGTCAATGG |
| | | | GAGTTTGTTTTGGCACCAAAATCAACGGGACTTTCCAAAATGTCGTAACAACTCCG |
| | | | CCCCATTGACGCAAATGGGCGGTAGGCGTGTACGGTGGGAGGTCTATATAAGCAGA |
| | | | GCTCTCTGGCTAACTAGAGAACCCACTGCTTACTGGCTTATCGAAATTAATACGAC |
| | | | TCACTATAGGGAGACCCAAGCTGGCTAGCGTTTAAACTTAAGCTTGGGGTACCGGC |
| | | | GGCGATGGCGGCGATGGCCGAGCGGCCCTTCGCCTGCGACATTTGTGGGAGAAAAT |
| | | | TTGCTGATCAGTCCGGCAACCTGACCCGGCATACCAAGATCCACACCGGCTCTCAG |
| | | | AAACCATTCCAGTGCAGGATCTGTATGCGCAACTTTTCTCGGTCCGACAACCTGTC |
| | | | CCAGCACATCAGAACCCATACAGGCGAAAAGCCTTTTGCTTGCGACATTTGTGGCA |
| | | | GGAAATTTGCTACCTCCGGCGACCTGTCCCAGCACACTAAGATCCATACTGGGTCA |
| | | | CAGAAACCTTTCCAGTGCCGCATTTGTATGCGGAATTTTTCCACCTCCGGCTCCCT |
| | | | GACCCGGCATATCCGCACTCACACCGGAGAGAAGCCCTTTGCATGCGACATTTGTG |
| | | | GACGGAAATTTGCTCGGTCCGACGCCCTGACCCGGCATACCAAGATTCACACTGGG |
| | | | TCTCAGAAACCTTTCCAGTGCAGGATTTGTATGAGAAATTTTTCCACCTCCGGCGA |
| | | | CCTGTCCGAGCACATCAGAACCCATACAGGCGAAAAGCCTTTTGCTTGCGACATTT |
| | | | GTGGCAGGAAATTTGCTCAGTCCGGCAACCTGTCCGAGCACACTAAGATCCATACT |
| | | | GGGTCACAGAAACCTTTCCAGTGCCGCATTTGTATGCGGAATTTTTCCCAGTCCGG |
| | | | CGACCTGTCCCAGCACATCAGAACCCATACAGGCGAAAAGCCTTTTGCTTGCGACA |
| | | | TTTGTGGCAGGAAATTTGCTCGGTCCTCCGCCCTGACCCGGCACACTAAGATCCAT |
| | | | ACTGGGTCACAGAAACCTTTCCAGTGCCGCATTTGTATGCGGAATTTTTCCCGGTC |
| | | | CGACGCCCTGTCCGAGCACATCAGAACACATACTGGGGAGAAGATCATCGCCCACT |
| | | | TCCTGATCCAGCAGTACCTGAAGGAGGACTACTCCTTCACCGCCTACGCCACCATC |
| | | | TCCTACCTGAAGATCGGCTGAGCGGCCGCTCGAGTCTAGAGGGCCCGTTTAAACCC |
| | | | GCTGATCAGCCTCGACTGTGCCTTCTAGTTGCCAGCCATCTGTTGTTTGCCCCTCC |
| | | | CCCGTGCCTTCCTTGACCCTGGAAGGTGCCACTCCCACTGTCCTTTCCTAATAAAA |
| | | | TGAGGAAATTGCATCGCATTGTCTGAGTAGGTGTCATTCTATTCTGGGGGGTGGGG |
| | | | TGGGGCAGGACAGCAAGGGGGAGGATTGGGAAGACAATAGCAGGCATGCTGGGGAT |
| | | | GCGGTGGGCTCTATGGCTTCTACTGGGCGGTTTTATGGACAGCAAGCGAACCGGAA |
| | | | TTGCCAGCTGGGGCGCCCTCTGGTAAGGTTGGGAAGCCCTGCAAAGTAAACTGGAT |
| | | | GGCTTTCTCGCCGCCAAGGATCTGATGGCGCAGGGGATCAAGCTCTGATCAAGAGA |
| | | | CAGGATGAGGATCGTTTCGCATGATTGAACAAGATGGATTGCACGCAGGTTCTCCG |
| | | | GCCGCTTGGGTGGAGAGGCTATTCGGCTATGACTGGGCACAACAGACAATCGGCTG |
| | | | CTCTGATGCCGCCGTGTTCCGGCTGTCAGCGCAGGGGCGCCCGGTTCTTTTTGTCA |
| | | | AGACCGACCTGTCCGGTGCCCTGAATGAACTGCAAGACGAGGCAGCGCGGCTATCG |
| | | | TGGCTGGCCACGACGGGCGTTCCTTGCGCAGCTGTGCTCGACGTTGTCACTGAAGC |
| | | | GGGAAGGGACTGGCTGCTATTGGGCGAAGTGCCGGGGCAGGATCTCCTGTCATCTC |
| | | | ACCTTGCTCCTGCCGAGAAAGTATCCATCATGGCTGATGCAATGCGGCGGCTGCAT |
| | | | ACGCTTGATCCGGCTACCTGCCCATTCGACCACCAAGCGAAACATCGCATCGAGCG |
| | | | AGCACGTACTCGGATGGAAGCCGGTCTTGTCGATCAGGATGATCTGGACGAAGAGC |
| | | | ATCAGGGGCTCGCGCCAGCCGAACTGTTCGCCAGGCTCAAGGCGAGCATGCCCGAC |
| | | | GGCGAGGATCTCGTCGTGACCCATGGCGATGCCTGCTTGCCGAATATCATGGTGGA |
| | | | AAATGGCCGCTTTTCTGGATTCATCGACTGTGGCCGGCTGGGTGTGGCGGACCGCT |
| | | | ATCAGGACATAGCGTTGGCTACCCGTGATATTGCTGAAGAGCTTGGCGGCGAATGG |
| | | | GCTGACCGCTTCCTCGTGCTTTACGGTATCGCCGCTCCCGATTCGCAGCGCATCGC |
| | | | CTTCTATCGCCTTCTTGACGAGTTCTTCTGAATTATTAACGCTTACAATTTCCTGA |
| | | | TGCGGTATTTTCTCCTTACGCATCTGTGCGGTATTTCACACCGCATACAGGTGGCA |
| | | | CTTTTCGGGGAAATGTGCGCGGAACCCCTATTTGTTTATTTTTCTAAATACATTCA |
| | | | AATATGTATCCGCTCATGAGACAATAACCCTGATAAATGCTTCAATAATAGCACGT |
| | | | GCTAAAACTTCATTTTTAATTTAAAAGGATCTAGGTGAAGATCCTTTTTGATAATC |
| | | | TCATGACCAAAATCCCTTAACGTGAGTTTTCGTTCCACTGAGCGTCAGACCCCGTA |
| | | | GAAAAGATCAAAGGATCTTCTTGAGATCCTTTTTTTCTGCGCGTAATCTGCTGCTT |
| | | | GCAAACAAAAAAACCACCGCTACCAGCGGTGGTTTGTTTGCCGGATCAAGAGCTAC |
| | | | CAACTCTTTTTCCGAAGGTAACTGGCTTCAGCAGAGCGCAGATACCAAATACTGTC |
| | | | CTTCTAGTGTAGCCGTAGTTAGGCCACCACTTCAAGAACTCTGTAGCACCGCCTAC |
| | | | ATACCTCGCTCTGCTAATCCTGTTACCAGTGGCTGCTGCCAGTGGCGATAAGTCGT |
| | | | GTCTTACCGGGTTGGACTCAAGACGATAGTTACCGGATAAGGCGCAGCGGTCGGGC |
| | | | TGAACGGGGGGTTCGTGCACACAGCCCAGCTTGGAGCGAACGACCTACACCGAACT |
| | | | GAGATACCTACAGCGTGAGCTATGAGAAAGCGCCACGCTTCCCGAAGGGAGAAAGG |
| | | | CGGACAGGTATCCGGTAAGCGGCAGGGTCGGAACAGGAGAGCGCACGAGGGAGCTT |
| | | | CCAGGGGGAAACGCCTGGTATCTTTATAGTCCTGTCGGGTTTCGCCACCTCTGACT |
| | | | TGAGCGTCGATTTTTGTGATGCTCGTCAGGGGGGCGGAGCCTATGGAAAAACGCCA |
| | | | GCAACGCGGCCTTTTTACGGTTCCTGGGCTTTTGCTGGCCTTTTGCTCACATGTTC |
| | | | TT |
| 117 | pb113 | CDNA | ATGGCGGCGATGGCCGAGCGGCCCTTCGCCTGCGACATTTGTGGGAGAAAATTTGC |
| | | | TGATCAGTCCGGCAACCTGACCCGGCATACCAAGATCCACACCGGCTCTCAGAAAC |
| | | | CATTCCAGTGCAGGATCTGTATGCGCAACTTTTCTCGGTCCGACAACCTGTCCCAG |
| | | | CACATCAGAACCCATACAGGCGAAAAGCCTTTTGCTTGCGACATTTGTGGCAGGAA |
| | | | ATTTGCTACCTCCGGCGACCTGTCCCAGCACACTAAGATCCATACTGGGTCACAGA |
| | | | AACCTTTCCAGTGCCGCATTTGTATGCGGAATTTTTCCACCTCCGGCTCCCTGACC |
| | | | CGGCATATCCGCACTCACACCGGAGAGAAGCCCTTTGCATGCGACATTTGTGGACG |
| | | | GAAATTTGCTCGGTCCGACGCCCTGACCCGGCATACCAAGATTCACACTGGGTCTC |
| | | | AGAAACCTTTCCAGTGCAGGATTTGTATGAGAAATTTTTCCACCTCCGGCGACCTG |
| | | | TCCGAGCACATCAGAACCCATACAGGCGAAAAGCCTTTTGCTTGCGACATTTGTGG |
| | | | CAGGAAATTTGCTCAGTCCGGCAACCTGTCCGAGCACACTAAGATCCATACTGGGT |
| | | | CACAGAAACCTTTCCAGTGCCGCATTTGTATGCGGAATTTTTCCCAGTCCGGCGAC |
| | | | CTGTCCCAGCACATCAGAACCCATACAGGCGAAAAGCCTTTTGCTTGCGACATTTG |
| | | | TGGCAGGAAATTTGCTCGGTCCTCCGCCCTGACCCGGCACACTAAGATCCATACTG |
| | | | GGTCACAGAAACCTTTCCAGTGCCGCATTTGTATGCGGAATTTTTCCCGGTCCGAC |
| | | | GCCCTGTCCGAGCACATCAGAACACATACTGGGGAGAAGATCATCGCCCACTTCCT |
| | | | GATCCAGCAGTACCTGAAGGAGGACTACTCCTTCACCGCCTACGCCACCATCTCCT |
| | | | ACCTGAAGATCGGCTGA |
| 118 | pb113 | AA | MAAMAERPFACDICGRKFADQSGNLTRHTKIHTGSQKPFQCRICMRNFSRSDNLSQ |
| | | | HIRTHTGEKPFACDICGRKFATSGDLSQHTKIHTGSQKPFQCRICMRNFSTSGSLT |
| | | | RHIRTHTGEKPFACDICGRKFARSDALTRHTKIHTGSQKPFQCRICMRNESTSGDL |
| | | | SEHIRTHTGEKPFACDICGRKFAQSGNLSEHTKIHTGSQKPFQCRICMRNFSQSGD |
| | | | LSQHIRTHTGEKPFACDICGRKFARSSALTRHTKIHTGSQKPFQCRICMRNFSRSD |
| | | | ALSEHIRTHTGEKIIAHFLIQQYLKEDYSFTAYATISYLKIG* |

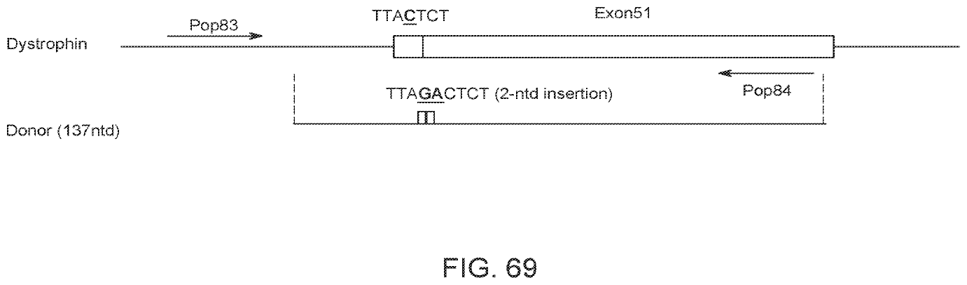

Detection of a genetic “GA” insertion after SSB-HbW-based gene editing was performed by Sanger sequencing and next generation sequencing. Relative positions of the sequence modification polynucleotide and position of a common primer pair (POP83, POP84, SEQ ID NO.: 120 and 121) are also indicated in FIG. 69. One common primer, POP83 was located outside the sequence modification polynucleotide sequence, while POP84, located inside. PCR amplification is used a pair of primer of POP83 and POP84. The protein of polynucleotide modification agent for SSB-HbW-based gene editing is derived from the DNA plasmid template of pb113. Protein-correction template complex is form in the condition described in Example 11 above. Three repeated treatments of protein-donor template are applied to human B cells in the presence of serum. Five days after the last treatment, cellular genomic DNA is extracted for genomic analysis to confirm genome editing effect through this protein-based delivery route.

```
POP83 dystrophin forward primer has a sequence of
  (SEQ ID NO.: 120)
TTGGCTCTTTAGCTTGTGTTTC.
POP84 dystrophin reverse primer has a sequence of
  (SEQ ID NO.: 121)
GGCATTTCTAGTTTGGAGATGG.
```

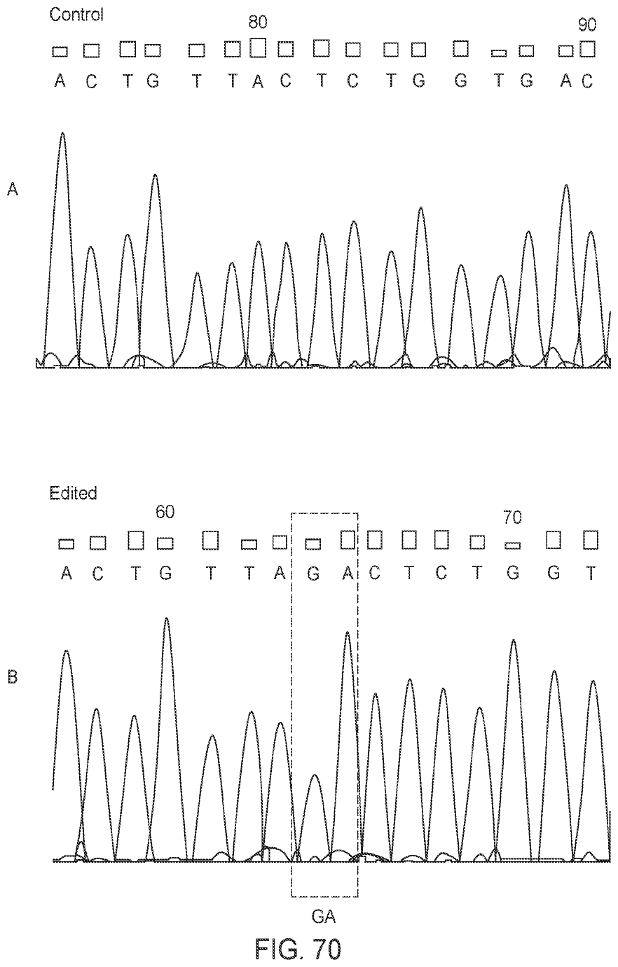

FIG. 70 panels A and B show Sanger sequencing results used to further confirm successful targeting and editing of exon 51 of this human dystrophin gene. Genomic DNA was isolated and used as a template on which a 151-bp PCR amplicon surrounding beginning region of exon 51 of dystrophin gene was generated by using a primer set of POP83 and POP84. FIG. 70 panel A shows an exemplary chromatogram of a wild-type “TTACT” sequence from untargeted B cells by Sanger sequencing. FIG. 70 panel B shows an edited “TTACT” sequence at this target site after protein-based SSB-HbW-based gene editing with protein and a sequence modification polynucleotide containing a two-nucleotide “GA” insertion relative to wild-type. Sequencing results confirm detection of this two-nucleotide “GA” insertion into this targeted location. These results confirm that SSB-HbW-based gene editing via protein-based delivery in combination with a sequence modification polynucleotide can successfully target and edit a sequence in an endogenous mammalian gene in mammalian cells to successfully modify a disease-causing genotype.

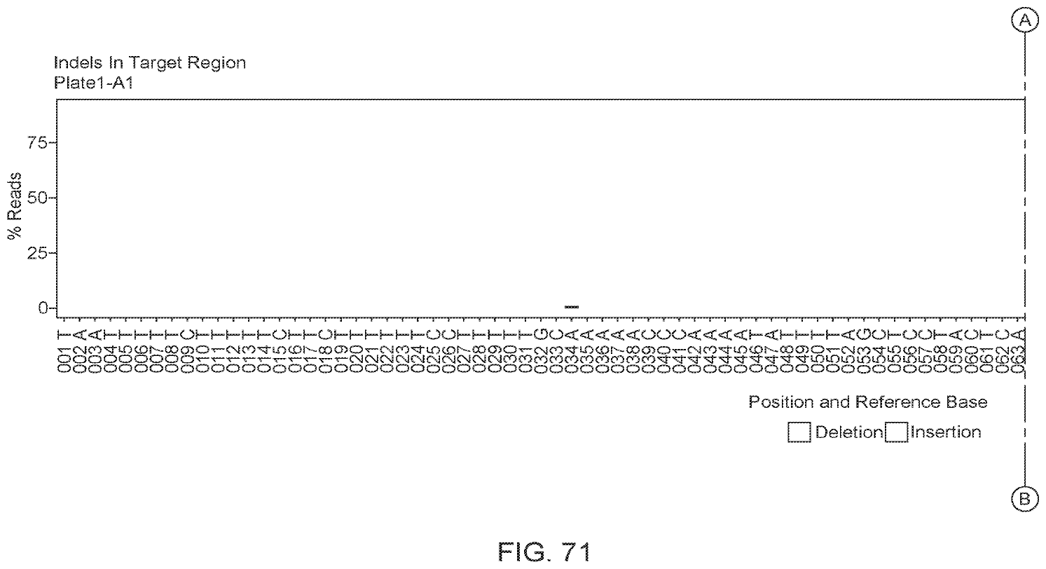

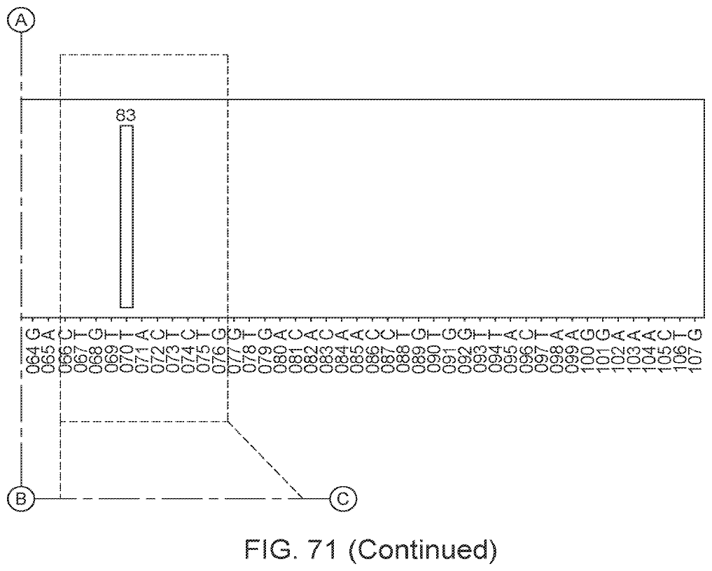

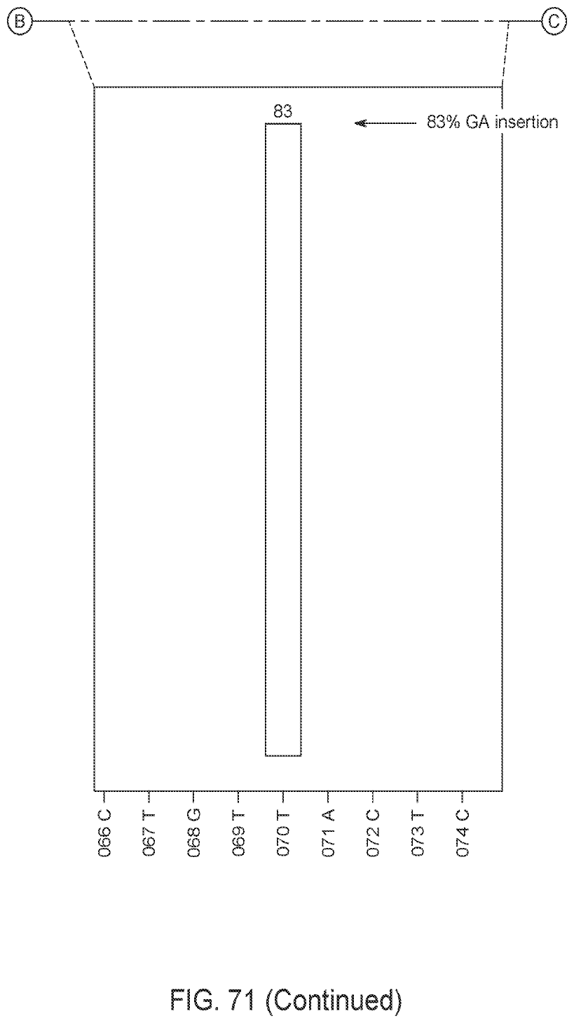

Further detailed validation of this genomic “GA” two-nucleotide insertion was evaluated by next generation sequencing. Next generation sequencing of targeted B pooled cells was performed after protein-based delivery of SSB-HbW-based gene editing. Genomic DNA was isolated and used as a template on which a 151-bp PCR amplicon was generated by using a primer set of POP83 and POP84 (in which is also the primer set used in Sanger sequencing analysis in this Example). Amplified PCR products from targeted B cells was analyzed for indels and SNPs on an Illumina next generation sequencing platform (GENEWIZ, South Plainfield, NJ). FIG. 71 shows overall insertions and deletion and editing frequencies of a pooled targeted B cells. After SSB-HbW-based gene editing with protein and a single stranded sequence modification polynucleotide, an overall “GA” insertion for editing frequency of 83% was achieved. Indel frequencies at this region remained very low. Taken together, SSB-HbW-based gene editing is able to achieve relatively high gene editing efficiency with very low indel frequencies through a protein-based delivery route.

### Example 13: Modification of an Endogenous Genomic Target: Codon 112 of ApoE Gene by SSB-HbW-Based Gene Editing In Vivo

In this Example, codon 112 of a human ApoE gene was modified using SSB-HbW-based gene editing in vivo in a living animal. Delivery of genome editing components into living animals, such as a mouse, is intended to induce genetic conversion. Genetic conversion can be aimed at (a) specific target tissue(s), cell type(s), organ(s) and/or it can be intended to achieve genetic conversion in any type of cell, tissue and/or organ in an living organism. Genetic conversion in a living animal, herein and hereinafter, is referred to as “in vivo” genome editing. In this example, SSB-HbW-based gene editing components, a polynucleotide modification agent and a single stranded oligonucleotide template, are formulated into a non-viral lipid nanoparticles (LNPs) and injected into blood streams of experimental mice. Following an incubation period, cells and genomic DNA is obtained from these animal and tested for genetic by analysis of (a) genomic target(s).

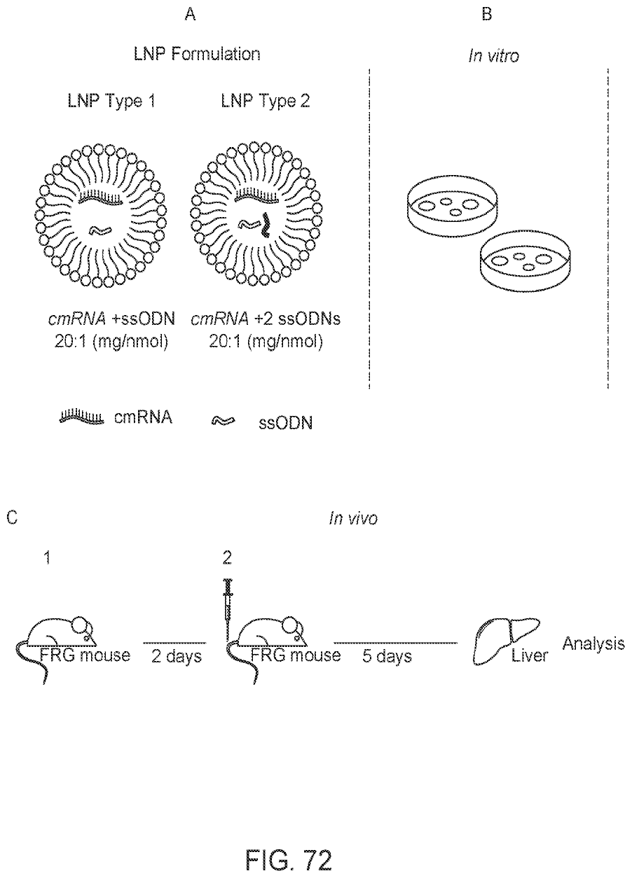

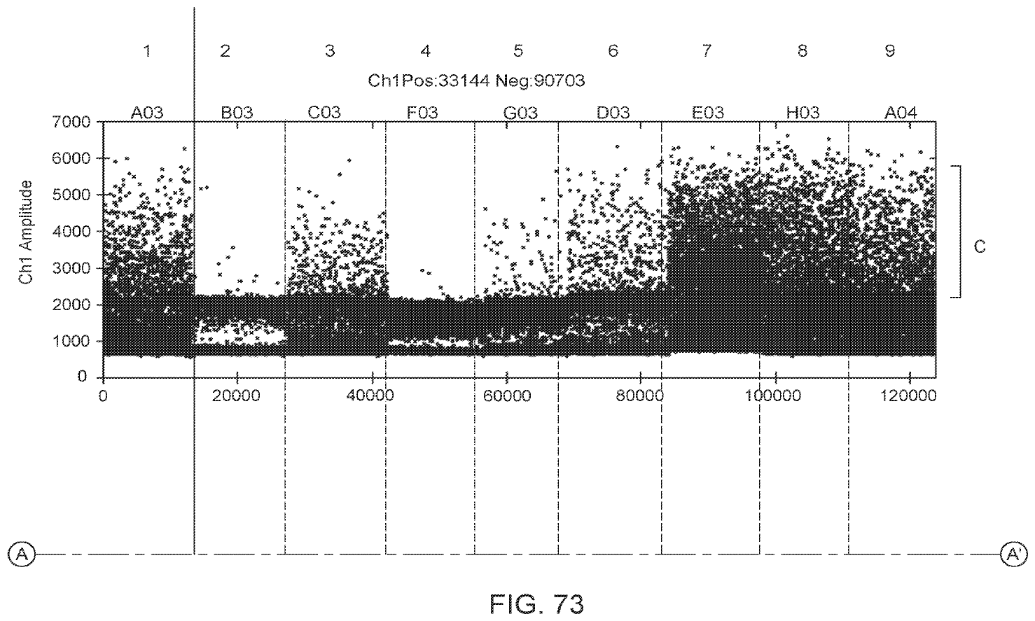

FIG. 72 depicts a schematic approach for in vivo genome editing using SSB-HbW-based gene editing. As a first step shown in FIG. 72 panel A, components for SSB-HbW-based gene editing are formulated into lipid nanoparticles, hereinafter referred as to LNPs. A polynucleotide modification agent encapsulated in LNPs, using methods known to those skilled in the art. In this example two components are included in LNPs: a mRNA molecule encoding a Zinc Finger Helicase and a single stranded DNA modification template. A method used in this example for the generation of mRNA is to transcribe it from it corresponding DNA plasmid template. Other methods to produce or generate mRNA, known to those skilled in the art, are contemplated to be potential alternatives for obtaining suitable mRNA. Single stranded oligonucleotide modification templates used in this example were obtained as generated by chemical synthesis, similar to those described in previous examples. This application contemplates that other methods of generating DNA modification templates, known to those skilled in the art, can provide alternatives for obtaining such molecules. In this example LNPs were produced using ingredients and equipment frequently used by those skilled in the art. As equipment a Precision Nanosystems (Vancouver, British Columbia, Canada) NanoAssemblir microfluids platform was used to produce LNPs. As lipid mixture Gen Voy components (Precision Nanosystems) were according to the suppliers' instructions.

Prior to application in vivo, formulated LNPs were evaluated in vitro, specifically in assaying with mammalian cells testing for genomic editing confirmation as shown in FIG. 72 panel B. After in vitro validation, LNPs were applied for in vivo experiments. FIG. 72 panel C shows that validated LNPs were injected into experimental mice via tail vein injection on two occasions with two days in between injections. Five days after receiving a final injection, livers were collected and subjected to genomic analysis for HbW-based gene editing confirmation. In this example, a “FRG KO” mouse model is used. This mouse model features a triple knockout of the Fah, Rag2 and Il2rg genes. As a consequence of these genes being inactivated, the FRG genotype enables these animals to be engrafted with human hepatocytes that can populate the liver in these mice. Such a “humanized liver” animal model can be of particular relevance when studying in vivo features of human hepatocytes relevant in many application areas including infectious diseases, NASH, gene editing/therapy, metabolism and pharmacology-toxicology. In this example each mouse received two doses of LNP treatment via tail vein injection. A first dose was administrated on day 1 of this experiment and a second LNP dose was injected on day 3. On day 8 of this experiment livers were obtained and subsequently genomic DNA was extracted for genetic conversion analysis.

This specific example aimed at gene editing of an endogenous genomic target around codon 112 of human ApoE in human B cells and HepG2 cells in vitro, followed by in vivo experiments in humanized FRG mice to demonstrate in vivo genome editing. In this example, mRNA is transcribed from plasmid pb 121 (SEQ ID NO.: 111), including a DNA recognition domain which was an array of 9 zinc-fingers, specifically designed to recognize 5′-GCGGCCGCCTGGTGCAGTACCGCGGCG-3′ (SEQ ID NO.: 22), a 27-nucleotide sequence on the leading strand of human ApoE, also described in Example 7 above. In this embodiment, a donor template, POP 33 (SEQ ID NO.: 11), was used: a 129-nucleotide single stranded DNA oligonucleotide with a desired T→C substitution roughly located in the middle of this oligonucleotide. This single stranded donor template used herein is provided below as a sequence with an underlined and bold “C” to for T→C conversion. In this embodiment, dual single stranded oligonucleotide templates, POP 358 and POP 362 (SEQ ID NO.: 123 and 124, respectively) are also used, in which they are 114-nucleotide in length. In this example these two oligonucleotides are complementary to each other and are added in single stranded format. In this embodiment, detection methods for in vivo gene editing confirmation and validation include ddPCR and next generation sequencing which have been described in Examples 2, 3 and 9 above. LNPs for in vivo editing were manufactured on a Precision Nanosystems microfluidic NanoAssemblir using Gen Voy lipid components.

```
POP358 ApoE oligo 6 has a sequence of
  (SEQ ID NO.: 123)
CCCCGGTGGCGGAGGAGACGCGGGCACGGCTGTCCAAGGAGCTGCAGGC
GGCGCAGGCCCGGCTGGGCGCGGACATGGAGGACGTGCGCGGCCGCCTG
GTGCAGTACCGCGGCG.
POP362 Apo ODN oligo has a sequence of
  (SEQ ID NO.: 124)
CGCCGCGGTACTGCACCAGGCGGCCGCGCACGTCCTCCATGTCCGCGCC
CAGCCGGGCCTGCGCCGCCTGCAGCTCCTTGGACAGCCGTGCCCGCGTC
TCCTCCGCCACCGGGG.
```

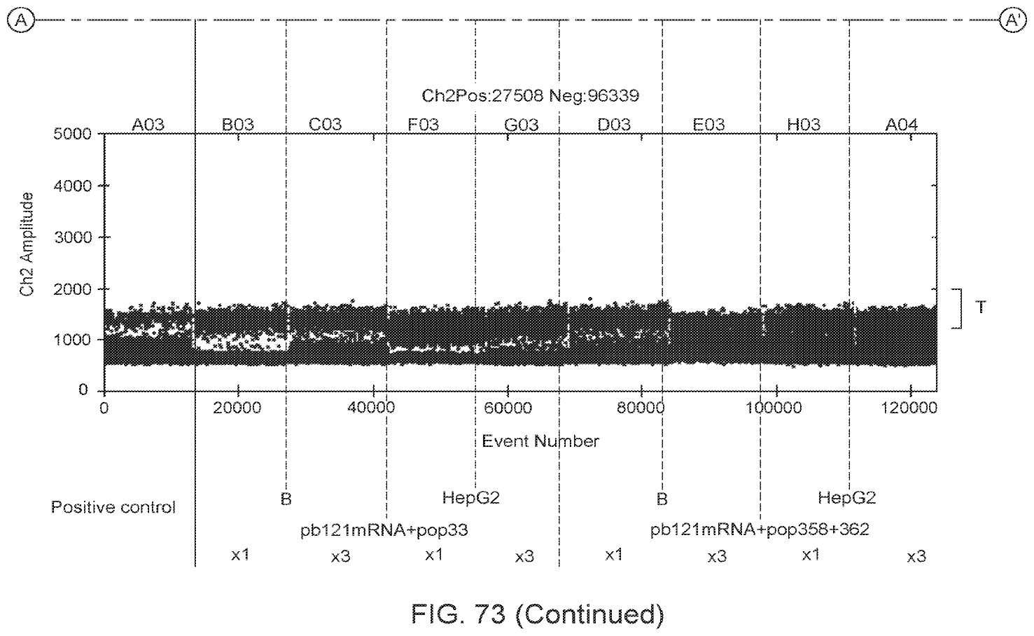

FIG. 73 demonstrates successful “T-to-C” genetic conversion at codon 112 of human ApoE as measured by ddPCR. In this example, to both B cells and HepG2 cells two formulations of LNPs were added to cell culture in independent experiments. One LNP composition comprised mRNA derived from pb 121 and POP 33, referred as to as LNP1. A second LNP composition contained mRNA derived from pb 121, POP 358 and POP 362, referred to as LNP2, hereinafter. Each cell type received either a single dose or 3-repeated-dose treatments in independent experiments. After five days genomic DNA was isolated and used in ddPCR analysis. Raw droplet data are shown as in FIG. 73 where these “C” droplets are displayed in the top panel in each set, while “T” droplets were in the lower one. Lane1 shows a positive “T-to-C” conversion as a positive control. Lane 2-9 show positive “C” droplets appeared after being treated LNPs. In this example, when cells were treated with LNP1, using a 3-repeated dosing treatment, they yielded more positive “C” droplets when comparing to a single dosing treatment (Lane 2, 3, 4, and 5). Both of B cells and HepG2 cells yielded more positive “C” droplets when treated three times with LNP1, compared to single LNP treatments. Likewise, Lane 6, 7, 8 and 9 show results from LNP2 treatments in human B cells and HepG2 cells. Three-repeat-dose treatment of LNP2 resulted in more positive “C” droplets compared to a single dose treatment for both human B and HepG2 cells. Together, this example illustrates that LNPs can be used as an effective delivery tool suitable for SSB-HbW-based gene editing, using a mRNA-based polynucleotide modification agent in combination with (a) single-stranded polynucleotide template(s).

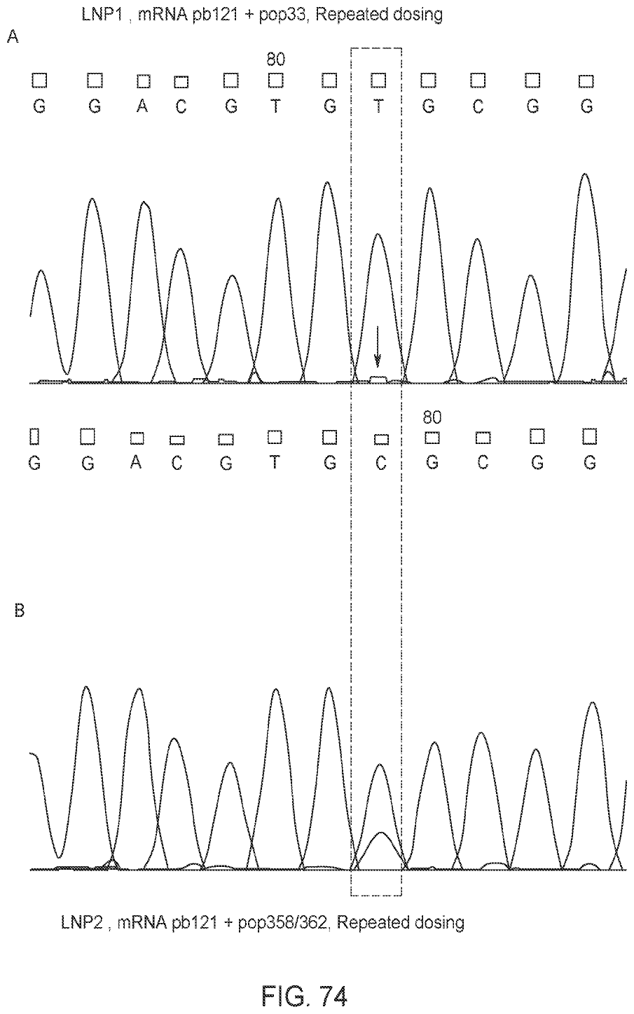

FIG. 74 panels A and B show exemplary Sanger sequencing results obtained to further confirm successful targeting and editing of codon 112 of the human ApoE gene, following treatment with LNP delivered SSB-HbW gene editing components. Genomic DNA was isolated and used as a template on which a 175-bp PCR amplicon surrounding codon 112 of ApoE gene was generated by using a primer set of POP 46 and POP 37 (SEQ ID NO.: 62 and 63, respectively). Amplified PCR products from targeted B cells were analyzed. FIG. 74 panel A shows an exemplary chromatogram from pooled B-cell population treated with LNP1 with 3 repeated doses, showing a small “C” spike under the “T” spike at the targeted position. FIG. 74 panel B shows genetic conversion identified by “C” at this targeted position when B cells were treated with 3 repeated doses of LNP2. In this chromatogram, a wild type “T” at codon 112 is shown as a signal under a converted “C” signal. These results illustrate that a SSB-HbW polynucleotide modification agent in combination with a sequence modification polynucleotide can be used to target and edit a sequence in an endogenous mammalian gene in mammalian cells to successfully modify a disease-causing genotype, also when they are delivered using an LNP encapsulated mRNA and single stranded oligonucleotide template.

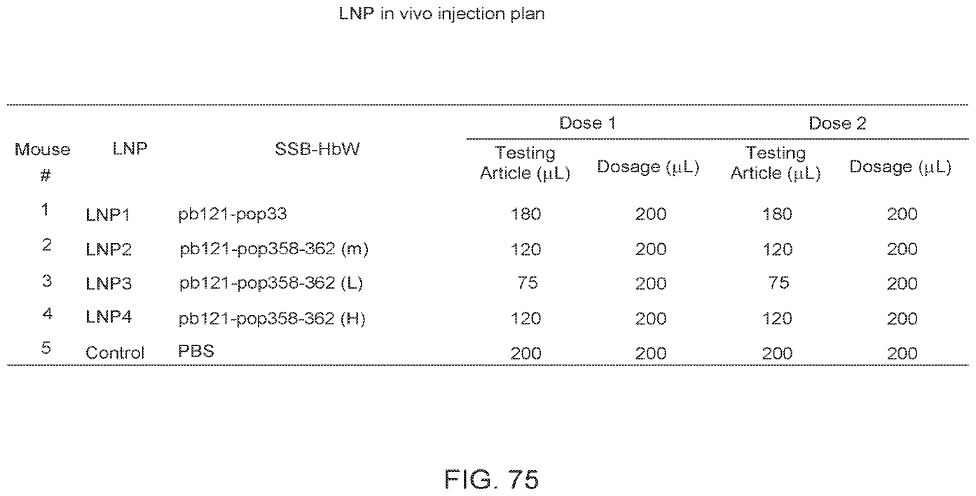

As exemplified above, LNP based delivery of SSB-HbW components can achieve gene editing results in vitro. In this example we also illustrate that in vivo genome editing can be achieved, using LNPs to deliver a mRNA-based polynucleotide modification agents and a single stranded oligonucleotide. In this example such a system is used to convert “T” into “C” at the codon 112 of ApoE gene, using FRZ mice for in vivo gene editing. FIG. 75 shows a table for an in vivo editing injection plan, as used in this example. Five male liver-humanized Fah “Rag2^{-/-}Il2rg^{-/-} mice were used in this example, each of them has ≥70% human hepatocyte repopulation of its liver. Each mouse was dosed with test articles via tail vein injection on Day 1 and on Day 3. An amount of LNP and dosing intervals are indicated in FIG. 10.4. Here, we used as target dose injection an volume of 200 μl for each of the tests. Amounts of LNPs for each experiment is shown in FIG. 75. All mice were monitored immediately after dosing to ensure that no unintended harm occurred during dosing.

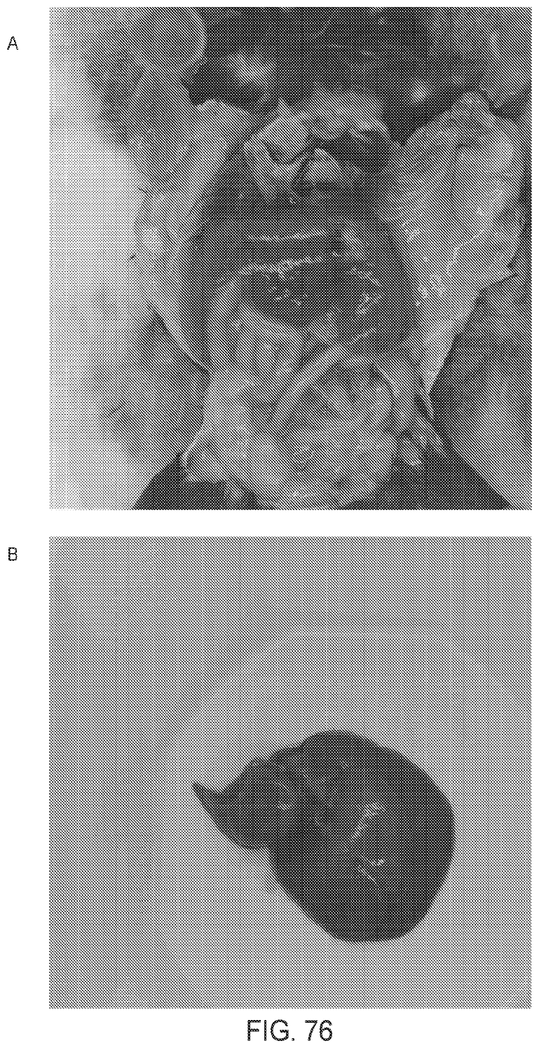

FIG. 76 shows exemplary pictures of terminal liver tissues obtained at day 8 of this experiment. The peritoneum and thoracic cavity were opened to expose the liver for evaluation of humanization by gross organ pathology as well as images captured of liver in situ (FIG. 76 panel A) and ex situ (FIG. 76 panel B). Approximately 10 μg liver tissues were further taken out from left, medium, and right lobes of each liver for genomic DNA extraction and in vivo gene editing analysis.

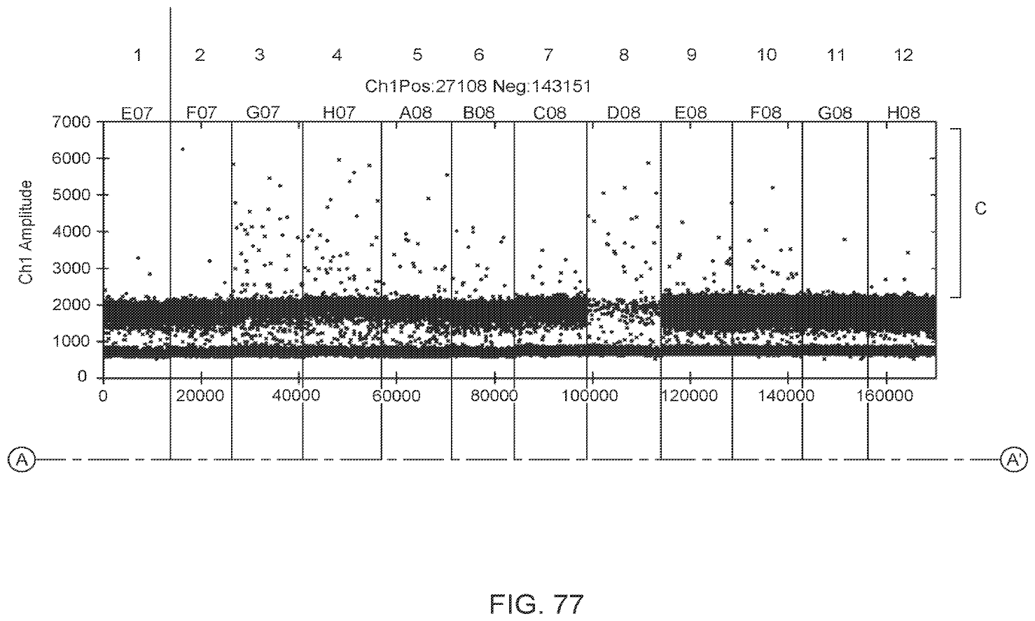

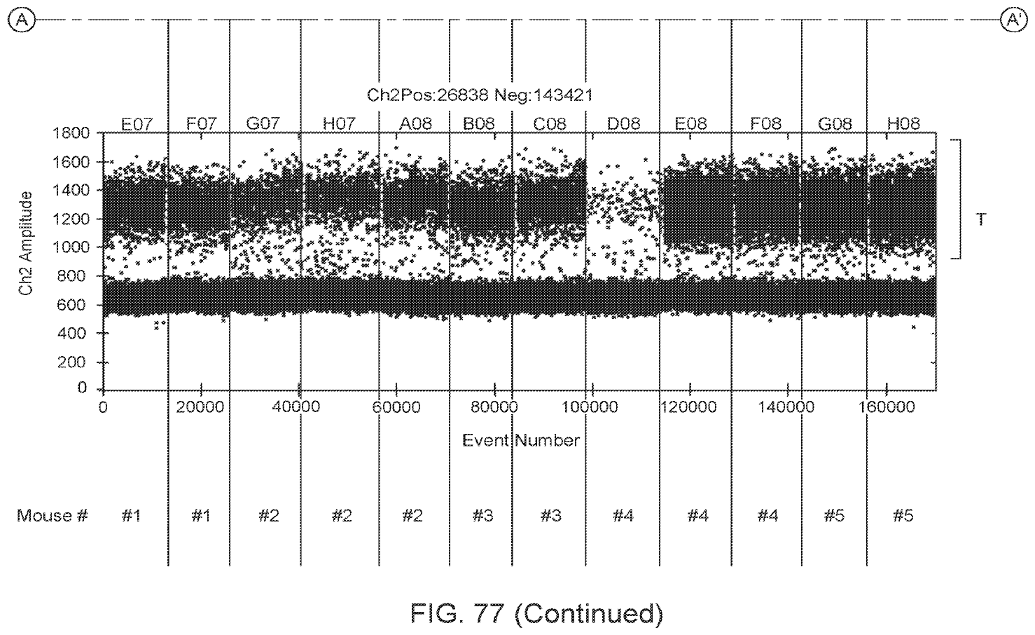

FIG. 77 demonstrates successful in vivo T→C genetic conversion at codon 112 of human ApoE as measured by ddPCR. In this example, genomic DNA from various sites of livers were extracted, after they had received injections of two doses of LNPs through tail veins. Lanes 1 and 2 correspond to mouse #1, which had received two doses of LNP1 injections. Lanes 3, 4 and 5 correspond to mouse #2, which had received two doses of LNP2 injections (with medium dosage). Lanes 6 and 7 correspond to mouse #3 which had received two doses of LNP2 injections (with medium dosage). Lanes 9, 10 and 11 correspond to mouse #4, which had received two doses of LNP2 injections (with high dosage). Lanes 12 and 13 correspond to mouse #5, which had received two injection of control articles. Raw droplet data are shown in FIG. 77 where “C” droplets are displayed in the top panel, while “T” droplets are shown in the lower panel. Mice that were injected with LNPs showed positive “C” droplets, demonstrating that SSB-HbW based in vivo genome editing can be achieved by using LNP, as illustrated here by T→C genetic conversion at codon 112 of human ApoE. Mice #2, #3 and #4 had more positive “C” droplets than mouse #1, suggesting that in this experiment LNP2 resulted in a higher in vivo editing efficiency than LNP1.

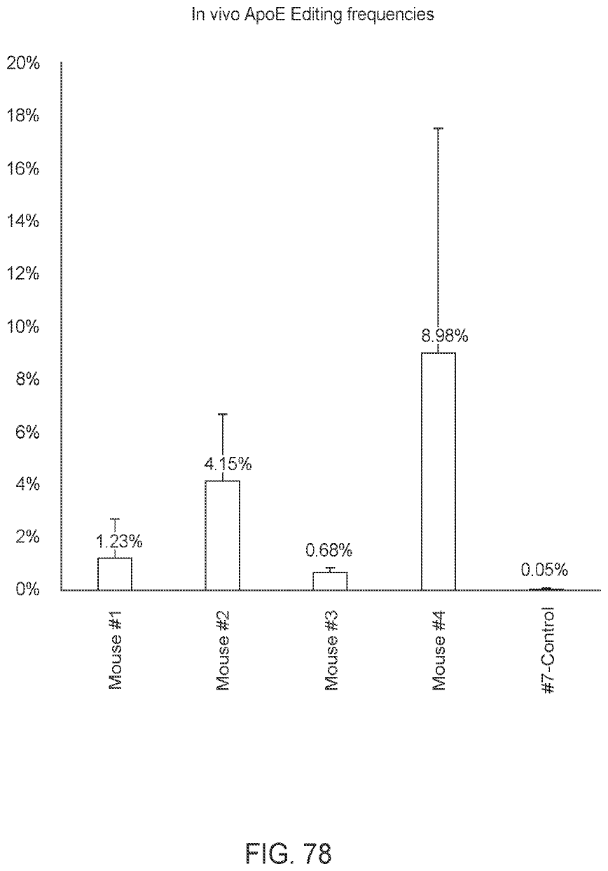

FIG. 78 shows genetic “T-to-C” conversion frequencies as measured by ddPCR after in vivo SSB-HbW-based gene editing. It shows editing frequencies as “T-to-C” conversion percentages, calculated as a percentage of “C” droplet events divided by the sum of “T” and “C” droplet events. In this experiment, in vivo SSB-HbW-based gene editing achieved genetic conversion frequencies of in 4 mice of 1.23%, 4.15%, 0.68% respectively 8.98% after receiving doses of LNP1 or LNP2. A background level of 0.05% of “T-to-C” conversion was observed, indicating that the observed effect was not due to background signal.

In this present Example, next generation sequencing was also performed to determine, in more detail, gene conversion frequencies and patterns. It was also used to detect potential generation of insertions, deletions, and/or unintended single nucleotide polymorphisms after in vivo SSB-HbW-based gene editing. To do so, next generation sequencing of mouse liver tissues was performed. Genomic DNA was isolated and used as a template on which a 175-bp PCR amplicon surrounding ApoE codon 112 was generated by using a primer set of POP 46 and POP 37. Amplified PCR products then was analyzed for indels and SNPs on an Illumina next generation sequencing platform (GENEWIZ, South Plainfield, NJ).

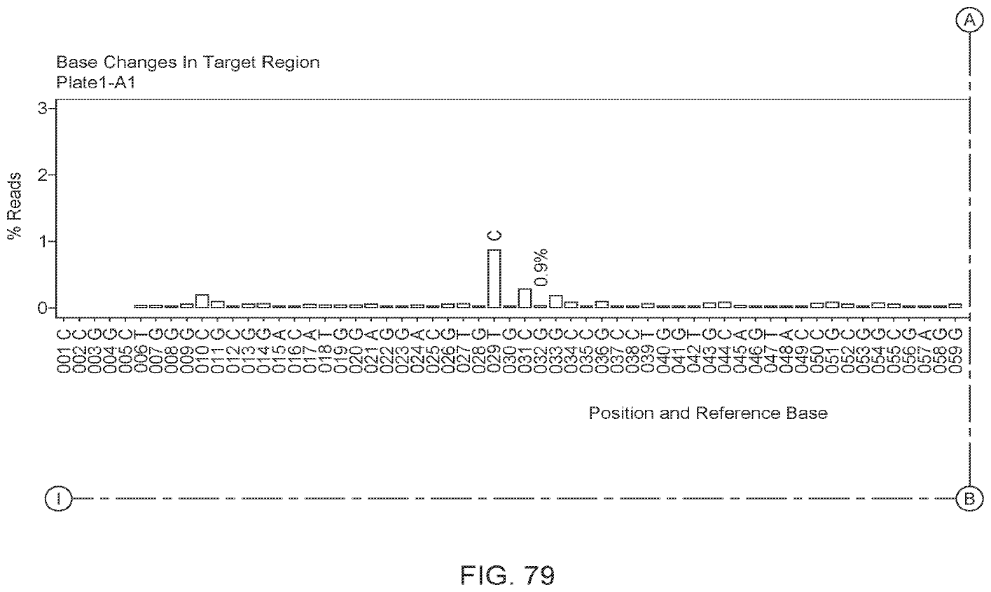

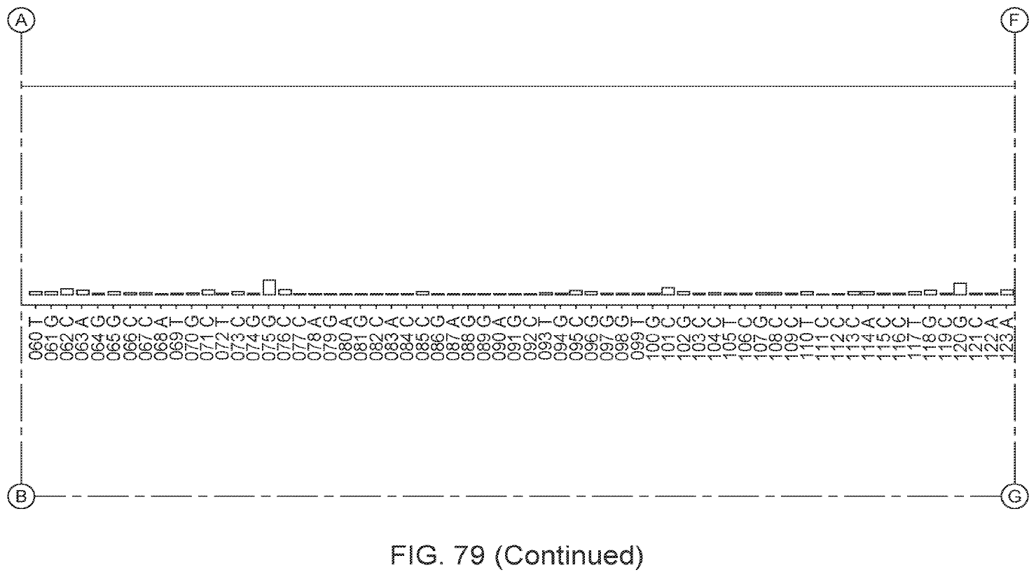

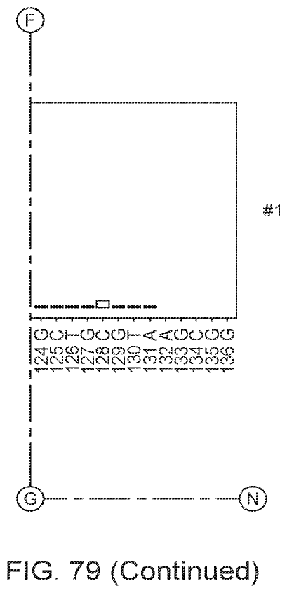

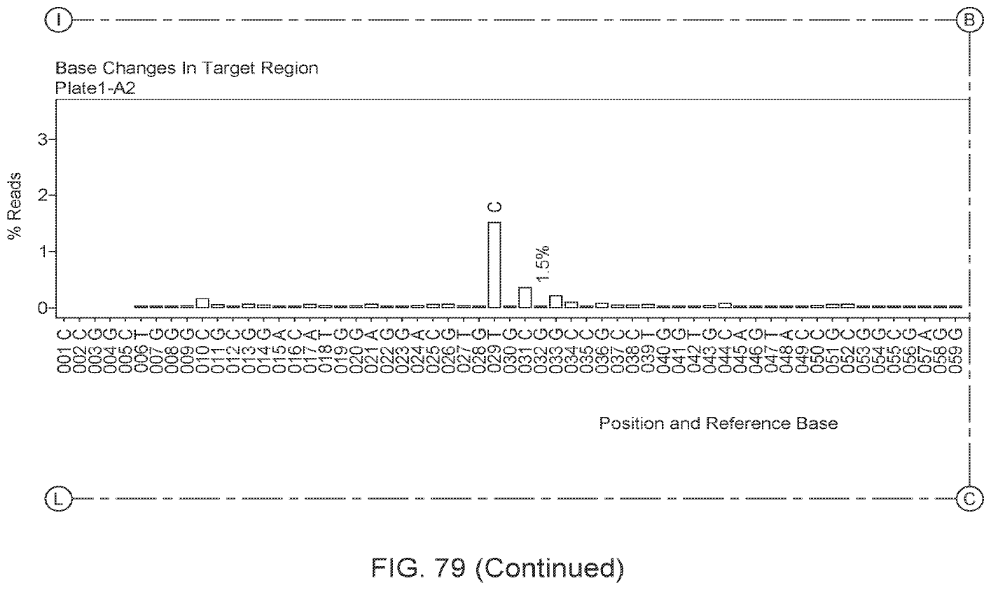

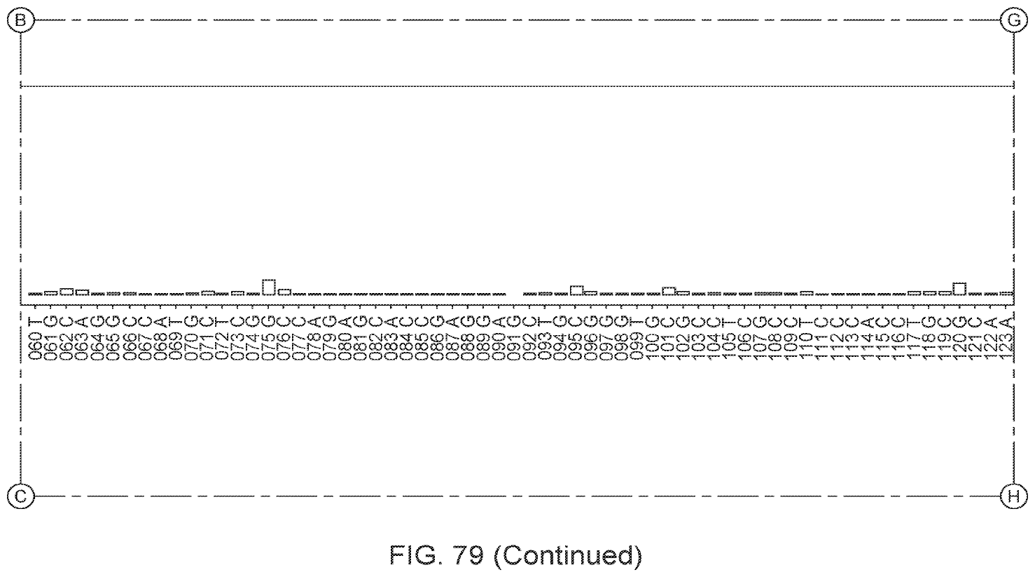

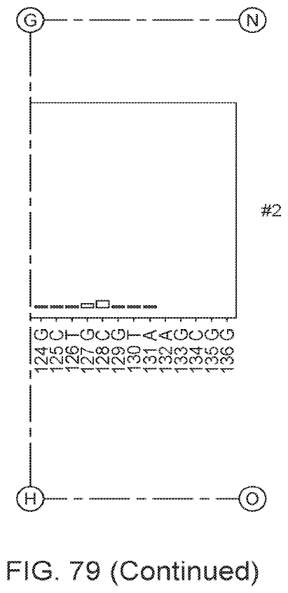

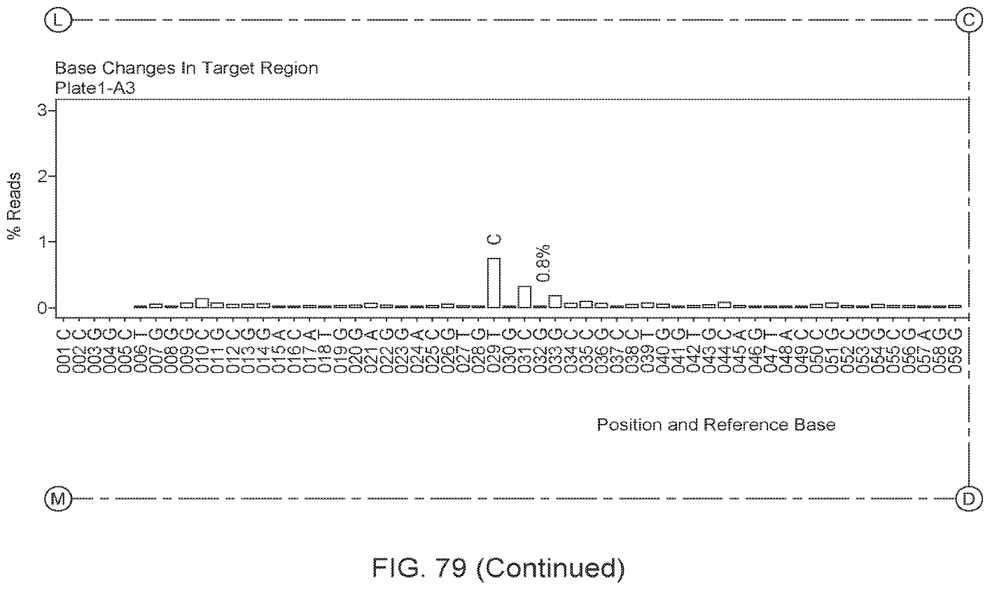

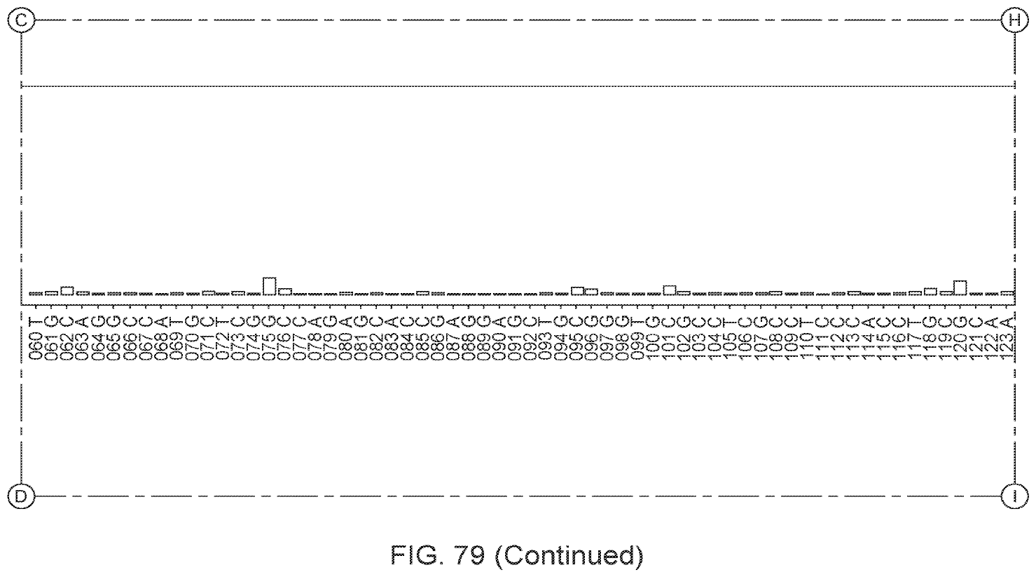

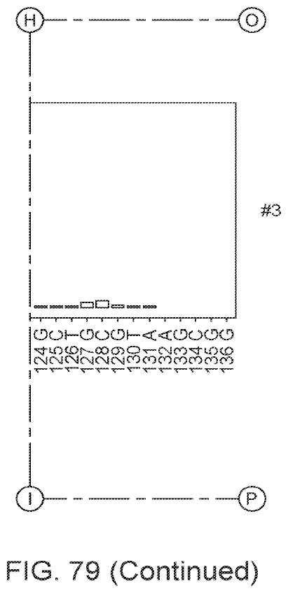

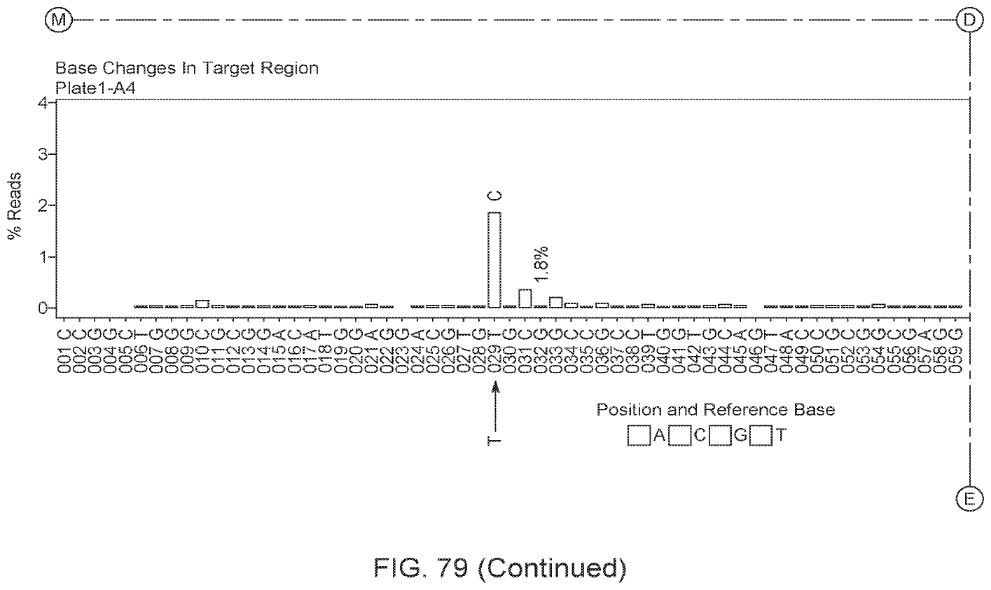

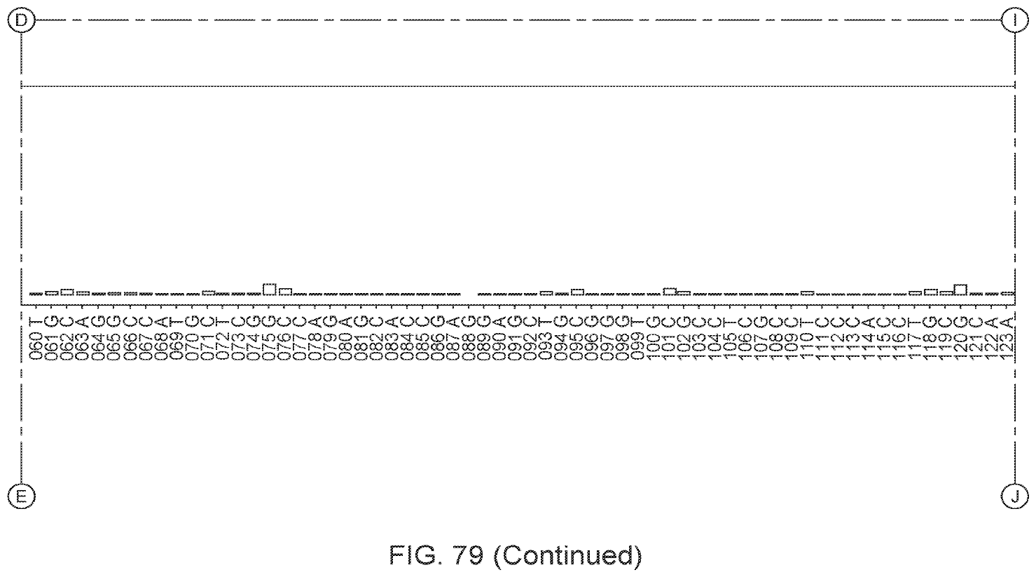

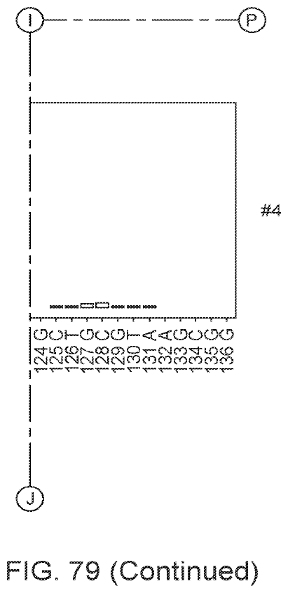

FIG. 79 shows confirmation of detection of single nucleotide T→C conversion at this target site as well as single nucleotide polymorphisms (SNPs) analysis within a target region surrounding codon 112 of this ApoE locus after in vivo SSB-HbW based gene editing. Bar graphs plot frequencies of SNPs at each nucleotide position in this 175 bp PCR amplification region. FIG. 10.8 shows a T-to-C conversion frequency of 0.9%, 1.5%, 0.8% and 1.8% for mouse #1 through #4, after they each received two doses of LNP injections. Within amplification regions analyzed, no significant unintended SNPs were observed.

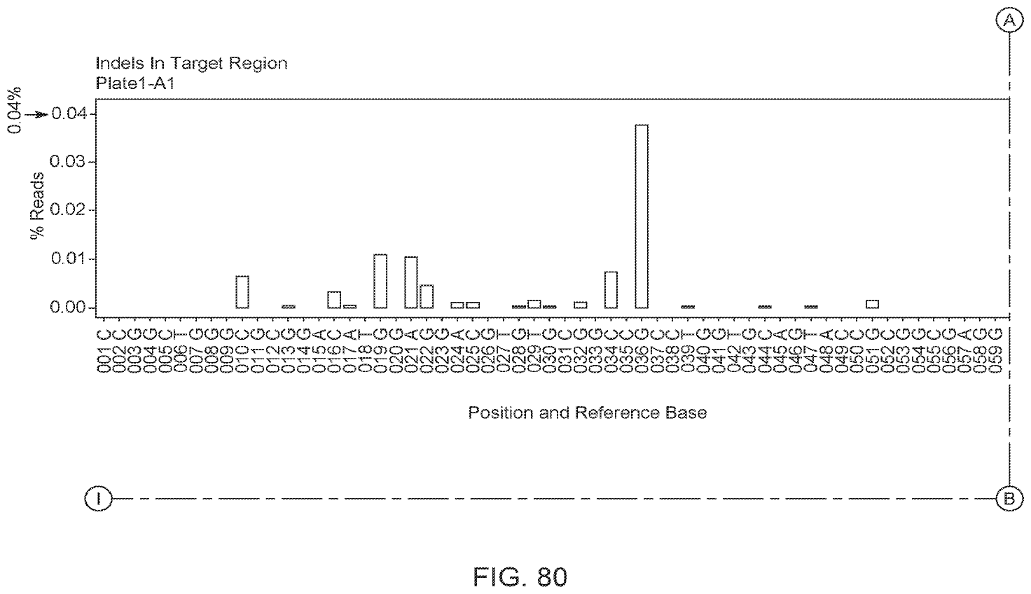

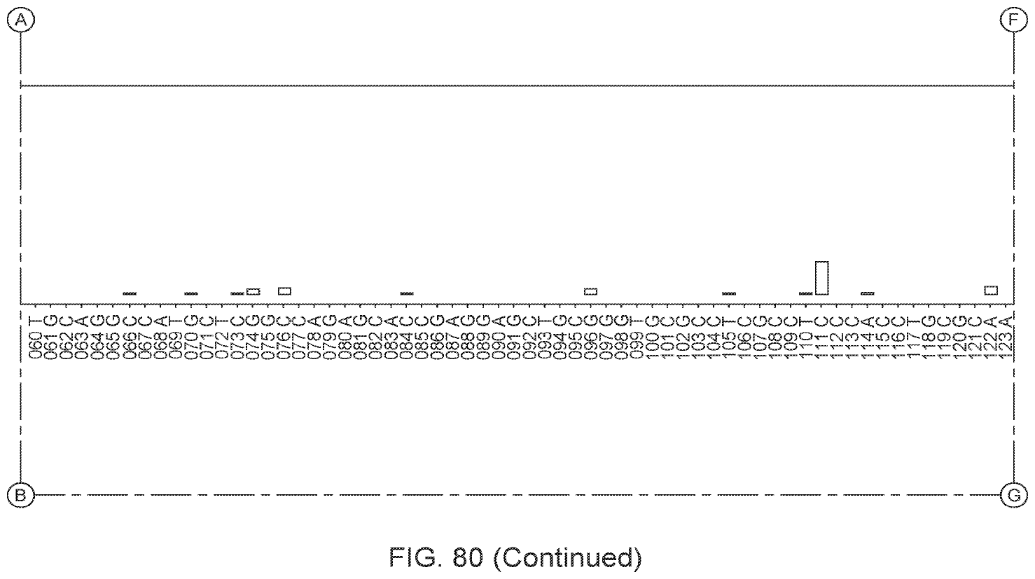

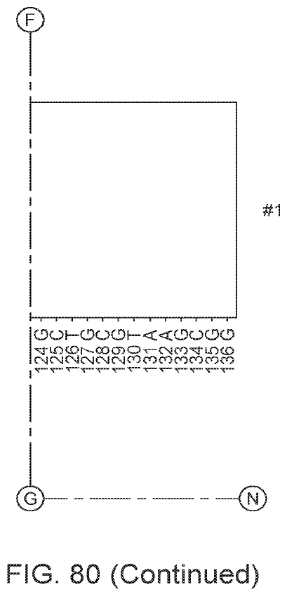

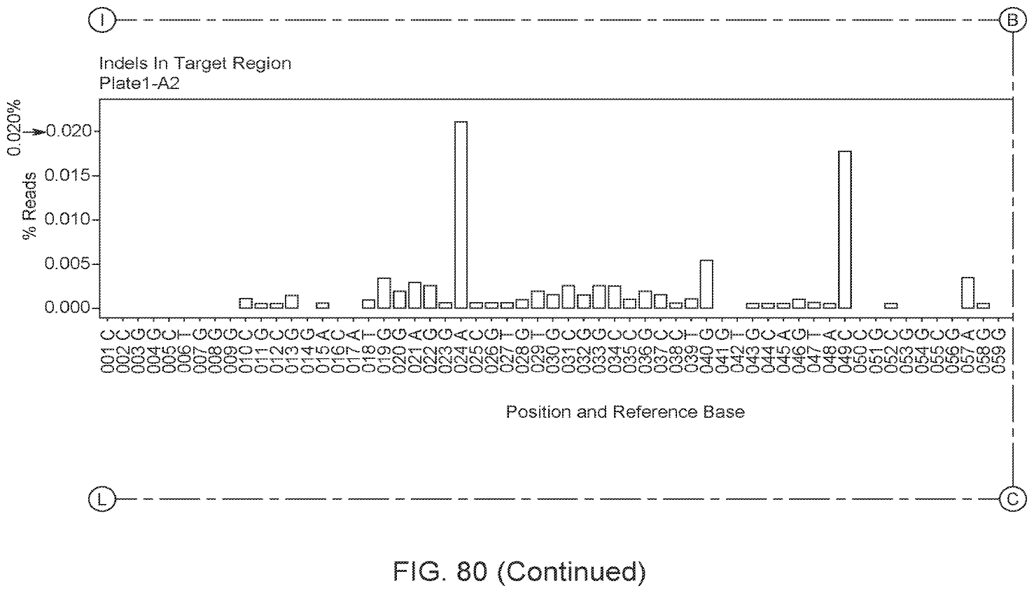

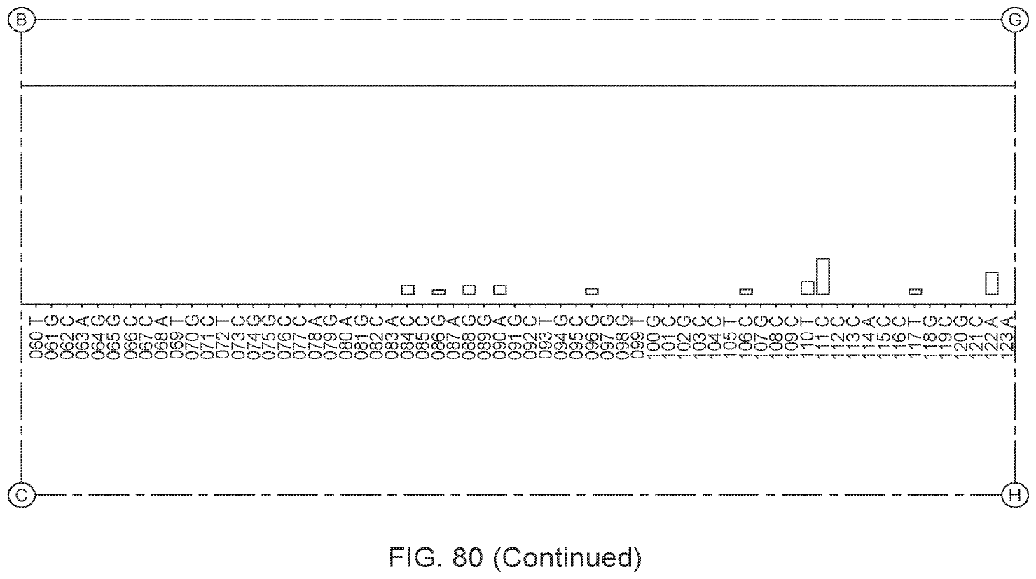

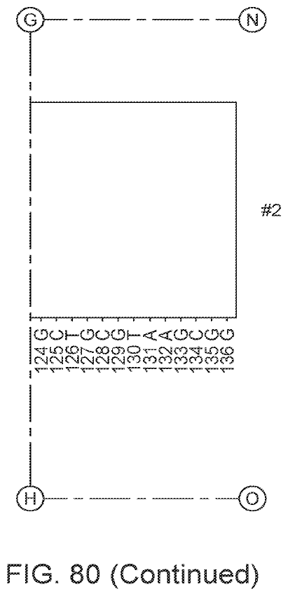

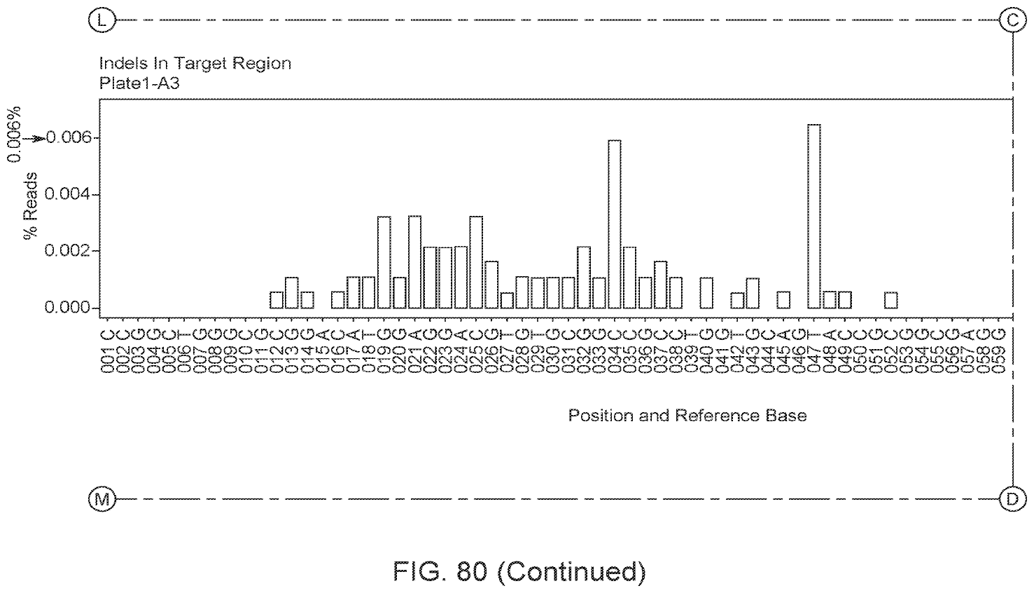

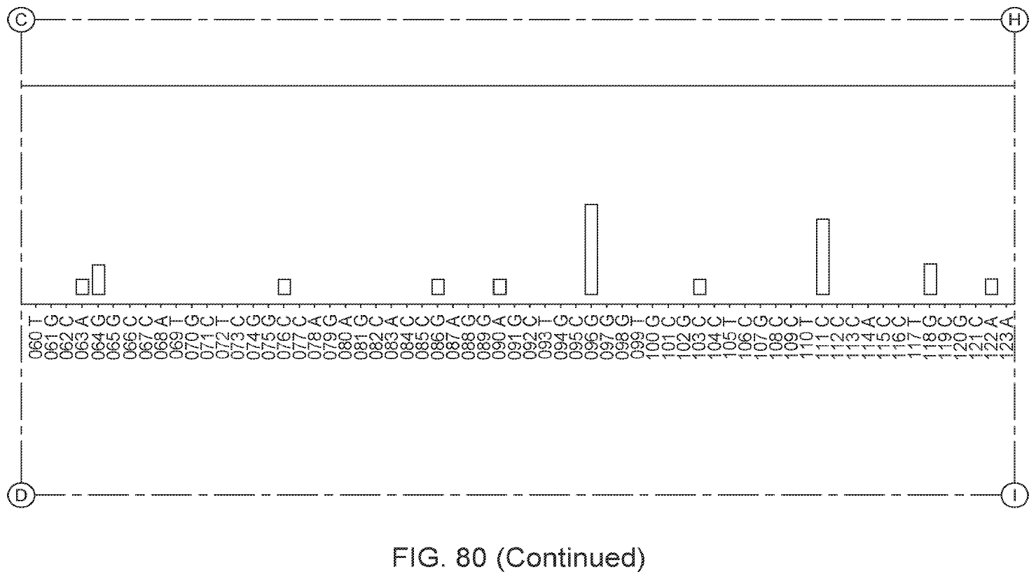

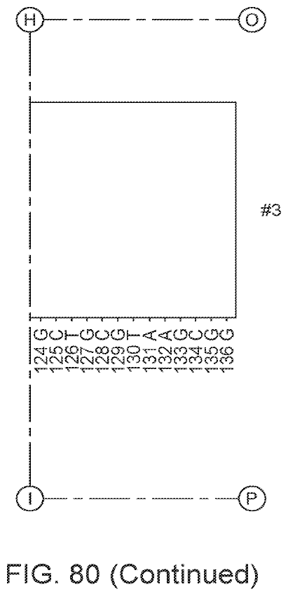

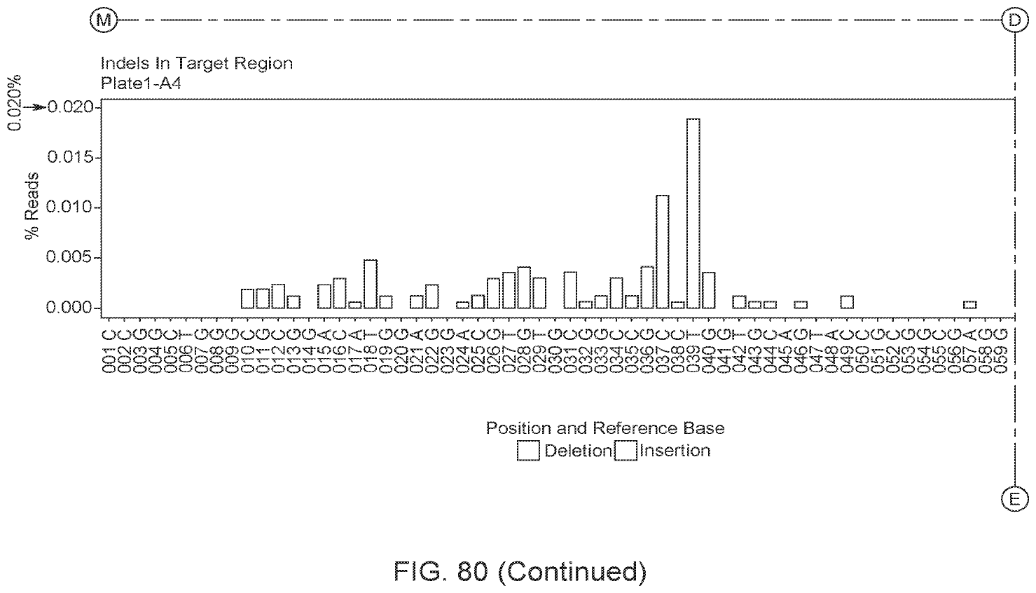

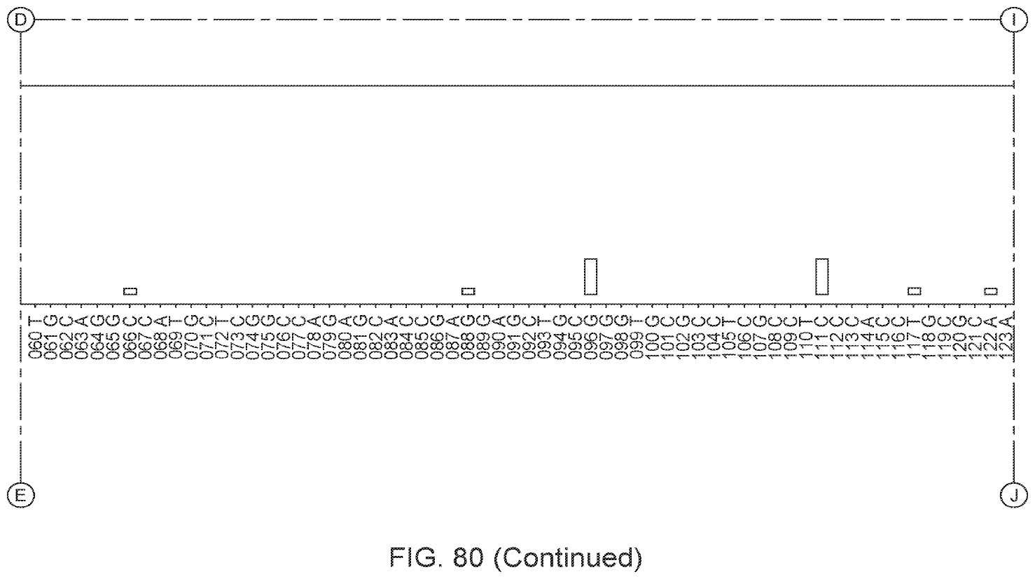

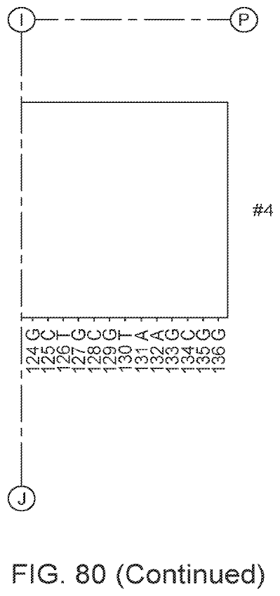

FIG. 80 shows insertion and deletion analysis around codon 112 of ApoE displayed as a frequency plot of insertions and deletions analysis for from in vivo SSB-HbW-based gene editing, bar graphs plot frequencies of insertions and deletions at each nucleotide position of this 175 bp PCR amplification region. This indels analysis showed, in general, a very low frequency (<0.5%) of insertions and/or deletions in each given position in this region of 175 bp PCR amplification, as obtained for mouse #1 to #4. As shown, the highest insertion and deletion frequency is below 0.04% for mouse #1, 0.02% for mouse #2, 0.006% for mouse #3, and 0.02% for mouse #4.

These results confirm that SSB-HbW based gene editing can successfully target and edit a sequence in an endogenous mammalian gene in vivo. In addition it also illustrates successful modification of a disease-causing genotype using LNP delivery in liver tissues. Notably, this example also demonstrates that provided technologies are capable of genetic modification with low levels of insertions and deletions. Thus, technologies of this present disclosure are capable of targeted gene conversion in vitro and/or in vivo. This can be achieved while significantly limiting generation of insertions, deletions and/or undesired single nucleotide polymorphisms.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A polynucleotide modification agent comprising a helicase beta-wing element (“HbW element”) and a sequence-specific binding element, wherein the HbW element is or comprises a helicase beta-wing and the sequence-specific binding element comprises a zinc finger polypeptide comprising one or more zinc finger arrays.

2. The polynucleotide modification agent of claim 1, wherein the HbW element is or comprises a helicase beta-wing polypeptide with a mammalian sequence derived from a mammalian helicase polypeptide.

3. The polynucleotide modification agent of claim 1, wherein the HbW element is or comprises a helicase beta-wing polypeptide with a human sequence derived from a human helicase polypeptide.

4. (canceled)

5. The polynucleotide modification agent of claim 1, wherein the HbW element is or comprises a polypeptide sequence that is at least 80%, at least 85%, at least 90%, at least 95%, at least 96%, at least 97%, at least 98% at least 99%, or 100% identical to a sequence set forth in any one of SEQ ID NOs: 1 to 3.

6. The polynucleotide modification agent of claim 1, wherein the sequence-specific binding element is or comprises a zinc finger polypeptide comprising at least five, six, seven, eight, nine, ten, or eleven, or more, zinc finger arrays.

7. The polynucleotide modification agent of claim 6, wherein the zinc finger arrays comprise at least one alpha helix engineered to comprise a modified amino acid sequence that differs from that of its corresponding wild type sequence.

8. The polynucleotide modification agent of claim 1, further comprising a linker.

9. (canceled)

10. The polynucleotide modification agent of claim 8, wherein the linker is or comprises a polypeptide between 2 and 100 amino acids in length or 0.2 kD and 10 kD in size.

11. The polynucleotide modification agent of claim 8, wherein the linker is or comprises:
(i) a polypeptide derived from a human helicase polypeptide; and/or
(ii) a polypeptide sequence that is at least 80%, at least 85%, at least 90%, at least 95%, at least 96%, at least 97%, at least 98% at least 99%, or 100% identical to a sequence set forth in any one of SEQ ID NOs: 7-9.

12. (canceled)

13. The polynucleotide modification agent of claim 1, wherein the HbW element interacts with a target site and wherein the sequence-specific binding element binds to a landing site adjacent to the target site.

14. (canceled)

15. The polynucleotide modification agent of claim 12, wherein the sequence-specific binding element binds to the landing site with a binding affinity characterized by a dissociation constant of 10 E−6 or lower.

16. A nucleic acid encoding the polynucleotide modification agent of claim 1.

17.-18. (canceled)

19. A combination comprising (i) the polynucleotide modification agent of claim 1 and (ii) a sequence modification polynucleotide.

20.-22. (canceled)

23. A method comprising:
contacting a cell or population of cells with (i) the polynucleotide modification agent of claim 1; and (ii) a sequence modification polynucleotide.

24. The method of claim 23, wherein the cell or population of cells comprise a DNA polynucleotide comprising at least one target site.

25. The method of claim 24, wherein the sequence modification polynucleotide:
(i) binds specifically to one strand of the DNA at a target site; and
(ii) has a mismatch or other DNA sequence difference relative to the target site, so that usage of the sequence modification polynucleotide incorporates the sequence modification into a complement of the one strand.

26. A method comprising:
contacting DNA with (i) the polynucleotide modification agent of claim 1;
and (ii) a sequence modification polynucleotide.

27.-28. (canceled)

29. A method comprising:
administering to a subject (i) the polynucleotide modification agent of claim 1; and
(ii) a sequence modification polynucleotide.

30.-33. (canceled)

34. The method of claim 23, wherein one target sequence is modified.

35. The method of claim 23, wherein two or more target sequences are modified.

36. (canceled)


SEQUENCE LISTING (186 sequences)

SEQ ID NO: 1 - protein, 17 aa, synthetic construct
VSRYNKFMKICALTKKG

SEQ ID NO: 2 - protein, 17 aa, synthetic construct
DLYINANDQAIAYVMLG

SEQ ID NO: 3 - protein, 16 aa, synthetic construct
DYSFTAYATISYLKIG

SEQ ID NO: 4 - protein, 261 aa, Homo sapiens
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTG

SEQ ID NO: 5 - protein, 23 aa, synthetic construct
  VARIANT 12..18: note = X can be any amino acid
FQCRICMRNFSXXXXXXXHIRTH

SEQ ID NO: 6 - protein, 23 aa, synthetic construct
  VARIANT 12..18: note = X can be any amino acid
FACDICGRKFAXXXXXXXHTKIH

SEQ ID NO: 7 - protein, 24 aa, synthetic construct
LRGSNSQRLADQYRRHSLFGTGVE

SEQ ID NO: 8 - protein, 19 aa, synthetic construct
SRHNERLFKKLILDKILDE

SEQ ID NO: 9 - protein, 15 aa, synthetic construct
EKIIAHFLIQQYLKE

SEQ ID NO: 10 - DNA, 150 bp, synthetic construct
  source 1..150: mol_type = other DNA
ccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagc

SEQ ID NO: 11 - DNA, 129 bp, synthetic construct
  source 1..129: mol_type = other DNA
ccccggtggcggaggagacgcgggcacggctgtccaaggagctgcaggcggcgcaggcccggctgggcgcggacatggaggacgtgcgcggccgcctggtgcagtaccgcggcgaggtgcaggccatgc

SEQ ID NO: 12 - DNA, 141 bp, synthetic construct
  source 1..141: mol_type = other DNA
ctcttagacataacacaccagggtcaatacaactttgaagctagtctagtgcaagctaacagttgcttgaattcacaggctccaggaagggtttggcctctgattagggtgggggcgtgggtggggtagaagaggactggc

SEQ ID NO: 13 - DNA, 138 bp, synthetic construct
  source 1..138: mol_type = other DNA
taatttttctttttcttcttttttcctttttgcaaaaacccaaaatattttagctcctactcagactgttagactctggtgacacaacctgtggttactaaggaaactgccatctccaaactagaaatgccatcttcc

SEQ ID NO: 14 - protein, 5 aa, Homo sapiens
TGEKP

SEQ ID NO: 15 - protein, 6 aa, Homo sapiens
TGSQKP

SEQ ID NO: 16 - protein, 23 aa, Homo sapiens
FQCRICMRNFSRSDHLTTHIRTH

SEQ ID NO: 17 - protein, 23 aa, Homo sapiens
FQCRICMRNFSRSSALTRHIRTH

SEQ ID NO: 18 - DNA, 15 bp, synthetic construct
  source 1..15: mol_type = other DNA
ggggaggacgcggtg

SEQ ID NO: 19 - protein, 139 aa, Homo sapiens
FQCRICMRNFSRSSALTRHIRTHTGEKPFACDICGRKFARSDTLTRHTKIHTGSQKPFQCRICMRNFSDRSNLTRHIRTHTGEKPFACDICGRKFARSDNLTRHTKIHTGSQKPFQCRICMRNFSRSDHLTRHIRTHTG

SEQ ID NO: 20 - DNA, 18 bp, synthetic construct
  source 1..18: mol_type = other DNA
gtggagctggacggggac

SEQ ID NO: 21 - protein, 165 aa, Homo sapiens
FQCRICMRNFSDRSNLTRHIRTHTGEKPFACDICGRKFARSDHLTRHTKIHTGSQKPFQCRICMRNFSDRSNLTRHIRTHTGEKPFACDICGRKFARSDSLSEHTKIHTGSQKPFQCRICMRNFSRSSNLTRHIRTHTGEKPFACDICGRKFARSDSLTRHTKIH

SEQ ID NO: 22 - DNA, 27 bp, Homo sapiens
  source 1..27: mol_type = other DNA
gcggccgcctggtgcagtaccgcggcg

SEQ ID NO: 23 - protein, 253 aa, Homo sapiens
FQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTG

SEQ ID NO: 24 - DNA, 33 bp, Homo sapiens
  source 1..33: mol_type = other DNA
ctggcagtgtaccaggccggggcccgcgagggc

SEQ ID NO: 25 - protein, 318 aa, Homo sapiens
MAAMAERPFQCRICMRNFSDRSHLTRHIRTHTGEKPFACDICGRKFARSDNLTRHTKIHTGSQKPFQCRICMRNFSDSSHLSEHIRTHTGEKPFACDICGRKFADRSDLTRHTKIHTGSQKPFQCRICMRNFSRSDHLTRHIRTHTGEKPFACDICGRKFADRSDLTRHTKIHTGSQKPFQCRICMRNFSRSDNLSEHIRTHTGEKPFACDICGRKFAESSNLTTHTKIHTGSQKPFQCRICMRNFSRSSSLTRHIRTHTGEKPFACDICGRKFAQSSDLTRHTKIHTGSQKPFQCRICMRNFSRSDSLSEHIRTHTG

SEQ ID NO: 26 - protein, 4 aa, synthetic construct
LRGS

SEQ ID NO: 27 - protein, 5 aa, synthetic construct
  REPEAT 1: note = The entire sequence of amino acids 1-5 can be repeated one to two times.
GGGGS

SEQ ID NO: 28 - protein, 5 aa, synthetic construct
TGERP

SEQ ID NO: 29 - protein, 5 aa, synthetic construct
TGDKP

SEQ ID NO: 30 - protein, 5 aa, synthetic construct
TGQKP

SEQ ID NO: 31 - protein, 7 aa, synthetic construct
FVGQQLK

SEQ ID NO: 32 - protein, 7 aa, synthetic construct
ACQKPFE

SEQ ID NO: 33 - protein, 7 aa, synthetic construct
AEERPYK

SEQ ID NO: 34 - protein, 7 aa, synthetic construct
TKEKPYQ

SEQ ID NO: 35 - protein, 7 aa, synthetic construct
NAKKSYQ

SEQ ID NO: 36 - protein, 7 aa, synthetic construct
TGQKPFQ

SEQ ID NO: 37 - protein, 7 aa, synthetic construct
TGEKPYK

SEQ ID NO: 38 - protein, 7 aa, synthetic construct
TGKRAYE

SEQ ID NO: 39 - protein, 7 aa, synthetic construct
TGEKPYE

SEQ ID NO: 40 - protein, 7 aa, synthetic construct
SGERTYR

SEQ ID NO: 41 - protein, 7 aa, synthetic construct
TGQKPYG

SEQ ID NO: 42 - protein, 21 aa, Homo sapiens
CNACRKLFSSKSRLQSHLRRH

SEQ ID NO: 43 - protein, 21 aa, Homo sapiens
CHSCGRAFGEKWKLDKHQKTH

SEQ ID NO: 44 - protein, 21 aa, Homo sapiens
CENCGNAYKQKSNLFQHQKMH

SEQ ID NO: 45 - protein, 21 aa, Homo sapiens
CKTCGKAFSWKSSCINHEKIH

SEQ ID NO: 46 - protein, 21 aa, Homo sapiens
CNECEKSFRQNSTLIQHKKVH

SEQ ID NO: 47 - protein, 21 aa, Homo sapiens
CTDCGKAFIYKSDLVKHQRIH

SEQ ID NO: 48 - protein, 21 aa, Homo sapiens
CSICEKAFSQKSNVIDHEKIH

SEQ ID NO: 49 - protein, 21 aa, Homo sapiens
CDLCGNTFIQKKNLIQHKKIH

SEQ ID NO: 50 - protein, 21 aa, Homo sapiens
CNRCGKAFFQKSNLHSHQKTH

SEQ ID NO: 51 - protein, 21 aa, Homo sapiens
CSECGKTFIRKLNLSLHKKTH

SEQ ID NO: 52 - protein, 24 aa, Homo sapiens
CSECGKAFADRSYLVRHQKRIHSR

SEQ ID NO: 53 - DNA, 3835 bp, synthetic construct
  source 1..3835: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggatcccggcacaacgccgagcggctgttcaagaagctgatcctggacaagatcctggacgaggacctgtacatcaacgccaacgaccaggccatcgcctacgtgatgctgggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 54 - DNA, 897 bp, synthetic construct
  source 1..897: mol_type = other DNA
atggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggatcccggcacaacgccgagcggctgttcaagaagctgatcctggacaagatcctggacgaggacctgtacatcaacgccaacgaccaggccatcgcctacgtgatgctgggctga

SEQ ID NO: 55 - protein, 298 aa, synthetic construct
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGSRHNAERLFKKLILDKILDEDLYINANDQAIAYVMLG

SEQ ID NO: 56 - DNA, 3847 bp, synthetic construct
  source 1..3847: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactgggctgagaggatccaactcccagcggctggccgaccagtaccggcggcactccctgttcggcaccggcgtggaggtgtcccggtacaacaagttcatgaagatctgcgccctgaccaagaagggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 57 - DNA, 909 bp, synthetic construct
  source 1..909: mol_type = other DNA
atggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactgggctgagaggatccaactcccagcggctggccgaccagtaccggcggcactccctgttcggcaccggcgtggaggtgtcccggtacaacaagttcatgaagatctgcgccctgaccaagaagggctga

SEQ ID NO: 58 - protein, 302 aa, synthetic construct
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGLRGSNSQRLADQYRRHSLFGTGVEVSRYNKFMKICALTKKG

SEQ ID NO: 59 - DNA, 3817 bp, synthetic construct
  source 1..3817: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggggagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 60 - DNA, 879 bp, synthetic construct
  source 1..879: mol_type = other DNA
atggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggggagaagatcatcgcccacttcctgatccagcagtacctgaaggaggactactccttcaccgcctacgccaccatctcctacctgaagatcggctga

SEQ ID NO: 61 - protein, 292 aa, synthetic construct
MAAMAERPFQCRICMRNFSRSSDLTRHIRTHTGEKPFACDICGRKFARSDTLTRHTKIHTGSQKPFQCRICMRNFSQSGDLSEHIRTHTGEKPFACDICGRKFATSGHLTTHTKIHTGSQKPFQCRICMRNFSDSSHLTTHIRTHTGEKPFACDICGRKFARSSHLTTHTKIHTGSQKPFQCRICMRNFSDRSDLTRHIRTHTGEKPFACDICGRKFADRSDLTRHTKIHTGSQKPFQCRICMRNFSRSDTLTRHIRTHTGEKIIAHFLIQQYLKEDYSFTAYATISYLKIG

SEQ ID NO: 62 - DNA, 17 bp, synthetic construct
  source 1..17: mol_type = other DNA
ctgcaggcggcgcaggc

SEQ ID NO: 63 - DNA, 22 bp, synthetic construct
  source 1..22: mol_type = other DNA
ggtcatcggcatcgcggaggag

SEQ ID NO: 64 - DNA, 21 bp, Homo sapiens
  source 1..21: mol_type = other DNA
gaggccaaacccttcctggag

SEQ ID NO: 65 - DNA, 3661 bp, synthetic construct
  source 1..3661: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggggtaccggcggcgatggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactggggagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 66 - DNA, 708 bp, synthetic construct
  source 1..708: mol_type = other DNA
atggcggcgatggccgagcggcccttccagtgcaggatctgtatgcgcaacttttctcggtcctccaacctgacccggcacatcagaacccatacaggcgaaaagcctttcgcctgcgacatttgtgggagaaaatttgctcggtccgacgccctgtccgagcataccaagatccacaccggctctcagaaaccattccagtgccgcatttgtatgcggaatttttccgactcctccgccctgaccacccatatccgcactcacaccggagagaagccctttgcttgcgacatttgtggcaggaaatttgctgactcctccgacctgtccgagcacactaagatccatactgggtcacagaaacctttccagtgccggatttgtatgagaaactttagccagtccggcaacctgtcccagcacatcagaacccatacaggcgaaaagcctttcgcctgcgacatttgtgggagaaaatttgctgaccggtccgacctgacccggcataccaagatccacaccggctctcagaaaccattccagtgccgcatttgtatgcggaatttttcccggtccgacaacctgacccggcacatcagaacacatactggggagaagatcatcgcccacttcctgatccagcagtacctgaaggaggactactccttcaccgcctacgccaccatctcctacctgaagatcggctga

SEQ ID NO: 67 - protein, 235 aa, synthetic construct
MAAMAERPFQCRICMRNFSRSSNLTRHIRTHTGEKPFACDICGRKFARSDALSEHTKIHTGSQKPFQCRICMRNFSDSSALTTHIRTHTGEKPFACDICGRKFADSSDLSEHTKIHTGSQKPFQCRICMRNFSQSGNLSQHIRTHTGEKPFACDICGRKFADRSDLTRHTKIHTGSQKPFQCRICMRNFSRSDNLTRHIRTHTGEKIIAHFLIQQYLKEDYSFTAYATISYLKIG

SEQ ID NO: 68 - DNA, 20 bp, synthetic construct
  source 1..20: mol_type = other DNA
actcttagacataacacacc

SEQ ID NO: 69 - DNA, 19 bp, synthetic construct
  source 1..19: mol_type = other DNA
aagagagccttccgaaaga

SEQ ID NO: 70 - protein, 204 aa, Homo sapiens
MAAMAERPFQCRICMRNFSRSSNLTRHIRTHTGEKPFACDICGRKFARSDALSEHTKIHTGSQKPFQCRICMRNFSDSSALTTHIRTHTGEKPFACDICGRKFADSSDLSEHTKIHTGSQKPFQCRICMRNFSQSGNLSQHIRTHTGEKPFACDICGRKFADRSDLTRHTKIHTGSQKPFQCRICMRNFSRSDNLTRHIRTHTG

SEQ ID NO: 71 - DNA, 27 bp, Homo sapiens
  source 1..27: mol_type = other DNA
cgggagatgaagaagtcgttgatggat

SEQ ID NO: 72 - DNA, 130 bp, synthetic construct
  source 1..130: mol_type = other DNA
tggcagaggcacagggcacttatgaggagctccagcgggagatgaagaagtcgttgatggacttaactaacgatgcctgccagctgctctcaggagagaggtagccaggccttgggtgggcaggatctag

SEQ ID NO: 73 - DNA, 3823 bp, synthetic construct
  source 1..3823: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctacctccggcaacctgacccggcacatcagaacccatacaggcgaaaagcctttcgcctgcgacatttgtgggagaaaatttgctcggtccgacgccctgtcccagcataccaagatccacaccggctctcagaaaccattccagtgccgcatttgtatgcggaatttttcccggtcctccgccctgaccacccatatccgcactcacaccggagagaagccctttgcttgcgacatttgtggcaggaaatttgctcggtcctccgacctgaccacccacactaagatccatactgggtcacagaaacctttccagtgccggatttgtatgagaaactttagccggtcctccaacctgtcccagcacatcagaacccatacaggcgaaaagcctttcgcctgcgacatttgtgggagaaaatttgctcggtcctccaacctgtcccagcataccaagatccacaccggctctcagaaaccattccagtgccgcatttgtatgcggaatttttcccggtcctccgccctgtcccagcatatccgcactcacaccggagagaagccctttgcttgcgacatttgtggcaggaaatttgctcggtcctccaacctgacccggcacactaagatccatactgggtcacagaaacctttccagtgccggatttgtatgagaaactttagccggtccgaccacctgtccgagcacatcagaacacatactgggctgagagagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 74 - DNA, 885 bp, synthetic construct
  source 1..885: mol_type = other DNA
atggcggcgatggccgagcggcccttccagtgcaggatctgtatgcgcaacttttctacctccggcaacctgacccggcacatcagaacccatacaggcgaaaagcctttcgcctgcgacatttgtgggagaaaatttgctcggtccgacgccctgtcccagcataccaagatccacaccggctctcagaaaccattccagtgccgcatttgtatgcggaatttttcccggtcctccgccctgaccacccatatccgcactcacaccggagagaagccctttgcttgcgacatttgtggcaggaaatttgctcggtcctccgacctgaccacccacactaagatccatactgggtcacagaaacctttccagtgccggatttgtatgagaaactttagccggtcctccaacctgtcccagcacatcagaacccatacaggcgaaaagcctttcgcctgcgacatttgtgggagaaaatttgctcggtcctccaacctgtcccagcataccaagatccacaccggctctcagaaaccattccagtgccgcatttgtatgcggaatttttcccggtcctccgccctgtcccagcatatccgcactcacaccggagagaagccctttgcttgcgacatttgtggcaggaaatttgctcggtcctccaacctgacccggcacactaagatccatactgggtcacagaaacctttccagtgccggatttgtatgagaaactttagccggtccgaccacctgtccgagcacatcagaacacatactgggctgagagagaagatcatcgcccacttcctgatccagcagtacctgaaggaggactactccttcaccgcctacgccaccatctcctacctgaagatcggctga

SEQ ID NO: 75 - protein, 294 aa, synthetic construct
MAAMAERPFQCRICMRNFSTSGNLTRHIRTHTGEKPFACDICGRKFARSDALSQHTKIHTGSQKPFQCRICMRNFSRSSALTTHIRTHTGEKPFACDICGRKFARSSDLTTHTKIHTGSQKPFQCRICMRNFSRSSNLSQHIRTHTGEKPFACDICGRKFARSSNLSQHTKIHTGSQKPFQCRICMRNFSRSSALSQHIRTHTGEKPFACDICGRKFARSSNLTRHTKIHTGSQKPFQCRICMRNFSRSDHLSEHIRTHTGLREKIIAHFLIQQYLKEDYSFTAYATISYLKIG

SEQ ID NO: 76 - DNA, 20 bp, synthetic construct
  source 1..20: mol_type = other DNA
aaccagaactgggagcgtta

SEQ ID NO: 77 - DNA, 20 bp, synthetic construct
  source 1..20: mol_type = other DNA
ctagatcctgcccacccaag

SEQ ID NO: 78 - protein, 261 aa, Homo sapiens
MAAMAERPFQCRICMRNFSTSGNLTRHIRTHTGEKPFACDICGRKFARSDALSQHTKIHTGSQKPFQCRICMRNFSRSSALTTHIRTHTGEKPFACDICGRKFARSSDLTTHTKIHTGSQKPFQCRICMRNFSRSSNLSQHIRTHTGEKPFACDICGRKFARSSNLSQHTKIHTGSQKPFQCRICMRNFSRSSALSQHIRTHTGEKPFACDICGRKFARSSNLTRHTKIHTGSQKPFQCRICMRNFSRSDHLSEHIRTHTG

SEQ ID NO: 79 - DNA, 30 bp, Homo sapiens
  source 1..30: mol_type = other DNA
gtggaccagtgtgtggcctaccatctgggc

SEQ ID NO: 80 - DNA, 131 bp, synthetic construct
  source 1..131: mol_type = other DNA
gctgccacctccgaatgctgactgacccagtggaccagtgtgtggcctaccatctggggagagtgaagagaggtgaggaaggctcagttttcccccagctcccaaacctacagctgcctccagttcctcca

SEQ ID NO: 81 - DNA, 3921 bp, synthetic construct
  source 1..3921: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggggagacccaagctggctagcgtttaaacttaagcttatggcggcgatggccgagcggcccttcgcctgcgacatttgtgggagaaaatttgctgatgaccggtcccacctgacccggcataccaagatccacaccggctctcagaaaccattccagtgcaggatctgtatgcgcaacttttctcggtccgacgccctgtccgagcacatcagaacccatacaggcgaaaagccttttgcttgcgacatttgtggcaggaaatttgctacctccggcaacctgtccgagcacactaagatccatactgggtcacagaaacctttccagtgccgcatttgtatgcggaatttttccgactcctccaacctgaccacccatatccgcactcacaccggagagaagccctttgcatgcgacatttgtggacggaaatttgctgaccggtccgacctgacccggcataccaagattcacactgggtctcagaaacctttccagtgcaggatttgtatgagaaatttttcccggtccgacgccctgacccggcacatcagaacccatacaggcgaaaagccttttgcttgcgacatttgtggcaggaaatttgctacctccggccacctgaccacccacactaagatccatactgggtcacagaaacctttccagtgccgcatttgtatgcggaatttttcccggtcctccaacctgtccgagcacatcagaacccatacaggcgaaaagccttttgcttgcgacatttgtggcaggaaatttgctgaccggtccaacctgacccggcacactaagatccatactgggtcacagaaacctttccagtgccgcatttgtatgcggaatttttcccggtccgacgccctgacccggcacatcagaacacatactgggctgagagagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctgcagcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 82 - DNA, 975 bp, synthetic construct
  source 1..975: mol_type = other DNA
atggcggcgatggccgagcggcccttcgcctgcgacatttgtgggagaaaatttgctgatgaccggtcccacctgacccggcataccaagatccacaccggctctcagaaaccattccagtgcaggatctgtatgcgcaacttttctcggtccgacgccctgtccgagcacatcagaacccatacaggcgaaaagccttttgcttgcgacatttgtggcaggaaatttgctacctccggcaacctgtccgagcacactaagatccatactgggtcacagaaacctttccagtgccgcatttgtatgcggaatttttccgactcctccaacctgaccacccatatccgcactcacaccggagagaagccctttgcatgcgacatttgtggacggaaatttgctgaccggtccgacctgacccggcataccaagattcacactgggtctcagaaacctttccagtgcaggatttgtatgagaaatttttcccggtccgacgccctgacccggcacatcagaacccatacaggcgaaaagccttttgcttgcgacatttgtggcaggaaatttgctacctccggccacctgaccacccacactaagatccatactgggtcacagaaacctttccagtgccgcatttgtatgcggaatttttcccggtcctccaacctgtccgagcacatcagaacccatacaggcgaaaagccttttgcttgcgacatttgtggcaggaaatttgctgaccggtccaacctgacccggcacactaagatccatactgggtcacagaaacctttccagtgccgcatttgtatgcggaatttttcccggtccgacgccctgacccggcacatcagaacacatactgggctgagagagaagatcatcgcccacttcctgatccagcagtacctgaaggaggactactccttcaccgcctacgccaccatctcctacctgaagatcggctga

SEQ ID NO: 83 - protein, 324 aa, synthetic construct
MAAMAERPFACDICGRKFADDRSHLTRHTKIHTGSQKPFQCRICMRNFSRSDALSEHIRTHTGEKPFACDICGRKFATSGNLSEHTKIHTGSQKPFQCRICMRNFSDSSNLTTHIRTHTGEKPFACDICGRKFADRSDLTRHTKIHTGSQKPFQCRICMRNFSRSDALTRHIRTHTGEKPFACDICGRKFATSGHLTTHTKIHTGSQKPFQCRICMRNFSRSSNLSEHIRTHTGEKPFACDICGRKFADRSNLTRHTKIHTGSQKPFQCRICMRNFSRSDALTRHIRTHTGLREKIIAHFLIQQYLKEDYSFTAYATISYLKIG

SEQ ID NO: 84 - DNA, 20 bp, synthetic construct
  source 1..20: mol_type = other DNA
acctccgaatgctgactgac

SEQ ID NO: 85 - DNA, 20 bp, synthetic construct
  source 1..20: mol_type = other DNA
tcacctttgaagtggctcct

SEQ ID NO: 86 - protein, 291 aa, Homo sapiens
MAAMAERPFACDICGRKFADDRSHLTRHTKIHTGSQKPFQCRICMRNFSRSDALSEHIRTHTGEKPFACDICGRKFATSGNLSEHTKIHTGSQKPFQCRICMRNFSDSSNLTTHIRTHTGEKPFACDICGRKFADRSDLTRHTKIHTGSQKPFQCRICMRNFSRSDALTRHIRTHTGEKPFACDICGRKFATSGHLTTHTKIHTGSQKPFQCRICMRNFSRSSNLSEHIRTHTGEKPFACDICGRKFADRSNLTRHTKIHTGSQKPFQCRICMRNFSRSDALTRHIRTHTG

SEQ ID NO: 87 - DNA, 36 bp, Homo sapiens
  source 1..36: mol_type = other DNA
ttggacggccagatattcttgtcatgtgtggagggg

SEQ ID NO: 88 - DNA, 130 bp, synthetic construct
  source 1..130: mol_type = other DNA
aaaaccctagttcctgaactcatcaaagaacttaactcccttggacggccagatattcttgttaaatgcggaggggtgataccacctcaggtattttttatctctatttttctagtactgtgatgggaat

SEQ ID NO: 89 - DNA, 4084 bp, synthetic construct
  source 1..4084: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttcgcctgcgacatttgtgggagaaaatttgctgatcggtcctcccacctgacccggcataccaagatccacaccggctctcagaaaccattccagtgcaggatctgtatgcgcaacttttctcggtccgacaacctgacccggcacatcagaacccatacaggcgaaaagccttttgcttgcgacatttgtggcaggaaatttgctcggtccgacgccctgacccggcacactaagatccatactgggtcacagaaacctttccagtgccgcatttgtatgcggaatttttccacctccggccacctgaccacccatatccgcactcacaccggagagaagccctttgcatgcgacatttgtggacggaaatttgctcagtccggcgacctgaccacccataccaagattcacactgggtctcagaaacctttccagtgcaggatttgtatgagaaatttttcccggtcctccgccctgaccacccacatcagaacccatacaggcgaaaagccttttgcttgcgacatttgtggcaggaaatttgctgactcctccgccctgaccacccacactaagatccatactgggtcacagaaacctttccagtgccgcatttgtatgcggaatttttcccagtccggctccctgtcccagcacatcagaacccatacaggcgaaaagccttttgcttgcgacatttgtggcaggaaatttgctcggtcctccaacctgtccgagcacactaagatccatactgggtcacagaaacctttccagtgccgcatttgtatgcggaatttttccgactcctcccacctgacccggcacatcagaacccatacaggcgaaaagccttttgcttgcgacatttgtggcaggaaatttgctgaccggtccaacctgacccggcacactaagatccatactgggtcacagaaacctttccagtgccgcatttgtatgcggaatttttcccggtccgacgccctgaccacccacatcagaacacatactgggctgagagagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 90 - DNA, 1146 bp, synthetic construct
  source 1..1146: mol_type = other DNA
atggcggcgatggccgagcggcccttcgcctgcgacatttgtgggagaaaatttgctgatcggtcctcccacctgacccggcataccaagatccacaccggctctcagaaaccattccagtgcaggatctgtatgcgcaacttttctcggtccgacaacctgacccggcacatcagaacccatacaggcgaaaagccttttgcttgcgacatttgtggcaggaaatttgctcggtccgacgccctgacccggcacactaagatccatactgggtcacagaaacctttccagtgccgcatttgtatgcggaatttttccacctccggccacctgaccacccatatccgcactcacaccggagagaagccctttgcatgcgacatttgtggacggaaatttgctcagtccggcgacctgaccacccataccaagattcacactgggtctcagaaacctttccagtgcaggatttgtatgagaaatttttcccggtcctccgccctgaccacccacatcagaacccatacaggcgaaaagccttttgcttgcgacatttgtggcaggaaatttgctgactcctccgccctgaccacccacactaagatccatactgggtcacagaaacctttccagtgccgcatttgtatgcggaatttttcccagtccggctccctgtcccagcacatcagaacccatacaggcgaaaagccttttgcttgcgacatttgtggcaggaaatttgctcggtcctccaacctgtccgagcacactaagatccatactgggtcacagaaacctttccagtgccgcatttgtatgcggaatttttccgactcctcccacctgacccggcacatcagaacccatacaggcgaaaagccttttgcttgcgacatttgtggcaggaaatttgctgaccggtccaacctgacccggcacactaagatccatactgggtcacagaaacctttccagtgccgcatttgtatgcggaatttttcccggtccgacgccctgaccacccacatcagaacacatactgggctgagagagaagatcatcgcccacttcctgatccagcagtacctgaaggaggactactccttcaccgcctacgccaccatctcctacctgaagatcggctga

SEQ ID NO: 91 - protein, 381 aa, synthetic construct
MAAMAERPFACDICGRKFADRSSHLTRHTKIHTGSQKPFQCRICMRNFSRSDNLTRHIRTHTGEKPFACDICGRKFARSDALTRHTKIHTGSQKPFQCRICMRNFSTSGHLTTHIRTHTGEKPFACDICGRKFAQSGDLTTHTKIHTGSQKPFQCRICMRNFSRSSALTTHIRTHTGEKPFACDICGRKFADSSALTTHTKIHTGSQKPFQCRICMRNFSQSGSLSQHIRTHTGEKPFACDICGRKFARSSNLSEHTKIHTGSQKPFQCRICMRNFSDSSHLTRHIRTHTGEKPFACDICGRKFADRSNLTRHTKIHTGSQKPFQCRICMRNFSRSDALTTHIRTHTGLREKIIAHFLIQQYLKEDYSFTAYATISYLKIG

SEQ ID NO: 92 - DNA, 18 bp, synthetic construct
  source 1..18: mol_type = other DNA
gtgcatgctgtgggcata

SEQ ID NO: 93 - DNA, 27 bp, synthetic construct
  source 1..27: mol_type = other DNA
ttcccatcacagtactagaaaaataga

SEQ ID NO: 94 - protein, 348 aa, Homo sapiens
MAAMAERPFACDICGRKFADRSSHLTRHTKIHTGSQKPFQCRICMRNFSRSDNLTRHIRTHTGEKPFACDICGRKFARSDALTRHTKIHTGSQKPFQCRICMRNFSTSGHLTTHIRTHTGEKPFACDICGRKFAQSGDLTTHTKIHTGSQKPFQCRICMRNFSRSSALTTHIRTHTGEKPFACDICGRKFADSSALTTHTKIHTGSQKPFQCRICMRNFSQSGSLSQHIRTHTGEKPFACDICGRKFARSSNLSEHTKIHTGSQKPFQCRICMRNFSDSSHLTRHIRTHTGEKPFACDICGRKFADRSNLTRHTKIHTGSQKPFQCRICMRNFSRSDALTTHIRTHTG

SEQ ID NO: 95 - DNA, 20 bp, Homo sapiens
  source 1..20: mol_type = other DNA
gtggttttggtttaggaact

SEQ ID NO: 96 - DNA, 130 bp, synthetic construct
  source 1..130: mol_type = other DNA
cccttcactcaagcctgtggttttggtcttaggaactttgctgccacaataccttggcccttctcagttcgctgcgacccatacacccaaaggattgaggtcttggacaatacccagcagcttaagattt

SEQ ID NO: 97 - DNA, 3646 bp, synthetic construct
  source 1..3646: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttccagtgcaggatctgtatgcgcaacttttctacctccggcgacctgtcccagcacatcagaacccatacaggcgaaaagcctttcgcctgcgacatttgtgggagaaaatttgctcagtccggccacctgacccggcataccaagatccacaccggctctcagaaaccattccagtgccgcatttgtatgcggaatttttcccagtccggctccctgaccacccatatccgcactcacaccggagagaagccctttgcttgcgacatttgtggcaggaaatttgctgaccgctccgccctgacccggcacactaagatccatactgggtcacagaaacctttccagtgccggatttgtatgagaaactttagccggtccgactccctgaccacccacatcagaacccatacaggcgaaaagcctttcgcctgcgacatttgtgggagaaaatttgctacctccggcgccctgacccggcataccaagatccacaccggctctcagaaaccattccagtgccgcatttgtatgcggaatttttcccggtccgactccctgacccggcacatcagaacacataccggtgagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 98 - DNA, 708 bp, synthetic construct
  source 1..708: mol_type = other DNA
atggcggcgatggccgagcggcccttccagtgcaggatctgtatgcgcaacttttctacctccggcgacctgtcccagcacatcagaacccatacaggcgaaaagcctttcgcctgcgacatttgtgggagaaaatttgctcagtccggccacctgacccggcataccaagatccacaccggctctcagaaaccattccagtgccgcatttgtatgcggaatttttcccagtccggctccctgaccacccatatccgcactcacaccggagagaagccctttgcttgcgacatttgtggcaggaaatttgctgaccgctccgccctgacccggcacactaagatccatactgggtcacagaaacctttccagtgccggatttgtatgagaaactttagccggtccgactccctgaccacccacatcagaacccatacaggcgaaaagcctttcgcctgcgacatttgtgggagaaaatttgctacctccggcgccctgacccggcataccaagatccacaccggctctcagaaaccattccagtgccgcatttgtatgcggaatttttcccggtccgactccctgacccggcacatcagaacacataccggtgagaagatcatcgcccacttcctgatccagcagtacctgaaggaggactactccttcaccgcctacgccaccatctcctacctgaagatcggctga

SEQ ID NO: 99 - protein, 235 aa, synthetic construct
MAAMAERPFQCRICMRNFSTSGDLSQHIRTHTGEKPFACDICGRKFAQSGHLTRHTKIHTGSQKPFQCRICMRNFSQSGSLTTHIRTHTGEKPFACDICGRKFADRSALTRHTKIHTGSQKPFQCRICMRNFSRSDSLTTHIRTHTGEKPFACDICGRKFATSGALTRHTKIHTGSQKPFQCRICMRNFSRSDSLTRHIRTHTGEKIIAHFLIQQYLKEDYSFTAYATISYLKIG

SEQ ID NO: 100 - DNA, 20 bp, synthetic construct
  source 1..20: mol_type = other DNA
ggaggtgtccgtgttcctaa

SEQ ID NO: 101 - DNA, 20 bp, synthetic construct
  source 1..20: mol_type = other DNA
gctgctgggtattgtccaag

SEQ ID NO: 102 - protein, 204 aa, Homo sapiens
MAAMAERPFQCRICMRNFSTSGDLSQHIRTHTGEKPFACDICGRKFAQSGHLTRHTKIHTGSQKPFQCRICMRNFSQSGSLTTHIRTHTGEKPFACDICGRKFADRSALTRHTKIHTGSQKPFQCRICMRNFSRSDSLTTHIRTHTGEKPFACDICGRKFATSGALTRHTKIHTGSQKPFQCRICMRNFSRSDSLTRHIRTHTG

SEQ ID NO: 103 - DNA, 24 bp, Homo sapiens
  source 1..24: mol_type = other DNA
atggtgccaggcataatccaggaa

SEQ ID NO: 104 - DNA, 130 bp, synthetic construct
  source 1..130: mol_type = other DNA
gaatttcattctgttctcagttttcctggattatgcctggcaccattaaagaaaatatcatatgtggtgtttcctatgatgaatatagatacagaagcgtcatcaaagcatgccaactagaagaggtaag

SEQ ID NO: 105 - DNA, 3736 bp, synthetic construct
  source 1..3736: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtccgagcacatcagaacccatacaggcgaaaagccttttgcttgcgacatttgtggcaggaaatttgctgactcctccgccctgtcccagcacactaagatccatactgggtcacagaaacctttccagtgccgcatttgtatgcggaatttttcccagtccggctccctgtcccagcatatccgcactcacaccggagagaagccctttgcatgcgacatttgtggacggaaatttgctgaccggtcccacctgacccggcataccaagattcacactgggtctcagaaacctttccagtgcaggatttgtatgagaaatttttcccagtccggcgacctgtccgagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgactccctgtcccagcacatcagaacacatactggggagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 106 - DNA, 795 bp, synthetic construct
  source 1..795: mol_type = other DNA
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtccgagcacatcagaacccatacaggcgaaaagccttttgcttgcgacatttgtggcaggaaatttgctgactcctccgccctgtcccagcacactaagatccatactgggtcacagaaacctttccagtgccgcatttgtatgcggaatttttcccagtccggctccctgtcccagcatatccgcactcacaccggagagaagccctttgcatgcgacatttgtggacggaaatttgctgaccggtcccacctgacccggcataccaagattcacactgggtctcagaaacctttccagtgcaggatttgtatgagaaatttttcccagtccggcgacctgtccgagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgactccctgtcccagcacatcagaacacatactggggagaagatcatcgcccacttcctgatccagcagtacctgaaggaggactactccttcaccgcctacgccaccatctcctacctgaagatcggc

SEQ ID NO: 107 - protein, 265 aa, synthetic construct
MAAMAERPFACDICGRKFADQSGNLTRHTKIHTGSQKPFQCRICMRNFSRSDNLSEHIRTHTGEKPFACDICGRKFADSSALSQHTKIHTGSQKPFQCRICMRNFSQSGSLSQHIRTHTGEKPFACDICGRKFADRSHLTRHTKIHTGSQKPFQCRICMRNFSQSGDLSEHIRTHTGEKPFACDICGRKFARSSALTRHTKIHTGSQKPFQCRICMRNFSRSDSLSQHIRTHTGEKIIAHFLIQQYLKEDYSFTAYATISYLKIG

SEQ ID NO: 108 - DNA, 20 bp, synthetic construct
  source 1..20: mol_type = other DNA
tggagccttcagagggtaaa

SEQ ID NO: 109 - DNA, 20 bp, synthetic construct
  source 1..20: mol_type = other DNA
agttggcatgctttgatgac

SEQ ID NO: 110 - protein, 234 aa, Homo sapiens
MAAMAERPFACDICGRKFADQSGNLTRHTKIHTGSQKPFQCRICMRNFSRSDNLSEHIRTHTGEKPFACDICGRKFADSSALSQHTKIHTGSQKPFQCRICMRNFSQSGSLSQHIRTHTGEKPFACDICGRKFADRSHLTRHTKIHTGSQKPFQCRICMRNFSQSGDLSEHIRTHTGEKPFACDICGRKFARSSALTRHTKIHTGSQKPFQCRICMRNFSRSDSLSQHIRTHTG

SEQ ID NO: 111 - DNA, 3825 bp, synthetic construct
  source 1..3825: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggggagacccaagctggctagcgtttaaacttaagcttatggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggggagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctgcagcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 112 - DNA, 879 bp, synthetic construct; identical to SEQ ID NO: 60
  source 1..879: mol_type = other DNA

SEQ ID NO: 113 - RNA, 879 nt, synthetic construct
  source 1..879: mol_type = other RNA
atggcggcgatggccgagcggcccttccagtgcaggatctgtatgcgcaacttttctcggtcctccgacctgacccggcacatcagaacccatacaggcgaaaagcctttcgcctgcgacatttgtgggagaaaatttgctcggtccgacaccctgacccggcataccaagatccacaccggctctcagaaaccattccagtgccgcatttgtatgcggaatttttcccagtccggcgacctgtccgagcatatccgcactcacaccggagagaagccctttgcttgcgacatttgtggcaggaaatttgctacctccggccacctgaccacccacactaagatccatactgggtcacagaaacctttccagtgccggatttgtatgagaaactttagcgactcctcccacctgaccacccacatcagaacccatacaggcgaaaagcctttcgcctgcgacatttgtgggagaaaatttgctcggtcctcccacctgaccacccataccaagatccacaccggctctcagaaaccattccagtgccgcatttgtatgcggaatttttccgaccggtccgacctgacccggcatatccgcactcacaccggagagaagccctttgcttgcgacatttgtggcaggaaatttgctgaccggtccgacctgacccggcacactaagatccatactgggtcacagaaacctttccagtgccggatttgtatgagaaactttagccggtccgacaccctgacccggcacatcagaacacatactggggagaagatcatcgcccacttcctgatccagcagtacctgaaggaggactactccttcaccgcctacgccaccatctcctacctgaagatcggctga

SEQ ID NO: 114 - protein, 192 aa, synthetic construct
MDYKDDDDKAAMAERPFQCRICMRNFSDRSNLTRHIRTHTGEKPFACDICGRKFARSDNLTRHTKIHTGSQKPFQCRICMRNFSRSDSLSQHIRTHTGEKPFACDICGRKFADRSNLTRHTKIHTGSQKPFQCRICMRNFSRSDTLTRHIRTHTGLRGSNSGDPRRHSLGGSRKPDLIAYKNFDLLVIELKP

SEQ ID NO: 115 - DNA, 15 bp, Homo sapiens
  source 1..15: mol_type = other DNA
gcggacatggaggac

SEQ ID NO: 116 - DNA, 3922 bp, synthetic construct
  source 1..3922: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggggtaccggcggcgatggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtcccagcacatcagaacccatacaggcgaaaagccttttgcttgcgacatttgtggcaggaaatttgctacctccggcgacctgtcccagcacactaagatccatactgggtcacagaaacctttccagtgccgcatttgtatgcggaatttttccacctccggctccctgacccggcatatccgcactcacaccggagagaagccctttgcatgcgacatttgtggacggaaatttgctcggtccgacgccctgacccggcataccaagattcacactgggtctcagaaacctttccagtgcaggatttgtatgagaaatttttccacctccggcgacctgtccgagcacatcagaacccatacaggcgaaaagccttttgcttgcgacatttgtggcaggaaatttgctcagtccggcaacctgtccgagcacactaagatccatactgggtcacagaaacctttccagtgccgcatttgtatgcggaatttttcccagtccggcgacctgtcccagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgacgccctgtccgagcacatcagaacacatactggggagaagatcatcgcccacttcctgatccagcagtacctgaaggaggactactccttcaccgcctacgccaccatctcctacctgaagatcggctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 117 - DNA, 969 bp, synthetic construct
  source 1..969: mol_type = other DNA
atggcggcgatggccgagcggcccttcgcctgcgacatttgtgggagaaaatttgctgatcagtccggcaacctgacccggcataccaagatccacaccggctctcagaaaccattccagtgcaggatctgtatgcgcaacttttctcggtccgacaacctgtcccagcacatcagaacccatacaggcgaaaagccttttgcttgcgacatttgtggcaggaaatttgctacctccggcgacctgtcccagcacactaagatccatactgggtcacagaaacctttccagtgccgcatttgtatgcggaatttttccacctccggctccctgacccggcatatccgcactcacaccggagagaagccctttgcatgcgacatttgtggacggaaatttgctcggtccgacgccctgacccggcataccaagattcacactgggtctcagaaacctttccagtgcaggatttgtatgagaaatttttccacctccggcgacctgtccgagcacatcagaacccatacaggcgaaaagccttttgcttgcgacatttgtggcaggaaatttgctcagtccggcaacctgtccgagcacactaagatccatactgggtcacagaaacctttccagtgccgcatttgtatgcggaatttttcccagtccggcgacctgtcccagcacatcagaacccatacaggcgaaaagccttttgcttgcgacatttgtggcaggaaatttgctcggtcctccgccctgacccggcacactaagatccatactgggtcacagaaacctttccagtgccgcatttgtatgcggaatttttcccggtccgacgccctgtccgagcacatcagaacacatactggggagaagatcatcgcccacttcctgatccagcagtacctgaaggaggactactccttcaccgcctacgccaccatctcctacctgaagatcggctga

SEQ ID NO: 118 - protein, 322 aa, synthetic construct
MAAMAERPFACDICGRKFADQSGNLTRHTKIHTGSQKPFQCRICMRNFSRSDNLSQHIRTHTGEKPFACDICGRKFATSGDLSQHTKIHTGSQKPFQCRICMRNFSTSGSLTRHIRTHTGEKPFACDICGRKFARSDALTRHTKIHTGSQKPFQCRICMRNFSTSGDLSEHIRTHTGEKPFACDICGRKFAQSGNLSEHTKIHTGSQKPFQCRICMRNFSQSGDLSQHIRTHTGEKPFACDICGRKFARSSALTRHTKIHTGSQKPFQCRICMRNFSRSDALSEHIRTHTGEKIIAHFLIQQYLKEDYSFTAYATISYLKIG

SEQ ID NO: 119 - DNA, 30 bp, Homo sapiens
  source 1..30: mol_type = other DNA
ctggtgacacaacctgtggttactaaggaa

SEQ ID NO: 120 - DNA, 22 bp, synthetic construct
  source 1..22: mol_type = other DNA
ttggctctttagcttgtgtttc

SEQ ID NO: 121 - DNA, 22 bp, synthetic construct
  source 1..22: mol_type = other DNA
ggcatttctagtttggagatgg

SEQ ID NO: 122 - protein, 291 aa, Homo sapiens
MAAMAERPFACDICGRKFADQSGNLTRHTKIHTGSQKPFQCRICMRNFSRSDNLSQHIRTHTGEKPFACDICGRKFATSGDLSQHTKIHTGSQKPFQCRICMRNFSTSGSLTRHIRTHTGEKPFACDICGRKFARSDALTRHTKIHTGSQKPFQCRICMRNFSTSGDLSEHIRTHTGEKPFACDICGRKFAQSGNLSEHTKIHTGSQKPFQCRICMRNFSQSGDLSQHIRTHTGEKPFACDICGRKFARSSALTRHTKIHTGSQKPFQCRICMRNFSRSDALSEHIRTHTG

SEQ ID NO: 123 - DNA, 114 bp, synthetic construct
  source 1..114: mol_type = other DNA
ccccggtggcggaggagacgcgggcacggctgtccaaggagctgcaggcggcgcaggcccggctgggcgcggacatggaggacgtgcgcggccgcctggtgcagtaccgcggcg

SEQ ID NO: 124 - DNA, 114 bp, synthetic construct
  source 1..114: mol_type = other DNA
cgccgcggtactgcaccaggcggccgcgcacgtcctccatgtccgcgcccagccgggcctgcgccgcctgcagctccttggacagccgtgcccgcgtctcctccgccaccgggg

SEQ ID NO: 125 - DNA, 10 bp, synthetic construct
  source 1..10: mol_type = other DNA
cttaactaac

SEQ ID NO: 126 - DNA, 10 bp, synthetic construct
  source 1..10: mol_type = other DNA
tctggccaat

SEQ ID NO: 127 - protein, 5 aa, synthetic construct
  REPEAT 1: note = The entire sequence of amino acids 1-5 can be repeated one to two times.
GGGGS

SEQ ID NO: 128 - protein, 30 aa, synthetic construct
GGGGSGGGGSGGGGSGGGGSGGGGSGGGGS

SEQ ID NO: 129 - protein, 4 aa, synthetic construct
TGEK

SEQ ID NO: 130 - protein, 43 aa, Homo sapiens
TGLRGSNSQRLADQYRRHSLFGTGVEVSRYNKFMKICALTKKG

SEQ ID NO: 131 - protein, 39 aa, Homo sapiens
TGSRHNAERLFKKLILSKILDEDLYINANDQAIAYVMLG

SEQ ID NO: 132 - protein, 33 aa, Homo sapiens
TGEKIIAHFLIQQYLKEDYSFTAYATISYLKIG

SEQ ID NO: 133 - DNA, 35 bp, synthetic construct
  source 1..35: mol_type = other DNA
ggacgtgtgcggccgcctggtgcagtaccgcggcg

SEQ ID NO: 134 - DNA, 35 bp, synthetic construct
  source 1..35: mol_type = other DNA
cctgcacacgccggcggaccacgtcatggcgccgc

SEQ ID NO: 135 - DNA, 10 bp, Homo sapiens
  source 1..10: mol_type = other DNA
tgtgcggccg

SEQ ID NO: 136 - DNA, 136 bp, Homo sapiens
  source 1..136: mol_type = other DNA
ccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcgg

SEQ ID NO: 137 - DNA, 12 bp, Homo sapiens
  source 1..12: mol_type = other DNA
cgtgtgcggccg

SEQ ID NO: 138 - DNA, 12 bp, Homo sapiens
  source 1..12: mol_type = other DNA
cctgtgcggccg

SEQ ID NO: 139 - DNA, 28 bp, Homo sapiens
  source 1..28: mol_type = other DNA
ccggctgggcgcggacatggaggacgtg

SEQ ID NO: 140 - DNA, 33 bp, Homo sapiens
  source 1..33: mol_type = other DNA
ccggctgggcgcggacatggaggacgtgtgcgg

SEQ ID NO: 141 - DNA, 26 bp, Homo sapiens
  source 1..26: mol_type = other DNA
ccggctgggcgcggacatggaggacg

SEQ ID NO: 142 - DNA, 13 bp, Homo sapiens
  source 1..13: mol_type = genomic DNA
gtgtgcggccgcc

SEQ ID NO: 143 - DNA, 13 bp, Homo sapiens
  source 1..13: mol_type = other DNA
gtgcgcggccgcc

SEQ ID NO: 144 - DNA, 16 bp, Homo sapiens
  source 1..16: mol_type = other DNA
gacgtgtgcggccgcc

SEQ ID NO: 145 - DNA, 16 bp, Homo sapiens
  source 1..16: mol_type = other DNA
acgtgtgcggccgcct

SEQ ID NO: 146 - DNA, 55 bp, Homo sapiens
  source 1..55: mol_type = other DNA
ccggctgggcgcggacatggaggacgtgtgcggccgcctggtgcagtaccgcggc

SEQ ID NO: 147 - DNA, 45 bp, Homo sapiens
  source 1..45: mol_type = other DNA
ccggctgggcgcggacatggaggacgtgtgcggccgcctggtgca

SEQ ID NO: 148 - DNA, 47 bp, Homo sapiens
  source 1..47: mol_type = other DNA
ttgcttttatcatcacaggctccaggaagggtttggcctctgattag

SEQ ID NO: 149 - DNA, 44 bp, Homo sapiens
  source 1..44: mol_type = other DNA
ctaatcagaggccaaacccttcctggagcctgtgataaaagcaa

SEQ ID NO: 150 - DNA, 18 bp, Homo sapiens
  source 1..18: mol_type = other DNA
ttgctgaattcacaggct

SEQ ID NO: 151 - DNA, 15 bp, Homo sapiens
  source 1..15: mol_type = genomic DNA
gcttttatcacaggc

SEQ ID NO: 152 - DNA, 16 bp, Homo sapiens
  source 1..16: mol_type = other DNA
gcttgaattcacaggc

SEQ ID NO: 153 - DNA, 53 bp, Homo sapiens
  source 1..53: mol_type = other DNA
ggagctccagcgggagatgaagaagtcgttgatggatctggccaatgatgcct

SEQ ID NO: 154 - DNA, 53 bp, Homo sapiens
  source 1..53: mol_type = other DNA
aggcatcattggccagatccatcaacgacttcttcatctcccgctggagctcc

SEQ ID NO: 155 - DNA, 19 bp, Homo sapiens
  source 1..19: mol_type = other DNA
tggacttaactaacgatgc

SEQ ID NO: 156 - DNA, 10 bp, Homo sapiens
  source 1..10: mol_type = other DNA
agaccggtta

SEQ ID NO: 157 - DNA, 10 bp, Homo sapiens
  source 1..10: mol_type = other DNA
gaattgattg

SEQ ID NO: 158 - DNA, 22 bp, Homo sapiens
  source 1..22: mol_type = other DNA
ttgatggacctngcnnangatg

SEQ ID NO: 159 - DNA, 66 bp, Homo sapiens
  source 1..66: mol_type = other DNA
actgacccagtggaccagtgtgtggcctaccatctgggccgtgtgagagaggtgaggaaggctcag

SEQ ID NO: 160 - DNA, 66 bp, Homo sapiens
  source 1..66: mol_type = other DNA
ctgagccttcctcacctctctaacacggcccagatggtaggccacacactggtccactgggtcagt

SEQ ID NO: 161 - DNA, 21 bp, Homo sapiens
  source 1..21: mol_type = other DNA
tctggggagagtgaagagagg

SEQ ID NO: 162 - DNA, 18 bp, Homo sapiens
  source 1..18: mol_type = other DNA
tgggccgtgttagagagg

SEQ ID NO: 163 - DNA, 70 bp, Homo sapiens
  source 1..70: mol_type = other DNA
taactcccttggacggccagatattcttgtcatgtgtggaggggtgataccacctcaggtattttttatc

SEQ ID NO: 164 - DNA, 70 bp, Homo sapiens
  source 1..70: mol_type = other DNA
gataaaaaatacctgaggtggtatcacccctccacacatgacaagaatatctggccgtccaagggagtta

SEQ ID NO: 165 - DNA, 22 bp, Homo sapiens
  source 1..22: mol_type = other DNA
attcttgttaaatgcggagggg

SEQ ID NO: 166 - DNA, 70 bp, Homo sapiens
  source 1..70: mol_type = other DNA
actcaagcctgtggttttggtcttaggaactttgctgccacaatacctcggcccttctcagttcgctacg

SEQ ID NO: 167 - DNA, 70 bp, Homo sapiens
  source 1..70: mol_type = other DNA
cgtagcgaactgagaagggccgaggtattgtggcagcaaagttcctaagaccaaaaccacaggcttgagt

SEQ ID NO: 168 - DNA, 29 bp, Homo sapiens
  source 1..29: mol_type = other DNA
ctcggcccttctcagttcgctacgaccca

SEQ ID NO: 169 - DNA, 28 bp, Homo sapiens
  source 1..28: mol_type = other DNA
tcggcccttctcagttcgctgcgaccca

SEQ ID NO: 170 - DNA, 126 bp, Homo sapiens
  source 1..126: mol_type = other DNA
ccggctgggcgcggacatggaggacgtgtgcggccgcctggtgcagtaccgcggcgaggtgcaggccatgctcggccagagcaccgaggagctgcgggtgcgcctcgccgcaagctgcgtaagcgg

SEQ ID NO: 171 - RNA, 12 nt, Homo sapiens
  source 1..12: mol_type = other RNA
ggacgtgtgcgg

SEQ ID NO: 172 - RNA, 12 nt, Homo sapiens
  source 1..12: mol_type = other RNA
ggacgtgcgcgg

SEQ ID NO: 173 - DNA, 107 bp, synthetic construct
  source 1..107: mol_type = other DNA
taatttttctttttcttcttttttcctttttgcaaaaacccaaaatattttagctcctactcagactgttactctggtgacacaacctgtggttactaaggaaactg

SEQ ID NO: 174 - DNA, 17 bp, Homo sapiens
  source 1..17: mol_type = genomic DNA
actgttactctggtgac

SEQ ID NO: 175 - DNA, 16 bp, Homo sapiens
  source 1..16: mol_type = other DNA
actgttagactctggt

SEQ ID NO: 176 - DNA, 60 bp, Homo sapiens
  source 1..60: mol_type = other DNA
attttagctcctactcagactgttactctggtgacacaacctgtggttactaaggaaact

SEQ ID NO: 177 - DNA, 60 bp, Homo sapiens
  source 1..60: mol_type = other DNA
agtttccttagtaaccacaggttgtgtcaccagagtaacagtctgagtaggagctaaaat

SEQ ID NO: 178 - DNA, 66 bp, synthetic construct
  source 1..66: mol_type = other DNA
ccggctgggcgcggacatggaggacgtgtgcggccgcctggtgcagtaccgcggcgaggtgcaggc

SEQ ID NO: 179 - DNA, 36 bp, Homo sapiens
  source 1..36: mol_type = other DNA
cggacatggaggacgtgtgcggccgcctggtgcagt

SEQ ID NO: 180 - DNA, 36 bp, synthetic construct
  source 1..36: mol_type = other DNA
ggacatggaggacgtgtgcggccgcctggtgcagta

SEQ ID NO: 181 - DNA, 114 bp, Homo sapiens
  source 1..114: mol_type = other DNA
attaagcacagtggaagaatttcattctgttctcagttttcctggattatgcctggcaccattaaagaaaatatcatctttggtgtttcctatgatgaatatagatacagaagc

SEQ ID NO: 182 - DNA, 70 bp, Homo sapiens
  source 1..70: mol_type = other DNA
ctgttctcagttttcctggattatgcctggcaccattaaagaaaatatcatctttggtgtttcctatgat

SEQ ID NO: 183 - DNA, 70 bp, Homo sapiens
  source 1..70: mol_type = other DNA
atcataggaaacaccaaagatgatattttctttaatggtgccaggcataatccaggaaaactgagaacag

SEQ ID NO: 184 - DNA, 19 bp, Homo sapiens
  source 1..19: mol_type = other DNA
aaaatatcatatgtggtgt

SEQ ID NO: 185 - DNA, 42 bp, synthetic construct
  source 1..42: mol_type = other DNA
ccacaatacctcggcccttctcagttcgctacgacccataca

SEQ ID NO: 186 - DNA, 157 bp, synthetic construct
  source 1..157: mol_type = other DNA
aaaagaagtaaaatgccactgagaactctcttaagactacctttctccaaatggtgcccttcactcaagcctgtggttttggtcttaggaactttgctgccacaatacctcggcccttctcagttcgctacgacccatacacccaaaggattgaggt